SEQUENCE LISTING (407 sequences)

SEQ ID NO: 1 - DNA, 357 bp, synthetic construct
  misc_feature 1..357: note = synthetic
  source 1..357: mol_type = other DNA
caggtgcagctggtggagtctgggggaggcgtggtccagcctggaaggtccctgagactctcctgtgtagcgtctggattcaccttcagtagttatggcattcactgggtccgccaggctccaggcaaggggctggagtgggtggcagttatatgggatgatggaaataatataaactattcagactccgtgaagggccgattcatcatctccagagacaattccaggaagacagtgtatctgcaaatgaacagcctgagaggcgaggacacggctgtttattactgtgcgagagatgcccccatagcaccagtccctgactattggggccagggaaccctggtcaccgtctcctca

SEQ ID NO: 2 - protein, 119 aa, synthetic construct
  REGION 1..119: note = synthetic
QVQLVESGGGVVQPGRSLRLSCVASGFTFSSYGIHWVRQAPGKGLEWVAVIWDDGNNINYSDSVKGRFIISRDNSRKTVYLQMNSLRGEDTAVYYCARDAPIAPVPDYWGQGTLVTVSS

SEQ ID NO: 3 - DNA, 24 bp, synthetic construct
  misc_feature 1..24: note = synthetic
  source 1..24: mol_type = other DNA
ggattcaccttcagtagttatggc

SEQ ID NO: 4 - protein, 8 aa, synthetic construct
  REGION 1..8: note = synthetic
GFTFSSYG

SEQ ID NO: 5 - DNA, 24 bp, synthetic construct
  misc_feature 1..24: note = synthetic
  source 1..24: mol_type = other DNA
atatgggatgatggaaataatata

SEQ ID NO: 6 - protein, 8 aa, synthetic construct
  REGION 1..8: note = synthetic
IWDDGNNI

SEQ ID NO: 7 - DNA, 36 bp, synthetic construct
  misc_feature 1..36: note = synthetic
  source 1..36: mol_type = other DNA
gcgagagatgcccccatagcaccagtccctgactat

SEQ ID NO: 8 - protein, 12 aa, synthetic construct
  REGION 1..12: note = synthetic
ARDAPIAPVPDY

SEQ ID NO: 9 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagacactggggtcccatcaaggttcagcggcagtggatctgggacagagttcactctcaccatcagcagcctgcagcctgatgattttgcaacttattactgccaacagtataatacttattcgtacacttttggcctggggaccaaactggagatcaaa

SEQ ID NO: 10 - protein, 107 aa, synthetic construct
  REGION 1..107: note = synthetic
DIQMTQSPSTLSASVGDRVTITCRASQSISSWLAWYQQKPGKAPKLLIYKASSLDTGVPSRFSGSGSGTEFTLTISSLQPDDFATYYCQQYNTYSYTFGLGTKLEIK

SEQ ID NO: 11 - DNA, 18 bp, synthetic construct
  misc_feature 1..18: note = synthetic
  source 1..18: mol_type = other DNA
cagagtattagtagttgg

SEQ ID NO: 12 - protein, 6 aa, synthetic construct
  REGION 1..6: note = synthetic
QSISSW

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagtataatacttattcgtacact

SEQ ID NO: 16 - protein, 9 aa, synthetic construct
  REGION 1..9: note = synthetic
QQYNTYSYT

SEQ ID NO: 17 - DNA, 357 bp, synthetic construct
  misc_feature 1..357: note = synthetic
  source 1..357: mol_type = other DNA
caggtgcaactggtggagtctgggggaggcttggtcaagcctggagggtccctgagactctcctgtgcagcttctggattcaccttcagtgactactacatgagctggatccgccaggctccagggaaggggctggagtgggtttcatatattagcagtagtggtaataccataaaatatgcagactctatgaagggccgattcaccatctccagggacaacgccaagaaatcactgtttgtggaaatgaacagcctgagagccgaggacacggccgtgtattactgtgcgaggtataaaagttcgtccgactactttgaccactggggccagggaaccctggtcaccgtctcctca

SEQ ID NO: 18 - protein, 119 aa, synthetic construct
  REGION 1..119: note = synthetic
QVQLVESGGGLVKPGGSLRLSCAASGFTFSDYYMSWIRQAPGKGLEWVSYISSSGNTIKYADSMKGRFTISRDNAKKSLFVEMNSLRAEDTAVYYCARYKSSSDYFDHWGQGTLVTVSS

SEQ ID NO: 19 - DNA, 24 bp, synthetic construct
  misc_feature 1..24: note = synthetic
  source 1..24: mol_type = other DNA
ggattcaccttcagtgactactac

SEQ ID NO: 20 - protein, 8 aa, synthetic construct
  REGION 1..8: note = synthetic
GFTFSDYY

SEQ ID NO: 21 - DNA, 24 bp, synthetic construct
  misc_feature 1..24: note = synthetic
  source 1..24: mol_type = other DNA
attagcagtagtggtaataccata

SEQ ID NO: 22 - protein, 8 aa, synthetic construct
  REGION 1..8: note = synthetic
ISSSGNTI

SEQ ID NO: 23 - DNA, 36 bp, synthetic construct
  misc_feature 1..36: note = synthetic
  source 1..36: mol_type = other DNA
gcgaggtataaaagttcgtccgactactttgaccac

SEQ ID NO: 24 - protein, 12 aa, synthetic construct
  REGION 1..12: note = synthetic
ARYKSSSDYFDH

SEQ ID NO: 25 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttaggagttacttagcctggtaccaacagaaacctggccaggctcccaggctcctcatctatgatgcatccaacagggccactgccatcccagccaggttcagtggcagtgggtctgggacagacttcactctcaccatcagcagcctagagcctgaagatttagcagtttattactgtcagcagtctggcaactggccgctcactttcggcggagggaccaaggtggagatcaaa

SEQ ID NO: 26 - protein, 107 aa, synthetic construct
  REGION 1..107: note = synthetic
EIVLTQSPATLSLSPGERATLSCRASQSVRSYLAWYQQKPGQAPRLLIYDASNRATAIPARFSGSGSGTDFTLTISSLEPEDLAVYYCQQSGNWPLTFGGGTKVEIK

SEQ ID NO: 27 - DNA, 18 bp, synthetic construct
  misc_feature 1..18: note = synthetic
  source 1..18: mol_type = other DNA
cagagtgttaggagttac

SEQ ID NO: 28 - protein, 6 aa, synthetic construct
  REGION 1..6: note = synthetic
QSVRSY

SEQ ID NO: 29-30 - skipped (2 entries)

SEQ ID NO: 31 - DNA, 27 bp, synthetic construct
  misc_feature 1..27: note = synthetic
  source 1..27: mol_type = other DNA
cagcagtctggcaactggccgctcact

SEQ ID NO: 32 - protein, 9 aa, synthetic construct
  REGION 1..9: note = synthetic
QQSGNWPLT

SEQ ID NO: 33 - DNA, 357 bp, synthetic construct
  misc_feature 1..357: note = synthetic
  source 1..357: mol_type = other DNA
caggtgcagctggtggagtctgggggaggcgtggtccagcctgggaggtccttgagactctcctgtggagcgtctggattcaccttcagtacttatggcatgcactgggtccgccaggctccaggcaaggggctggagtgggtggcagttatctgggatgatggaaataataaatattatgcagactccgtgaagggccgattcaccatctccagagacaattcgaagaacacgctgtatctgcagatgaacagcctgagagccgaggacacggctgtttattactgtgcgagagattcagaggtcgccccagttggggactactggggccagggcaccctggtcaccgtctcctca

SEQ ID NO: 34 - protein, 119 aa, synthetic construct
  REGION 1..119: note = synthetic
QVQLVESGGGVVQPGRSLRLSCGASGFTFSTYGMHWVRQAPGKGLEWVAVIWDDGNNKYYADSVKGRFTISRDNSKNTLYLQMNSLRAEDTAVYYCARDSEVAPVGDYWGQGTLVTVSS

SEQ ID NO: 35 - DNA, 24 bp, synthetic construct
  misc_feature 1..24: note = synthetic
  source 1..24: mol_type = other DNA
ggattcaccttcagtacttatggc

SEQ ID NO: 36 - protein, 8 aa, synthetic construct
  REGION 1..8: note = synthetic
GFTFSTYG

SEQ ID NO: 37 - DNA, 24 bp, synthetic construct
  misc_feature 1..24: note = synthetic
  source 1..24: mol_type = other DNA
atctgggatgatggaaataataaa

SEQ ID NO: 38 - protein, 8 aa, synthetic construct
  REGION 1..8: note = synthetic
IWDDGNNK

SEQ ID NO: 39 - DNA, 36 bp, synthetic construct
  misc_feature 1..36: note = synthetic
  source 1..36: mol_type = other DNA
gcgagagattcagaggtcgccccagttggggactac

SEQ ID NO: 40 - protein, 12 aa, synthetic construct
  REGION 1..12: note = synthetic
ARDSEVAPVGDY

SEQ ID NO: 41 - DNA, 321 bp, synthetic construct
  misc_feature 1..321: note = synthetic
  source 1..321: mol_type = other DNA
gacatccagatgacccagtctccttccaccctgtctgcatctgtaggagacagagtcactatcatttgccgggccagtcagagtattaacaggtggttggcctggtatcagcagaaaccagggaaggcccctaaactcctgatctataaggcgtctagtttagaaagtggggtcccatcaaggttcagcggcagtggatctgggacagaattcactctcaccatcagcagcctgcagcctgatgattttgcagcttattactgccaacagtataatgattattcgtacacttttggccaggggaccaagctggagatcaaa

SEQ ID NO: 42 - protein, 107 aa, synthetic construct
  REGION 1..107: note = synthetic
DIQMTQSPSTLSASVGDRVTIICRASQSINRWLAWYQQKPGKAPKLLIYKASSLESGVPSRFSGSGSGTEFTLTISSLQPDDFAAYYCQQYNDYSYTFGQGTKLEIK

SEQ ID NO: 43 - DNA, 18 bp, synthetic construct
  misc_feature 1..18: note = synthetic
  source 1..18: mol_type = other DNA
cagagtattaacaggtgg

SEQ ID NO: 44 - protein, 6 aa, synthetic construct
  REGION 1..6: note = synthetic
QSINRW

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
caacagtataatgattattcgtacact

SEQ ID NO: 48 - protein, 9 aa, synthetic construct
  REGION 1..9: note = synthetic
QQYNDYSYT

SEQ ID NO: 49 - DNA, 366 bp, synthetic construct
  misc_feature 1..366: note = synthetic
  source 1..366: mol_type = other DNA
gaggtgcagctggtggagtctgggggagacttggtccagcctggagggtccctgagactctcctgtgcagcctctggattcaccttcagtgaccactatatggactgggtccgccaggctccagggaaggggctggactggattggccgtattagaaacaaagctaacgcttataacacagaatacgccgcgtctgtgagaggcagattcaccatctcaagagatgattcacagaatttactgtatctgcaaatgaacagcctgaaaaccgatgacacggccgtatattattgtgttagagtctggaactacgcctacttcgctatggacgtctggggccaagggaccacggtcaccgtctcctca

SEQ ID NO: 50 - protein, 122 aa, synthetic construct
  REGION 1..122: note = synthetic
EVQLVESGGDLVQPGGSLRLSCAASGFTFSDHYMDWVRQAPGKGLDWIGRIRNKANAYNTEYAASVRGRFTISRDDSQNLLYLQMNSLKTDDTAVYYCVRVWNYAYFAMDVWGQGTTVTVSS

SEQ ID NO: 51 - DNA, 24 bp, synthetic construct
  misc_feature 1..24: note = synthetic
  source 1..24: mol_type = other DNA
ggattcaccttcagtgaccactat

SEQ ID NO: 52 - protein, 8 aa, synthetic construct
  REGION 1..8: note = synthetic
GFTFSDHY

SEQ ID NO: 53 - DNA, 30 bp, synthetic construct
  misc_feature 1..30: note = synthetic
  source 1..30: mol_type = other DNA
attagaaacaaagctaacgcttataacaca

SEQ ID NO: 54 - protein, 10 aa, synthetic construct
  REGION 1..10: note = synthetic
IRNKANAYNT

SEQ ID NO: 55 - DNA, 39 bp, synthetic construct
  misc_feature 1..39: note = synthetic
  source 1..39: mol_type = other DNA
gttagagtctggaactacgcctacttcgctatggacgtc

SEQ ID NO: 56 - protein, 13 aa, synthetic construct
  REGION 1..13: note = synthetic
VRVWNYAYFAMDV

SEQ ID NO: 57 - DNA, 321 bp, synthetic construct
  misc_feature 1..321: note = synthetic
  source 1..321: mol_type = other DNA
gacatccagatgacccagtctccatcctccctatctgcatctgtgggagacagagtcaccatcacttgccggtcaagtcagaacattggaatctttttaaactggtatcaacaaaaaccaggggaagcccctaacctcctgatctccgctgcatccagtttacacagtggggtcccttcaaggttcagtggcagtgggtctgggacagatttcactctcaccatcggcagtctgcagcctgaagattttgcgacttactactgtcaacagacgtacaataccatattcactttcggccctgggaccaaagtggatatcaaa

SEQ ID NO: 58 - protein, 107 aa, synthetic construct
  REGION 1..107: note = synthetic
DIQMTQSPSSLSASVGDRVTITCRSSQNIGIFLNWYQQKPGEAPNLLISAASSLHSGVPSRFSGSGSGTDFTLTIGSLQPEDFATYYCQQTYNTIFTFGPGTKVDIK

SEQ ID NO: 59 - DNA, 18 bp, synthetic construct
  misc_feature 1..18: note = synthetic
  source 1..18: mol_type = other DNA
cagaacattggaatcttt

SEQ ID NO: 60 - protein, 6 aa, synthetic construct
  REGION 1..6: note = synthetic
QNIGIF

SEQ ID NO: 61-62 - skipped (2 entries)

SEQ ID NO: 63 - DNA, 27 bp, synthetic construct
  misc_feature 1..27: note = synthetic
  source 1..27: mol_type = other DNA
caacagacgtacaataccatattcact

SEQ ID NO: 64 - protein, 9 aa, synthetic construct
  REGION 1..9: note = synthetic
QQTYNTIFT

SEQ ID NO: 65 - DNA, 363 bp, synthetic construct
  misc_feature 1..363: note = synthetic
  source 1..363: mol_type = other DNA
gaggtgcagctggtggagtctgggggagacttggtacagcctggggggtccctgagactctcctgtgcagcctctggattcacctttagcagctatgccatgaactgggtccgccagggtccagggaagggactggagtgggtctcagctattagtggtcgtggtgatagtacatactacgcagactccgtgaagggccggctcaccatctccagagacaattccaagaacacgctgtatctgcaaatgaacagcctgagagccgaggacacggccgtatattactgtgtgaaagagggggagcaactcgtctactggtacttcgatctctggggccgtggcaccctggtcaccgtctcctca

SEQ ID NO: 66 - protein, 121 aa, synthetic construct
  REGION 1..121: note = synthetic
EVQLVESGGDLVQPGGSLRLSCAASGFTFSSYAMNWVRQGPGKGLEWVSAISGRGDSTYYADSVKGRLTISRDNSKNTLYLQMNSLRAEDTAVYYCVKEGEQLVYWYFDLWGRGTLVTVSS

SEQ ID NO: 67 - DNA, 24 bp, synthetic construct
  misc_feature 1..24: note = synthetic
  source 1..24: mol_type = other DNA
ggattcacctttagcagctatgcc

SEQ ID NO: 68 - protein, 8 aa, synthetic construct
  REGION 1..8: note = synthetic
GFTFSSYA

SEQ ID NO: 69 - DNA, 24 bp, synthetic construct
  misc_feature 1..24: note = synthetic
  source 1..24: mol_type = other DNA
attagtggtcgtggtgatagtaca

SEQ ID NO: 70 - protein, 8 aa, synthetic construct
  REGION 1..8: note = synthetic
ISGRGDST

SEQ ID NO: 71 - DNA, 42 bp, synthetic construct
  misc_feature 1..42: note = synthetic
  source 1..42: mol_type = other DNA
gtgaaagagggggagcaactcgtctactggtacttcgatctc

SEQ ID NO: 72 - protein, 14 aa, synthetic construct
  REGION 1..14: note = synthetic
VKEGEQLVYWYFDL

SEQ ID NO: 73 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagaccattagcaactttttacattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatttttcaacttacttctgtcaacagagttacactaccccgctcactttcggcggagggaccaaggtggagatcaaa

SEQ ID NO: 74 - protein, 107 aa, synthetic construct
  REGION 1..107: note = synthetic
DIQMTQSPSSLSASVGDRVTITCRASQTISNFLHWYQQKPGKAPKLLIYAASSLQSGVPSRFSGSGSGTDFTLTISSLQPEDFSTYFCQQSYTTPLTFGGGTKVEIK

SEQ ID NO: 75 - DNA, 18 bp, synthetic construct
  misc_feature 1..18: note = synthetic
  source 1..18: mol_type = other DNA
cagaccattagcaacttt

SEQ ID NO: 76 - protein, 6 aa, synthetic construct
  REGION 1..6: note = synthetic
QTISNF

SEQ ID NO: 77-78 - skipped (2 entries)

SEQ ID NO: 79 - DNA, 27 bp, synthetic construct
  misc_feature 1..27: note = synthetic
  source 1..27: mol_type = other DNA
caacagagttacactaccccgctcact

SEQ ID NO: 80 - protein, 9 aa, synthetic construct
  REGION 1..9: note = synthetic
QQSYTTPLT

SEQ ID NO: 81 - DNA, 363 bp, synthetic construct
  misc_feature 1..363: note = synthetic
  source 1..363: mol_type = other DNA
gaggtgcagctggtggagtctgggggaggcttggtgaggtcgggggggtccctgagactctcctgtgcagcctctggattcacctttaacagatatgccatgacctgggtccgccaggctccagggaaggggctggagtgggtctcagctataagtggtagtggtagcagcacatactacacagactccgtgaaaggccggttcaccatctccagagacaattccaagaattcggtggatctgcaaatgcacagcctgagagtcgaagacacggccatatattattgtgcgagagggactacagtcactacggggtacggtatggacgtctggggccaagggaccacggtcaccgtctcctca

SEQ ID NO: 82 - protein, 121 aa, synthetic construct
  REGION 1..121: note = synthetic
EVQLVESGGGLVRSGGSLRLSCAASGFTFNRYAMTWVRQAPGKGLEWVSAISGSGSSTYYTDSVKGRFTISRDNSKNSVDLQMHSLRVEDTAIYYCARGTTVTTGYGMDVWGQGTTVTVSS

SEQ ID NO: 83 - DNA, 24 bp, synthetic construct
  misc_feature 1..24: note = synthetic
  source 1..24: mol_type = other DNA
ggattcacctttaacagatatgcc

SEQ ID NO: 84 - protein, 8 aa, synthetic construct
  REGION 1..8: note = synthetic
GFTFNRYA

SEQ ID NO: 85 - DNA, 24 bp, synthetic construct
  misc_feature 1..24: note = synthetic
  source 1..24: mol_type = other DNA
ataagtggtagtggtagcagcaca

SEQ ID NO: 86 - protein, 8 aa, synthetic construct
  REGION 1..8: note = synthetic
ISGSGSST

SEQ ID NO: 87 - DNA, 42 bp, synthetic construct
  misc_feature 1..42: note = synthetic
  source 1..42: mol_type = other DNA
gcgagagggactacagtcactacggggtacggtatggacgtc

SEQ ID NO: 88 - protein, 14 aa, synthetic construct
  REGION 1..14: note = synthetic
ARGTTVTTGYGMDV

SEQ ID NO: 89 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccttcacttgccaggcgagtcaggacattaccaattctttaaattggtatcaacagaaacctgggagagcccctaagctcctgatctacgatgcatcgtatttgaaggcaggggtcccatcaagattcagtggaagtggatctgggacagattttactttcaccatcagcagcctgcagcctgaagatattgcaacatattactgtcaacaatatgatgatctcccatacacttttggccaggggaccaagctggagatcaaa

SEQ ID NO: 90 - protein, 107 aa, synthetic construct
  REGION 1..107: note = synthetic
DIQMTQSPSSLSASVGDRVTFTCQASQDITNSLNWYQQKPGRAPKLLIYDASYLKAGVPSRFSGSGSGTDFTFTISSLQPEDIATYYCQQYDDLPYTFGQGTKLEIK

SEQ ID NO: 91 - DNA, 18 bp, synthetic construct
  misc_feature 1..18: note = synthetic
  source 1..18: mol_type = other DNA
caggacattaccaattct

SEQ ID NO: 92 - protein, 6 aa, synthetic construct
  REGION 1..6: note = synthetic
QDITNS

SEQ ID NO: 93-94 - skipped (2 entries)

SEQ ID NO: 95 - DNA, 27 bp, synthetic construct
  misc_feature 1..27: note = synthetic
  source 1..27: mol_type = other DNA
caacaatatgatgatctcccatacact

SEQ ID NO: 96 - protein, 9 aa, synthetic construct
  REGION 1..9: note = synthetic
QQYDDLPYT

SEQ ID NO: 97 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatatttgactactggggccagggaaccctggtcaccgtctcctca

SEQ ID NO: 98 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 99 - DNA, 24 bp, synthetic construct
  misc_feature 1..24: note = synthetic
  source 1..24: mol_type = other DNA
ggtgactccgtcagtagttcctac

SEQ ID NO: 100 - protein, 8 aa, synthetic construct
  REGION 1..8: note = synthetic
GDSVSSSY

SEQ ID NO: 101 - DNA, 21 bp, synthetic construct
  misc_feature 1..21: note = synthetic
  source 1..21: mol_type = other DNA
atctattacagtgggagttcc

SEQ ID NO: 102 - protein, 7 aa, synthetic construct
  REGION 1..7: note = synthetic
IYYSGSS

SEQ ID NO: 103 - DNA, 42 bp, synthetic construct
  misc_feature 1..42: note = synthetic
  source 1..42: mol_type = other DNA
gcgagagaagggaacgtggatacaactatgatatttgactac

SEQ ID NO: 104 - protein, 14 aa, synthetic construct
  REGION 1..14: note = synthetic
AREGNVDTTMIFDY

SEQ ID NO: 105 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggacgttcggccaagggaccaaggtggaaatcaaa

SEQ ID NO: 106 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 107 - DNA, 18 bp, synthetic construct
  misc_feature 1..18: note = synthetic
  source 1..18: mol_type = other DNA
cagggcattagaaatgat

SEQ ID NO: 108 - protein, 6 aa, synthetic construct
  REGION 1..6: note = synthetic
QGIRND

SEQ ID NO: 109-110 - skipped (2 entries)

SEQ ID NO: 111 - DNA, 27 bp, synthetic construct
  misc_feature 1..27: note = synthetic
  source 1..27: mol_type = other DNA
ctacaagatttcaattacccgtggacg

SEQ ID NO: 112 - protein, 9 aa, synthetic construct
  REGION 1..9: note = synthetic
LQDFNYPWT

SEQ ID NO: 113 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtcatcaagatttcaattacccgtggacgttcggccaagggaccaaggtggaaatcaaa

SEQ ID NO: 114 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCHQDFNYPWTFGQGTKVEIK

SEQ ID NO: 115 - DNA, 18 bp, synthetic construct
  misc_feature 1..18: note = synthetic
  source 1..18: mol_type = other DNA
cagggcattagaaatgat

SEQ ID NO: 116 - protein, 6 aa, synthetic construct
  REGION 1..6: note = synthetic
QGIRND

SEQ ID NO: 117-118 - skipped (2 entries)

SEQ ID NO: 119 - DNA, 27 bp, synthetic construct
  misc_feature 1..27: note = synthetic
  source 1..27: mol_type = other DNA
catcaagatttcaattacccgtggacg

SEQ ID NO: 120 - protein, 9 aa, synthetic construct
  REGION 1..9: note = synthetic
HQDFNYPWT

SEQ ID NO: 121 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaacataacgtggatacaactatgatatttgactactggggccagggaaccctggtcaccgtctcctca

SEQ ID NO: 122 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREHNVDTTMIFDYWGQGTLVTVSS

SEQ ID NO: 123 - DNA, 24 bp, synthetic construct
  misc_feature 1..24: note = synthetic
  source 1..24: mol_type = other DNA
ggtgactccgtcagtagttcctac

SEQ ID NO: 124 - protein, 8 aa, synthetic construct
  REGION 1..8: note = synthetic
GDSVSSSY

SEQ ID NO: 125 - DNA, 21 bp, synthetic construct
  misc_feature 1..21: note = synthetic
  source 1..21: mol_type = other DNA
atctattacagtgggagttcc

SEQ ID NO: 126 - protein, 7 aa, synthetic construct
  REGION 1..7: note = synthetic
IYYSGSS

SEQ ID NO: 127 - DNA, 42 bp, synthetic construct
  misc_feature 1..42: note = synthetic
  source 1..42: mol_type = other DNA
gcgagagaacataacgtggatacaactatgatatttgactac

SEQ ID NO: 128 - protein, 14 aa, synthetic construct
  REGION 1..14: note = synthetic
AREHNVDTTMIFDY

SEQ ID NO: 129 - DNA, 321 bp, synthetic construct
  misc_feature 1..321: note = synthetic
  source 1..321: mol_type = other DNA
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggcacttcggccaagggaccaaggtggaaatcaaa

SEQ ID NO: 130 - protein, 107 aa, synthetic construct
  REGION 1..107: note = synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWHFGQGTKVEIK

SEQ ID NO: 131 - DNA, 18 bp, synthetic construct
  misc_feature 1..18: note = synthetic
  source 1..18: mol_type = other DNA
cagggcattagaaatgat

SEQ ID NO: 132 - protein, 6 aa, synthetic construct
  REGION 1..6: note = synthetic
QGIRND

SEQ ID NO: 133-134 - skipped (2 entries)

SEQ ID NO: 135 - DNA, 27 bp, synthetic construct
  misc_feature 1..27: note = synthetic
  source 1..27: mol_type = other DNA
ctacaagatttcaattacccgtggcac

SEQ ID NO: 136 - protein, 9 aa, synthetic construct
  REGION 1..9: note = synthetic
LQDFNYPWH

SEQ ID NO: 137 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatacatgactactggggccagggaaccctggtcaccgtctcctca

SEQ ID NO: 138 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIHDYWGQGTLVTVSS

SEQ ID NO: 139 - DNA, 24 bp, synthetic construct
  misc_feature 1..24: note = synthetic
  source 1..24: mol_type = other DNA
ggtgactccgtcagtagttcctac

SEQ ID NO: 140 - protein, 8 aa, synthetic construct
  REGION 1..8: note = synthetic
GDSVSSSY

SEQ ID NO: 141 - DNA, 21 bp, synthetic construct
  misc_feature 1..21: note = synthetic
  source 1..21: mol_type = other DNA
atctattacagtgggagttcc

SEQ ID NO: 142 - protein, 7 aa, synthetic construct
  REGION 1..7: note = synthetic
IYYSGSS

SEQ ID NO: 143 - DNA, 42 bp, synthetic construct
  misc_feature 1..42: note = synthetic
  source 1..42: mol_type = other DNA
gcgagagaagggaacgtggatacaactatgatacatgactac

SEQ ID NO: 144 - protein, 14 aa, synthetic construct
  REGION 1..14: note = synthetic
AREGNVDTTMIHDY

SEQ ID NO: 145 - DNA, 360 bp, synthetic construct
  misc_feature 1..360: note = synthetic
  source 1..360: mol_type = other DNA
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatcacactatgatatttgactactggggccagggaaccctggtcaccgtctcctca

SEQ ID NO: 146 - protein, 120 aa, synthetic construct
  REGION 1..120: note = synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDHTMIFDYWGQGTLVTVSS

SEQ ID NO: 147 - DNA, 24 bp, synthetic construct
  misc_feature 1..24: note = synthetic
  source 1..24: mol_type = other DNA
ggtgactccgtcagtagttcctac

SEQ ID NO: 148 - protein, 8 aa, synthetic construct
  REGION 1..8: note = synthetic
GDSVSSSY

SEQ ID NO: 149 - DNA, 21 bp, synthetic construct
  misc_feature 1..21: note = synthetic
  source 1..21: mol_type = other DNA
atctattacagtgggagttcc

SEQ ID NO: 150 - protein, 7 aa, synthetic construct
  REGION 1..7: note = synthetic
IYYSGSS

SEQ ID NO: 151 - DNA, 42 bp, synthetic construct
  misc_feature 1..42: note = synthetic
  source 1..42: mol_type = other DNA
gcgagagaagggaacgtggatcacactatgatatttgactac

SEQ ID NO: 152 - protein, 14 aa, synthetic construct
  REGION 1..14: note = synthetic
AREGNVDHTMIFDY

SEQ ID NO: 153 - DNA, 360 bp, synthetic construct
  misc_feature 1..360: note = synthetic
  source 1..360: mol_type = other DNA
caggtgcagctggtggagtctgggggaggcttggtcaagcctggagggtccctgagactctcctgtgcagcctctggattcaccttcagtgactcctacatgtcctggatccgtcaggctccagggaagggactagagtggatttcatacattggtagtagtggtaataccttttactacgcagactctgtgaagggccggttcaccatttccagagacaacgccaacaatttactgtatctgcaaatgaccagcctgagagccgaggacacggccgtgtattactgtgcgagagaagaaggcgatttttggagtgccgttgactcctggggccagggaaccctggtcaccgtctcctca

SEQ ID NO: 154 - protein, 120 aa, synthetic construct
  REGION 1..120: note = synthetic
QVQLVESGGGLVKPGGSLRLSCAASGFTFSDSYMSWIRQAPGKGLEWISYIGSSGNTFYYADSVKGRFTISRDNANNLLYLQMTSLRAEDTAVYYCAREEGDFWSAVDSWGQGTLVTVSS

SEQ ID NO: 155 - DNA, 24 bp, synthetic construct
  misc_feature 1..24: note = synthetic
  source 1..24: mol_type = other DNA
ggattcaccttcagtgactcctac

SEQ ID NO: 156 - protein, 8 aa, synthetic construct
  REGION 1..8: note = synthetic
GFTFSDSY

SEQ ID NO: 157 - DNA, 24 bp, synthetic construct
  misc_feature 1..24: note = synthetic
  source 1..24: mol_type = other DNA
attggtagtagtggtaataccttt

SEQ ID NO: 158 - protein, 8 aa, synthetic construct
  REGION 1..8: note = synthetic
IGSSGNTF

SEQ ID NO: 159 - DNA, 39 bp, synthetic construct
  misc_feature 1..39: note = synthetic
  source 1..39: mol_type = other DNA
gcgagagaagaaggcgatttttggagtgccgttgactcc

SEQ ID NO: 160 - protein, 13 aa, synthetic construct
  REGION 1..13: note = synthetic
AREEGDFWSAVDS

SEQ ID NO: 161 - DNA, 321 bp, synthetic construct
  misc_feature 1..321: note = synthetic
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccaggtaaagcccctaaactcctgatccatactgcatccactttgcaaagtggggtcccatcaaggttcagcggcagtggatctgggacagaattcactctcacaatcagcaacctgcagcctgaagattttgcaacttattactgtcaacagcttaatagttacccattcactttcggccctgggaccaaagtggatatcaaa

SEQ ID NO: 162 - protein, 107 aa, synthetic construct
  REGION 1..107: note = synthetic
DIQLTQSPSFLSASVGDRVTITCWASQGISSYLAWYQQKPGKAPKLLIHTASTLQSGVPSRFSGSGSGTEFTLTISNLQPEDFATYYCQQLNSYPFTFGPGTKVDIK

SEQ ID NO: 163 - DNA, 18 bp, synthetic construct
  misc_feature 1..18: note = synthetic
  source 1..18: mol_type = other DNA
cagggcattagcagttat

SEQ ID NO: 164 - protein, 6 aa, synthetic construct
  REGION 1..6: note = synthetic
QGISSY

SEQ ID NO: 165-166 - skipped (2 entries)

SEQ ID NO: 167 - DNA, 27 bp, synthetic construct
  misc_feature 1..27: note = synthetic
  source 1..27: mol_type = other DNA
caacagcttaatagttacccattcact

SEQ ID NO: 168 - protein, 9 aa, synthetic construct
  REGION 1..9: note = synthetic
QQLNSYPFT

SEQ ID NO: 169 - DNA, 366 bp, synthetic construct
  misc_feature 1..366: note = synthetic
  source 1..366: mol_type = other DNA
caggtgcagctggtggagtctgggggaggcgtggtccagcctggggggtccctgagactctcctgtgcagcctctggattcaccttcggtggccatgccatgcactgggtccgccaggctccaggcaaggggctggagtggctggcagttatatcatctgatggcagtaataaacagtatgcagattctgtgaagggccgattcaccatctccagggacaatcccaagaacacgctgtatctgcaaatgaacagtctgagagttggggacacggctatttattactgtgcgaaagaggtggcacctcgttattattattacggtctggacgtctggggccaagggaccacggtcaccgtctcctca

SEQ ID NO: 170 - protein, 122 aa, synthetic construct
  REGION 1..122: note = synthetic
QVQLVESGGGVVQPGGSLRLSCAASGFTFGGHAMHWVRQAPGKGLEWLAVISSDGSNKQYADSVKGRFTISRDNPKNTLYLQMNSLRVGDTAIYYCAKEVAPRYYYYGLDVWGQGTTVTVSS

SEQ ID NO: 171 - DNA, 24 bp, synthetic construct
  misc_feature 1..24: note = synthetic
  source 1..24: mol_type = other DNA
ggattcaccttcggtggccatgcc

SEQ ID NO: 172 - protein, 8 aa, synthetic construct
  REGION 1..8: note = synthetic
GFTFGGHA

SEQ ID NO: 173 - DNA, 24 bp, synthetic construct
  misc_feature 1..24: note = synthetic
  source 1..24: mol_type = other DNA
atatcatctgatggcagtaataaa

SEQ ID NO: 174 - protein, 8 aa, synthetic construct
  REGION 1..8: note = synthetic
ISSDGSNK

SEQ ID NO: 175 - DNA, 45 bp, synthetic construct
  misc_feature 1..45: note = synthetic
  source 1..45: mol_type = other DNA
gcgaaagaggtggcacctcgttattattattacggtctggacgtc

SEQ ID NO: 176 - protein, 15 aa, synthetic construct
  REGION 1..15: note = synthetic
AKEVAPRYYYYGLDV

SEQ ID NO: 177 - DNA, 318 bp, synthetic construct
  misc_feature 1..318: note = synthetic
  source 1..318: mol_type = other DNA
gacatccagatgacccagtctccatcctccctgtctgcatctgtgggagacagagtcaccatcacttgccgggcgagtcaggacattagcaattttttagcctggtatcagcagaaaccagggaaggttcctaaactcctgatctatactgcatccactttacaatcaggggtcccatctcggttcagtggcagtggatctgggacagatttcactctcaccgtcagcagcctacagcctgaagatgttgcaacttattactgtcaaaagtatgccggcgccctcactttcggccctgggaccaaagtggatatcaaa

SEQ ID NO: 178 - protein, 106 aa, synthetic construct
  REGION 1..106: note = synthetic
DIQMTQSPSSLSASVGDRVTITCRASQDISNFLAWYQQKPGKVPKLLIYTASTLQSGVPSRFSGSGSGTDFTLTVSSLQPEDVATYYCQKYAGALTFGPGTKVDIK

SEQ ID NO: 179 - DNA, 18 bp, synthetic construct
  misc_feature 1..18: note = synthetic
  source 1..18: mol_type = other DNA
caggacattagcaatttt

SEQ ID NO: 180 - protein, 6 aa, synthetic construct
  REGION 1..6: note = synthetic
QDISNF

SEQ ID NO: 181-182 - skipped (2 entries)

SEQ ID NO: 183 - DNA, 24 bp, synthetic construct
  misc_feature 1..24: note = synthetic
  source 1..24: mol_type = other DNA
caaaagtatgccggcgccctcact

SEQ ID NO: 184 - protein, 8 aa, synthetic construct
  REGION 1..8: note = synthetic
QKYAGALT

SEQ ID NO: 185 - DNA, 357 bp, synthetic construct
  misc_feature 1..357: note = synthetic
  source 1..357: mol_type = other DNA
gaggtgcagctggtggagtctgggggaggcttggcacagcctggggggtccctgagactctcctgtgcagcctctggattcacgtttagaagctatgccatgagctgggtccgccaggctccagggaaggggccggagtgggtctcaggtataggtggtaatggtgttaccacatactacgcagactccgtgaagggccggttcaccatctccagagacaattccaagaacacgctgtttctgcaaatgaatagcctgagagccgaggacacggccgtatattattgtgtgcaggggggtttaggtggttattttacaggctactggggccagggaaccctggtcaccgtctcctca

SEQ ID NO: 186 - protein, 119 aa, synthetic construct
  REGION 1..119: note = synthetic
EVQLVESGGGLAQPGGSLRLSCAASGFTFRSYAMSWVRQAPGKGPEWVSGIGGNGVTTYYADSVKGRFTISRDNSKNTLFLQMNSLRAEDTAVYYCVQGGLGGYFTGYWGQGTLVTVSS

SEQ ID NO: 187 - DNA, 24 bp, synthetic construct
  misc_feature 1..24: note = synthetic
  source 1..24: mol_type = other DNA
ggattcacgtttagaagctatgcc

SEQ ID NO: 188 - protein, 8 aa, synthetic construct
  REGION 1..8: note = synthetic
GFTFRSYA

SEQ ID NO: 189 - DNA, 24 bp, synthetic construct
  misc_feature 1..24: note = synthetic
  source 1..24: mol_type = other DNA
ataggtggtaatggtgttaccaca

SEQ ID NO: 190 - protein, 8 aa, synthetic construct
  REGION 1..8: note = synthetic
IGGNGVTT

SEQ ID NO: 191 - DNA, 36 bp, synthetic construct
  misc_feature 1..36: note = synthetic
  source 1..36: mol_type = other DNA
gtgcaggggggtttaggtggttattttacaggctac

SEQ ID NO: 192 - protein, 12 aa, synthetic construct
  REGION 1..12: note = synthetic
VQGGLGGYFTGY

SEQ ID NO: 193 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagtattagtacctatttaaattggtatcagcagaatccagggaaagcccctaaactcctgatctttgatgcatccagtttgcaaagtggggtcccatcaaggttcagtggcagtggatctgggacagatttcactctcaccatcagaggtctgcaacctgaagattttgcaacttactactgtcaacagagttacagtgccccgctcactttcggcggagggaccaaggtggagatcaaa

SEQ ID NO: 194 - protein, 107 aa, synthetic construct
  REGION 1..107: note = synthetic
DIQMTQSPSSLSASVGDRVTITCRASQSISTYLNWYQQNPGKAPKLLIFDASSLQSGVPSRFSGSGSGTDFTLTIRGLQPEDFATYYCQQSYSAPLTFGGGTKVEIK

SEQ ID NO: 195 - DNA, 18 bp, synthetic construct
  misc_feature 1..18: note = synthetic
  source 1..18: mol_type = other DNA
cagagtattagtacctat

SEQ ID NO: 196 - protein, 6 aa, synthetic construct
  REGION 1..6: note = synthetic
QSISTY

SEQ ID NO: 197-198 - skipped (2 entries)

SEQ ID NO: 199 - DNA, 27 bp, synthetic construct
  misc_feature 1..27: note = synthetic
  source 1..27: mol_type = other DNA
caacagagttacagtgccccgctcact

SEQ ID NO: 200 - protein, 9 aa, synthetic construct
  REGION 1..9: note = synthetic
QQSYSAPLT

SEQ ID NO: 201 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcgtctggattcaccttcagtggttatggcatgcactgggtccgccaggctccaggcaaggggctggagtgggtggcacttatatggcttgatggaagtaatgactactatgcagactccgtgaagggccgattcaccatctccagagacaattccaagaacacgttatatctgcaaatgaacagactgagagccgaggacacggctgtgtattactgtgcgagagatggcccggttgctgctatacccgactactggggccagggaaccctggtcaccgtctcctca

SEQ ID NO: 202 - protein, 119 aa, synthetic construct
  REGION 1..119: note = synthetic
QVQLVESGGGVVQPGRSLRLSCAASGFTFSGYGMHWVRQAPGKGLEWVALIWLDGSNDYYADSVKGRFTISRDNSKNTLYLQMNRLRAEDTAVYYCARDGPVAAIPDYWGQGTLVTVSS

SEQ ID NO: 203 - DNA, 24 bp, synthetic construct
  misc_feature 1..24: note = synthetic
  source 1..24: mol_type = other DNA
ggattcaccttcagtggttatggc

SEQ ID NO: 204 - protein, 8 aa, synthetic construct
  REGION 1..8: note = synthetic
GFTFSGYG

SEQ ID NO: 205 - DNA, 24 bp, synthetic construct
  misc_feature 1..24: note = synthetic
  source 1..24: mol_type = other DNA
atatggcttgatggaagtaatgac

SEQ ID NO: 206 - protein, 8 aa, synthetic construct
  REGION 1..8: note = synthetic
IWLDGSND

SEQ ID NO: 207 - DNA, 36 bp, synthetic construct
  misc_feature 1..36: note = synthetic
  source 1..36: mol_type = other DNA
gcgagagatggcccggttgctgctatacccgactac

SEQ ID NO: 208 - protein, 12 aa, synthetic construct
  REGION 1..12: note = synthetic
ARDGPVAAIPDY

SEQ ID NO: 209 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgccgggccagtcagagtattagtaggtggttggcctggtatcagctgaaaccagggaaagcccctaagctcctgatctataaggcgtctagtttagaaagtggggtcccatcaaggttcagcggcagtggatctgggacagacttcactctcaccatcagcagcctgcaacctgatgattttgcaacttattactgccaacagtataatacttattcgtacacttttggccaggggaccaagctggagatcaaa

SEQ ID NO: 210 - protein, 107 aa, synthetic construct
  REGION 1..107: note = synthetic
DIQMTQSPSTLSASVGDRVTITCRASQSISRWLAWYQLKPGKAPKLLIYKASSLESGVPSRFSGSGSGTDFTLTISSLQPDDFATYYCQQYNTYSYTFGQGTKLEIK

SEQ ID NO: 211 - DNA, 18 bp, synthetic construct
  misc_feature 1..18: note = synthetic
  source 1..18: mol_type = other DNA
cagagtattagtaggtgg

SEQ ID NO: 212 - protein, 6 aa, synthetic construct
  REGION 1..6: note = synthetic
QSISRW

SEQ ID NO: 213-214 - skipped (2 entries)

SEQ ID NO: 215 - DNA, 27 bp, synthetic construct
  misc_feature 1..27: note = synthetic
  source 1..27: mol_type = other DNA
caacagtataatacttattcgtacact

SEQ ID NO: 216 - protein, 9 aa, synthetic construct
  REGION 1..9: note = synthetic
QQYNTYSYT

SEQ ID NO: 217 - DNA, 363 bp, synthetic construct
  misc_feature 1..363: note = synthetic
  source 1..363: mol_type = other DNA
gaggtgcagctggtggagtctgggggaggtgtggtacggcctggggggtccctgagactctcctgtgcagcctctggattcacctttgatgaatatggcatgacttgggtccgccaagttccagggaaggggctggagtgggtctctggtattacttggaatggtggtttcacagattatacagactctgtgaagggccgattcaccagctccagagacaacgccaagaactccctgtatctgcaaatgaacagtctgagagccgaggacacggccttgtattactgtgcgagagatggatatagcagctcgtggggggcttatgatatatggggccaagggacaatggtcaccgtctcttca

SEQ ID NO: 218 - protein, 121 aa, synthetic construct
  REGION 1..121: note = synthetic
EVQLVESGGGVVRPGGSLRLSCAASGFTFDEYGMTWVRQVPGKGLEWVSGITWNGGFTDYTDSVKGRFTSSRDNAKNSLYLQMNSLRAEDTALYYCARDGYSSSWGAYDIWGQGTMVTVSS

SEQ ID NO: 219 - DNA, 24 bp, synthetic construct
  misc_feature 1..24: note = synthetic
  source 1..24: mol_type = other DNA
ggattcacctttgatgaatatggc

SEQ ID NO: 220 - protein, 8 aa, synthetic construct
  REGION 1..8: note = synthetic
GFTFDEYG

SEQ ID NO: 221 - DNA, 24 bp, synthetic construct
  misc_feature 1..24: note = synthetic
  source 1..24: mol_type = other DNA
attacttggaatggtggtttcaca

SEQ ID NO: 222 - protein, 8 aa, synthetic construct
  REGION 1..8: note = synthetic
ITWNGGFT

SEQ ID NO: 223 - DNA, 42 bp, synthetic construct
  misc_feature 1..42: note = synthetic
  source 1..42: mol_type = other DNA
gcgagagatggatatagcagctcgtggggggcttatgatata

SEQ ID NO: 224 - protein, 14 aa, synthetic construct
  REGION 1..14: note = synthetic
ARDGYSSSWGAYDI

SEQ ID NO: 225 - DNA, 321 bp, synthetic construct
  misc_feature 1..321: note = synthetic
  source 1..321: mol_type = other DNA
gacatccagatgacccagtctccatcatccctgtctgcatctgtgggagacagagtcaccatcacttgccgggcaagtcagagcattagcacctatttaaattggtatcagcagaaaccagggaaagcccctaagctcctgatctatgctgcatccagtttgcaaagtggggtcccattaaggttcagtggcagtggatctgggactgatttcactctcaccatcagcagtctgcaacctgaagattttgcaagttatttctgtcaacagagttacagtaccccgtacacttttggccaggggaccaagctggagatcaaa

SEQ ID NO: 226 - protein, 107 aa, synthetic construct
  REGION 1..107: note = synthetic
DIQMTQSPSSLSASVGDRVTITCRASQSISTYLNWYQQKPGKAPKLLIYAASSLQSGVPLRFSGSGSGTDFTLTISSLQPEDFASYFCQQSYSTPYTFGQGTKLEIK

SEQ ID NO: 227 - DNA, 18 bp, synthetic construct
  misc_feature 1..18: note = synthetic
  source 1..18: mol_type = other DNA
cagagcattagcacctat

SEQ ID NO: 228 - protein, 6 aa, synthetic construct
  REGION 1..6: note = synthetic
QSISTY

SEQ ID NO: 229-230 - skipped (2 entries)

SEQ ID NO: 231 - DNA, 27 bp, synthetic construct
  misc_feature 1..27: note = synthetic
  source 1..27: mol_type = other DNA
caacagagttacagtaccccgtacact

SEQ ID NO: 232 - protein, 9 aa, synthetic construct
  REGION 1..9: note = synthetic
QQSYSTPYT

SEQ ID NO: 233 - DNA, 360 bp, synthetic construct
  misc_feature 1..360: note = synthetic
  source 1..360: mol_type = other DNA
gaagtgcagctggtggagtctgggggaggcgtggtacagcctggggggtccctgagactctcctgtgcagcctctggattcacctttaatgattatgccatgcactgggtccgtcaagctccagggaagggtctggagtgggtctctcttattagtggagatggtggtaacacatactatgcagactctgtgaagggccgactcaccatctccagagacaacagcaaaaactccctgtatctgcaaatgaacagtctgagaacagaggacaccgccttatattactgtgcaaaagataagggctggaacttcggttacttcgatctctggggccgtggcaccctggtcactgtctcctca

SEQ ID NO: 234 - protein, 120 aa, synthetic construct
  REGION 1..120: note = synthetic
EVQLVESGGGVVQPGGSLRLSCAASGFTFNDYAMHWVRQAPGKGLEWVSLISGDGGNTYYADSVKGRLTISRDNSKNSLYLQMNSLRTEDTALYYCAKDKGWNFGYFDLWGRGTLVTVSS

SEQ ID NO: 235 - DNA, 24 bp, synthetic construct
  misc_feature 1..24: note = synthetic
  source 1..24: mol_type = other DNA
ggattcacctttaatgattatgcc

SEQ ID NO: 236 - protein, 8 aa, synthetic construct
  REGION 1..8: note = synthetic
GFTFNDYA

SEQ ID NO: 237 - DNA, 24 bp, synthetic construct
  misc_feature 1..24: note = synthetic
  source 1..24: mol_type = other DNA
attagtggagatggtggtaacaca

SEQ ID NO: 238 - protein, 8 aa, synthetic construct
  REGION 1..8: note = synthetic
ISGDGGNT

SEQ ID NO: 239 - DNA, 39 bp, synthetic construct
  misc_feature 1..39: note = synthetic
  source 1..39: mol_type = other DNA
gcaaaagataagggctggaacttcggttacttcgatctc

SEQ ID NO: 240 - protein, 13 aa, synthetic construct
  REGION 1..13: note = synthetic
AKDKGWNFGYFDL

SEQ ID NO: 241 - DNA, 327 bp, synthetic construct
  misc_feature 1..327: note = synthetic
  source 1..327: mol_type = other DNA
gacatccagatgacccagtctccatcctccctgtctacatctgtgggagacagagtcaccatcacttgccgggcaagtcagaacattgacacctatttaaattggtatcagcagaaaccagggaaagcccctaaactcctgatctatgatgcatccagtttacaaagtggggtcccatcacggttcagtggcagcggatctgggacagatttcactctcaccatcaccagtctgcaacctgaagattttgccacttactactgtcaacagaatgacaatattcttcaccctctcactttcggcggagggaccaaggtggagatcaaa

SEQ ID NO: 242 - protein, 109 aa, synthetic construct
  REGION 1..109: note = synthetic
DIQMTQSPSSLSTSVGDRVTITCRASQNIDTYLNWYQQKPGKAPKLLIYDASSLQSGVPSRFSGSGSGTDFTLTITSLQPEDFATYYCQQNDNILHPLTFGGGTKVEIK

SEQ ID NO: 243 - DNA, 18 bp, synthetic construct
  misc_feature 1..18: note = synthetic
  source 1..18: mol_type = other DNA
cagaacattgacacctat

SEQ ID NO: 244 - protein, 6 aa, synthetic construct
  REGION 1..6: note = synthetic
QNIDTY

SEQ ID NO: 245-246 - skipped (2 entries)

SEQ ID NO: 247 - DNA, 33 bp, synthetic construct
  misc_feature 1..33: note = synthetic
  source 1..33: mol_type = other DNA
caacagaatgacaatattcttcaccctctcact

SEQ ID NO: 248 - protein, 11 aa, synthetic construct
  REGION 1..11: note = synthetic
QQNDNILHPLT

SEQ ID NO: 249 - DNA, 369 bp, synthetic construct
  misc_feature 1..369: note = synthetic
  source 1..369: mol_type = other DNA
gaggtgcagctggtggagtctgggggaggcttggtccaaccgggggggtccctgagactctcctgtgcagcctctggattccactctaatagatattggatggactgggtccgccaggctccagggaaggggctggagtgggtggccaacataaagcaagatggaagtgaggaaaactatgtggactctgtgaagggccgattcaccatctccagagacaacgccaagaactcactttatctgcaaatgaacagcctgagagccgaggacacggctgtgtattactgtgcgagagatcgaagcacctcgtgggtcccttactggttcttcgatctctggggccgtggcaccctggtcactgtctcctca

SEQ ID NO: 250 - protein, 123 aa, synthetic construct
  REGION 1..123: note = synthetic
EVQLVESGGGLVQPGGSLRLSCAASGFHSNRYWMDWVRQAPGKGLEWVANIKQDGSEENYVDSVKGRFTISRDNAKNSLYLQMNSLRAEDTAVYYCARDRSTSWVPYWFFDLWGRGTLVTVSS

SEQ ID NO: 251 - DNA, 24 bp, synthetic construct
  misc_feature 1..24: note = synthetic
  source 1..24: mol_type = other DNA
ggattccactctaatagatattgg

SEQ ID NO: 252 - protein, 8 aa, synthetic construct
  REGION 1..8: note = synthetic
GFHSNRYW

SEQ ID NO: 253 - DNA, 24 bp, synthetic construct
  misc_feature 1..24: note = synthetic
  source 1..24: mol_type = other DNA
ataaagcaagatggaagtgaggaa

SEQ ID NO: 254 - protein, 8 aa, synthetic construct
  REGION 1..8: note = synthetic
IKQDGSEE

SEQ ID NO: 255 - DNA, 48 bp, synthetic construct
  misc_feature 1..48: note = synthetic
  source 1..48: mol_type = other DNA
gcgagagatcgaagcacctcgtgggtcccttactggttcttcgatctc

SEQ ID NO: 256 - protein, 16 aa, synthetic construct
  REGION 1..16: note = synthetic
ARDRSTSWVPYWFFDL

SEQ ID NO: 257 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 258 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 259 - DNA, 18 bp, synthetic construct
  misc_feature 1..18: note = synthetic
  source 1..18: mol_type = other DNA
cagagcattagcagctat

SEQ ID NO: 260 - protein, 6 aa, synthetic construct
  REGION 1..6: note = synthetic
QSISSY

SEQ ID NO: 261-262 - skipped (2 entries)

SEQ ID NO: 263 - DNA, 30 bp, synthetic construct
  misc_feature 1..30: note = synthetic
  source 1..30: mol_type = other DNA
caacagagttacagtacccctccgatcacc

SEQ ID NO: 264 - protein, 10 aa, synthetic construct
  REGION 1..10: note = synthetic
QQSYSTPPIT

SEQ ID NO: 265 - DNA, 360 bp, synthetic construct
  misc_feature 1..360: note = synthetic
  source 1..360: mol_type = other DNA
gaagtgcagctggtggagtctgggggaggcgtggtacagcggggggagtccctgagactctcctgttcagcctctgacttcatctttaaagattatgccatgtactgggtccgtcaaattccagggaagggtctagagtggatctctcttattagtggtgatggtgacactacatggtatggagactctgtgaagggccgattcaccatctccagagacaacaacgaaaactccctctttctgcaaatgaacgatctgagaactgaggacaccgccatgtactactgtgcaagagatatggggtggaacttctttcagttgcaatactggggccagggaaccctggtcaccgtctcctca

SEQ ID NO: 266 - protein, 120 aa, synthetic construct
  REGION 1..120: note = synthetic
EVQLVESGGGVVQRGESLRLSCSASDFIFKDYAMYWVRQIPGKGLEWISLISGDGDTTWYGDSVKGRFTISRDNNENSLFLQMNDLRTEDTAMYYCARDMGWNFFQLQYWGQGTLVTVSS

SEQ ID NO: 267 - DNA, 24 bp, synthetic construct
  misc_feature 1..24: note = synthetic
  source 1..24: mol_type = other DNA
gacttcatctttaaagattatgcc

SEQ ID NO: 268 - protein, 8 aa, synthetic construct
  REGION 1..8: note = synthetic
DFIFKDYA

SEQ ID NO: 269 - DNA, 24 bp, synthetic construct
  misc_feature 1..24: note = synthetic
  source 1..24: mol_type = other DNA
attagtggtgatggtgacactaca

SEQ ID NO: 270 - protein, 8 aa, synthetic construct
  REGION 1..8: note = synthetic
ISGDGDTT

SEQ ID NO: 271 - DNA, 39 bp, synthetic construct
  misc_feature 1..39: note = synthetic
  source 1..39: mol_type = other DNA
gcaagagatatggggtggaacttctttcagttgcaatac

SEQ ID NO: 272 - protein, 13 aa, synthetic construct
  REGION 1..13: note = synthetic
ARDMGWNFFQLQY

SEQ ID NO: 273 - DNA, 361 bp, synthetic construct
  misc_feature 1..361: note = synthetic
  source 1..361: mol_type = other DNA
dcaggtgcagctgcaggagtcgggccccgcactggtgaagccttcacagaccctgtccctcacctgcactgtctctggtggctccatcatcagaggtagtacctactggagttgggtccgccaattcccagggaagggcctggagtggattggatacagttattacagtgggaccgcctactataatccgtccctcgagagtcgagctaccatttctgtagacacgtctaagaaccagttctccctgaacctgaagtctgtgacggccgcggacacggccgtgtattattgtacaagagaaataggagtggctggtctctttgacatctggggccagggaaccctggtcaccgtctcctca

SEQ ID NO: 274 - protein, 120 aa, synthetic construct
  REGION 1..120: note = synthetic
QVQLQESGPALVKPSQTLSLTCTVSGGSIIRGSTYWSWVRQFPGKGLEWIGYSYYSGTAYYNPSLESRATISVDTSKNQFSLNLKSVTAADTAVYYCTREIGVAGLFDIWGQGTLVTVSS

SEQ ID NO: 275 - DNA, 30 bp, synthetic construct
  misc_feature 1..30: note = synthetic
  source 1..30: mol_type = other DNA
ggtggctccatcatcagaggtagtacctac

SEQ ID NO: 276 - protein, 10 aa, synthetic construct
  REGION 1..10: note = synthetic
GGSIIRGSTY

SEQ ID NO: 277 - DNA, 21 bp, synthetic construct
  misc_feature 1..21: note = synthetic
  source 1..21: mol_type = other DNA
agttattacagtgggaccgcc

SEQ ID NO: 278 - protein, 7 aa, synthetic construct
  REGION 1..7: note = synthetic
SYYSGTA

SEQ ID NO: 279 - DNA, 36 bp, synthetic construct
  misc_feature 1..36: note = synthetic
  source 1..36: mol_type = other DNA
acaagagaaataggagtggctggtctctttgacatc

SEQ ID NO: 280 - protein, 12 aa, synthetic construct
  REGION 1..12: note = synthetic
TREIGVAGLFDI

SEQ ID NO: 281 - DNA, 324 bp, synthetic construct
  misc_feature 1..324: note = synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 282 - protein, 108 aa, synthetic construct
  REGION 1..108: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 283 - DNA, 36 bp, synthetic construct
  misc_feature 1..36: note = synthetic
  source 1..36: mol_type = other DNA
agagcaagtcagtcagtctctagctcttatctcgcc

SEQ ID NO: 284 - protein, 12 aa, synthetic construct
  REGION 1..12: note = synthetic
RASQSVSSSYLA

SEQ ID NO: 285 - DNA, 21 bp, synthetic construct
  misc_feature 1..21: note = synthetic
  source 1..21: mol_type = other DNA
ggggcaagttccagggccacc

SEQ ID NO: 286 - protein, 7 aa, synthetic construct
  REGION 1..7: note = synthetic
GASSRAT

SEQ ID NO: 287 - DNA, 27 bp, synthetic construct
  misc_feature 1..27: note = synthetic
  source 1..27: mol_type = other DNA
caacagtacggaagcagcccgtggacg

SEQ ID NO: 288 - protein, 9 aa, synthetic construct
  REGION 1..9: note = synthetic
QQYGSSPWT

SEQ ID NO: 289 - DNA, 357 bp, synthetic construct
  misc_feature 1..357: note = synthetic
  source 1..357: mol_type = other DNA
caggagcagttggtgcagtctggggctgaggtgaagaagcctggggcctcagtgaaggtctcctgtaaggcttctggatacaccttcaccggctactatatacattgggtgcgacaggcccctggactagggcttgaatggatgggatggatcaaccctaacagtggtggcacaaaatatgcacagaagtttcagggcagggtcaccatgaccagggacacgtccatcaatacagcctacatggagctgaaaagactgaaatctgacgactcggccgtatattactgtgcgagagacgcccctccccatgatgtttttgatatctggggccaagggacattggtcaccgtctcttca

SEQ ID NO: 290 - protein, 119 aa, synthetic construct
  REGION 1..119: note = synthetic
QEQLVQSGAEVKKPGASVKVSCKASGYTFTGYYIHWVRQAPGLGLEWMGWINPNSGGTKYAQKFQGRVTMTRDTSINTAYMELKRLKSDDSAVYYCARDAPPHDVFDIWGQGTLVTVSS

SEQ ID NO: 291 - DNA, 24 bp, synthetic construct
  misc_feature 1..24: note = synthetic
  source 1..24: mol_type = other DNA
ggatacaccttcaccggctactat

SEQ ID NO: 292 - protein, 8 aa, synthetic construct
  REGION 1..8: note = synthetic
GYTFTGYY

SEQ ID NO: 293 - DNA, 24 bp, synthetic construct
  misc_feature 1..24: note = synthetic
  source 1..24: mol_type = other DNA
atcaaccctaacagtggtggcaca

SEQ ID NO: 294 - protein, 8 aa, synthetic construct
  REGION 1..8: note = synthetic
INPNSGGT

SEQ ID NO: 295 - DNA, 36 bp, synthetic construct
  misc_feature 1..36: note = synthetic
  source 1..36: mol_type = other DNA
gcgagagacgcccctccccatgatgtttttgatatc

SEQ ID NO: 296 - protein, 12 aa, synthetic construct
  REGION 1..12: note = synthetic
ARDAPPHDVFDI

SEQ ID NO: 297 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaattggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 298 - protein, 107 aa, synthetic construct
  REGION 1..107: note = synthetic
DIQMTQSPSSLSASVGDRVTITCRASQGIRNDLGWYQQKPGKAPKRLIYAASSLQIGVPSRFSGSGSGTEFTLTISSLQPEDFATYYCLQHNSYPLTFGGGTKVEIK

SEQ ID NO: 299 - DNA, 18 bp, synthetic construct
  misc_feature 1..18: note = synthetic
  source 1..18: mol_type = other DNA
cagggcattagaaatgat

SEQ ID NO: 300 - protein, 6 aa, synthetic construct
  REGION 1..6: note = synthetic
QGIRND

SEQ ID NO: 301-302 - skipped (2 entries)

SEQ ID NO: 303 - DNA, 27 bp, synthetic construct
  misc_feature 1..27: note = synthetic
  source 1..27: mol_type = other DNA
ctacagcataatagttacccgctcact

SEQ ID NO: 304 - protein, 9 aa, synthetic construct
  REGION 1..9: note = synthetic
LQHNSYPLT

SEQ ID NO: 305 - DNA, 357 bp, synthetic construct
  misc_feature 1..357: note = synthetic
  source 1..357: mol_type = other DNA
caggtgcagctgcaagagtcgggcccaggactggtgaagccttcacagaccctgtccctcacctgcactgtctctggtggctccatcagtagtggtgcttaccactggagctggatccgccagcacccagggaagggcctagagtggattggatacatctattacaatggggacacctactataatccgtccctcaagagtcgcgttaccatttcagtggacacgtctaagaaccaattcttcctgaaggtgacctctgtgactgccgcggacacggccatgtattactgtgcgggagaaaagcagctgactgcttttgatatctggggccaagggacattggtcaccgtctcttca

SEQ ID NO: 306 - protein, 119 aa, synthetic construct
  REGION 1..119: note = synthetic
QVQLQESGPGLVKPSQTLSLTCTVSGGSISSGAYHWSWIRQHPGKGLEWIGYIYYNGDTYYNPSLKSRVTISVDTSKNQFFLKVTSVTAADTAMYYCAGEKQLTAFDIWGQGTLVTVSS

SEQ ID NO: 307 - DNA, 30 bp, synthetic construct
  misc_feature 1..30: note = synthetic
  source 1..30: mol_type = other DNA
ggtggctccatcagtagtggtgcttaccac

SEQ ID NO: 308 - protein, 10 aa, synthetic construct
  REGION 1..10: note = synthetic
GGSISSGAYH

SEQ ID NO: 309 - DNA, 21 bp, synthetic construct
  misc_feature 1..21: note = synthetic
  source 1..21: mol_type = other DNA
atctattacaatggggacacc

SEQ ID NO: 310 - protein, 7 aa, synthetic construct
  REGION 1..7: note = synthetic
IYYNGDT

SEQ ID NO: 311 - DNA, 33 bp, synthetic construct
  misc_feature 1..33: note = synthetic
  source 1..33: mol_type = other DNA
gcgggagaaaagcagctgactgcttttgatatc

SEQ ID NO: 312 - protein, 11 aa, synthetic construct
  REGION 1..11: note = synthetic
AGEKQLTAFDI

SEQ ID NO: 313 - DNA, 321 bp, synthetic construct
  misc_feature 1..321: note = synthetic
  source 1..321: mol_type = other DNA
gtcatccagatgacccagtctccatcctccctgtctgcatctgttggagacagagtcaccataacttgccgggcgagtcaggacattaataattttttaaattggtatcaacagaaattagggaaagcccctaaactcctgatctccgatgcatccaatttgcagacaggagtcccgtcaaggttcagtggaagtggatctgggacagattttactttcaccatcagcagcctgcagcctgaagatattgctgcatattactgtcaacaatatgatcatttcccgtatacttttggccaggggaccagactggagaacaat

SEQ ID NO: 314 - protein, 107 aa, synthetic construct
  REGION 1..107: note = synthetic
VIQMTQSPSSLSASVGDRVTITCRASQDINNFLNWYQQKLGKAPKLLISDASNLQTGVPSRFSGSGSGTDFTFTISSLQPEDIAAYYCQQYDHFPYTFGQGTRLENN

SEQ ID NO: 315 - DNA, 18 bp, synthetic construct
  misc_feature 1..18: note = synthetic
  source 1..18: mol_type = other DNA
caggacattaataatttt

SEQ ID NO: 316 - protein, 6 aa, synthetic construct
  REGION 1..6: note = synthetic
QDINNF

SEQ ID NO: 317-318 - skipped (2 entries)

SEQ ID NO: 319 - DNA, 27 bp, synthetic construct
  misc_feature 1..27: note = synthetic
  source 1..27: mol_type = other DNA
caacaatatgatcatttcccgtatact

SEQ ID NO: 320 - protein, 9 aa, synthetic construct
  REGION 1..9: note = synthetic
QQYDHFPYT

SEQ ID NO: 321 - DNA, 357 bp, synthetic construct
  misc_feature 1..357: note = synthetic
  source 1..357: mol_type = other DNA
gaggtgcagttggtggagtctgggggaggtgtggttcggcctggggggtccctgagactctcctgtgcagcctctggattcacctttgatgattatggcatgacctgggtccgccaagctccagggaaggggctggagtgggtctctggtattaattggaatggcgatagcacagagtattcagactctgtgaagggccgattcaccatctccagagacaacgccaagaactccctgtatctgcaaatgaacagtctgagagccgaggacacggccttctatcactgtgcgagagagaataactggaacttctactttgactactggggccagggaaccctggtcaccgtctcctca

SEQ ID NO: 322 - protein, 119 aa, synthetic construct
  REGION 1..119: note = synthetic
EVQLVESGGGVVRPGGSLRLSCAASGFTFDDYGMTWVRQAPGKGLEWVSGINWNGDSTEYSDSVKGRFTISRDNAKNSLYLQMNSLRAEDTAFYHCARENNWNFYFDYWGQGTLVTVSS

SEQ ID NO: 323 - DNA, 24 bp, synthetic construct
  misc_feature 1..24: note = synthetic
  source 1..24: mol_type = other DNA
ggattcacctttgatgattatggc

SEQ ID NO: 324 - protein, 8 aa, synthetic construct
  REGION 1..8: note = synthetic
GFTFDDYG

SEQ ID NO: 325 - DNA, 24 bp, synthetic construct
  misc_feature 1..24: note = synthetic
  source 1..24: mol_type = other DNA
attaattggaatggcgatagcaca

SEQ ID NO: 326 - protein, 8 aa, synthetic construct
  REGION 1..8: note = synthetic
INWNGDST

SEQ ID NO: 327 - DNA, 36 bp, synthetic construct
  misc_feature 1..36: note = synthetic
  source 1..36: mol_type = other DNA
gcgagagagaataactggaacttctactttgactac

SEQ ID NO: 328 - protein, 12 aa, synthetic construct
  REGION 1..12: note = synthetic
ARENNWNFYFDY

SEQ ID NO: 329 - DNA, 321 bp, synthetic construct
  misc_feature 1..321: note = synthetic
  source 1..321: mol_type = other DNA
gaaatagtgatgacgcagtctccagccaccctgtctgtgtctcgaggggaaagagccaccctctcctgtagggccagtcagagtgttagcagcaacttagcctggtaccagcagaaacttggccaggctcccaggctcctcatctatggtgcatccaccagggccactggtatcccagccaggttcagtggcagtgggtctgggacagagttcactctcaccatcagcagcctgcagtctgaagattttgcagtttattattgtcagcagtataataactggccgtggacgttcggccaagggaccaaggtggaaatcaaa

SEQ ID NO: 330 - protein, 107 aa, synthetic construct
  REGION 1..107: note = synthetic
EIVMTQSPATLSVSRGERATLSCRASQSVSSNLAWYQQKLGQAPRLLIYGASTRATGIPARFSGSGSGTEFTLTISSLQSEDFAVYYCQQYNNWPWTFGQGTKVEIK

SEQ ID NO: 331 - DNA, 18 bp, synthetic construct
  misc_feature 1..18: note = synthetic
  source 1..18: mol_type = other DNA
cagagtgttagcagcaac

SEQ ID NO: 332 - protein, 6 aa, synthetic construct
  REGION 1..6: note = synthetic
QSVSSN

SEQ ID NO: 333-334 - skipped (2 entries)

SEQ ID NO: 335 - DNA, 27 bp, synthetic construct
  misc_feature 1..27: note = synthetic
  source 1..27: mol_type = other DNA
cagcagtataataactggccgtggacg

SEQ ID NO: 336 - protein, 9 aa, synthetic construct
  REGION 1..9: note = synthetic
QQYNNWPWT

SEQ ID NO: 337 - DNA, 351 bp, synthetic construct
  misc_feature 1..351: note = synthetic
  source 1..351: mol_type = other DNA
caggtccacctggtacagtctggggctgaggtgaagaagcctggggcctcagtgaaggtctcctgcaaggtttccggaaacaccctcactgaattatccatgcactgggtgcgacaggctcctggaaaagggcttgagtggatgggaggttttgatcctgaagatggtgacacaatctactcacagaagttccagggcagagtcaccttgaccgaggacacatctacagacacagcctacatggagctgagcagcctgagatctgaggacacggccgtgtattactgttcaacagtggggggacctacctctgactgctggggccagggaaccctggtcaccgtctcctca

SEQ ID NO: 338 - protein, 117 aa, synthetic construct
  REGION 1..117: note = synthetic
QVHLVQSGAEVKKPGASVKVSCKVSGNTLTELSMHWVRQAPGKGLEWMGGFDPEDGDTIYSQKFQGRVTLTEDTSTDTAYMELSSLRSEDTAVYYCSTVGGPTSDCWGQGTLVTVSS

SEQ ID NO: 339 - DNA, 24 bp, synthetic construct
  misc_feature 1..24: note = synthetic
  source 1..24: mol_type = other DNA
ggaaacaccctcactgaattatcc

SEQ ID NO: 340 - protein, 8 aa, synthetic construct
  REGION 1..8: note = synthetic
GNTLTELS

SEQ ID NO: 341 - DNA, 24 bp, synthetic construct
  misc_feature 1..24: note = synthetic
  source 1..24: mol_type = other DNA
tttgatcctgaagatggtgacaca

SEQ ID NO: 342 - protein, 8 aa, synthetic construct
  REGION 1..8: note = synthetic
FDPEDGDT

SEQ ID NO: 343 - DNA, 30 bp, synthetic construct
  misc_feature 1..30: note = synthetic
  source 1..30: mol_type = other DNA
tcaacagtggggggacctacctctgactgc

SEQ ID NO: 344 - protein, 10 aa, synthetic construct
  REGION 1..10: note = synthetic
STVGGPTSDC

SEQ ID NO: 345 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaaggtcctgatcttcgatgcatccaatttagaaccaggggtcccatcaaggttcagtggaagtggatctgggacagattttactttcaccatcatcagcctgcagcctgaagatattgcaacatattactgtcaacaatatgataatctcccgatcaccttcggccaggggacacgactggacattaaa

SEQ ID NO: 346 - protein, 107 aa, synthetic construct
  REGION 1..107: note = synthetic
DIQMTQSPSSLSASVGDRVTITCQASQDISNYLNWYQQKPGKAPKVLIFDASNLEPGVPSRFSGSGSGTDFTFTIISLQPEDIATYYCQQYDNLPITFGQGTRLDIK

SEQ ID NO: 347 - DNA, 18 bp, synthetic construct
  misc_feature 1..18: note = synthetic
  source 1..18: mol_type = other DNA
caggacattagcaactat

SEQ ID NO: 348 - protein, 6 aa, synthetic construct
  REGION 1..6: note = synthetic
QDISNY

SEQ ID NO: 349-350 - skipped (2 entries)

SEQ ID NO: 351 - DNA, 27 bp, synthetic construct
  misc_feature 1..27: note = synthetic
  source 1..27: mol_type = other DNA
caacaatatgataatctcccgatcacc

SEQ ID NO: 352 - protein, 9 aa, synthetic construct
  REGION 1..9: note = synthetic
QQYDNLPIT

SEQ ID NO: 353 - protein, 9 aa, synthetic construct
NMATGMDSW

SEQ ID NO: 354 - protein, 20 aa, synthetic construct
WEVHLVPRRKQLQFALPDSL

SEQ ID NO: 355 - protein, 7 aa, synthetic construct
ATGMDSW

SEQ ID NO: 356 - protein, 13 aa, synthetic construct
WEVHLVPRRKQLQ

SEQ ID NO: 357 - protein, 9 aa, synthetic construct
LVPRRKQLQ

SEQ ID NO: 358 - protein, 447 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 214 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - DNA, 5480 bp, Homo sapiens
  source 1..5480: mol_type = unassigned DNA
tatatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaaaaaaaaaaaaaaaaa

SEQ ID NO: 361 - DNA, 5480 bp, Homo sapiens
  source 1..5480: mol_type = unassigned DNA
ttttttttttttttttttgaaagcaaactcaggctttaatgatcagtttcctgttccttggtattttctttcaagcaaaggccatgtttttgtaatactccctttcaatggactgttctttcggccccagcaaacattcctgagtggtaggtttgaggaggtgttccaatttattgtttccggtgtccaataaccttggaggagtatctgtcttcatgccctccaaggccatgttatttcagaaagttacagcatttgaaatcattctctaataaaagcaagtgccactaattctaagtaaagtgagctttacaaataagaccagctatgaatgtttaaaaaaagaagaaaacaaaaaaacgaacttcaacaacaggagtccataagtgcaaactgtatgcagctgaacttcaggaattttagcatccatttaaaaagatatcttcggcaaattcatctaaattagctaaaaatgcttgacacgatgaacatgttgtgtctctaggccagtattcaatccaggtcaaggaatctaaagggtagatgtacctgaaactgaaattgtattttatctggagggcttctttacccataattaagtactgtcttccttttaccagctcagcgttagtacaggttacctttttaatgaaggtaatctcagagtctttctcagcaacagcttccccagttttgtagatatccagaagggttgccttgtacttgacaaaaacattttctacagtgatggatgtgatgctaactttataagcatatgcaatctctggtttacatgctgtttgttttcttgtctctgcagagattgtcagatccaattcttcctgcatttgcccacaatcagcttctacacacttgcacgcggctccttcacagactttctgaattttgatattggaagtgctataaaacatggtacactgtttatctggtctgtggtattcgtacactgtgaaagtggcaggactgagaaacccaacttcaaagagttcaaatatccggaatcgtacacaaaggaaatcactggagggaatcgaattcagttgcagaataacatgtccatctttgatttggtaatcagtgaatagttgatccaccccttccacaagggcttttaagtcttcttcatttgcactgattccagtaggcaaggagatgtccatcaccgcatgagaggatccagatgatgattcttccctgctgggcttgtagctggcacatgctactatgcgtttgtaatcagagtttccgtagcctctgtagtgggatgcttcaatatcctgagtatcgattttcaaataaaagctgcaaacttcctcagaggtactggttttgtgaactacagttgttacatgtactgtagccaagccactgccaaatcctgtactgacaatgaggtcatcattgagaagcacctctactggcctcccaaggaaattcttgtctgtcattttataattatgtaaggcacctttatgcttgtaagaaacatcgatgtccatactcaagcggagttgtttaaccaggagtgaatattccgtcaggccctcaatggcattgattgtgtcctgggttgaataaaagccacctccatacctctgctcttctgatagccatttgatgactgggttaacataatttatatctttcaagttcagactggtgagtaaagcataggcagttgtttctaccatacgtgccgtaccagtgttaggtacagagctgtctttatgctgaagattgtctttccaaaaacgataaatgggtggattacctttaaccaaagcttctctcttcaaagctgaaacaattgaacgaaactgtgggtgagttttatctcccagggaaagagcatacgcagaaatggccaatgtaaaggtgctctgggctggcagtgtattttcaagcagaaagttgtcagctttaattagagctgtgtcgattttcaccagggggcatatatcgaaagcctttctaattccaatcacagtaaaggctgtaagatataagctgttctctcgggcttcaacaggcaaggtaccctgtaattttattggttgatactgtgaattttccttgaaagatccattatctaattgataattctcaactagccacaataaagaattacaaattgaattttggttctgctctacgtatttatttacttgtccaagtactcttaaagcaaaagctgttaaccaagtgctagcacttccacccttccacacactgtaagagtagtcagcatttctgtaggacataatgctcaacatcccttcttttaattttttcttcagtttctgcttttcaattaatgggtcagaatgaaaaatgttccaatgatttcctgtttccaggtagtgaaaaacatagaatactgggacaacgctcatcagctccgcctctgcactccctttggggaggtgggttaggatattgatgccttcctgacttagaactgcagacaagatctcacctacaagcagtccttttacactcaaaatccttttgatttctgttttggggaccaaatctaagggtatcctgtatgggaactcctttcgtctgctaatggtaccataaatacccctaggatccaaagtaacaccagaatagctttcccttttgacaccttctggcaccactcgtaatgtttttactaagatttcttttccaaaccaagtctccagtgaaaaattgatgttgtgaaggccaatttccagaggaagcacagtgaatgtcaccaagtgactggaggagccctctactttctggcgcacacatttggaggactttgtgccctgatgatcaatgactgggctttccgaagtgcagattccctccacagcagacattttaacacagaactgcatcccagaagtcctatagttgtaaacagttcctttcaattggatctgttctcctcgtacaacagaatatggtatattcatttccaggaagacatctttgaacacctttgccttgacagtatcagcaacacatataccagtgtttgaaatgccaacgccttgaatttcccaggtggttagagaatcaggtagggcaaactgcaactgttttcttctgggaacaagatgaacttcccacaaccagctttctggaaaataactccgaatttctggcttgcttactggtaacagggtcttcatgtgtagccttcccaattgcatgtctttatgagagatattagcacggagctggcttgcgacgacacaacattcagtgaaagctttgatgcatcttggccctaaactaatccgtgcagctcgctgctcacaggtttcatcattattaacgcaggctccatcgtaacaacatttcttcactactgaatgtttatatttagcagctatttcttctatcttcttttgcagcgttcttcttggcctgagaatttctttacaaggttcatcattttcttgggagtcatctgcatttgcattagtgaggaaggtaagtccagctaggtggaacacattggcattgttgaggccaccacctgccccacagcccagatcactcttctctaagaattgaaatactctttccaagggctttttggctcctctttggactccatacacagcactgtccactgctgctaatgccacccaggaatccattccagttgccatattaagagacacagtttggcctggagaatatgcatctgcatcaggagacagatgaacctggagctggttgccacatttttcttcaatatttaaccagactgaatcagacactaattctgctgtctgttctcctgtgacgatgtaatagaccagaagtcgggatgaaggaaccatgttctgtgttactggaatgtttatactttgataagatgcatctgaaaatttctccctcgtgccaaagtggataattttgcccttggataaaatcaagtaattatagtgagttattttgtcaatatatgggcttttgggggtaacaataatattcagatgttctcccactagcaaagccttatggttatcagtccaatcaatataaaggtaactttggctgagagatgagtatgctattgctcggtaaccttccctggcctgattttcttctggaagatctggagcatcagttttgacattaaactccagcaccgtcactccagatgggagattaagcacaaaggaagctactccatcatcaacacgtgttacacttttgcttggatccaagtcagatgtctcttggtttacatcaattgtttgtgcattcagtgttactgggactcctcctaccaactggtcaagcgaatctttaacctgcaccttgatgggatatggaatcccaggcttcaggaaaagaggagtagcaaccaaattcagtttgtagggagagaggacatatttgatgccaggtatttctgcctcttcagaaaatccacctgtagactctatgactgttacagcaatataaaggtacttgttgtttaaatcttctaaactgtagtatgacagttctttgactgctgtttcagaatcaaatgtgacttgagcaattccatttatcaacattgtgttttgcattgctgtttgcatcatttctttttgatcatcttttaagtcttctcttattccaaatgtgatataaacgtcagcctcagtgactactttattataaaaatatcttgcttttatagtaatttcaaaattcttaaagttcttgtaaccaatgaaattatattctggctcgattgagacagaaaaatgtggcaagacatattctttaacttcaaaatatgcggttccagttgttgaaaagtcctctttatatttagccttgatcgtccacataccatatctaggattagacggaatcttgaagtcaggaaaagagataattccaatatgatcaatttcttctaccatgtcaacttctgatccttcaggatctatgaaagttaagacagtttctcttttggctggcttcaagtcgtcattcaacgaataaactctaacttttactgactggtctggagtataaacaggtttgtctgtatgaatgaagagaaatccattgtcataggttattggcattctttttgattttgaaaaatgctttgatacaacttccaaatacacataagaaactgggttttgtcctccaggcaattgttttggttgtattgttaagattgcagagttttggaatttattctctgaggataaatgaacatggcctgaggagtaactaaattttttatcaggataacttttaatagagattgttgcatcaaatgcttcagtgtatccataaacttgaatcacaatattttcagatgctccaacacggaatatttttggtgctgaaatgacatatgtttgctcctgtccccaggttttccccaggaagattaaaaaacaaagtattcccaaaaggcccatggttggaggtagcaggaaaccacggatata

SEQ ID NO: 362-363 - skipped (2 entries)

SEQ ID NO: 364 - RNA, 23 nt, synthetic construct
  source 1..23: mol_type = other RNA
tattataaaaatatcttgctttt

SEQ ID NO: 365 - RNA, 21 nt, synthetic construct
  source 1..21: mol_type = other RNA
aagcaagatatttttataata

SEQ ID NO: 366 - RNA, 21 nt, synthetic construct
  source 1..21: mol_type = other RNA
aagcaagatatttttataata

SEQ ID NO: 367 - DNA, 25 bp, synthetic construct
  source 1..25: mol_type = other DNA
  misc_feature 1..23: note = RNA
  misc_feature 24..25: note = DNA
tattataaaaatatcttgctttttt

SEQ ID NO: 368 - skipped

SEQ ID NO: 369 - DNA, 25 bp, synthetic construct
  source 1..25: mol_type = other DNA
  misc_feature 1..23: note = RNA
  misc_feature 24..25: note = DNA
  modified_base 1: mod_base = OTHER; note = 2'-O-methyl uridine
  modified_base 1^2: mod_base = OTHER; note = phosphorothioate linkage
  modified_base 2: mod_base = OTHER; note = 2'-fluoro adenosine
  modified_base 2^3: mod_base = OTHER; note = phosphorothioate linkage
  modified_base 3: mod_base = OTHER; note = 2'-fluoro uridine
  modified_base 4: mod_base = OTHER; note = 2'-O-methyl uridine
  modified_base 5: mod_base = OTHER; note = 2'-fluoro adenosine
  modified_base 6: mod_base = OTHER; note = 2'-O-methyl uridine
  modified_base 7: mod_base = OTHER; note = 2'-O-methyl adenosine
  modified_base 8: mod_base = OTHER; note = 2'-fluoro adenosine
  modified_base 9: mod_base = OTHER; note = 2'-O-methyl adenosine
  modified_base 10: mod_base = OTHER; note = 2'-fluoro adenosine
  modified_base 11: mod_base = OTHER; note = 2'-O-methyl adenosine
  modified_base 12: mod_base = OTHER; note = 2'-O-methyl uridine
  modified_base 13: mod_base = OTHER; note = 2'-O-methyl adenosine
  modified_base 14: mod_base = OTHER; note = 2'-fluoro uridine
  modified_base 15: mod_base = OTHER; note = 2'-O-methyl cytidine
  modified_base 16: mod_base = OTHER; note = 2'-fluoro uridine
  modified_base 17: mod_base = OTHER; note = 2'-O-methyl uridine
  modified_base 18: mod_base = OTHER; note = 2'-fluoro guanosine
  modified_base 19: mod_base = OTHER; note = 2'-O-methyl cytidine
  modified_base 20..23: mod_base = OTHER; note = 2'-O-methyl uridine
  modified_base 21^22: mod_base = OTHER; note = phosphorothioate linkage
  modified_base 22^23: mod_base = OTHER; note = phosphorothioate linkage
tattataaaaatatcttgctttttt

SEQ ID NO: 370 - RNA, 21 nt, synthetic construct
  source 1..21: mol_type = other RNA
aagcaagatatttttataata

SEQ ID NO: 371 - RNA, 23 nt, synthetic construct
  source 1..23: mol_type = other RNA
tattataaaaatatcttgctttt

SEQ ID NO: 372 - RNA, 21 nt, synthetic construct
  source 1..21: mol_type = other RNA
gacaaaataactcactataat

SEQ ID NO: 373 - RNA, 23 nt, synthetic construct
  source 1..23: mol_type = other RNA
attatagtgagttattttgtcaa

SEQ ID NO: 374 - RNA, 21 nt, synthetic construct
  source 1..21: mol_type = other RNA
aggattttgagtgtaaaagga

SEQ ID NO: 375 - RNA, 23 nt, synthetic construct
  source 1..23: mol_type = other RNA
tccttttacactcaaaatccttt

SEQ ID NO: 376 - RNA, 21 nt, synthetic construct
  source 1..21: mol_type = other RNA
gttccggatatttgaactttt

SEQ ID NO: 377 - RNA, 23 nt, synthetic construct
  source 1..23: mol_type = other RNA
aaaagttcaaatatccggaaccg

SEQ ID NO: 378 - RNA, 21 nt, synthetic construct
  source 1..21: mol_type = other RNA
cagatcaaacacaatttcagt

SEQ ID NO: 379 - RNA, 23 nt, synthetic construct
  source 1..23: mol_type = other RNA
actgaaattgtgtttgatctgca

SEQ ID NO: 380 - RNA, 21 nt, synthetic construct
  source 1..21: mol_type = other RNA
tgacaaaataactcactataa

SEQ ID NO: 381 - RNA, 23 nt, synthetic construct
  source 1..23: mol_type = other RNA
ttatagtgagttattttgtcaat

SEQ ID NO: 382 - RNA, 21 nt, synthetic construct
  source 1..21: mol_type = other RNA
atttaaacaacaagtaccttt

SEQ ID NO: 383 - RNA, 23 nt, synthetic construct
  source 1..23: mol_type = other RNA
aaaggtacttgttgtttaaatct

SEQ ID NO: 384 - RNA, 21 nt, synthetic construct
  source 1..21: mol_type = other RNA
gacaaaataactcactataat

SEQ ID NO: 385 - RNA, 23 nt, synthetic construct
  source 1..23: mol_type = other RNA
attatagtgagttattttgtcaa

SEQ ID NO: 386 - RNA, 21 nt, synthetic construct
  source 1..21: mol_type = other RNA
cagatcaaacacaatttcagt

SEQ ID NO: 387 - RNA, 23 nt, synthetic construct
  source 1..23: mol_type = other RNA
actgaaattgtgtttgatctgca

SEQ ID NO: 388 - RNA, 21 nt, synthetic construct
  source 1..21: mol_type = other RNA
atttaaacaacaagtaccttt

SEQ ID NO: 389 - RNA, 23 nt, synthetic construct
  source 1..23: mol_type = other RNA
aaaggtacttgttgtttaaatct

SEQ ID NO: 390 - RNA, 21 nt, synthetic construct
  source 1..21: mol_type = other RNA
tgacaaaataactcactataa

SEQ ID NO: 391 - RNA, 23 nt, synthetic construct
  source 1..23: mol_type = other RNA
ttatagtgagttattttgtcaat

SEQ ID NO: 392 - RNA, 21 nt, synthetic construct
  source 1..21: mol_type = other RNA
gacaaaataactcactataat

SEQ ID NO: 393 - RNA, 23 nt, synthetic construct
  source 1..23: mol_type = other RNA
attatagtgagttattttgtcaa

SEQ ID NO: 394 - RNA, 21 nt, synthetic construct
  source 1..21: mol_type = other RNA
gttccggatatttgaactttt

SEQ ID NO: 395 - RNA, 23 nt, synthetic construct
  source 1..23: mol_type = other RNA
aaaagttcaaatatccggaaccg

SEQ ID NO: 396 - RNA, 21 nt, synthetic construct
  source 1..21: mol_type = other RNA
tgcagatcaaacacaatttca

SEQ ID NO: 397 - RNA, 23 nt, synthetic construct
  source 1..23: mol_type = other RNA
tgaaattgtgtttgatctgcaga

SEQ ID NO: 398 - RNA, 21 nt, synthetic construct
  source 1..21: mol_type = other RNA
aagcaagatatttttataata

SEQ ID NO: 399 - RNA, 23 nt, synthetic construct
  source 1..23: mol_type = other RNA
tattataaaaatatcttgctttt

SEQ ID NO: 400 - RNA, 21 nt, synthetic construct
  source 1..21: mol_type = other RNA
aagcaagatatttttataata

SEQ ID NO: 401 - DNA, 25 bp, synthetic construct
  source 1..25: mol_type = other DNA
  misc_feature 1..23: note = RNA
  misc_feature 24..25: note = DNA
tattataaaaatatcttgctttttt

SEQ ID NO: 402 - DNA, 25 bp, synthetic construct
  source 1..25: mol_type = other DNA
  misc_feature 1..23: note = RNA
  misc_feature 24..25: note = DNA
tattataaaaatatcttgctttttt

SEQ ID NO: 403 - RNA, 21 nt, synthetic construct
  source 1..21: mol_type = other RNA
aagcaagatatttttataata

SEQ ID NO: 404 - DNA, 25 bp, synthetic construct
  source 1..25: mol_type = other DNA
  misc_feature 1..23: note = RNA
  misc_feature 24..25: note = DNA
tattataaaaatatcttgctttttt

SEQ ID NO: 405 - RNA, 21 nt, synthetic construct
  source 1..21: mol_type = other RNA
aagcaagatatttttataata

SEQ ID NO: 406 - RNA, 21 nt, synthetic construct
  source 1..21: mol_type = other RNA
  modified_base 1: mod_base = OTHER; note = 2'-O-methyl adenosine
  modified_base 1^2: mod_base = OTHER; note = phosphorothioate linkage
  modified_base 2: mod_base = OTHER; note = 2'-O-methyl adenosine
  modified_base 2^3: mod_base = OTHER; note = phosphorothioate linkage
  modified_base 3: mod_base = OTHER; note = 2'-fluoro guanosine
  modified_base 4: mod_base = OTHER; note = 2'-O-methyl cytidine
  modified_base 5: mod_base = OTHER; note = 2'-fluoro adenosine
  modified_base 6: mod_base = OTHER; note = 2'-O-methyl adenosine
  modified_base 7: mod_base = OTHER; note = 2'-fluoro guanosine
  modified_base 8: mod_base = OTHER; note = 2'-O-methyl adenosine
  modified_base 9: mod_base = OTHER; note = 2'-fluoro uridine
  modified_base 10: mod_base = OTHER; note = 2'-fluoro adenosine
  modified_base 11: mod_base = OTHER; note = 2'-fluoro uridine
  modified_base 12: mod_base = OTHER; note = u = 2'-O-methyl uridine
  modified_base 13: mod_base = OTHER; note = 2'-fluoro uridine
  modified_base 14..15: mod_base = OTHER; note = 2'-O-methyl uridine
  modified_base 16: mod_base = OTHER; note = 2'-fluoro adenosine
  modified_base 17: mod_base = OTHER; note = 2'-O-methyl uridine
  modified_base 18: mod_base = OTHER; note = 2'-fluoro adenosine
  modified_base 19: mod_base = OTHER; note = 2'-O-methyl adenosine
  modified_base 20: mod_base = OTHER; note = 2'-O-methyl uridine
  modified_base 21: mod_base = OTHER; note = 2'-O-methyl adenosine
aagcaagatatttttataata

SEQ ID NO: 407 - DNA, 21 bp, Homo sapiens
  source 1..21: mol_type = unassigned DNA
aagcaagatatttttataata

(19) United States
(12) Patent Application Publication
(10) Pub. No.: US 2026/0097063 A1
(43) Pub. Date: Apr. 9, 2026

(54) ANTI-C5 ANTIBODY/C5 IRNA DOSING REGIMENS FOR TREATING C5-ASSOCIATED DISEASES

(71) Applicant: Regeneron Pharmaceuticals, Inc., Tarrytown, NY (US)

(72) Inventors: Umesh Chaudhari, Kendall Park, NJ (US); Kuan-Ju Lin, Stamford, CT (US); Lorah Perlee, Westport, CT (US); George D. Yancopoulos, Yorktown Heights, NY (US)

(21) Appl. No.: 19/347,304

(22) Filed: Oct. 1, 2025

Related U.S. Application Data
(60) Provisional application No. 63/701,665, filed on Oct. 1, 2024.
(60) Provisional application No. 63/758,703, filed on Feb. 14, 2025.
(60) Provisional application No. 63/784,847, filed on Apr. 7, 2025.
(60) Provisional application No. 63/869,961, filed on Aug. 25, 2025.
(60) Provisional application No. 63/870,664, filed on Aug. 26, 2025.

(51) Int. Cl. A61K 31/7125; A61K 31/122; A61K 31/198; A61K 31/4402; A61K 31/4709; A61K 31/475; A61K 31/519; A61K 31/616; A61K 31/727; A61K 33/06; A61K 38/13; A61K 39/00; A61K 39/395; A61P 21/04; C07K 16/18; C12N 15/113

(52) U.S. Cl. CPC A61K 31/7125; A61K 31/122; A61K 31/198; A61K 31/4402; A61K 31/4709; A61K 31/475; A61K 31/519; A61K 31/616; A61K 31/727; A61K 33/06; A61K 38/13; A61K 39/3955; A61P 21/04; C07K 16/18; C12N 15/113; A61K 2039/54; A61K 2039/545; A61K 2039/55; C12N 2310/14; C12N 2310/315; C12N 2310/321; C12N 2310/322; C12N 2310/351

(57) ABSTRACT

The present disclosure includes methods of treating C5-associated disease or disorder, such as myasthenia gravis, with a combination that includes an antibody or antigen-binding fragment thereof that binds specifically to C5 and a C5 iRNA which is a glycoconjugate that includes a ligand having terminal N-Acetylgalactosamine (GalNAc) residues and/or N-acetylglucosamine (GlcNAc) residues; or with the C5 iRNA monotherapy.

This application claims the benefit of U.S. provisional patent application No. 63/870,664, filed Aug. 26, 2025; U.S. provisional patent application No. 63/869,961, filed Aug. 25, 2025; U.S. provisional patent application No. 63/784,847, filed Apr. 7, 2025; U.S. provisional patent application No. 63/758,703, filed Feb. 14, 2025; and U.S. provisional patent application No. 63/701,665, filed Oct. 1, 2024; each of which is herein incorporated by reference in its entirety.

### SEQUENCE LISTING

The sequence listing of the present application is submitted electronically as an ST.26 formatted xml file with a file name “SeqList11936”, creation date of Sep. 25, 2024, and a size of 503,576 bytes. This sequence listing submitted is part of the specification and is herein incorporated by reference in its entirety.

### FIELD

The field of the present disclosure relates to C5 iRNA monotherapies as well as co-formulations and combination therapies that include an antibody or antigen-binding fragment thereof that specifically binds complement component 5 (C5) and a C5 iRNA for use in methods of treating or preventing a C5-associated disease or disorder, such as myasthenia gravis.

### BACKGROUND

C5 is a target for several rare diseases, including myasthenia gravis (MG), paroxysmal nocturnal hemoglobinuria (PNH), atypical hemolytic uremic syndrome, and neuromyelitis.

MG is a rare autoimmune disease of the neuromuscular junction (NMJ) due to the production of antibodies that bind to the acetylcholine receptor (AChR), often leading to complement-mediated damage of the NMJ. Worldwide prevalence of the disease is approximately 200 per million persons, and there are approximately 60,000 patients in the United States. Clinically, the disease is characterized by fatigable muscle weakness. Patients may present with diplopia/ptosis or peripheral muscle fatigable weakness that is often multifocal or generalized. Generalized myasthenia gravis (gMG) comprises 85% of patients. When gMG affects the intercostal muscles or diaphragm, breathing is affected and the disease can become life-threatening, requiring hospitalization and treatment with mechanical ventilation (myasthenic crisis). Up to 20% of patients have a myasthenic crisis within the first 2 years after diagnosis (Stetefeld et al., Fortschr Neurol Psychiatr 2018; 86 (5): 301-307; Wendell, Neurohospitalist 2011; 1 (1): 16-22).

Evidence suggests that in order to effectively treat paroxysmal nocturnal hemoglobinuria and avoid undesirable hemolysis breakthrough, complete inhibition of complement is necessary. See Peffault de Latour et al., Assessing complement blockade in patients with paroxysmal nocturnal hemoglobinuria receiving eculizumab. Blood. 2015 Jan. 29; 125 (5): 775-83-Epub 2014 Dec. 4; Hill et al., Thrombosis in paroxysmal nocturnal hemoglobinuria. Blood 2013; 121 (25): 4985-96; and Nakayama et al., Eculizumab Dosing Intervals Longer than 17 Days May Be Associated with Greater Risk of Breakthrough Hemolysis in Patients with Paroxysmal Nocturnal Hemoglobinuria. Biol Pharm Bull 2016; 39 (2): 285-8.

Evidence has suggested that in order to achieve therapeutic efficacy for treating myasthenia gravis, with a complement 5 inhibitor, complete complement inhibition was necessary. A Phase 2 clinical trial designed to determine whether near-complete complement inhibition is necessary to achieve maximal clinical benefit in patients with generalized myasthenia gravis was conducted. A conclusion reached from the trial was that near-complete complement inhibition was superior to submaximal complement inhibition. See Howard et al., Clinical Effects of the Self-administered Subcutaneous Complement Inhibitor Zilucoplan in Patients With Moderate to Severe Generalized Myasthenia Gravis: Results of a Phase 2 Randomized, Double-Blind, Placebo-Controlled, Multicenter Clinical Trial. JAMA Neurol. 2020 May 1; 77 (5): 582-592. The dosing of the therapeutic anti-C5 antibodies eculizumab and ravulizumab also suggested that complete complement inhibition in an MG patient, like PNH, was needed for efficacy. Eculizumab (sold as Soliris) and Ravulizumab (sold as Ultomiris) are each approved in the US for treatment of PNH and MG employing the same dosing regimens.

### SUMMARY

The present invention provides methods for treating myasthenia gravis where superior efficacy and safety is achieved without complete complement blockade. This aspect of the present invention is surprising and unexpected. As mentioned herein, other C5-mediated diseases, such as paroxysmal nocturnal hemoglobinuria (PNH), are believed to require complete inhibition of complement for efficacy. In addition, clinical data from a Phase 2 trial using Zilucoplan (a C5 inhibitor) suggested that better efficacy was associated with greater levels of complement inhibition. This suggested that, to treat MG effectively, maximal complement inhibition was necessary. As stated in the report of the Phase 2 Zilucoplan trial, “Near-complete complement inhibition was associated with a faster onset and greater magnitude of benefit than submaximal complement inhibition, and favorable safety and tolerability were observed”. Howard et al., Clinical Effects of the Self-administered Subcutaneous Complement Inhibitor Zilucoplan in Patients With Moderate to Severe Generalized Myasthenia Gravis: Results of a Phase 2 Randomized, Double-Blind, Placebo-Controlled, Multicenter Clinical Trial. JAMA Neurol. 2020 May 1; 77 (5): 582-592). The Cemdisiran monotherapy discussed herein demonstrated superior efficacy in the absence of complete complement inhibition. This aspect is favorable since achieving a high degree of efficacy in treating MG without full complement inhibition may lead to a favorable safety profile. Complement inhibition can increase the risk of infection.

The present invention provides a method for treating or preventing a C5-associated disease or disorder (e.g., myasthenia gravis) in a subject in need thereof comprising administering to the subject a therapeutically effective amount of an antibody or antigen-binding fragment thereof that binds specifically to C5 (e.g., Pozelimab) in combination with a therapeutically effective amount of a C5 iRNA (e.g., Cemdisiran Na⁺ form, other salt form or free form), wherein the combination of the antibody or antigen-binding fragment thereof and the C5 iRNA (e.g., Cemdisiran Na⁺ form, other salt form or free form) is administered to the subject by: (a) a subcutaneous injection or intravenous infusion of a co-formulation that comprises both (i) the anti-C5 antibody or antigen-binding fragment thereof and (ii) the C5 iRNA (e.g., Cemdisiran Na⁺ form, other salt form or free form); (b) subcutaneous injections or intravenous infusions of separate formulations that each comprise either (i) the anti-C5 antibody or antigen-binding fragment thereof or (ii) the C5 iRNA (e.g., Cemdisiran Na⁺ form, other salt form or free form); (c) a subcutaneous injection or intravenous infusion of an anti-C5 antibody or antigen-binding fragment thereof; or (d) a subcutaneous injection or intravenous infusion of C5 iRNA (e.g., Cemdisiran Na⁺ form, other salt form or free form). In an embodiment of the invention, the method comprises administering, to the subject, one or more: (a) approximately 200 mg (±20 mg) doses of the anti-C5 antibody or antigen-binding fragment thereof (e.g., Pozelimab), and/or approximately 200 mg (±20 mg) C5 iRNA (e.g., Cemdisiran Na⁺ form, other salt form or free form) or a molar equivalent amount thereof or 10-13 (±2) micromoles of C5 iRNA (e.g., Cemdisiran Na⁺ form, other salt form or free form); (b) approximately 200 mg (±20 mg) doses of C5 iRNA (e.g., Cemdisiran Na⁺ form, other salt form or free form) or a molar equivalent amount thereof or 10-13 (±2) micromoles of C5 iRNA (e.g., Cemdisiran Na⁺ form, other salt form or free form); (c) approximately 600 mg (±60 mg) doses of C5 iRNA (e.g., Cemdisiran Na⁺ form, other salt form or free form) or a molar equivalent amount thereof or about 32-35 or 33.8 (±4) micromoles of C5 iRNA (e.g., Cemdisiran Na⁺ form, other salt form or free form); (d) approximately 200 mg (±20 mg) doses of anti-C5 antibody or antigen-binding fragment thereof (e.g., Pozelimab); (e) approximately 200 mg (±20 mg) doses of the anti-C5 antibody or antigen-binding fragment thereof (e.g., Pozelimab), about every 4 weeks (±7 days), and approximately 200 mg (±20 mg) C5 iRNA (e.g., Cemdisiran Na⁺ form, other salt form or free form) or a molar equivalent amount thereof or 10-13 (±2) micromoles C5 iRNA (e.g., Cemdisiran Na⁺ form, other salt form or free form), about every 4 weeks (±7 days); (e) approximately 200 mg (±20 mg) doses of C5 iRNA (e.g., Cemdisiran Na⁺ form, other salt form or free form) or a molar equivalent amount thereof or 10-13 (±2) micromoles C5 iRNA (e.g., Cemdisiran Na⁺ form, other salt form or free form) about every 4 weeks (±7 days); (f) approximately 600 mg (±60 mg) doses of C5 iRNA (e.g., Cemdisiran Na⁺ form, other salt form or free form) or a molar equivalent amount thereof about every 12 weeks (±7 days) or 84 days (±7 days) or 3 months (±7 days) or 4 times per year (±7 days); (g) approximately 200 mg (±20 mg) doses of anti-C5 antibody or antigen-binding fragment thereof (e.g., Pozelimab) about every 4 weeks (±7 days); or (h) approximately 32-35 or 33.8 (±4) micromole doses of C5 iRNA (e.g., Cemdisiran Na⁺ form, other salt form or free form) about every 12 weeks (±7 days) or 84 days (±7 days) or 3 months (±7 days) or 4 times per year (±7 days). In an embodiment of the invention, the method (e.g., for treating MG) comprises administering (e.g., in a saline composition; e.g., by subcutaneous injection, for example, with an autoinjector) one or more doses of approximately 32-35 or 33.8 (±4) micromoles Cemdisiran salt form; administering one or more doses of approximately 32-35 or 33.8 (±4) micromoles Cemdisiran salt form once every 12 weeks (±7 days); administering one or more doses of approximately 32-35 or 33.8 micromoles (±4) Cemdisiran salt form once every 84 days (±7 days); administering one or more doses of approximately 32-35 or 33.8 micromoles (±4) Cemdisiran salt form once every 3 months (±7 days); administering one or more doses of approximately 600 mg (±60 mg) Cemdisiran salt form; administering one or more doses of approximately 600 mg (±60 mg) Cemdisiran salt form once every 12 weeks (±7 days); administering one or more doses of approximately 600 mg (±60 mg) Cemdisiran salt form once every 84 days (±7 days); administering one or more doses of approximately 600 mg (±60 mg) Cemdisiran salt form once every 3 months (±7 days); administering one or more doses of approximately 32-35 or 33.8 (±4) micromoles Cemdisiran Na⁺ salt form; administering one or more doses of approximately 32-35 or 33.8 (±4) micromoles Cemdisiran Na⁺ salt form once every 12 weeks (±7 days); administering one or more doses of approximately 32-35 or 33.8 micromoles (±4) Cemdisiran Na⁺ salt form once every 84 days (±7 days); administering one or more doses of approximately 32-35 or 33.8 micromoles (±4) Cemdisiran Na⁺ salt form once every 3 months (±7 days); administering one or more doses of approximately 600 mg (±60 mg) Cemdisiran Na⁺ salt form; administering one or more doses of approximately 600 mg (±60 mg) Cemdisiran Na⁺ salt form once every 12 weeks (±7 days); administering one or more doses of approximately 600 mg (±60 mg) Cemdisiran Na⁺ salt form once every 84 days (±7 days); administering one or more doses of approximately 600 mg (±60 mg) Cemdisiran Na⁺ salt form once every 3 months (±7 days); administering one or more doses of an amount of Cemdisiran salt form that is the molar equivalent of 600 mg (±60 mg) Cemdisiran Na⁺ salt form; administering one or more doses of an amount of Cemdisiran salt form that is the molar equivalent of 600 mg (±60 mg) Cemdisiran Na⁺ salt form once every 12 weeks (±7 days); administering one or more doses of an amount of Cemdisiran salt form that is the molar equivalent of 600 mg (±60 mg) Cemdisiran Na⁺ salt form once every 84 days (±7 days); and/or administering one or more doses of an amount of Cemdisiran salt form that is the molar equivalent of 600 mg (±60 mg) Cemdisiran Na⁺ salt form once every 3 months (±7 days).

In an embodiment of the invention, the anti-C5 antibody or antigen-binding fragment thereof and the C5 iRNA (e.g., Cemdisiran Na⁺ form, other salt form or free form) are in a single co-formulation.

In an embodiment of the invention, one or more doses of the anti-C5 antibody or antigen-binding fragment thereof are administered to the subject subcutaneously; and/or one or more doses of the C5 iRNA (e.g., Cemdisiran Na⁺ form, other salt form or free form) are administered to the subject subcutaneously. In an embodiment of the invention, the anti-C5 antibody or antigen-binding fragment thereof and the C5 iRNA (e.g., Cemdisiran Na⁺ form, other salt form or free form) are in separate formulations wherein the C5 iRNA (e.g., Cemdisiran Na⁺ form, other salt form or free form) is administered before, after, or simultaneously with the anti-C5 antibody or antigen-binding fragment thereof. For example, in an embodiment of the invention, the C5 iRNA (e.g., Cemdisiran Na⁺ form, other salt form or free form) and the anti-C5 antibody or antigen-binding fragment thereof are administered within 30 minutes or within 1 hour of each other.

In an embodiment of the invention, the ratio of concentrations (mg/ml) of anti-C5 antibody or antigen-binding fragment thereof and the C5 iRNA (e.g., Cemdisiran Na⁺ form, other salt form or free form) in the co-formulation is 1:1; e.g., wherein the co-formulation includes about 100 mg/ml (±10 mg/mg) anti-C5 antibody or antigen-binding fragment thereof and the concentration of the C5 iRNA (e.g., Cemdisiran Na⁺ form, other salt form or free form) in the co-formulation is about 100 mg/ml (±10 mg/mg). In an embodiment of the invention, a co-formulation that includes the anti-C5 antibody or antigen-binding fragment and C5 iRNA (e.g., Cemdisiran Na⁺ form, other salt form or free form) is administered in a single approximately 2 ml (±0.2 ml) injection. In an embodiment of the invention, the method of the present invention includes steps wherein each dose of the anti-C5 antibody or antigen-binding fragment thereof is administered about every 4 weeks (±1, 2, 3, 4, 5, 6, or 7 days); and/or each dose of the C5 iRNA (e.g., Cemdisiran Na⁺ form, other salt form or free form) is administered about every 4 weeks (±1, 2, 3, 4, 5, 6, or 7 days). In an embodiment of the invention, a combination administered to a subject includes (i) a co-formulation comprises: the C5 iRNA (e.g., Cemdisiran Na⁺ form, other salt form or free form) conjugated to a ligand that comprises one or more terminal N-Acetylgalactosamine (GalNAc) and/or N-acetylglucosamine (GlcNAc) residues, the anti-C5 antibody or antigen-binding fragment thereof, and a pharmaceutically acceptable carrier; wherein the co-formulation has a pH of greater than or less than about 6; or (ii) a first formulation comprising the anti-C5 antibody or antigen-binding fragment thereof and a pharmaceutically acceptable carrier; and a second formulation comprising the C5 iRNA (e.g., Cemdisiran Na⁺ form, other salt form or free form) conjugated to a ligand that comprises one or more terminal N-Acetylgalactosamine (GalNAc) and/or N-acetylglucosamine (GlcNAc) residues, and a pharmaceutically acceptable carrier.

In an embodiment of the invention, a method the present invention includes the step of receiving, after two or more doses of the C5 iRNA (e.g., Cemdisiran Na⁺ form, other salt form or free form) which separated by a 12 week (±7 days), 84 day (±7 days) or 3 month (±7 days) interval, administering one or more doses of the C5 iRNA (e.g., Cemdisiran Na⁺ form, other salt form or free form) separated by an interval of greater than or less than said 12 weeks (±7 days), 84 days (±7 days) or 3 months (±7 days), for example, if based on clinical outcomes of the subject and based on the treating physician's judgment, taking such a step is needed.

In an embodiment of the invention, the C5 iRNA (e.g., Cemdisiran Na⁺ form, other salt form or free form) and the anti-C5 antibody or antigen-binding fragment are in a co-formulation, for example, that has a pH of about 6.5 or a pH within not less than 0.5 to 6.0 or of 6.5±0.2;
that comprises a buffer selected from a histidine-based buffer, a citrate-based buffer, a phosphate-based buffer an acetate-based buffer, and combinations thereof;
that comprises about 10-35, 35-45, 20-50, 20, 25, 30, 35, 40, 45 or 50 mM buffer;
that comprises a viscosity reducer selected from an inorganic salt, an amino acid, and a combination thereof;
that comprises a viscosity reducer selected from (D- or L-) arginine, L-arginine HCl, (D- or L-) alanine, proline, (D- or L-) valine, glycine, (D- or L-) serine, (D- or L-) phenylalanine, (D- or L-) lysine, (D- or L-) glutamate and salts thereof, an inorganic salt, NaCl, pyridoxamine, L-ornithine, thiamine phosphoric acid ester chloride dihydrate, benzenesulfonic acid, pyridoxine, and a combination thereof;
that comprises about 20-140, 20, 25, 30, 35, 40, 45, 50, 55, 60, 65, 70, 75, 80, 85, 90, 95, 100, 105, 110, 115, 120, 125, 130, 135 or 140 mM viscosity reducer;
that comprises a stabilizer which is a polyol or sugar;
that comprises a stabilizer selected from trehalose, sorbitol, mannitol, taurine, propane sulfonic acid, L-proline, sucrose, glycerol, threitol, maltitol, polyethylene glycol (PEG), PEG3350, and combinations thereof;
that comprises about 0.8-3.6, 0.8, 0.9, 1.0, 1.25, 1.50, 2.0, 2.25, 2.50, 2.75, 3.00, 3.1, 3.2, 3.3, 3.4, 3.5 or 3.6% (w/v) stabilizer;
that comprises a non-ionic surfactant selected from a polyoxyethylene glycol alkyl ether; glucoside alkyl ether; polyoxyethylene glycol octylphenol ether; polyoxyethylene glycol alkylphenol ether; glycerol alkyl ester; polyoxyethylene glycol sorbitan alkyl ester; sorbitan alkyl ester; block copolymer of polypropylene glycol; block copolymer of polyethylene glycol; polysorbate; and combinations thereof;
that comprises a non-ionic surfactant selected from octaethylene glycol monododecyl ether; pentaethylene glycol monododecyl ether; polyoxypropylene glycol alkyl ether; decyl glucoside, lauryl glucoside, octyl glucoside; triton X-100; nonoxynol-9; glyceryl laurate; cocamide MEA, cocamide DEA, dodecyldimethylamine oxide; poloxamer; polyethoxylated tallow amine (POEA); polysorbate-20 (PS20); polysorbate-80 (PS80); and combinations thereof;
that comprises about 0.025, 0.05, 0.075, 0.1, 0.125, 0.15, or 0.175% (w/v) non-ionic surfactant;
that comprises one or more viscosity reducers;
that comprises one or more viscosity reducers at a concentration of about 5 mM to about 100 mM each;
that comprises one or more viscosity reducers at a concentration of about 50 mM to about 75 mM each;
that comprises a viscosity reducer which is selected from an amino acid, a dicarboxylic acid, an inorganic salt, an ester of citric acid, a xanthine, and combinations thereof;
that comprises a viscosity reducer which is selected from arginine, adipic acid, NaCl, lysine, proline, histidine, caffeine, phenylalanine, triethyl citrate; and combinations thereof;
that comprises a viscosity reducer which is an amino acid which is the L-enantiomer thereof or the D-enantiomer thereof;
that comprises a viscosity reducer which is the conjugate base or salt thereof of an acid viscosity reducer;
that comprises about 96% or more anti-C5 antibody or antigen-binding fragment thereof purity as assessed by size exclusion chromatography after about 1 month of storage at 2-8° C.;
that comprises about 94% or more C5 iRNA (e.g., Cemdisiran Na⁺ form, other salt form or free form) purity as assessed by anion exchange chromatography after about 1 month of storage at 2-8° C.;
that comprises a 1:1 ratio of concentration of C5 iRNA (e.g., Cemdisiran Na⁺ form, other salt form or free form) to anti-C5 antibody or antigen-binding fragment thereof in milligrams per milliliter;
that comprises a viscosity reducer selected from arginine, adipic acid, NaCl, lysine, proline, histidine, caffeine, phenylalanine, triethyl citrate, and combinations thereof; and/or that comprises a viscosity reducer which is 75 mM arginine, 75 mM adipate, 75 mM NaCl, 75 mM lysine, 75 mM aspartate, 75 mM proline, 50 mM histidine, 50 mM caffeine, 50 mM phenylalanine, 75 mM triethyl citrate, and combinations thereof; wherein, if the buffer is histidine-based, the total histidine concentration of the co-formulation is 50 mM. In an embodiment of the invention, the co-formulation comprises: the C5 iRNA (e.g., Cemdisiran Na⁺ form, other salt form or free form); the anti-C5 antibody or antigen-binding fragment thereof; a buffer; a viscosity reducer; a stabilizer; and a non-ionic surfactant; and pH of greater than or less than about 6. In an embodiment of the invention, said combination is administered as a co-formulation, which comprises: Cemdisiran; Pozelimab that was expressed in and isolated from a mammalian host cell that includes beta-hexosaminidase; a buffer; a viscosity reducer; a stabilizer; and a non-ionic surfactant; at a pH of about 6.5; and/or the Cemdisiran is in an aqueous pharmaceutical formulation comprising a buffer and/or NaCl.

In an embodiment of the invention, the anti-C5 antibody or antigen-binding fragment is Pozelimab which was expressed in a mammalian host cell (e.g., a CHO cell) and/or the C5 iRNA is Cemdisiran (e.g., Cemdisiran Na⁺ form, other salt form or free form) which was chemically synthesized. In an embodiment of the invention, a combination that is administered as a co-formulation, comprises:
no more than about 2.1 parts per million molar ratio of beta-hexosaminidase to anti-C5 antibody or antigen-binding fragment;
no more than about 0.170 micrograms/ml beta-hexosaminidase;
no more than about 0.04 micrograms/ml beta-hexosaminidase; and/or
about 0.04; 0.05; 0.06; 0.0605; 0.063; 0.07; 0.0765; 0.078; 0.08; 0.14; 0.141; 0.15; 0.1525; 0.166; or 0.17 micrograms/ml beta-hexosaminidase; or no more than any of such concentrations.

In an embodiment of the invention, the anti-C5 antibody or antigen-binding fragment thereof comprises: a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10; a HCVR that comprises the HCDR1, HCDR2 and HCDR3 of a HCVR that comprises the amino acid sequence set forth in SEQ ID NO: 18, and a LCVR that comprises the LCDR1, LCDR2 and LCDR3 of a LCVR that comprises the amino acid sequence set forth in SEQ ID NO: 26; a HCVR that comprises the HCDR1, HCDR2 and HCDR3 of a HCVR that comprises the amino acid sequence set forth in SEQ ID NO: 34, and a LCVR that comprises the LCDR1, LCDR2 and LCDR3 of a LCVR that comprises the amino acid sequence set forth in SEQ ID NO: 42; a HCVR that comprises the HCDR1, HCDR2 and HCDR3 of a HCVR that comprises the amino acid sequence set forth in SEQ ID NO: 50, and a LCVR that comprises the LCDR1, LCDR2 and LCDR3 of a LCVR that comprises the amino acid sequence set forth in SEQ ID NO: 58; a HCVR that comprises the HCDR1, HCDR2 and HCDR3 of a HCVR that comprises the amino acid sequence set forth in SEQ ID NO: 66, and a LCVR that comprises the LCDR1, LCDR2 and LCDR3 of a LCVR that comprises the amino acid sequence set forth in SEQ ID NO: 74; a HCVR that comprises the HCDR1, HCDR2 and HCDR3 of a HCVR that comprises the amino acid sequence set forth in SEQ ID NO: 82, and a LCVR that comprises the LCDR1, LCDR2 and LCDR3 of a LCVR that comprises the amino acid sequence set forth in SEQ ID NO: 90; a HCVR that comprises the HCDR1, HCDR2 and HCDR3 of a HCVR that comprises the amino acid sequence set forth in SEQ ID NO: 98, and a LCVR that comprises the LCDR1, LCDR2 and LCDR3 of a LCVR that comprises the amino acid sequence set forth in SEQ ID NO: 106; a HCVR that comprises the HCDR1, HCDR2 and HCDR3 of a HCVR that comprises the amino acid sequence set forth in SEQ ID NO: 98, and a LCVR that comprises the LCDR1, LCDR2 and LCDR3 of a LCVR that comprises the amino acid sequence set forth in SEQ ID NO: 114; a HCVR that comprises the HCDR1, HCDR2 and HCDR3 of a HCVR that comprises the amino acid sequence set forth in SEQ ID NO: 122, and a LCVR that comprises the LCDR1, LCDR2 and LCDR3 of a LCVR that comprises the amino acid sequence set forth in SEQ ID NO: 106; a HCVR that comprises the HCDR1, HCDR2 and HCDR3 of a HCVR that comprises the amino acid sequence set forth in SEQ ID NO: 98, and a LCVR that comprises the LCDR1, LCDR2 and LCDR3 of a LCVR that comprises the amino acid sequence set forth in SEQ ID NO: 130; a HCVR that comprises the HCDR1, HCDR2 and HCDR3 of a HCVR that comprises the amino acid sequence set forth in SEQ ID NO: 138, and a LCVR that comprises the LCDR1, LCDR2 and LCDR3 of a LCVR that comprises the amino acid sequence set forth in SEQ ID NO: 106; a HCVR that comprises the HCDR1, HCDR2 and HCDR3 of a HCVR that comprises the amino acid sequence set forth in SEQ ID NO: 146, and a LCVR that comprises the LCDR1, LCDR2 and LCDR3 of a LCVR that comprises the amino acid sequence set forth in SEQ ID NO: 106; a HCVR that comprises the HCDR1, HCDR2 and HCDR3 of a HCVR that comprises the amino acid sequence set forth in SEQ ID NO: 122, and a LCVR that comprises the LCDR1, LCDR2 and LCDR3 of a LCVR that comprises the amino acid sequence set forth in SEQ ID NO: 130; a HCVR that comprises the HCDR1, HCDR2 and HCDR3 of a HCVR that comprises the amino acid sequence set forth in SEQ ID NO: 146, and a LCVR that comprises the LCDR1, LCDR2 and LCDR3 of a LCVR that comprises the amino acid sequence set forth in SEQ ID NO: 114; a HCVR that comprises the HCDR1, HCDR2 and HCDR3 of a HCVR that comprises the amino acid sequence set forth in SEQ ID NO: 146, and a LCVR that comprises the LCDR1, LCDR2 and LCDR3 of a LCVR that comprises the amino acid sequence set forth in SEQ ID NO: 130; a HCVR that comprises the HCDR1, HCDR2 and HCDR3 of a HCVR that comprises the amino acid sequence set forth in SEQ ID NO: 138, and a LCVR that comprises the LCDR1, LCDR2 and LCDR3 of a LCVR that comprises the amino acid sequence set forth in SEQ ID NO: 130; a HCVR that comprises the HCDR1, HCDR2 and HCDR3 of a HCVR that comprises the amino acid sequence set forth in SEQ ID NO: 154, and a LCVR that comprises the LCDR1, LCDR2 and LCDR3 of a LCVR that comprises the amino acid sequence set forth in SEQ ID NO: 162; a HCVR that comprises the HCDR1, HCDR2 and HCDR3 of a HCVR that comprises the amino acid sequence set forth in SEQ ID NO: 170, and a LCVR that comprises the LCDR1, LCDR2 and LCDR3 of a LCVR that comprises the amino acid sequence set forth in SEQ ID NO: 178; a HCVR that comprises the HCDR1, HCDR2 and HCDR3 of a HCVR that comprises the amino acid sequence set forth in SEQ ID NO: 186, and a LCVR that comprises the LCDR1, LCDR2 and LCDR3 of a LCVR that comprises the amino acid sequence set forth in SEQ ID NO: 194; a HCVR that comprises the HCDR1, HCDR2 and HCDR3 of a HCVR that comprises the amino acid sequence set forth in SEQ ID NO: 202, and a LCVR that comprises the LCDR1, LCDR2 and LCDR3 of a LCVR that comprises the amino acid sequence set forth in SEQ ID NO: 210; a HCVR that comprises the HCDR1, HCDR2 and HCDR3 of a HCVR that comprises the amino acid sequence set forth in SEQ ID NO: 218, and a LCVR that comprises the LCDR1, LCDR2 and LCDR3 of a LCVR that comprises the amino acid sequence set forth in SEQ ID NO: 226; a HCVR that comprises the HCDR1, HCDR2 and HCDR3 of a HCVR that comprises the amino acid sequence set forth in SEQ ID NO: 234, and a LCVR that comprises the LCDR1, LCDR2 and LCDR3 of a LCVR that comprises the amino acid sequence set forth in SEQ ID NO: 242; a HCVR that comprises the HCDR1, HCDR2 and HCDR3 of a HCVR that comprises the amino acid sequence set forth in SEQ ID NO: 250, and a LCVR that comprises the LCDR1, LCDR2 and LCDR3 of a LCVR that comprises the amino acid sequence set forth in SEQ ID NO: 258; a HCVR that comprises the HCDR1, HCDR2 and HCDR3 of a HCVR that comprises the amino acid sequence set forth in SEQ ID NO: 266, and a LCVR that comprises the LCDR1, LCDR2 and LCDR3 of a LCVR that comprises the amino acid sequence set forth in SEQ ID NO: 258; a HCVR that comprises the HCDR1, HCDR2 and HCDR3 of a HCVR that comprises the amino acid sequence set forth in SEQ ID NO: 274, and a LCVR that comprises the LCDR1, LCDR2 and LCDR3 of a LCVR that comprises the amino acid sequence set forth in SEQ ID NO: 282; a HCVR that comprises the HCDR1, HCDR2 and HCDR3 of a HCVR that comprises the amino acid sequence set forth in SEQ ID NO: 290, and a LCVR that comprises the LCDR1, LCDR2 and LCDR3 of a LCVR that comprises the amino acid sequence set forth in SEQ ID NO: 298; a HCVR that comprises the HCDR1, HCDR2 and HCDR3 of a HCVR that comprises the amino acid sequence set forth in SEQ ID NO: 306, and a LCVR that comprises the LCDR1, LCDR2 and LCDR3 of a LCVR that comprises the amino acid sequence set forth in SEQ ID NO: 314; a HCVR that comprises the HCDR1, HCDR2 and HCDR3 of a HCVR that comprises the amino acid sequence set forth in SEQ ID NO: 322, and a LCVR that comprises the LCDR1, LCDR2 and LCDR3 of a LCVR that comprises the amino acid sequence set forth in SEQ ID NO: 330; or a HCVR that comprises the HCDR1, HCDR2 and HCDR3 of a HCVR that comprises the amino acid sequence set forth in SEQ ID NO: 338, and a LCVR that comprises the LCDR1, LCDR2 and LCDR3 of a LCVR that comprises the amino acid sequence set forth in SEQ ID NO: 346; a heavy chain variable region (HCVR) including an HCDR1 that comprises the amino acid sequence set forth in SEQ ID NO: 4, an HCDR2 that comprises the amino acid sequence set forth in SEQ ID NO: 6, an HCDR3 that comprises the amino acid sequence set forth in SEQ ID NO: 8, and a light chain variable region (LCVR) including an LCDR1 that comprises the amino acid sequence set forth in SEQ ID NO: 12, an LCDR2 that comprises the amino acid sequence set forth in SEQ ID NO: 14, an LCDR3 that comprises the amino acid sequence set forth in SEQ ID NO: 16; a HCVR including an HCDR1 that comprises the amino acid sequence set forth in SEQ ID NO: 20, an HCDR2 that comprises the amino acid sequence set forth in SEQ ID NO: 22, an HCDR3 that comprises the amino acid sequence set forth in SEQ ID NO: 24, and a LCVR including an LCDR1 that comprises the amino acid sequence set forth in SEQ ID NO: 28, an LCDR2 that comprises the amino acid sequence set forth in SEQ ID NO: 30, an LCDR3 that comprises the amino acid sequence set forth in SEQ ID NO: 32; a HCVR including an HCDR1 that comprises the amino acid sequence set forth in SEQ ID NO: 36, an HCDR2 that comprises the amino acid sequence set forth in SEQ ID NO: 38, an HCDR3 that comprises the amino acid sequence set forth in SEQ ID NO: 40, and a LCVR including an LCDR1 that comprises the amino acid sequence set forth in SEQ ID NO: 44, an LCDR2 that comprises the amino acid sequence set forth in SEQ ID NO: 46, an LCDR3 that comprises the amino acid sequence set forth in SEQ ID NO: 48; a HCVR including an HCDR1 that comprises the amino acid sequence set forth in SEQ ID NO: 52, an HCDR2 that comprises the amino acid sequence set forth in SEQ ID NO: 54, an HCDR3 that comprises the amino acid sequence set forth in SEQ ID NO: 56, and a LCVR including an LCDR1 that comprises the amino acid sequence set forth in SEQ ID NO: 60, an LCDR2 that comprises the amino acid sequence set forth in SEQ ID NO: 62, an LCDR3 that comprises the amino acid sequence set forth in SEQ ID NO: 64; a HCVR including an HCDR1 that comprises the amino acid sequence set forth in SEQ ID NO: 68, an HCDR2 that comprises the amino acid sequence set forth in SEQ ID NO: 70, an HCDR3 that comprises the amino acid sequence set forth in SEQ ID NO: 72, and a LCVR including an LCDR1 that comprises the amino acid sequence set forth in SEQ ID NO: 76, an LCDR2 that comprises the amino acid sequence set forth in SEQ ID NO: 78, an LCDR3 that comprises the amino acid sequence set forth in SEQ ID NO: 80; a HCVR including an HCDR1 that comprises the amino acid sequence set forth in SEQ ID NO: 84, an HCDR2 that comprises the amino acid sequence set forth in SEQ ID NO: 86, an HCDR3 that comprises the amino acid sequence set forth in SEQ ID NO: 88, and a LCVR including an LCDR1 that comprises the amino acid sequence set forth in SEQ ID NO: 92, an LCDR2 that comprises the amino acid sequence set forth in SEQ ID NO: 94, an LCDR3 that comprises the amino acid sequence set forth in SEQ ID NO: 96; a HCVR including an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a LCVR including an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a HCVR including an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a LCVR including an LCDR1 that comprises the amino acid sequence set forth in SEQ ID NO: 116, an LCDR2 that comprises the amino acid sequence set forth in SEQ ID NO: 118, an LCDR3 that comprises the amino acid sequence set forth in SEQ ID NO: 120; a HCVR including an HCDR1 that comprises the amino acid sequence set forth in SEQ ID NO: 124, an HCDR2 that comprises the amino acid sequence set forth in SEQ ID NO: 126, an HCDR3 that comprises the amino acid sequence set forth in SEQ ID NO: 128, and a LCVR including an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a HCVR including an HCDR1 that comprises the amino acid sequence set forth in SEQ ID NO: 100, an HCDR2 that comprises the amino acid sequence set forth in SEQ ID NO: 102, an HCDR3 that comprises the amino acid sequence set forth in SEQ ID NO: 104, and a LCVR including an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a HCVR including an HCDR1 that comprises the amino acid sequence set forth in SEQ ID NO: 140, an HCDR2 that comprises the amino acid sequence set forth in SEQ ID NO: 142, an HCDR3 that comprises the amino acid sequence set forth in SEQ ID NO: 144, and a LCVR including an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a HCVR including an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a LCVR including an LCDR1 that comprises the amino acid sequence set forth in SEQ ID NO: 108, an LCDR2 that comprises the amino acid sequence set forth in SEQ ID NO: 110, an LCDR3 that comprises the amino acid sequence set forth in SEQ ID NO: 112; a HCVR including an HCDR1 that comprises the amino acid sequence set forth in SEQ ID NO: 124, an HCDR2 that comprises the amino acid sequence set forth in SEQ ID NO: 126, an HCDR3 that comprises the amino acid sequence set forth in SEQ ID NO: 128, and a LCVR including an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a HCVR including an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a LCVR including an LCDR1 that comprises the amino acid sequence set forth in SEQ ID NO: 116, an LCDR2 that comprises the amino acid sequence set forth in SEQ ID NO: 118, an LCDR3 that comprises the amino acid sequence set forth in SEQ ID NO: 120; a HCVR including an HCDR1 that comprises the amino acid sequence set forth in SEQ ID NO: 148, an HCDR2 that comprises the amino acid sequence set forth in SEQ ID NO: 150, an HCDR3 that comprises the amino acid sequence set forth in SEQ ID NO: 152, and a LCVR including an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a HCVR including an HCDR1 that comprises the amino acid sequence set forth in SEQ ID NO: 140, an HCDR2 that comprises the amino acid sequence set forth in SEQ ID NO: 142, an HCDR3 that comprises the amino acid sequence set forth in SEQ ID NO: 144, and a LCVR including an LCDR1 that comprises the amino acid sequence set forth in SEQ ID NO: 132, an LCDR2 that comprises the amino acid sequence set forth in SEQ ID NO: 134, an LCDR3 that comprises the amino acid sequence set forth in SEQ ID NO: 136; a HCVR including an HCDR1 that comprises the amino acid sequence set forth in SEQ ID NO: 156, an HCDR2 that comprises the amino acid sequence set forth in SEQ ID NO: 158, an HCDR3 that comprises the amino acid sequence set forth in SEQ ID NO: 160, and a LCVR including an LCDR1 that comprises the amino acid sequence set forth in SEQ ID NO: 164, an LCDR2 that comprises the amino acid sequence set forth in SEQ ID NO: 166, an LCDR3 that comprises the amino acid sequence set forth in SEQ ID NO: 168; a HCVR including an HCDR1 that comprises the amino acid sequence set forth in SEQ ID NO: 172, an HCDR2 that comprises the amino acid sequence set forth in SEQ ID NO: 174, an HCDR3 that comprises the amino acid sequence set forth in SEQ ID NO: 176, and a LCVR including an LCDR1 that comprises the amino acid sequence set forth in SEQ ID NO: 180, an LCDR2 that comprises the amino acid sequence set forth in SEQ ID NO: 182, an LCDR3 that comprises the amino acid sequence set forth in SEQ ID NO: 184; a HCVR including an HCDR1 that comprises the amino acid sequence set forth in SEQ ID NO: 188, an HCDR2 that comprises the amino acid sequence set forth in SEQ ID NO: 190, an HCDR3 that comprises the amino acid sequence set forth in SEQ ID NO: 192, and a LCVR including an LCDR1 that comprises the amino acid sequence set forth in SEQ ID NO: 196, an LCDR2 that comprises the amino acid sequence set forth in SEQ ID NO: 198, an LCDR3 that comprises the amino acid sequence set forth in SEQ ID NO: 200; a HCVR including an HCDR1 that comprises the amino acid sequence set forth in SEQ ID NO: 204, an HCDR2 that comprises the amino acid sequence set forth in SEQ ID NO: 206, an HCDR3 that comprises the amino acid sequence set forth in SEQ ID NO: 208, and a LCVR including an LCDR1 that comprises the amino acid sequence set forth in SEQ ID NO: 212, an LCDR2 that comprises the amino acid sequence set forth in SEQ ID NO: 214, an LCDR3 that comprises the amino acid sequence set forth in SEQ ID NO: 216; a HCVR including an HCDR1 that comprises the amino acid sequence set forth in SEQ ID NO: 220, an HCDR2 that comprises the amino acid sequence set forth in SEQ ID NO: 222, an HCDR3 that comprises the amino acid sequence set forth in SEQ ID NO: 224, and a LCVR including an LCDR1 that comprises the amino acid sequence set forth in SEQ ID NO: 228, an LCDR2 that comprises the amino acid sequence set forth in SEQ ID NO: 230, an LCDR3 that comprises the amino acid sequence set forth in SEQ ID NO: 232; a HCVR including an HCDR1 that comprises the amino acid sequence set forth in SEQ ID NO: 236, an HCDR2 that comprises the amino acid sequence set forth in SEQ ID NO: 238, an HCDR3 that comprises the amino acid sequence set forth in SEQ ID NO: 240, and a LCVR including an LCDR1 that comprises the amino acid sequence set forth in SEQ ID NO: 244, an LCDR2 that comprises the amino acid sequence set forth in SEQ ID NO: 246, an LCDR3 that comprises the amino acid sequence set forth in SEQ ID NO: 248; a HCVR including an HCDR1 that comprises the amino acid sequence set forth in SEQ ID NO: 252, an HCDR2 that comprises the amino acid sequence set forth in SEQ ID NO: 254, an HCDR3 that comprises the amino acid sequence set forth in SEQ ID NO: 256, and a LCVR including an LCDR1 that comprises the amino acid sequence set forth in SEQ ID NO: 260, an LCDR2 that comprises the amino acid sequence set forth in SEQ ID NO: 262, an LCDR3 that comprises the amino acid sequence set forth in SEQ ID NO: 264; a HCVR including an HCDR1 that comprises the amino acid sequence set forth in SEQ ID NO: 268, an HCDR2 that comprises the amino acid sequence set forth in SEQ ID NO: 270, an HCDR3 that comprises the amino acid sequence set forth in SEQ ID NO: 272, and a LCVR including an LCDR1 that comprises the amino acid sequence set forth in SEQ ID NO: 260, an LCDR2 that comprises the amino acid sequence set forth in SEQ ID NO: 262, an LCDR3 that comprises the amino acid sequence set forth in SEQ ID NO: 264; a HCVR including an HCDR1 that comprises the amino acid sequence set forth in SEQ ID NO: 276, an HCDR2 that comprises the amino acid sequence set forth in SEQ ID NO: 278, an HCDR3 that comprises the amino acid sequence set forth in SEQ ID NO: 280, and a LCVR including an LCDR1 that comprises the amino acid sequence set forth in SEQ ID NO: 284, an LCDR2 that comprises the amino acid sequence set forth in SEQ ID NO: 286, an LCDR3 that comprises the amino acid sequence set forth in SEQ ID NO: 288; a HCVR including an HCDR1 that comprises the amino acid sequence set forth in SEQ ID NO: 292, an HCDR2 that comprises the amino acid sequence set forth in SEQ ID NO: 294, an HCDR3 that comprises the amino acid sequence set forth in SEQ ID NO: 296, and a LCVR including an LCDR1 that comprises the amino acid sequence set forth in SEQ ID NO: 300, an LCDR2 that comprises the amino acid sequence set forth in SEQ ID NO: 302, an LCDR3 that comprises the amino acid sequence set forth in SEQ ID NO: 304; a HCVR including an HCDR1 that comprises the amino acid sequence set forth in SEQ ID NO: 308, an HCDR2 that comprises the amino acid sequence set forth in SEQ ID NO: 310, an HCDR3 that comprises the amino acid sequence set forth in SEQ ID NO: 312, and a LCVR including an LCDR1 that comprises the amino acid sequence set forth in SEQ ID NO: 316, an LCDR2 that comprises the amino acid sequence set forth in SEQ ID NO: 318, an LCDR3 that comprises the amino acid sequence set forth in SEQ ID NO: 320; a HCVR including an HCDR1 that comprises the amino acid sequence set forth in SEQ ID NO: 324, an HCDR2 that comprises the amino acid sequence set forth in SEQ ID NO: 326, an HCDR3 that comprises the amino acid sequence set forth in SEQ ID NO: 328, and a LCVR including an LCDR1 that comprises the amino acid sequence set forth in SEQ ID NO: 332, an LCDR2 that comprises the amino acid sequence set forth in SEQ ID NO: 334, an LCDR3 that comprises the amino acid sequence set forth in SEQ ID NO: 336; or a HCVR including an HCDR1 that comprises the amino acid sequence set forth in SEQ ID NO: 340, an HCDR2 that comprises the amino acid sequence set forth in SEQ ID NO: 342, an HCDR3 that comprises the amino acid sequence set forth in SEQ ID NO: 344, and a LCVR including an LCDR1 that comprises the amino acid sequence set forth in SEQ ID NO: 348, an LCDR2 that comprises the amino acid sequence set forth in SEQ ID NO: 350, an LCDR3 that comprises the amino acid sequence set forth in SEQ ID NO: 352; a heavy chain variable region (HCVR) that comprises the amino acid sequence set forth in SEQ ID NO: 2, a light chain variable region (LCVR) that comprises the amino acid sequence set forth in SEQ ID NO: 10; a HCVR that comprises the amino acid sequence set forth in SEQ ID NO: 18, a LCVR that comprises the amino acid sequence set forth in SEQ ID NO: 26; a HCVR that comprises the amino acid sequence set forth in SEQ ID NO: 34, a LCVR that comprises the amino acid sequence set forth in SEQ ID NO: 42; a HCVR that comprises the amino acid sequence set forth in SEQ ID NO: 50, a LCVR that comprises the amino acid sequence set forth in SEQ ID NO: 58; a HCVR that comprises the amino acid sequence set forth in SEQ ID NO: 66, a LCVR that comprises the amino acid sequence set forth in SEQ ID NO: 74; a HCVR that comprises the amino acid sequence set forth in SEQ ID NO: 82, a LCVR that comprises the amino acid sequence set forth in SEQ ID NO: 90; a HCVR that comprises the amino acid sequence set forth in SEQ ID NO: 98, a LCVR that comprises the amino acid sequence set forth in SEQ ID NO: 106; a HCVR that comprises the amino acid sequence set forth in SEQ ID NO: 98, a LCVR that comprises the amino acid sequence set forth in SEQ ID NO: 114; a HCVR that comprises the amino acid sequence set forth in SEQ ID NO: 122, a LCVR that comprises the amino acid sequence set forth in SEQ ID NO: 106; a HCVR that comprises the amino acid sequence set forth in SEQ ID NO: 98, a LCVR that comprises the amino acid sequence set forth in SEQ ID NO: 130; a HCVR that comprises the amino acid sequence set forth in SEQ ID NO: 138, a LCVR that comprises the amino acid sequence set forth in SEQ ID NO: 106; a HCVR that comprises the amino acid sequence set forth in SEQ ID NO: 146, a LCVR that comprises the amino acid sequence set forth in SEQ ID NO: 106; a HCVR that comprises the amino acid sequence set forth in SEQ ID NO: 122, a LCVR that comprises the amino acid sequence set forth in SEQ ID NO: 130; a HCVR that comprises the amino acid sequence set forth in SEQ ID NO: 146, a LCVR that comprises the amino acid sequence set forth in SEQ ID NO: 114; a HCVR that comprises the amino acid sequence set forth in SEQ ID NO: 146, a LCVR that comprises the amino acid sequence set forth in SEQ ID NO: 130; a HCVR that comprises the amino acid sequence set forth in SEQ ID NO: 138, a LCVR that comprises the amino acid sequence set forth in SEQ ID NO: 130; a HCVR that comprises the amino acid sequence set forth in SEQ ID NO: 154, a LCVR that comprises the amino acid sequence set forth in SEQ ID NO: 162; a HCVR that comprises the amino acid sequence set forth in SEQ ID NO: 170, a LCVR that comprises the amino acid sequence set forth in SEQ ID NO: 178; a HCVR that comprises the amino acid sequence set forth in SEQ ID NO: 186, a LCVR that comprises the amino acid sequence set forth in SEQ ID NO: 194; a HCVR that comprises the amino acid sequence set forth in SEQ ID NO: 202, a LCVR that comprises the amino acid sequence set forth in SEQ ID NO: 210; a HCVR that comprises the amino acid sequence set forth in SEQ ID NO: 218, a LCVR that comprises the amino acid sequence set forth in SEQ ID NO: 226; a HCVR that comprises the amino acid sequence set forth in SEQ ID NO: 234, a LCVR that comprises the amino acid sequence set forth in SEQ ID NO: 242; a HCVR that comprises the amino acid sequence set forth in SEQ ID NO: 250, a LCVR that comprises the amino acid sequence set forth in SEQ ID NO: 258; a HCVR that comprises the amino acid sequence set forth in SEQ ID NO: 266, a LCVR that comprises the amino acid sequence set forth in SEQ ID NO: 258; a HCVR that comprises the amino acid sequence set forth in SEQ ID NO: 274, a LCVR that comprises the amino acid sequence set forth in SEQ ID NO: 282; a HCVR that comprises the amino acid sequence set forth in SEQ ID NO: 290, a LCVR that comprises the amino acid sequence set forth in SEQ ID NO: 298; a HCVR that comprises the amino acid sequence set forth in SEQ ID NO: 306, a LCVR that comprises the amino acid sequence set forth in SEQ ID NO: 314; a HCVR that comprises the amino acid sequence set forth in SEQ ID NO: 322, a LCVR that comprises the amino acid sequence set forth in SEQ ID NO: 330; or a HCVR that comprises the amino acid sequence set forth in SEQ ID NO: 338, a LCVR that comprises the amino acid sequence set forth in SEQ ID NO: 346.

In an embodiment of the invention, the anti-C5 antibody or antigen-binding fragment comprises a heavy chain including the amino acid sequence: QVQLQESGPGLVKPSETLSLTCTVSGDSVSSSYWTWIRQPPGKGLEWIGYIYYSGSSNYN PSLKSRATISVDTSKNQFSLKLSSVTAADTAVYYCAREGNVDTTMIFDYWGQGTLVTVSSA STKGPSVFPLAPCSRSTSESTAALGCLVKDYFPEPVTVSWNSGALTSGVHTFPAVLQSSG LYSLSSVVTVPSSSLGTKTYTCNVDHKPSNTKVDKRVESKYGPPCPPCPAPEFLGGPSVFL FPPKPKDTLMISRTPEVTCVVVDVSQEDPEVQFNWYVDGVEVHNAKTKPREEQFNSTYRV VSVLTVLHQDWLNGKEYKCKVSNKGLPSSIEKTISKAKGQPREPQVYTLPPSQEEMTKNQ VSLTCLVKGFYPSDIAVEWESNGQPENNYKTTPPVLDSDGSFFLYSRLTVDKSRWQEGNV FSCSVMHEALHNHYTQKSLSLSLGK (SEQ ID NO: 358); optionally, lacking the C-terminal lysine; and a light chain including the amino acid sequence: AIQMTQSPSSLSASVGDRVTITCRASQGIRNDLGWYQQKPGKAPKLLIYAASSLQSGVPSR FAGRGSGTDFTLTISSLOPEDFATYYCLQDFNYPWTFGQGTKVEIKRTVAAPSVFIFPPSDE QLKSGTASVVCLLNNFYPREAKVQWKVDNALQSGNSQESVTEQDSKDSTYSLSSTLTLSK ADYEKHKVYACEVTHQGLSSPVTKSFNRGEC (SEQ ID NO: 359); for example, wherein the anti-C5 antibody or antigen-binding fragment thereof is Pozelimab.

In an embodiment of the invention, the C5 iRNA comprises an RNA strand that is complementary to an mRNA transcribed from the C5 gene sense strand DNA sequence AAGCAAGATATTTTTATAATA (SEQ ID NO: 407; nucleotides 782-802 of SEQ ID NO: 360). In an embodiment of the invention, the C5 iRNA is a double-stranded ribonucleic acid (dsRNA) agent including a sense strand and an antisense strand; wherein the antisense strand comprises a region of complementarity including at least 17 contiguous nucleotides differing by no more than 3 nucleotides from the nucleotide sequence of 5′UAUUAUAAAAAUAUCUUGCUUUU3′ (SEQ ID NO: 364); and wherein the dsRNA agent comprises at least one modified nucleotide. In an embodiment of the invention, the C5 iRNA is a double-stranded ribonucleic acid (dsRNA) agent including a sense strand and an antisense strand; wherein the antisense strand comprises 5′-asasGfcAfaGfaUfAfUfuUfuuAfuAfaua-3′, and the antisense strand comprises 5′-usAfsUfuAfuaAfaAfauaUfcUfuGfcuususudTdT-3′; wherein
a, g, c and u are 2′-O-methyl (2′-OMe) A, G, C, and U, respectively;
Af, Gf, Cf and Uf are 2′-fluoro A, G, C and U, respectively;
dT is a deoxy-thymine nucleotide;
s is a phosphorothioate linkage; and
   wherein the sense strand is conjugated at the 3′-terminus to the ligand

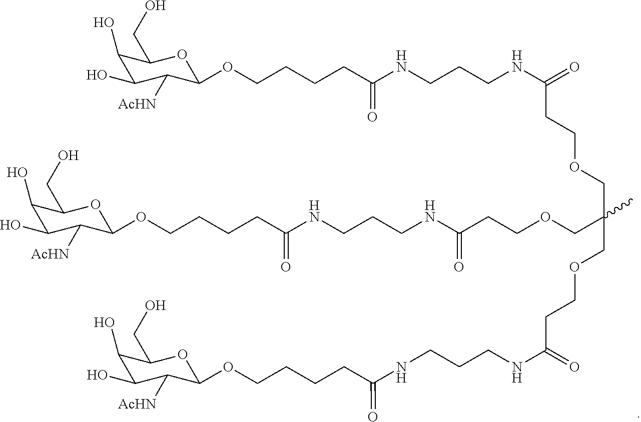 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In an embodiment of the invention, the C5 iRNA is a duplex comprising [(2S,4R)-1-{1-[(2-acetamido-2-deoxy-β-Dgalactopyranosyl)oxy]-16,16-bis({3-[(3-{5-[(2-acetamido-2-deoxy-β-D-galactopyranosyl)oxy]pentanamido}propyl)amino]-3-oxopropoxy}methyl)-5,11,18-trioxo-14-oxa-6,10,17-triazanonacosan-29-oyl}-4-hydroxypyrrolidin-2-yl]methyl hydrogen all-P-ambo-2′-O-methyl-P-thioadenylyl-(3′→5′)-2′-Omethyl-P-thioadenylyl-(3′→5′)-2′-deoxy-2′-fluoroguanylyl-(3′→5′)-2′-O-methylcytidylyl-(3′→5′)-2′-deoxy-2′-fluoroadenylyl-(3′→5′)-2′-O-methyladenylyl-(3′→5′)-2′-deoxy-2′-fluoroguanylyl-(3′→5′)-2′-O-methyladenylyl-(3′→5′)-2′-deoxy-2′-fluorouridylyl-(3′→5′)-2′-deoxy-2′-fluoroadenylyl-(3′→5′)-2′-deoxy-2′-fluorouridylyl-(3′→5′)-2′-O-methyluridylyl-(3′→5′)-2′-deoxy-2′-fluorouridylyl-(3′→5′)-2′-O-methyluridylyl-(3′→5′)-2′-Omethyluridylyl-(3′→5′)-2′-deoxy-2′-fluoroadenylyl-(3′→5′)-2′-Omethyluridylyl-(3′→5′)-2′-deoxy-2′-fluoroadenylyl-(3′→5′)-2′-Omethyladenylyl-(3′→5′)-2′-O-methyluridylyl-(3′→5′)-2′-Omethyl-3′-adenylate and all-P-ambo-thymidylyl-(5′→3′)-thymidylyl-(5′→3′)-2′-O-methyl-P-thiouridylyl-(5′→3′)-2′-Omethyl-P-thiouridylyl-(5′→3′)-2′-O-methyluridylyl-(5′→3′)-2′-Omethyluridylyl-(5′→3′)-2′-O-methylcytidylyl-(5′→3′)-2′-deoxy-2′-fluoroguanylyl-(5′→3′)-2′-O-methyluridylyl-(5′→3′)-2′-deoxy-2′-fluorouridylyl-(5′→3′)-2′-O-methylcytidylyl-(5′→3′)-2′-deoxy-2′-fluorouridylyl-(5′→3′)-2′-O-methyladenylyl-(5′→3′)-2′-Omethyluridylyl-(5′→3′)-2′-O-methyladenylyl-(5′→3′)-2′-deoxy-2′-fluoroadenylyl-(5′→3′)-2′-O-methyladenylyl-(5′→3′)-2′-deoxy-2′-fluoroadenylyl-(5′→3′)-2′-O-methyladenylyl-(5′→3′)-2′-Omethyluridylyl-(5′→3′)-2′-deoxy-2′-fluoroadenylyl-(5′→3′)-2′-Omethyluridylyl-(5′→3′)-2′-deoxy-2′-fluoro-P-thiouridylyl-(5′→3′)-2′-deoxy-2′-fluoro-P-thioadenylyl-(5′→3′)-2′-O-methyluridine. In an embodiment of the invention, the C5 iRNA is Cemdisiran, for example, the Na⁺ salt form, another salt form or free form. In an embodiment of the invention, the anti-C5 antibody or antigen-binding fragment thereof is Pozelimab and/or the C5 iRNA is Cemdisiran (preferably Na⁺ form).

The methods of the present invention relate to treatment or prevention of a C5-associated disease or disorder. In an embodiment of the invention, the C5-associated disease or disorder is selected from adult respiratory distress syndrome (ARDS); age-related macular degeneration (AMD); allergy; Alport's syndrome; Alzheimer's disease; an autoimmune disease; an immune complex disorder; an inflammatory disorder; angiopathic thrombosis and protein-losing enteropathy); asthma; atherosclerosis; bronchoconstriction; bullous pemphigoid; C3 glomerulopathy; capillary leak syndrome; CHAPLE disease (CD55 deficiency with hyperactivation of complement); a chemical injury due to irritant gasses and/or chemicals; chronic obstructive pulmonary disease (COPD); complement activation due to a burn; complement activation due to frostbite; complement activation due to obesity; complement activation due to sepsis; Crohn's disease; diabetes; diabetic macular edema (DME); diabetic nephropathy; diabetic retinopathy; a disorder of inappropriate or undesirable complement activation; dry AMD; dyspnea; emphysema; epilepsy; fibrogenic dust disease; geographic atrophy (GA); glomerulopathy; Goodpasture's Syndrome; Guillain-Barre Syndrome; a hemodialysis complication; hemolytic anemia; hemoptysis; hereditary angioedema; hyperacute allograft rejection; hypersensitivity pneumonitis; immune complex-associated inflammation; an infectious disease; inflammation of an autoimmune disease; inherited CD59 deficiency; an injury due to inert dusts and/or minerals; interleukin-2 induced toxicity during IL-2 therapy; a lung disease or disorder; lupus nephritis; membranoproliferative glomerulonephritis; membranoproliferative nephritis; mesenteric artery reperfusion after aortic reconstruction; multiple sclerosis; myasthenia gravis; myocardial infarction; a neurological disorder; neuromyelitis optica; ocular angiogenesis; an ocular disease; an organic dust disease; a parasitic disease; Parkinson's disease; paroxysmal nocturnal hemoglobinuria (PNH); pneumonia; a post-ischemic reperfusion condition; a proteinuric kidney disease; progressive kidney failure; psoriasis; pulmonary embolisms and infarcts; pulmonary fibrosis; pulmonary vasculitis; a renal disorder; renal ischemia; renal ischemia-reperfusion injury; rheumatoid arthritis; schizophrenia; SLE nephritis; a smoke injury; stroke; systemic inflammatory response in post-pump syndrome due to cardiopulmonary bypass or renal bypass; systemic lupus erythematosus (SLE); a thermal injury; a traumatic brain injury; uveitis; vasculitis; wet AMD; xenograft rejection; and combinations thereof. Preferably, the C5-associated disease or disorder is myasthenia gravis; for example, the myasthenia gravis is generalized myasthenia gravis, ocular myasthenia gravis, Class I myasthenia gravis, Class II myasthenia gravis, Class IIa myasthenia gravis, Class IIb myasthenia gravis, Class III myasthenia gravis, Class IIIa myasthenia gravis, Class IIIb myasthenia gravis, Class IV myasthenia gravis, Class IVa myasthenia gravis, Class IVb myasthenia gravis, Class V myasthenia gravis, Early-onset myasthenia gravis, Late-onset myasthenia gravis, Thymoma-associated myasthenia gravis, myasthenia gravis with anti-MuSK antibodies, Ocular myasthenia gravis characterized by symptoms from periocular muscles, and/or myasthenia gravis with no detectable AChR and MuSK antibodies.

The present invention includes embodiments wherein the subject has been diagnosed with myasthenia gravis (e.g., generalized myasthenia gravis, ocular myasthenia gravis, Class I myasthenia gravis, Class II myasthenia gravis, Class IIa myasthenia gravis, Class IIb myasthenia gravis, Class III myasthenia gravis, Class IIIa myasthenia gravis, Class IIIb myasthenia gravis, Class IV myasthenia gravis, Class IVa myasthenia gravis, Class IVb myasthenia gravis, Class V myasthenia gravis, Early-onset myasthenia gravis, Late-onset myasthenia gravis, Thymoma-associated myasthenia gravis, myasthenia gravis with anti-MuSK antibodies, Ocular myasthenia gravis characterized by symptoms from periocular muscles, and/or myasthenia gravis with no detectable AChR and MuSK antibodies).

The present invention provides a method for treating or preventing a C5-associated disease or disorder (e.g., MG) in a subject in need thereof comprising administering, to the subject, a dose of C5 iRNA (e.g., Cemdisiran Na⁺ form, other salt form or free form) wherein one week after said administering, the subject serum CH50 decreases by about 33%, two weeks after said administering, the subject serum CH50 decreases by about 56%, four week after said administering, the subject serum CH50 decreases by about 77%; relative to before the administration.

The present invention provides a method for treating or preventing a C5-associated disease or disorder in a subject in need thereof comprising administering, to the subject, a dose of C5 iRNA (e.g., Cemdisiran Na⁺ form, other salt form or free form) wherein the subject serum CH50 decreases by at least 33, 56, 74 or 77%, for example, wherein the CH50 decreases by 1, 2, 4, 8, 16, 20 or 24 weeks after treatment initiation.

In an embodiment of the invention, a subject in a method set forth herein has been diagnosed with myasthenia gravis by a method comprising an electrophysiologic test, a repetitive nerve stimulation (RNS) test, a single-fiber electromyography (SFEMG) test, an Edrophonium (Tensilon) test, an Ice-pack test, Chest computed tomography (CT), and/or Magnetic resonance imaging (MRI). In an embodiment of the invention, a method of the present invention further includes the step of diagnosing the subject with myasthenia gravis.

In an embodiment of the invention, a subject has myasthenia gravis and one or more criteria selected from: (a) a history of abnormal neuromuscular transmission by repetitive nerve stimulation; (b) a history of abnormal neuromuscular transmission by single-fiber electromyography; (c) a history of a positive anticholinesterase test; and (d) improvement in symptoms with anticholinesterase treatment. In an embodiment of the invention, the subject has myasthenia gravis and is positive for anti-acetylcholinesterase (anti-AChR) antibodies or anti-Lipoprotein receptor-related protein 4 (anti-LRP4) antibodies.

In an embodiment of the invention, a method of the present invention further includes administering to the subject one or more further therapeutic agents (e.g., a chemotherapeutic agent, an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and an anti-thrombotic agent). In an embodiment of the invention, the further therapeutic agent is not an anti-CD20 antibody, an anti-FcRN antibody, and/or an androgen deprivation therapy. The present invention may include a step of administering to the subject an initial loading dose (e.g., by intravenous infusion) of C5 iRNA (e.g., Cemdisiran Na⁺ form, other salt form or free form) and/or antibody or fragment thereof that binds specifically to C5.

In an embodiment of the invention, a method of the present invention is continued up until or terminated when: the subject becomes pregnant; the subject contracts a serious meningococcal infection; the subject suffers a liver impairment as confirmed by a repeat test on 1 or more of the following criteria and no other reason can be found to explain the following lab abnormalities:—ALT (Alanine aminotransferase) or AST (Aspartate aminotransferase) level >8×ULN (upper limit of normal), or—ALT or AST level >5×ULN for >2 weeks, or—ALT or AST level >3×ULN and total bilirubin >2×ULN (or INR (international normalized ratio) >1.5); and/or the subject makes a suicide attempt.

In an embodiment of the invention, the subject has received (previously or during the course of treatment) one or more of: an anticholinesterase agent, an immunosuppressive drug, a C5 complement inhibitor, a neonatal Fc receptor blocker, prednisone, tacrolimus, rituximab, eculizumab, ravulizumab, efgartigimod, rozanolixizumab, pyridostigmine, plasmapheresis (PLEX) and/or intravenous IgG.

In an embodiment of the invention, during said treatment, the subject is monitored for: Meningococcal infection; Serious infection; Sepsis; Embryofetal toxicity; Immunogenicity; Anti-drug antibody formation; Any clinical Consequences of large Drug-Target-Drug immune complexes in subjects who switch from Eculizumab or Ravulizumab to Pozelimab; Liver transaminase elevations; and/or Injection site reaction; and, optionally, treatment is terminated or suspended if one or more are observed.

In an embodiment of the invention, during or after treatment, the subject achieves one or more of: Improvement in Myasthenia Gravis-Activities of Daily Living (MG-ADL) total score from baseline to week 24; Improvement in Quantitative Myasthenia Gravis (QMG) score from baseline to week 24; ≥3-point improvement from baseline to week 24 on the MG-ADL; ≥5-point improvement from baseline to week 24 on the QMG; At least a 2-point MG-ADL improvement on 2 or more consecutive assessments on the MG-ADL spanning 4 or more weeks during the double-blind treatment period (DBTP); Minimal symptom expression (MSE), defined by a score of 0 to 1 on the MG-ADL at week 24; Improvement in the Myasthenia Gravis Composite (MGC) total score from baseline to week 24; Improvement in Myasthenia Gravis Quality of Life (MG-QOL15r) total score from baseline to week 24; Improvement point threshold of ≥2, 4, 5, 6, 7, 8, 9, or 10 on MG-ADL at week 24; Improvement point threshold of ≥3, 4, 6, 7, 8, 9, or 10 on QMG at week 24; Reduced incidence and severity of treatment-related adverse events (TEAEs), serious adverse events (SAEs), and adverse events of special interest (AESIs) in comparison to placebo through week 24; Improvement from baseline in MG-ADL total score by visit; Improvement from baseline in QMG total score by visit; Reduced time to achievement of a 3-point improvement from baseline on the MG-ADL in comparison to placebo; Reduced time to achievement of a 5-point improvement from baseline on the QMG in comparison to placebo; Improvement from baseline in the physical performance measures of hand grip strength, supine leg lift, supine head lift, onset of dysarthria, and all timed items of the QMG; Improvement from baseline in individual domains (ocular, bulbar, respiratory, and gross motor/limb impairment) of the MG-ADL; Improvement from baseline in sentinel domains (ocular, facial, bulbar, gross motor, axial, and respiratory) of the QMG; Improvement from baseline in continuous variable measures from the MGC; Improvement from baseline in timed step count, cadence, and other exploratory measures using a wearable device; Increased total MG-ADL score and secondary efficacy endpoints in comparison to placebo through week 48 and through week 120; Improvement in patient-reported fatigue as measured by the Neuro-QoL-Fatigue from baseline to week 24; Improvement from baseline in patient-reported health status as measured by the EuroQol-5 Dimensions-5 Levels (EQ-5D-5L); Reduced number of myasthenic crises (including impending crises), days of hospitalization due to MG, and/or procedures related to MG from time periods prior to randomization; Improvement from baseline and percent change from baseline in alternative pathway hemolytic activity assay (AH50) and total C5 over time; Reduced use of rescue therapy from baseline to week 24; Increase in corticosteroid dosage from baseline to week 24; Decrease in corticosteroid dosage from baseline to week 24; Reduced incidence and severity of adverse events through the end of study in comparison to placebo; Improvement from baseline in Modified MGFA Post-Intervention Status (MG-PIS) at week 24, week 48, and week 120; A percent change in serum CH50 by week 1, week 2, week 4, week 12 or week 24, after treatment initiation with Pozelimab monotherapy (200 mg, q4w) of about −18.49, −9.19, −7.06, −6 or −7.31, U/ml, respectively; A percent change in serum CH50 by week 1, week 2, week 4, week 12 or week 24 after treatment initiation with Pozelimab (PZ) and Cemdisiran (CMD) combination (200 mg PZ+200 CMD, q4w) of about −63.31, −83.53, −84.62, −98.91 or −99.57, U/ml, respectively; A percent change in serum CH50 by week 1, week 2, week 4, week 12 or week 24 after treatment initiation with Cemdisiran monotherapy (600 mg, q12w) of about −32.62, −55.75, −76.77, −73.53 or −76.61, U/ml, respectively; A change in MG-ADL total score by week 2, 4, 8, 12, 16, 20 or 24 after treatment initiation with Pozelimab monotherapy (200 mg, q4w) of about −1.31, −1.98, −2.59, −2.66, −2.80, −2.59 or −2.77, respectively; A change in MG-ADL total score by week 2, 4, 8, 12, 16, 20 or 24 after treatment initiation with Pozelimab (PZ)+Cemdisiran (CMD) combination (200 mg PZ+200 mg CMD, q4w) of about −2.56, −3.08, −3.38, −3.76, −3.75, −3.91 or −3.96, respectively; A change in MG-ADL total score by week 2, 4, 8, 12, 16, 20 or 24 after treatment initiation with Cemdisiran monotherapy (600 mg, q12w) of about −2.50, −3.22, −3.96, −4.31, −4.14, −4.59 or −4.52, respectively; An absolute MD-ADL score, by week, 2, 4, 8, 12, 16, 20 or 24 after treatment initiation with Pozelimab (200 mg, q4w), of about 8.50, 7.87, 7.08, 7.13, 6.94, 7.15 or 6.92, respectively; An absolute MD-ADL score, by week, 2, 4, 8, 12, 16, 20 or 24 after treatment initiation with Pozelimab (PZ)+Cemdisiran (CMD) (200 mg PZ+200 mg CMD, q4w), of about 6.91, 6.25, 5.84, 5.45, 5.33, 5.22 or 5.08, respectively; An absolute MD-ADL score, by week, 2, 4, 8, 12, 16, 20 or 24 after treatment initiation with Cemdisiran (600 mg, q12w), of about 6.52, 5.81, 5.08, 4.70, 4.81, 4.44 or 4.53, respectively; A change in QMG total score by week 2, 4, 8, 12, 16, 20 or 24 after treatment initiation with Pozelimab monotherapy (200 mg, q4w) of about −0.83, −0.73, −1.58, −1.78, −1.44, −1.77 or −1.55, respectively; A change in QMG total score by week 2, 4, 8, 12, 16, 20 or 24 after treatment initiation with Pozelimab (PZ) and Cemdisiran (CMD) combination (200 mg PZ+200 CMD, q4w) of about −2.11, −2.77, −2.56, −3.07, −3.27, −3.18 or −3.32, respectively; A change in QMG total score by week 2, 4, 8, 12, 16, 20 or 24 after treatment initiation with Cemdisiran monotherapy (600 mg, q12w) of about −1.95, −2.60, −3.91, −3.94, −4.11, −3.92 or −4.24, respectively; An absolute MD-ADL score, by week, 2, 4, 8, 12, 16, 20 or 24 after treatment initiation with Pozelimab monotherapy (200 mg, q4w), of about 15.33, 15.54, 14.38, 14.41, 14.47, 14.19 or 14.31, respectively; An absolute MD-ADL score, by week, 2, 4, 8, 12, 16, 20 or 24 after treatment initiation with Pozelimab (PZ)+Cemdisiran (CMD) (200 mg PZ+200 mg CMD, q4w) (200 mg, q4w), of about 13.84, 12.85, 12.94, 12.33, 11.97, 11.98 or 11.94, respectively; An absolute MD-ADL score, by week, 2, 4, 8, 12, 16, 20 or 24 after treatment initiation with Cemdisiran monotherapy (600 mg, q12w), of about 13.64, 12.98, 11.69, 11.64, 11.45, 11.76 or 11.44, respectively; A change in MGC score by week 2, 4, 8, 12, 16, 20 or 24 after treatment initiation with Pozelimab (PZ)+Cemdisiran (CMD) combination (200 mg PZ+200 mg CMD, q4w) of about −4.31, −4.77, −5.49, −5.89, −5.85, −6.01 or −6.04, respectively; A change in MGC score by week 2, 4, 8, 12, 16, 20 or 24 after treatment initiation with Cemdisiran monotherapy (600 mg, q12w) of about −4.49, −5.22, −6.55, −7.55, −7.57, −7.69 or −7.78, respectively; A change in MGC score by week 2, 4, 8, 12, 16, 20 or 24 after treatment initiation with Pozelimab (PZ) monotherapy (200 mg, q4w) of about −1.64, −2.13, −3.34, −3.47, −3.30, −3.57 or −3.19, respectively; A change in MG-QoL15r score by week 2, 4, 8, 12, 16, 20 or 24 after treatment initiation with Pozelimab (PZ)+Cemdisiran (CMD) combination (200 mg PZ+200 mg CMD, q4w) of about −2.44, −2.86, −4.00, −3.79, −3.86, −3.91 or −4.25, respectively; A change in MG-QoL15r score by week 2, 4, 8, 12, 16, 20 or 24 after treatment initiation with Cemdisiran monotherapy (600 mg, q12w) of about −1.43, −2.67, −4.08, −4.31, −4.52, −4.65 or −4.68, respectively; and/or A change in MG-QoL15r score by week 2, 4, 8, 12, 16, 20 or 24 after treatment initiation with Pozelimab (PZ) monotherapy (200 mg, q4w) of about −1.65, −1.26, −0.92, −1.76, −1.49, −0.70 or −1.58, respectively.

The present invention provides a method for treating myasthenia gravis, preferably, generalized myasthenia gravis (gMG) in an adult patient who is anti-acetylcholine receptor (AChR) or anti-low-density lipoprotein receptor-related protein 4 (LRP4) antibody positive comprising administering about 600 mg Cemdisiran sodium form or a molar equivalent of another salt form of Cemdisiran or Cemdisiran free form, by subcutaneous injection every about once every 12 weeks or 3 months wherein the Cemdisiran Na⁺ form is represented by the chemical structure:

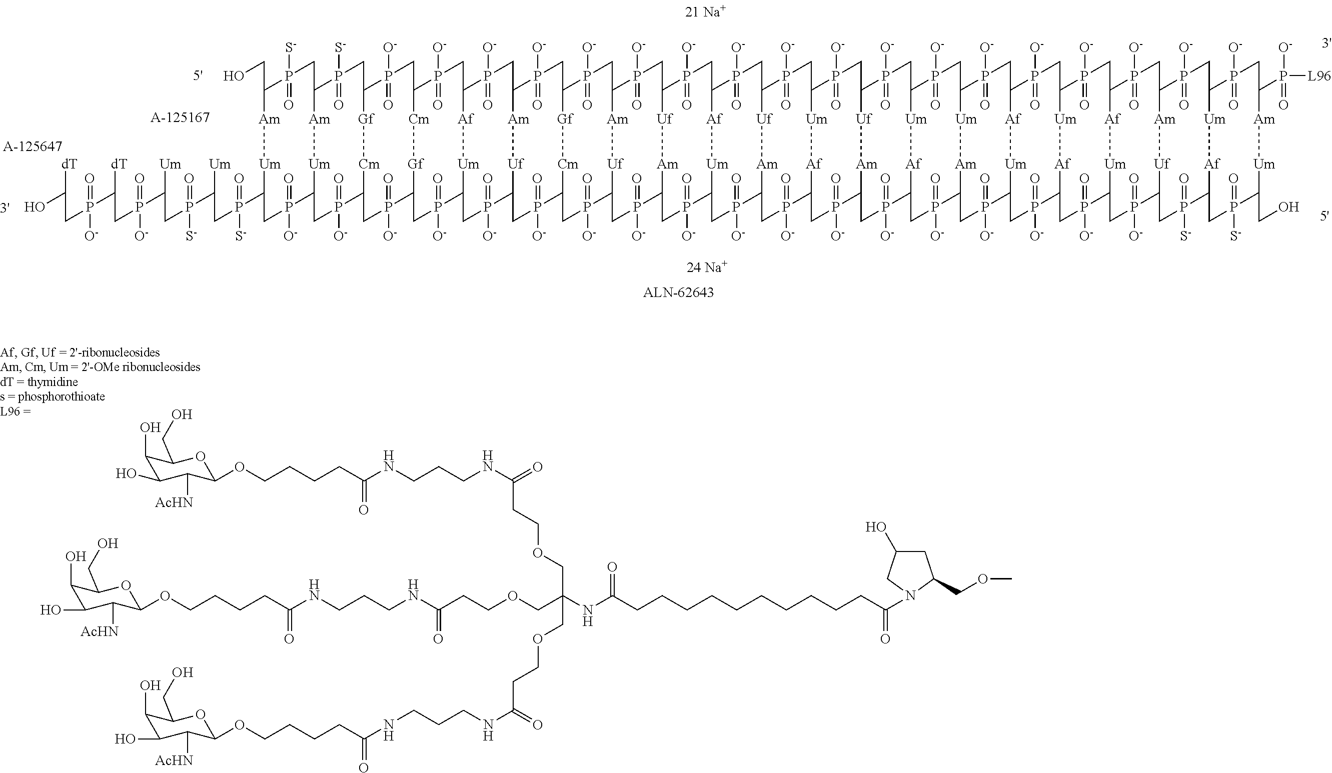

wherein the Cemdisiran Na⁺ form is in an aqueous composition which is an aqueous pharmaceutical formulation comprising a pharmaceutically acceptable carrier which is, for example, water for Injection, USP and sodium hydroxide and/or phosphoric acid, pH ˜7.0. In an embodiment of the invention, the Cemdisiran Na⁺ form is in a sterile, preservative-free, clear, colorless to yellow solution in glass vials, free from visible particulates. In an embodiment of the invention, the Cemdisiran is in one or more vials. In an embodiment of the invention, the 600 mg Cemdisiran Na⁺ form or molar equivalent is in three separate vials including about 200 mg Cemdisiran Na⁺ form or molar equivalent. In an embodiment of the invention, the 600 mg Cemdisiran Na⁺ form or molar equivalent is administered in 2 injections with a volume of 1.5 ml each or 3 injections of 1 ml each or a single injection with a full dose. In an embodiment of the invention, the full 600 mg Cemdisiran Na⁺ form dose, or molar equivalent, is in three separate vials. In an embodiment of the invention, vials containing the Cemdisiran are refrigerated, wherein the method includes the steps of removing the vials from refrigeration and allowing the vials to sit for at least 15 minutes at 20° C. to 25° C. (68° F. to 77° F.) before use. In an embodiment of the invention, the method includes one or more of the steps of inspecting the aqueous composition, visually, for particulate matter and discoloration prior to administration discarding the aqueous composition if it is cloudy, discolored or contains particulate matter; gently swirling the vials comprising the aqueous composition in an upright position but not shaking the vials; subcutaneously administering the Cemdisiran, e.g., with a 25 gauge to 27 gauge, ½ inch or ⅝-inch stainless steel needle, e.g., with Luer-Lok; withdrawing 3 ml of the aqueous composition, comprising the Cemdisiran Na⁺ form, with a 21 gauge withdrawal needle, e.g., with Luer-Lok, equally into two syringes each containing about 1.5 mL or 3 syringes with 1 ml each; changing the withdrawal needle on each syringe to an injection needle fulfilling the following criteria: 25 gauge to 27 gauge and ½ or ⅝-inch stainless steel needle, e.g., with Luer-Lok; administering the subcutaneous injections, each at a different injection site, for example, into the thigh, back of the upper arm, or abdomen, on opposite sides or at least 2 cm apart, but not injecting into moles, scars, or areas where the skin is tender, bruised, red, hard, or not intact; observing the subject for 30 minutes following completion of the first 600 mg Cemdisiran Na⁺ form dose; and/or administering the subcutaneous injection within 4 hours of preparation.

Water for injection (WFI) is an excipient in the production of parenteral and other preparations where the product endotoxin content must be controlled. See U.S. Pharmacopeia (USP) 37, General Information/<1231> Water for Pharmaceutical Purposes.

For example, the present invention provides a method for treating generalized myasthenia gravis (gMG) in an adult patient who is anti-acetylcholine receptor (AChR) or anti-low-density lipoprotein receptor-related protein 4 (LRP4) antibody positive comprising: removing the 3 glass vials containing the Cemdisiran Na⁺ form which is in a sterile, preservative-free, clear, colorless to yellow solution free from visible particulates wherein each vial contains 200 mg Cemdisiran Na⁺ form in 1 mL with water for injection, USP and sodium hydroxide and/or phosphoric acid, pH ˜7.0, from refrigeration and allowing the vials to sit for at least 15 minutes at room temperature 20° C. to 25° C. (68° F. to 77° F.) before use; inspecting the solution visually for particulate matter and discoloration prior to any administration; discarding the solution if it is cloudy, discolored or contains particulate matter; gently swirling the vials in an upright position, but not shaking the vials; withdrawing 3 mL of the solution equally into two syringes (each containing 1.5 mL) or 3 syringes with 1 ml each with a withdrawal needle (21 gauge needle with Luer-Lok); changing the withdrawal needle on each syringe to an injection needle fulfilling the following criteria:
25 gauge to 27 gauge; and
½ or ⅝-inch stainless steel needle with Luer-Lok;
   and, within 4 hours, administering 600 mg Cemdisiran Na⁺ form by two separate subcutaneous 1.5 ml injections or three separate 1 ml injections of the solution consecutively, each at a different injection site, into the thigh, back of the upper arm, or abdomen, on opposite sides or at least 2 cm apart, but not injecting into moles, scars, or areas where the skin is tender, bruised, red, hard, or not intact, every 12 weeks or 3 months; wherein the Cemdisiran Na⁺ form is represented by the chemical structure:

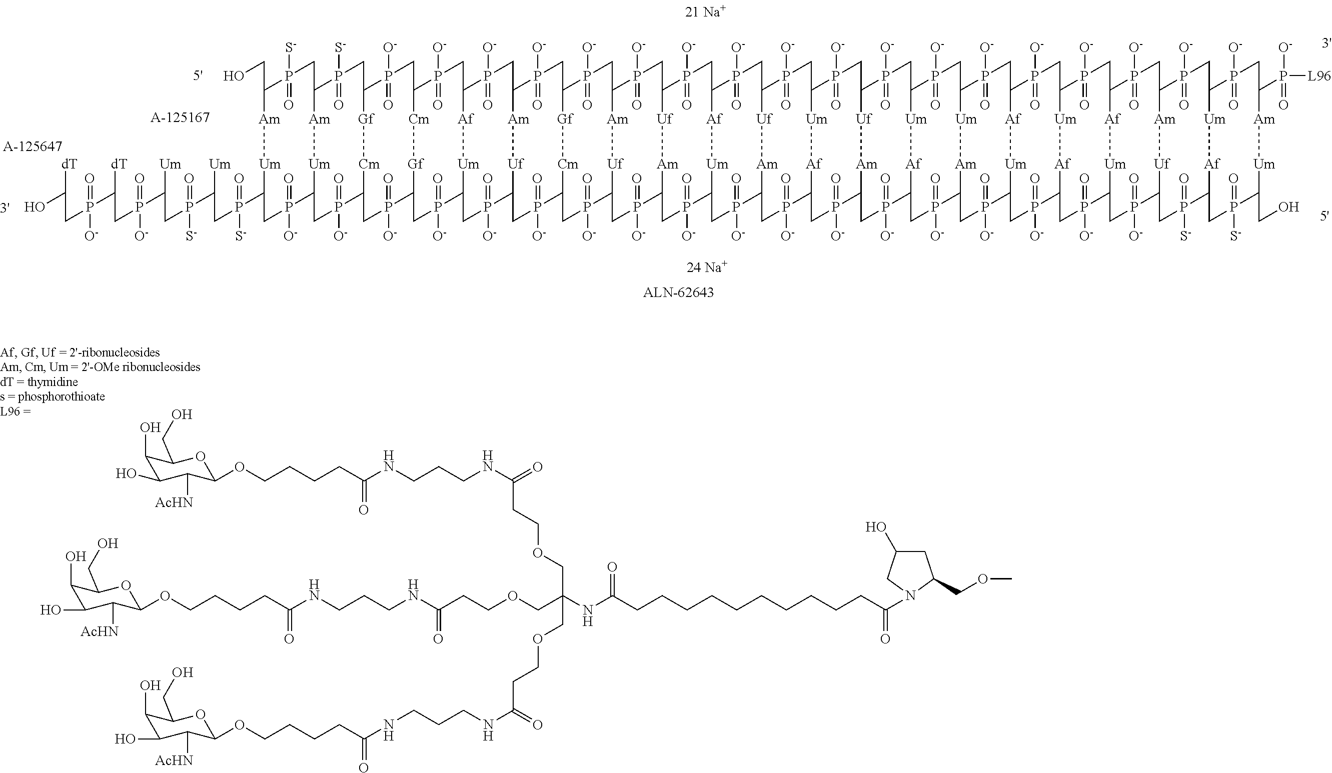

and observing the subject for 30 minutes following administering of the first dose of 600 mg (3 ml) Cemdisiran Na⁺ form.

### BRIEF DESCRIPTION OF THE DRAWINGS

The accompanying drawings, which are incorporated herein and constitute part of this specification, are illustrative of particular embodiments of the present disclosure and do not limit the scope of the present disclosure. The drawings are intended for use in conjunction with the explanations in the following detailed description. The patent or application file contains at least one drawing executed in color. Copies of this patent or patent application publication with color drawings will be provided by the Office upon request and payment of the necessary fee.

FIG. 1. Study Flow Diagram for the study described in Example 1. Abbreviations are as follows: Q4W: every 4 weeks; Q12W: every 12 weeks; SC: subcutaneous administration; “Combination”: Pozelimab about 200 mg Q4W+Cemdisiran about 200 mg Q4W. Day 1 is the start of double-blind SC Q4W treatment. W24 is primary endpoint: Myasthenia Gravis-Activities of Daily Living (MG-ADL). From Day 1 to W36 all treatments are added onto the patient's existing treatment, as applicable, for MG. W52 is start of open-label combination SC Q4W. W60 is start of open-label Cemdisiran. W108 is last dose of Cemdisiran. W116 is last dose of combination. During the extension period (after week 24 PEP) PBO patients were re-randomize to cemdi mono (Cemdisiran monotherpay) or combo (combination pozelimab+Cemdisiran therapy).

FIG. 2. Cemdisiran Structure

FIG. 3. Comparison of Treatment Efficacy (primary and secondary endpoints) between Patients Receiving Cemdisiran Monotherapy and Combination Treatment (Forest Plot). MSE: Minimal symptom expression (score of 0 or 1 on MG-ADL); Stable responders: Achievement of a consistent response on the MG-ADL, defined as a patient with a ≥2-point reduction (improvement) in MG-ADL total score on 2 or more consecutive assessments spanning 4 or more weeks during the DBTP. MGC: Myasthenia Gravis Composite; MG-QOL15r: Myasthenia Gravis Quality of Life revised. See Example 1.

FIG. 4. Mean CH50 Change (%) from Baseline over Time in Placebo, Pozelimab, Combination and Cemdisiran arms. See Example 1.

FIG. 5. Total C5 concentration in Plasma over Time in Placebo, Pozelimab, Combination and Cemdisiran arms. LLOQ is indicated by the dashed line. Below LOQ values are set to LLOQ/2. See Example 1.

FIG. 6. Least Squares Mean Change in Myasthenia Gravis-Activities of Daily Living (MG-ADL) Score from Baseline over Time in Placebo, Pozelimab, Combination and Cemdisiran arms. Least square means are adjusted for intercurrent events (i.e., rescue therapy, and discontinue study due to AE) and baseline differences. The number of subjects per arm is based on the raw data prior to addressing intercurrent events. See Example 1.

FIG. 7. Myasthenia Gravis-Activities of Daily Living (MG-ADL) Score over Time (mean absolute values) in Placebo, Pozelimab, Combination and Cemdisiran arms. Raw observed data without adjusting for intercurrent events (i.e., includes data post-rescue) and without any imputation. No adjustments for baseline differences (i.e., not LS means). See Example 1.

FIG. 8. Observed Mean Change in Myasthenia Gravis-Activities of Daily Living (MG-ADL) Score from Baseline over Time in Placebo, Pozelimab, Combination and Cemdisiran arms. Raw observed data without adjusting for intercurrent events (i.e., includes data post-rescue) and without any imputation. No adjustments for baseline differences (i.e., not LS means). See Example 1.

FIG. 9. Least Squares Mean Change in Quantitative Myasthenia Gravis (QMG) Score from Baseline over Time in Placebo, Pozelimab, Combination and Cemdisiran arms. Least square means are adjusted for intercurrent events (i.e., rescue therapy, and discontinue study due to AE) and baseline differences. The number of subjects per arm is based on the raw data prior to addressing intercurrent events. See Example 1.

FIG. 10. Quantitative Myasthenia Gravis (QMG) Total Score (mean) over Time in Placebo, Pozelimab, Combination and Cemdisiran arms. Raw observed data without adjusting for intercurrent events (i.e., includes data post-rescue) and without any imputation. No adjustments for baseline differences (i.e., not LS means). See Example 1.

FIG. 11. Observed Mean Change in Quantitative Myasthenia Gravis (QMG) Score from Baseline over Time in Placebo, Pozelimab, Combination and Cemdisiran arms. Raw observed data without adjusting for intercurrent events (i.e., includes data post-rescue) and without any imputation. No adjustments for baseline differences (i.e., not LS means). See Example 1.

FIG. 12. Percentage of Responders Achieving a Change in Myasthenia Gravis-Activities of Daily Living (MG-ADL) Score (of 2, 3, 4, 5, 6, 7, 8, 9 or 10) in Cemdisiran, Combination and Placebo Arms. Percents on top of the bars are the proportion of participants who had at least the indicated point reduction (improvement) in their MG-ADL total score. MCID: Minimal clinically important difference. See Example 1.

FIG. 13. Percentage of Responders Achieving a Change in Quantitative Myasthenia Gravis (QMG) Score (of 3, 4, 5, 6, 7, 8, 9 or 10) in Cemdisiran, Combination and Placebo Arms. Percents on top of the bars are the proportion of participants who had at least the indicated point reduction (improvement) in their QMG total score. MCID: Minimal clinically important difference. See Example 1.

FIG. 14. Serum concentration of Pozelimab over time in patients receiving Pozelimab or Pozelimab and Cemdisiran. *Mean (+SD) Concentrations of Total Pozelimab in Serum Over Time (Log-Scaled). **The concentration records that were collected the first time after the participants did not receive the dose per protocol were excluded. LLOQ indicated with dashed line. See Example 1.

FIG. 15 (A&B). Serum concentration of (A) Cemdisiran and (B) Cemdisiran metabolite, AS(N-2)3′Cemdisiran, over time in Pozelimab+Cemdisiran arm and Cemdisiran arm. (A) ***The concentration records that were collected the first time after the participants did not receive the dose per protocol were excluded. ****Predose and postdose concentrations are jittered to show peak and trough concentrations separately. (B) *Mean (+SD) Concentrations of Cemdisiran and metabolite in Plasma Over Time (Log-Scaled). **Below LLOQ value is set to LLOQ/2. See Example 1.

FIG. 16. Mean (±SD) Percent Change from Baseline in Concentrations of Total C5 in Plasma by Nominal Time, Treatment Group and Study (Study ALN-CC5-001, part A, single ascending dose (SAD) and R3918-MG-2018). See Example 1.

FIG. 17. Mean (±SD) Concentrations of Total C5 in Plasma by Nominal Time, Treatment Group and Study (Study ALN-CC5-001, part A, single ascending dose (SAD) and R3918-MG-2018). See Example 1.

FIG. 18. Total C5 Concentration (+SD) in Plasma in Placebo, Combination, Cemdisiran and Pozelimab Arms. *Below LOQ values are set to LLOQ/2. See Example 1.

FIG. 19. Mean (±SD) Percent Change from Baseline in CH50 in Serum Over Time in Placebo, Combination, Cemdisiran and Pozelimab Arms. See Example 1.

FIG. 20. Mean (±SD) CH50 in Serum by Nominal Time in Participants with gMG in Placebo, Combination, Cemdisiran and Pozelimab Arms. See Example 1.

### DETAILED DESCRIPTION

The present invention provides, in part, a method for treating or preventing a C5-associated disease or disorder (e.g., generalized myasthenia gravis (gMG)), for example in an adult subject in need thereof who is anti-acetylcholine receptor (AChR) or anti-low-density lipoprotein receptor-related protein 4 (LRP4) antibody positive) including the steps of administering, to the subject, a therapeutically effective amount of C5 iRNA (preferably, Cemdisiran, for example, at a dose of about 600 mg which may be administered one or more times every 12 weeks) or a combination of an antibody or antigen-binding fragment thereof that binds specifically to C5 (preferably Pozelimab) and C5 iRNA (preferably, Cemdisiran).

The following discussion omits or only briefly describes conventional features of the disclosed technology that are apparent to those skilled in the art. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations. A person of ordinary skill in the art would know how to use the instant invention, in combination with routine experiments, to achieve other outcomes not specifically disclosed in the examples or the embodiments.

Unless otherwise specifically defined herein, all terms are to be given their broadest possible interpretation including meanings implied from the specification as well as meanings understood by those skilled in the art and/or as defined in dictionaries, treatises, etc. Unless defined otherwise, all technical and scientific terms used herein have the same meaning as commonly understood by one of ordinary skill in the art in the field of the disclosed technology. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. Additionally, methods, equipment, and materials similar or equivalent to those described herein can also be used in the practice or testing of the disclosed technology.

### Antigen-Binding Proteins

The present invention includes methods and compositions including anti-C5 antigen-binding proteins such as antibodies and antigen-binding fragments thereof.

The term “antibody”, as used herein, refers to immunoglobulin molecules including four polypeptide chains, two heavy chains (HCs) and two light chains (LCs), inter-connected by disulfide bonds (e.g., IgG)—for example H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; Crovalimab; Eculizumab, Tesidolumab, Mubodina or Ravulizumab; preferably, Pozelimab. In an embodiment of the disclosure, each antibody heavy chain (HC) comprises a heavy chain variable region (“HCVR” or “V_{H}”) (e.g., any of SEQ ID NOs: 2; 18; 34; 50; 66; 82; 98; 122; 138; 146; 154; 170; 186; 202; 218; 234; 250; 266; 274; 290; 306; 322; or 338; or a variant thereof) and a heavy chain constant region; and each antibody light chain (LC) comprises a light chain variable region (“LCVR or “V_{L}”) (e.g., any of SEQ ID NOs: 10; 26; 42; 58; 74; 90; 106; 114; 130; 162; 178; 194; 210; 226; 242; 258; 282; 298; 314; 330; or 346; or a variant thereof) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Preferably an anti-C5 antibody or antigen-binding fragment thereof in a co-formulation of the present disclosure was expressed and isolated from a mammalian host cell such as a Chinese hamster ovary (CHO) cell.

Antibodies as set forth herein include, for example, monoclonal, recombinant, chimeric, human and/or humanized antibodies.

In an embodiment of the disclosure, the assignment of amino acids to each framework or CDR domain is in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J. Mol. Biol. 196:901-917 or Chothia, et al., (1989) Nature 342:878-883. Thus, the present disclosure includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

In an embodiment of the disclosure, an anti-C5 antigen-binding protein, e.g., anti-C5 antibody or antigen-binding fragment thereof, comprises a heavy chain constant domain, e.g., of the type IgA (e.g., IgA1 or IgA2), IgD, IgE, IgG (e.g., IgG1, IgG2, IgG3 and IgG4 (e.g., including a S228P and/or S108P mutation)) or IgM. In an embodiment of the disclosure, an antigen-binding protein, e.g., antibody or antigen-binding fragment thereof, comprises a light chain constant domain, e.g., of the type kappa or lambda. The present disclosure includes antigen-binding proteins including the variable domains set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; Crovalimab; Eculizumab, Tesidolumab, Mubodina or Ravulizumab; preferably, Pozelimab) which are linked to a heavy and/or light chain constant domain, e.g., as set forth above.

Pozelimab is sold commercially as Veopoz.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments thereof, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments thereof).

In an embodiment of the disclosure, an antibody or antigen-binding fragment thereof that binds specifically to complement factor 5 (C5) protein, interacts with one or more amino acids contained within NMATGMDSW (SEQ ID NO: 353) (or at least 1, 2, 3, 4 or 5 amino acids therein); or WEVHLVPRRKQLQFALPDSL (SEQ ID NO: 354) (or at least 1, 2, 3, 4 or 5 amino acids therein), as determined by hydrogen/deuterium exchange. In an embodiment of the disclosure, an antibody or antigen-binding fragment thereof that binds specifically to complement factor 5 (C5) protein interacts with one or more amino acids contained within the alpha chain and/or the beta chain of C5, as determined by hydrogen/deuterium exchange. For example, in an embodiment of the disclosure, the antibody or antigen-binding fragment thereof does not interact with an amino acid of the C5a anaphylatoxin region of C5, as determined by hydrogen/deuterium exchange. In an embodiment of the disclosure, an antibody or antigen-binding fragment thereof that binds specifically to complement factor 5 (C5) protein interacts with an amino acid sequence selected from the group consisting of

(SEQ ID NO: 353)
(a) NMATGMDSW;
(SEQ ID NO: 355)
(b) ATGMDSW;
(SEQ ID NO: 356)
(c) WEVHLVPRRKQLQ;
(SEQ ID NO: 354)
(d) WEVHLVPRRKQLQFALPDSL;
and
(SEQ ID NO: 357)
(e) LVPRRKQLQ.

The sequence of anti-C5 antibodies and antigen-binding fragments thereof (e.g., LCVRs and HCVRs or LCDRs and HCDRs thereof) that may be included in a co-formulation or used in a method are set forth below.

TABLE A
Anti-C5 Antibody Chain Amino Acid Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 2 | 4 | 6 | 8 | 10 | 12 | 14 | 16 |
| H2M11686N | 18 | 20 | 22 | 24 | 26 | 28 | 30 | 32 |
| H4H12159P | 34 | 36 | 38 | 40 | 42 | 44 | 46 | 48 |
| H4H12161P | 50 | 52 | 54 | 56 | 58 | 60 | 62 | 64 |
| H4H12163P | 66 | 68 | 70 | 72 | 74 | 76 | 78 | 80 |
| H4H12164P | 82 | 84 | 86 | 88 | 90 | 92 | 94 | 96 |
| H4H12166P | 98 | 100 | 102 | 104 | 106 | 108 | 110 | 112 |
| H4H12166P2 | 98 | 100 | 102 | 104 | 114 | 116 | 118 | 120 |
| H4H12166P3 | 122 | 124 | 126 | 128 | 106 | 108 | 110 | 112 |
| H4H12166P4 | 98 | 100 | 102 | 104 | 130 | 132 | 134 | 136 |
| H4H12166P5 | 138 | 140 | 142 | 144 | 106 | 108 | 110 | 112 |
| H4H12166P6 | 146 | 148 | 150 | 152 | 106 | 108 | 110 | 112 |
| H4H12166P7 | 122 | 124 | 126 | 128 | 130 | 132 | 134 | 136 |
| H4H12166P8 | 146 | 148 | 150 | 152 | 114 | 116 | 118 | 120 |
| H4H12166P9 | 146 | 148 | 150 | 152 | 130 | 132 | 134 | 136 |
| H4H12166P10 | 138 | 140 | 142 | 144 | 130 | 132 | 134 | 136 |
| H4H12167P | 154 | 156 | 158 | 160 | 162 | 164 | 166 | 168 |
| H4H12168P | 170 | 172 | 174 | 176 | 178 | 180 | 182 | 184 |
| H4H12169P | 186 | 188 | 190 | 192 | 194 | 196 | 198 | 200 |
| H4H12170P | 202 | 204 | 206 | 208 | 210 | 212 | 214 | 216 |
| H4H12171P | 218 | 220 | 222 | 224 | 226 | 228 | 230 | 232 |
| H4H12175P | 234 | 236 | 238 | 240 | 242 | 244 | 246 | 248 |
| H4H12176P2 | 250 | 252 | 254 | 256 | 258 | 260 | 262 | 264 |
| H4H12177P2 | 266 | 268 | 270 | 272 | 258 | 260 | 262 | 264 |
| H4H12183P2 | 274 | 276 | 278 | 280 | 282 | 284 | 286 | 288 |
| H2M11682N | 290 | 292 | 294 | 296 | 298 | 300 | 302 | 304 |
| H2M11684N | 306 | 308 | 310 | 312 | 314 | 316 | 318 | 320 |
| H2M11694N | 322 | 324 | 326 | 328 | 330 | 332 | 334 | 336 |
| H2M11695N | 338 | 340 | 342 | 344 | 346 | 348 | 350 | 352 |

*Antibodies and fragments may include one or more variants of the sequences.
See WO2017/218515

Polynucleotides encoding the chains set forth in Table A are set forth below in Table B.

TABLE B
Anti-C5 Antibody Chain Nucleotide Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 1 | 3 | 5 | 7 | 9 | 11 | 13 | 15 |
| H2M11686N | 17 | 19 | 21 | 23 | 25 | 27 | 29 | 31 |
| H4H12159P | 33 | 35 | 37 | 39 | 41 | 43 | 45 | 47 |
| H4H12161P | 49 | 51 | 53 | 55 | 57 | 59 | 61 | 63 |
| H4H12163P | 65 | 67 | 69 | 71 | 73 | 75 | 77 | 79 |
| H4H12164P | 81 | 83 | 85 | 87 | 89 | 91 | 93 | 95 |
| H4H12166P | 97 | 99 | 101 | 103 | 105 | 107 | 109 | 111 |
| H4H12166P2 | 97 | 99 | 101 | 103 | 113 | 115 | 117 | 119 |
| H4H12166P3 | 121 | 123 | 125 | 127 | 105 | 107 | 109 | 111 |
| H4H12166P4 | 97 | 99 | 101 | 103 | 129 | 131 | 133 | 135 |
| H4H12166P5 | 137 | 139 | 141 | 143 | 105 | 107 | 109 | 111 |
| H4H12166P6 | 145 | 147 | 149 | 151 | 105 | 107 | 109 | 111 |
| H4H12166P7 | 121 | 123 | 125 | 127 | 129 | 131 | 133 | 135 |
| H4H12166P8 | 145 | 147 | 149 | 151 | 113 | 115 | 117 | 119 |
| H4H12166P9 | 145 | 147 | 149 | 151 | 129 | 131 | 133 | 135 |
| H4H12166P10 | 137 | 139 | 141 | 143 | 129 | 131 | 133 | 135 |
| H4H12167P | 153 | 155 | 157 | 159 | 161 | 163 | 165 | 167 |
| H4H12168P | 169 | 171 | 173 | 175 | 177 | 179 | 181 | 183 |
| H4H12169P | 185 | 187 | 189 | 191 | 193 | 195 | 197 | 199 |
| H4H12170P | 201 | 203 | 205 | 207 | 209 | 211 | 213 | 215 |
| H4H12171P | 217 | 219 | 221 | 223 | 225 | 227 | 229 | 231 |
| H4H12175P | 233 | 235 | 237 | 239 | 241 | 243 | 245 | 247 |
| H4H12176P2 | 249 | 251 | 253 | 255 | 257 | 259 | 261 | 263 |
| H4H12177P2 | 265 | 267 | 269 | 271 | 257 | 259 | 261 | 263 |
| H4H12183P2 | 273 | 275 | 277 | 279 | 281 | 283 | 285 | 287 |
| H2M11682N | 289 | 291 | 293 | 295 | 297 | 299 | 301 | 303 |
| H2M11684N | 305 | 307 | 309 | 311 | 313 | 315 | 317 | 319 |
| H2M11694N | 321 | 323 | 325 | 327 | 329 | 331 | 333 | 335 |
| H2M11695N | 337 | 339 | 341 | 343 | 345 | 347 | 349 | 351 |

*Antibodies and fragments may include one or more variants of the sequences.
See WO2017/218515

H2M11683N
HCVR
(SEQ ID NO: 2)
QVQLVESGGGVVQPGRSLRLSCVASGFTFSSYGIHWVRQAPGKGLEWVAVIWDDGNNINY
SDSVKGRFIISRDNSRKTVYLQMNSLRGEDTAVYYCARDAPIAPVPDYWGQGTLVTVSS
HCDR1, HCDR2 and HCDR3 are set forth below, respectively:
Gly Phe Thr Phe Ser Ser Tyr Gly;
Ile Trp Asp Asp Gly Asn Asn Ile;
and
Ala Arg Asp Ala Pro Ile Ala Pro Val Pro Asp Tyr
LCVR
(SEQ ID NO: 10)
DIQMTQSPSTLSASVGDRVTITCRASQSISSWLAWYQQKPGKAPKLLIYKASSLDTGVPS
RFSGSGSGTEFTLTISSLQPDDFATYYCQQYNTYSYTFGLGTKLEIK
LCDR1, LCDR2 and LCDR3 are set forth below, respectively:
Gln Ser Ile Ser Ser Trp;
Lys Ala Ser;
and
Gln Gln Tyr Asn Thr Tyr Ser Tyr Thr
H2M11686N
HCVR
(SEQ ID NO: 18)
QVQLVESGGGLVKPGGSLRLSCAASGFTFSDYYMSWIRQAPGKGLEWVSYISSSGNTIKY
ADSMKGRFTISRDNAKKSLFVEMNSLRAEDTAVYYCARYKSSSDYFDHWGQGTLVTVSS
HCDR1, HCDR2 and HCDR3 are set forth below, respectively:
Gly Phe Thr Phe Ser Asp Tyr Tyr;
Ile Ser Ser Ser Gly Asn Thr Ile;
and
Ala Arg Tyr Lys Ser Ser Ser Asp Tyr Phe Asp His
LCVR
(SEQ ID NO: 26)
EIVLTQSPATLSLSPGERATLSCRASQSVRSYLAWYQQKPGQAPRLLIYDASNRATAIPA
RFSGSGSGTDFTLTISSLEPEDLAVYYCQQSGNWPLTFGGGTKVEIK
LCDR1, LCDR2 and LCDR3 are set forth below, respectively:
Gln Ser Val Arg Ser Tyr;
Asp Ala Ser;
and
Gln Gln Ser Gly Asn Trp Pro Leu Thr
H4H12159P
HCVR
(SEQ ID NO: 34)
QVQLVESGGGVVQPGRSLRLSCGASGFTFSTYGMHWVRQAPGKGLEWVAVIWDDGNNKYY
ADSVKGRFTISRDNSKNTLYLQMNSLRAEDTAVYYCARDSEVAPVGDYWGQGTLVTVSS
HCDR1, HCDR2 and HCDR3 are set forth below, respectively:
Gly Phe Thr Phe Ser Thr Tyr Gly;
Ile Trp Asp Asp Gly Asn Asn Lys;
and
Ala Arg Asp Ser Glu Val Ala Pro Val Gly Asp Tyr
LCVR
(SEQ ID NO: 42)
DIQMTQSPSTLSASVGDRVTIICRASQSINRWLAWYQQKPGKAPKLLIYKASSLESGVPS
RFSGSGSGTEFTLTISSLQPDDFAAYYCQQYNDYSYTFGQGTKLEIK
LCDR1, LCDR2 and LCDR3 are set forth below, respectively:
Gln Ser Ile Asn Arg Trp;
Lys Ala Ser;
and
Gln Gln Tyr Asn Asp Tyr Ser Tyr Thr
H4H12161P
HCVR
(SEQ ID NO: 50)
EVQLVESGGDLVQPGGSLRLSCAASGFTFSDHYMDWVRQAPGKGLDWIGRIRNKANAYNT
EYAASVRGRFTISRDDSQNLLYLQMNSLKTDDTAVYYCVRVWNYAYFAMDVWGQGTTVTV
SS
HCDR1, HCDR2 and HCDR3 are set forth below, respectively:
Gly Phe Thr Phe Ser Asp His Tyr;
Ile Arg Asn Lys Ala Asn Ala Tyr Asn Thr;
and
Val Arg Val Trp Asn Tyr Ala Tyr Phe Ala Met Asp Val
LCVR
(SEQ ID NO: 58)
DIQMTQSPSSLSASVGDRVTITCRSSQNIGIFLNWYQQKPGEAPNLLISAASSLHSGVPS
RFSGSGSGTDFTLTIGSLQPEDFATYYCQQTYNTIFTFGPGTKVDIK
LCDR1, LCDR2 and LCDR3 are set forth below, respectively:
Gln Asn Ile Gly Ile Phe;
Ala Ala Ser;
and
Gln Gln Thr Tyr Asn Thr Ile Phe Thr
H4H12163P
HCVR
(SEQ ID NO: 66)
EVQLVESGGDLVQPGGSLRLSCAASGFTFSSYAMNWVRQGPGKGLEWVSAISGRGDSTYY
ADSVKGRLTISRDNSKNTLYLQMNSLRAEDTAVYYCVKEGEQLVYWYFDLWGRGTLVTVSS
HCDR1, HCDR2 and HCDR3 are set forth below, respectively:
Gly Phe Thr Phe Ser Ser Tyr Ala;
Ile Ser Gly Arg Gly Asp Ser Thr;
and
Val Lys Glu Gly Glu Gln Leu Val Tyr Trp Tyr Phe Asp Leu
LCVR
(SEQ ID NO: 74)
DIQMTQSPSSLSASVGDRVTITCRASQTISNFLHWYQQKPGKAPKLLIYAASSLQSGVPS
RFSGSGSGTDFTLTISSLQPEDFSTYFCQQSYTTPLTFGGGTKVEIK
LCDR1, LCDR2 and LCDR3 are set forth below, respectively:
Gln Thr Ile Ser Asn Phe;
Ala Ala Ser;
and
Gln Gln Ser Tyr Thr Thr Pro Leu Thr
H4H12164P
HCVR
(SEQ ID NO: 82)
EVQLVESGGGLVRSGGSLRLSCAASGFTFNRYAMTWVRQAPGKGLEWVSAISGSGSSTYY
TDSVKGRFTISRDNSKNSVDLQMHSLRVEDTAIYYCARGTTVTTGYGMDVWGQGTTVTVS
S
HCDR1, HCDR2 and HCDR3 are set forth below, respectively:
Gly Phe Thr Phe Asn Arg Tyr Ala;
Ile Ser Gly Ser Gly Ser Ser Thr;
and
Ala Arg Gly Thr Thr Val Thr Thr Gly Tyr Gly Met Asp Val
LCVR
(SEQ ID NO: 90)
DIQMTQSPSSLSASVGDRVTFTCQASQDITNSLNWYQQKPGRAPKLLIYDASYLKAGVPS
RFSGSGSGTDFTFTISSLQPEDIATYYCQQYDDLPYTFGQGTKLEIK
LCDR1, LCDR2 and LCDR3 are set forth below, respectively:
Gln Asp Ile Thr Asn Ser;
Asp Ala Ser;
and
Gln Gln Tyr Asp Asp Leu Pro Tyr Thr
H4H12166P
HCVR
(SEQ ID NO: 98)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp Thr Thr Met Ile Phe Asp Tyr
LCVR
(SEQ ID NO: 106)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp Thr
H4H12166P2
HCVR
(SEQ ID NO: 98)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp Thr Thr Met Ile Phe Asp Tyr
LCVR
(SEQ ID NO: 114)
AIQMTQSPSSLSASVGDRVTITCRASQGIRNDLGWYQQKPGKAPKLLIYAASSLQSGVPS
RFAGRGSGTDFTLTISSLQPEDFATYYCHQDFNYPWTFGQGTKVEIK
LCDR1, LCDR2 and LCDR3 are set forth below, respectively:
Gln Gly Ile Arg Asn Asp;
Ala Ala Ser;
and
His Gln Asp Phe Asn Tyr Pro Trp Thr
H4H12166P3
HCVR
(SEQ ID NO: 122)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREHNVDTTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu His Asn Val Asp Thr Thr Met Ile Phe Asp Tyr
LCVR
(SEQ ID NO: 106)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp Thr
H4H12166P4
HCVR
(SEQ ID NO: 98)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp Thr Thr Met Ile Phe Asp Tyr
LCVR
(SEQ ID NO: 130)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp His
H4H12166P5
HCVR
(SEQ ID NO: 138)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIHDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp Thr Thr Met Ile His Asp Tyr
LCVR
(SEQ ID NO: 106)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp Thr
H4H12166P6
HCVR
(SEQ ID NO: 146)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp His Thr Met Ile Phe Asp Tyr
LCVR
(SEQ ID NO: 106)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWTFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp Thr
H4H12166P7
HCVR
(SEQ ID NO: 122)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREHNVDTTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu His Asn Val Asp Thr Thr Met Ile Phe Asp Tyr
LCVR
(SEQ ID NO: 130)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp His
H4H12166P8
HCVR
(SEQ ID NO: 146)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp His Thr Met Ile Phe Asp Tyr
LCVR
(SEQ ID NO: 114)
AIQMTQSPSSLSASVGDRVTITCRASQGIRNDLGWYQQKPGKAPKLLIYAASSLQSGVPS
RFAGRGSGTDFTLTISSLQPEDFATYYCHQDFNYPWTFGQGTKVEIK
LCDR1, LCDR2 and LCDR3 are set forth below, respectively:
Gln Gly Ile Arg Asn Asp;
Ala Ala Ser;
and
His Gln Asp Phe Asn Tyr Pro Trp Thr
H4H12166P9
HCVR
(SEQ ID NO: 146)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDHTMIFDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp His Thr Met Ile Phe Asp Tyr
LCVR
(SEQ ID NO: 130)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp His
H4H12166P10
HCVR
(SEQ ID NO: 138)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIHDYWGQGTLVTVSS
HCDR1, HCDR2 and HCDR3 are set forth below, respectively:
Gly Asp Ser Val Ser Ser Ser Tyr;
Ile Tyr Tyr Ser Gly Ser Ser;
and
Ala Arg Glu Gly Asn Val Asp Thr Thr Met Ile His Asp Tyr
LCVR
(SEQ ID NO: 130)
AIQMTQSPSSLSASVGDRVTITCRASQGIRNDLGWYQQKPGKAPKLLIYAASSLQSGVPS
RFAGRGSGTDFTLTISSLQPEDFATYYCLQDFNYPWHFGQGTKVEIK
LCDR1, LCDR2 and LCDR3 are set forth below, respectively:
Gln Gly Ile Arg Asn Asp;
Ala Ala Ser;
and
Leu Gln Asp Phe Asn Tyr Pro Trp His
H4H12167P
HCVR
(SEQ ID NO: 154)
QVQLVESGGGLVKPGGSLRLSCAASGFTFSDSYMSWIRQAPGKGLEWISYIGSSGNTFYY
ADSVKGRFTISRDNANNLLYLQMTSLRAEDTAVYYCAREEGDFWSAVDSWGQGTLVTVSS
HCDR1, HCDR2 and HCDR3 are set forth below, respectively:
Gly Phe Thr Phe Ser Asp Ser Tyr;
Ile Gly Ser Ser Gly Asn Thr Phe;
and
Ala Arg Glu Glu Gly Asp Phe Trp Ser Ala Val Asp Ser
LCVR
(SEQ ID NO: 162)
DIQLTQSPSFLSASVGDRVTITCWASQGISSYLAWYQQKPGKAPKLLIHTASTLQSGVPS
RFSGSGSGTEFTLTISNLQPEDFATYYCQQLNSYPFTFGPGTKVDIK
LCDR1, LCDR2 and LCDR3 are set forth below, respectively:
Gln Gly Ile Ser Ser Tyr;
Thr Ala Ser;
and
Gln Gln Leu Asn Ser Tyr Pro Phe Thr
H4H12168P
HCVR
(SEQ ID NO: 170)
QVQLVESGGGVVQPGGSLRLSCAASGFTFGGHAMHWVRQAPGKGLEWLAVISSDGSNKQY
ADSVKGRFTISRDNPKNTLYLQMNSLRVGDTAIYYCAKEVAPRYYYYGLDVWGQGTTVTV
SS
HCDR1, HCDR2 and HCDR3 are set forth below, respectively:
Gly Phe Thr Phe Gly Gly His Ala;
Ile Ser Ser Asp Gly Ser Asn Lys;
and
Ala Lys Glu Val Ala Pro Arg Tyr Tyr Tyr Tyr Gly Leu Asp Val
LCVR
(SEQ ID NO: 178)
DIQMTQSPSSLSASVGDRVTITCRASQDISNFLAWYQQKPGKVPKLLIYTASTLQSGVPS
RFSGSGSGTDFTLTVSSLQPEDVATYYCQKYAGALTFGPGTKVDIK
LCDR1, LCDR2 and LCDR3 are set forth below, respectively:
Gln Asp Ile Ser Asn Phe;
Thr Ala Ser;
and
Gln Lys Tyr Ala Gly Ala Leu Thr
H4H12169P
HCVR
(SEQ ID NO: 186)
EVQLVESGGGLAQPGGSLRLSCAASGFTFRSYAMSWVRQAPGKGPEWVSGIGGNGVTTYY
ADSVKGRFTISRDNSKNTLFLQMNSLRAEDTAVYYCVQGGLGGYFTGYWGQGTLVTVSS
HCDR1, HCDR2 and HCDR3 are set forth below, respectively:
Gly Phe Thr Phe Arg Ser Tyr Ala;
Ile Gly Gly Asn Gly Val Thr Thr;
and
Val Gln Gly Gly Leu Gly Gly Tyr Phe Thr Gly Tyr
LCVR
(SEQ ID NO: 194)
DIQMTQSPSSLSASVGDRVTITCRASQSISTYLNWYQQNPGKAPKLLIFDASSLQSGVPS
RFSGSGSGTDFTLTIRGLQPEDFATYYCQQSYSAPLTFGGGTKVEIK
LCDR1, LCDR2 and LCDR3 are set forth below, respectively:
Gln Ser Ile Ser Thr Tyr;
Asp Ala Ser;
and
Gln Gln Ser Tyr Ser Ala Pro Leu Thr
H4H12170P
HCVR
(SEQ ID NO: 202)
QVQLVESGGGVVQPGRSLRLSCAASGFTFSGYGMHWVRQAPGKGLEWVALIWLDGSNDYY
ADSVKGRFTISRDNSKNTLYLQMNRLRAEDTAVYYCARDGPVAAIPDYWGQGTLVTVSS
HCDR1, HCDR2 and HCDR3 are set forth below, respectively:
Gly Phe Thr Phe Ser Gly Tyr Gly;
Ile Trp Leu Asp Gly Ser Asn Asp;
and
Ala Arg Asp Gly Pro Val Ala Ala Ile Pro Asp Tyr
LCVR
(SEQ ID NO: 210)
DIQMTQSPSTLSASVGDRVTITCRASQSISRWLAWYQLKPGKAPKLLIYKASSLESGVPS
RFSGSGSGTDFTLTISSLQPDDFATYYCQQYNTYSYTFGQGTKLEIK
LCDR1, LCDR2 and LCDR3 are set forth below, respectively:
Gln Ser Ile Ser Arg Trp;
Lys Ala Ser;
and
Gln Gln Tyr Asn Thr Tyr Ser Tyr Thr
H4H12171P
HCVR
(SEQ ID NO: 218)
EVQLVESGGGVVRPGGSLRLSCAASGFTFDEYGMTWVRQVPGKGLEWVSGITWNGGFTDY
TDSVKGRFTSSRDNAKNSLYLQMNSLRAEDTALYYCARDGYSSSWGAYDIWGQGTMVTVSS
HCDR1, HCDR2 and HCDR3 are set forth below, respectively:
Gly Phe Thr Phe Asp Glu Tyr Gly;
Ile Thr Trp Asn Gly Gly Phe Thr;
and
Ala Arg Asp Gly Tyr Ser Ser Ser Trp Gly Ala Tyr Asp Ile
LCVR
(SEQ ID NO: 226)
DIQMTQSPSSLSASVGDRVTITCRASQSISTYLNWYQQKPGKAPKLLIYAASSLQSGVPL
RFSGSGSGTDFTLTISSLQPEDFASYFCQQSYSTPYTFGQGTKLEIK
LCDR1, LCDR2 and LCDR3 are set forth below, respectively:
Gln Ser Ile Ser Thr Tyr;
Ala Ala Ser;
and
Gln Gln Ser Tyr Ser Thr Pro Tyr Thr
H4H12175P
HCVR
(SEQ ID NO: 234)
EVQLVESGGGVVQPGGSLRLSCAASGFTFNDYAMHWVRQAPGKGLEWVSLISGDGGNTYY
ADSVKGRLTISRDNSKNSLYLQMNSLRTEDTALYYCAKDKGWNFGYFDLWGRGTLVTVSS
HCDR1, HCDR2 and HCDR3 are set forth below, respectively:
Gly Phe Thr Phe Asn Asp Tyr Ala;
Ile Ser Gly Asp Gly Gly Asn Thr;
and
Ala Lys Asp Lys Gly Trp Asn Phe Gly Tyr Phe Asp Leu
LCVR
(SEQ ID NO: 242)
DIQMTQSPSSLSTSVGDRVTITCRASQNIDTYLNWYQQKPGKAPKLLIYDASSLQSGVPS
RFSGSGSGTDFTLTITSLQPEDFATYYCQQNDNILHPLTFGGGTKVEIK
LCDR1, LCDR2 and LCDR3 are set forth below, respectively:
Gln Asn Ile Asp Thr Tyr;
Asp Ala Ser;
and
Gln Gln Asn Asp Asn Ile Leu His Pro Leu Thr
H4H12176P2
HCVR
(SEQ ID NO: 250)
EVQLVESGGGLVQPGGSLRLSCAASGFHSNRYWMDWVRQAPGKGLEWVANIKQDGSEENY
VDSVKGRFTISRDNAKNSLYLQMNSLRAEDTAVYYCARDRSTSWVPYWFFDLWGRGTLVTVSS
HCDR1, HCDR2 and HCDR3 are set forth below, respectively:
Gly Phe His Ser Asn Arg Tyr Trp;
Ile Lys Gln Asp Gly Ser Glu Glu;
and
Ala Arg Asp Arg Ser Thr Ser Trp Val Pro Tyr Trp Phe Phe Asp Leu
LCVR
(SEQ ID NO: 258)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
LCDR1, LCDR2 and LCDR3 are set forth below, respectively:
Gln Ser Ile Ser Ser Tyr;
Ala Ala Ser;
and
Gln Gln Ser Tyr Ser Thr Pro Pro Ile Thr
H4H12177P2
HCVR
(SEQ ID NO: 266)
EVQLVESGGGVVQRGESLRLSCSASDFIFKDYAMYWVRQIPGKGLEWISLISGDGDTTWY
GDSVKGRFTISRDNNENSLFLQMNDLRTEDTAMYYCARDMGWNFFQLQYWGQGTLVTVSS
HCDR1, HCDR2 and HCDR3 are set forth below, respectively:
Asp Phe Ile Phe Lys Asp Tyr Ala;
Ile Ser Gly Asp Gly Asp Thr Thr;
and
Ala Arg Asp Met Gly Trp Asn Phe Phe Gln Leu Gln Tyr
LCVR
(SEQ ID NO: 258)
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
LCDR1, LCDR2 and LCDR3 are set forth below, respectively:
Gln Ser Ile Ser Ser Tyr;
Ala Ala Ser;
and
Gln Gln Ser Tyr Ser Thr Pro Pro Ile Thr
H4H12183P2
HCVR
(SEQ ID NO: 274)
QVQLQESGPALVKPSQTLSLTCTVSGGSIIRGSTYWSWVRQFPGKGLEWIGYSYYSGTAY
YNPSLESRATISVDTSKNQFSLNLKSVTAADTAVYYCTREIGVAGLFDIWGQGTLVTVSS
HCDR1, HCDR2 and HCDR3 are set forth below, respectively:
Gly Gly Ser Ile Ile Arg Gly Ser Thr Tyr;
Ser Tyr Tyr Ser Gly Thr Ala;
and
Thr Arg Glu Ile Gly Val Ala Gly Leu Phe Asp Ile
LCVR
(SEQ ID NO: 282)
EIVLTQSPGTLSLSPGERATLSCRASQSVSSSYLAWYQQKPGQAPRLLIYGASSRATGIP
DRFSGSGSGTDFTLTISRLEPEDFAVYYCQQYGSSPWTFGQGTKVEIK
LCDR1, LCDR2 and LCDR3 are set forth below, respectively:
Arg Ala Ser Gln Ser Val Ser Ser Ser Tyr Leu Ala;
Gly Ala Ser Ser Arg Ala Thr;
and
Gln Gln Tyr Gly Ser Ser Pro Trp Thr
H2M11682N
HCVR
(SEQ ID NO: 290)
QEQLVQSGAEVKKPGASVKVSCKASGYTFTGYYIHWVRQAPGLGLEWMGWINPNSGGTKY
AQKFQGRVTMTRDTSINTAYMELKRLKSDDSAVYYCARDAPPHDVFDIWGQGTLVTVSS
HCDR1, HCDR2 and HCDR3 are set forth below, respectively:
Gly Tyr Thr Phe Thr Gly Tyr Tyr;
Ile Asn Pro Asn Ser Gly Gly Thr;
and
Ala Arg Asp Ala Pro Pro His Asp Val Phe Asp Ile
LCVR
(SEQ ID NO: 298)
DIQMTQSPSSLSASVGDRVTITCRASQGIRNDLGWYQQKPGKAPKRLIYAASSLQIGVPS
RFSGSGSGTEFTLTISSLQPEDFATYYCLQHNSYPLTFGGGTKVEIK
LCDR1, LCDR2 and LCDR3 are set forth below, respectively:
Gln Gly Ile Arg Asn Asp;
Ala Ala Ser;
and
Leu Gln His Asn Ser Tyr Pro Leu Thr
H2M11684N
HCVR
(SEQ ID NO: 306)
QVQLQESGPGLVKPSQTLSLTCTVSGGSISSGAYHWSWIRQHPGKGLEWIGYIYYNGDTY
YNPSLKSRVTISVDTSKNQFFLKVTSVTAADTAMYYCAGEKQLTAFDIWGQGTLVTVSS
HCDR1, HCDR2 and HCDR3 are set forth below, respectively:
Gly Gly Ser Ile Ser Ser Gly Ala Tyr His;
Ile Tyr Tyr Asn Gly Asp Thr;
and
Ala Gly Glu Lys Gln Leu Thr Ala Phe Asp Ile
LCVR
(SEQ ID NO: 314)
VIQMTQSPSSLSASVGDRVTITCRASQDINNFLNWYQQKLGKAPKLLISDASNLQTGVPS
RFSGSGSGTDFTFTISSLQPEDIAAYYCQQYDHFPYTFGQGTRLENN
LCDR1, LCDR2 and LCDR3 are set forth below, respectively:
Gln Asp Ile Asn Asn Phe;
Asp Ala Ser;
and
Gln Gln Tyr Asp His Phe Pro Tyr Thr
H2M11694N
HCVR
(SEQ ID NO: 322)
EVQLVESGGGVVRPGGSLRLSCAASGFTFDDYGMTWVRQAPGKGLEWVSGINWNGDSTEY
SDSVKGRFTISRDNAKNSLYLQMNSLRAEDTAFYHCARENNWNFYFDYWGQGTLVTVSS
HCDR1, HCDR2 and HCDR3 are set forth below, respectively:
Gly Phe Thr Phe Asp Asp Tyr Gly;
Ile Asn Trp Asn Gly Asp Ser Thr;
and
Ala Arg Glu Asn Asn Trp Asn Phe Tyr Phe Asp Tyr
LCVR
(SEQ ID NO: 330)
EIVMTQSPATLSVSRGERATLSCRASQSVSSNLAWYQQKLGQAPRLLIYGASTRATGIPA
RFSGSGSGTEFTLTISSLQSEDFAVYYCQQYNNWPWTFGQGTKVEIK
LCDR1, LCDR2 and LCDR3 are set forth below, respectively:
Gln Ser Val Ser Ser Asn;
Gly Ala Ser;
and
Gln Gln Tyr Asn Asn Trp Pro Trp Thr
H2M11695N
HCVR
(SEQ ID NO: 338)
QVHLVQSGAEVKKPGASVKVSCKVSGNTLTELSMHWVRQAPGKGLEWMGGFDPEDGDTIY
SQKFQGRVTLTEDTSTDTAYMELSSLRSEDTAVYYCSTVGGPTSDCWGQGTLVTVSS
HCDR1, HCDR2 and HCDR3 are set forth below, respectively:
Gly Asn Thr Leu Thr Glu Leu Ser;
Phe Asp Pro Glu Asp Gly Asp Thr;
and
Ser Thr Val Gly Gly Pro Thr Ser Asp Cys
LCVR
(SEQ ID NO: 346)
DIQMTQSPSSLSASVGDRVTITCQASQDISNYLNWYQQKPGKAPKVLIFDASNLEPGVPS
RFSGSGSGTDFTFTIISLQPEDIATYYCQQYDNLPITFGQGTRLDIK
LCDR1, LCDR2 and LCDR3 are set forth below, respectively:
Gln Asp Ile Ser Asn Tyr;
Asp Ala Ser;
and
Gln Gln Tyr Asp Asn Leu Pro Ile Thr

### CDRs Underscored

In an embodiment of the disclosure, an antibody or antigen-binding fragment thereof that binds specifically to C5, which is in a co-formulation of the present disclosure comprises:
(1)
a HCVR that comprises the HCDR1, HCDR2 and HCDR3 of a HCVR that comprises the amino acid sequence set forth in SEQ ID NO: 2 (or a variant thereof),
and
a LCVR that comprises the LCDR1, LCDR2 and LCDR3 of a LCVR that comprises the amino acid sequence set forth in SEQ ID NO: 10 (or a variant thereof);
(2)
a HCVR that comprises the HCDR1, HCDR2 and HCDR3 of a HCVR that comprises the amino acid sequence set forth in SEQ ID NO: 18 (or a variant thereof),
and
a LCVR that comprises the LCDR1, LCDR2 and LCDR3 of a LCVR that comprises the amino acid sequence set forth in SEQ ID NO: 26 (or a variant thereof);
(3)
a HCVR that comprises the HCDR1, HCDR2 and HCDR3 of a HCVR that comprises the amino acid sequence set forth in SEQ ID NO: 34 (or a variant thereof),
and
a LCVR that comprises the LCDR1, LCDR2 and LCDR3 of a LCVR that comprises the amino acid sequence set forth in SEQ ID NO: 42 (or a variant thereof);
(4)
a HCVR that comprises the HCDR1, HCDR2 and HCDR3 of a HCVR that comprises the amino acid sequence set forth in SEQ ID NO: 50 (or a variant thereof),
and
a LCVR that comprises the LCDR1, LCDR2 and LCDR3 of a LCVR that comprises the amino acid sequence set forth in SEQ ID NO: 58 (or a variant thereof);
(5)
a HCVR that comprises the HCDR1, HCDR2 and HCDR3 of a HCVR that comprises the amino acid sequence set forth in SEQ ID NO: 66 (or a variant thereof),
and
a LCVR that comprises the LCDR1, LCDR2 and LCDR3 of a LCVR that comprises the amino acid sequence set forth in SEQ ID NO: 74 (or a variant thereof);
(6)
a HCVR that comprises the HCDR1, HCDR2 and HCDR3 of a HCVR that comprises the amino acid sequence set forth in SEQ ID NO: 82 (or a variant thereof),
and
a LCVR that comprises the LCDR1, LCDR2 and LCDR3 of a LCVR that comprises the amino acid sequence set forth in SEQ ID NO: 90 (or a variant thereof);
(7)
a HCVR that comprises the HCDR1, HCDR2 and HCDR3 of a HCVR that comprises the amino acid sequence set forth in SEQ ID NO: 98 (or a variant thereof),
and
a LCVR that comprises the LCDR1, LCDR2 and LCDR3 of a LCVR that comprises the amino acid sequence set forth in SEQ ID NO: 106 (or a variant thereof);
(8)
a HCVR that comprises the HCDR1, HCDR2 and HCDR3 of a HCVR that comprises the amino acid sequence set forth in SEQ ID NO: 98 (or a variant thereof),
and
a LCVR that comprises the LCDR1, LCDR2 and LCDR3 of a LCVR that comprises the amino acid sequence set forth in SEQ ID NO: 114 (or a variant thereof);
(9)
a HCVR that comprises the HCDR1, HCDR2 and HCDR3 of a HCVR that comprises the amino acid sequence set forth in SEQ ID NO: 122 (or a variant thereof),
and
a LCVR that comprises the LCDR1, LCDR2 and LCDR3 of a LCVR that comprises the amino acid sequence set forth in SEQ ID NO: 106 (or a variant thereof);
(10)
a HCVR that comprises the HCDR1, HCDR2 and HCDR3 of a HCVR that comprises the amino acid sequence set forth in SEQ ID NO: 98 (or a variant thereof),
and
a LCVR that comprises the LCDR1, LCDR2 and LCDR3 of a LCVR that comprises the amino acid sequence set forth in SEQ ID NO: 130 (or a variant thereof);
(11)
a HCVR that comprises the HCDR1, HCDR2 and HCDR3 of a HCVR that comprises the amino acid sequence set forth in SEQ ID NO: 138 (or a variant thereof),
and
a LCVR that comprises the LCDR1, LCDR2 and LCDR3 of a LCVR that comprises the amino acid sequence set forth in SEQ ID NO: 106 (or a variant thereof);
(12)
a HCVR that comprises the HCDR1, HCDR2 and HCDR3 of a HCVR that comprises the amino acid sequence set forth in SEQ ID NO: 146 (or a variant thereof),
and
a LCVR that comprises the LCDR1, LCDR2 and LCDR3 of a LCVR that comprises the amino acid sequence set forth in SEQ ID NO: 106 (or a variant thereof);
(13)
a HCVR that comprises the HCDR1, HCDR2 and HCDR3 of a HCVR that comprises the amino acid sequence set forth in SEQ ID NO: 122 (or a variant thereof),
and
a LCVR that comprises the LCDR1, LCDR2 and LCDR3 of a LCVR that comprises the amino acid sequence set forth in SEQ ID NO: 130 (or a variant thereof);
(14)
a HCVR that comprises the HCDR1, HCDR2 and HCDR3 of a HCVR that comprises the amino acid sequence set forth in SEQ ID NO: 146 (or a variant thereof),
and
a LCVR that comprises the LCDR1, LCDR2 and LCDR3 of a LCVR that comprises the amino acid sequence set forth in SEQ ID NO: 114 (or a variant thereof);
(15)
a HCVR that comprises the HCDR1, HCDR2 and HCDR3 of a HCVR that comprises the amino acid sequence set forth in SEQ ID NO: 146 (or a variant thereof),
and
a LCVR that comprises the LCDR1, LCDR2 and LCDR3 of a LCVR that comprises the amino acid sequence set forth in SEQ ID NO: 130 (or a variant thereof);
(16)
a HCVR that comprises the HCDR1, HCDR2 and HCDR3 of a HCVR that comprises the amino acid sequence set forth in SEQ ID NO: 138,
and
a LCVR that comprises the LCDR1, LCDR2 and LCDR3 of a LCVR that comprises the amino acid sequence set forth in SEQ ID NO: 130 (or a variant thereof);
(17)
a HCVR that comprises the HCDR1, HCDR2 and HCDR3 of a HCVR that comprises the amino acid sequence set forth in SEQ ID NO: 154 (or a variant thereof),
and
a LCVR that comprises the LCDR1, LCDR2 and LCDR3 of a LCVR that comprises the amino acid sequence set forth in SEQ ID NO: 162 (or a variant thereof);
(18)
a HCVR that comprises the HCDR1, HCDR2 and HCDR3 of a HCVR that comprises the amino acid sequence set forth in SEQ ID NO: 170 (or a variant thereof),
and
a LCVR that comprises the LCDR1, LCDR2 and LCDR3 of a LCVR that comprises the amino acid sequence set forth in SEQ ID NO: 178 (or a variant thereof);
(19)
a HCVR that comprises the HCDR1, HCDR2 and HCDR3 of a HCVR that comprises the amino acid sequence set forth in SEQ ID NO: 186 (or a variant thereof),
and
a LCVR that comprises the LCDR1, LCDR2 and LCDR3 of a LCVR that comprises the amino acid sequence set forth in SEQ ID NO: 194 (or a variant thereof);
(20)
a HCVR that comprises the HCDR1, HCDR2 and HCDR3 of a HCVR that comprises the amino acid sequence set forth in SEQ ID NO: 202 (or a variant thereof),
and
a LCVR that comprises the LCDR1, LCDR2 and LCDR3 of a LCVR that comprises the amino acid sequence set forth in SEQ ID NO: 210 (or a variant thereof);
(21)
a HCVR that comprises the HCDR1, HCDR2 and HCDR3 of a HCVR that comprises the amino acid sequence set forth in SEQ ID NO: 218 (or a variant thereof),
and
a LCVR that comprises the LCDR1, LCDR2 and LCDR3 of a LCVR that comprises the amino acid sequence set forth in SEQ ID NO: 226 (or a variant thereof);
(22)
a HCVR that comprises the HCDR1, HCDR2 and HCDR3 of a HCVR that comprises the amino acid sequence set forth in SEQ ID NO: 234 (or a variant thereof),
and
a LCVR that comprises the LCDR1, LCDR2 and LCDR3 of a LCVR that comprises the amino acid sequence set forth in SEQ ID NO: 242 (or a variant thereof);
(23)
a HCVR that comprises the HCDR1, HCDR2 and HCDR3 of a HCVR that comprises the amino acid sequence set forth in SEQ ID NO: 250 (or a variant thereof),
and
a LCVR that comprises the LCDR1, LCDR2 and LCDR3 of a LCVR that comprises the amino acid sequence set forth in SEQ ID NO: 258 (or a variant thereof);
(24)
a HCVR that comprises the HCDR1, HCDR2 and HCDR3 of a HCVR that comprises the amino acid sequence set forth in SEQ ID NO: 266 (or a variant thereof),
and
a LCVR that comprises the LCDR1, LCDR2 and LCDR3 of a LCVR that comprises the amino acid sequence set forth in SEQ ID NO: 258 (or a variant thereof);
(25)
a HCVR that comprises the HCDR1, HCDR2 and HCDR3 of a HCVR that comprises the amino acid sequence set forth in SEQ ID NO: 274 (or a variant thereof),
and
a LCVR that comprises the LCDR1, LCDR2 and LCDR3 of a LCVR that comprises the amino acid sequence set forth in SEQ ID NO: 282 (or a variant thereof);
(26)
a HCVR that comprises the HCDR1, HCDR2 and HCDR3 of a HCVR that comprises the amino acid sequence set forth in SEQ ID NO: 290 (or a variant thereof),
and
a LCVR that comprises the LCDR1, LCDR2 and LCDR3 of a LCVR that comprises the amino acid sequence set forth in SEQ ID NO: 298 (or a variant thereof);
(27)
a HCVR that comprises the HCDR1, HCDR2 and HCDR3 of a HCVR that comprises the amino acid sequence set forth in SEQ ID NO: 306 (or a variant thereof),
and
a LCVR that comprises the LCDR1, LCDR2 and LCDR3 of a LCVR that comprises the amino acid sequence set forth in SEQ ID NO: 314 (or a variant thereof);
(28)
a HCVR that comprises the HCDR1, HCDR2 and HCDR3 of a HCVR that comprises the amino acid sequence set forth in SEQ ID NO: 322 (or a variant thereof),
and
a LCVR that comprises the LCDR1, LCDR2 and LCDR3 of a LCVR that comprises the amino acid sequence set forth in SEQ ID NO: 330 (or a variant thereof);
and/or
(29)
a HCVR that comprises the HCDR1, HCDR2 and HCDR3 of a HCVR that comprises the amino acid sequence set forth in SEQ ID NO: 338 (or a variant thereof),
and
a LCVR that comprises the LCDR1, LCDR2 and LCDR3 of a LCVR that comprises the amino acid sequence set forth in SEQ ID NO: 346 (or a variant thereof).

In an embodiment of the disclosure, an antibody or antigen-binding fragment thereof that binds specifically to C5, which is in a co-formulation of the present disclosure comprises:
(a)
a heavy chain variable region including
an HCDR1 that comprises the amino acid sequence set forth in SEQ ID NO: 4 (or a variant thereof),
an HCDR2 that comprises the amino acid sequence set forth in SEQ ID NO: 6 (or a variant thereof),
an HCDR3 that comprises the amino acid sequence set forth in SEQ ID NO: 8 (or a variant thereof),
and a light chain variable region including
an LCDR1 that comprises the amino acid sequence set forth in SEQ ID NO: 12 (or a variant thereof),
an LCDR2 that comprises the amino acid sequence set forth in SEQ ID NO: 14 (or a variant thereof),
an LCDR3 that comprises the amino acid sequence set forth in SEQ ID NO: 16 (or a variant thereof);
(b)
a heavy chain variable region including
an HCDR1 that comprises the amino acid sequence set forth in SEQ ID NO: 20 (or a variant thereof),
an HCDR2 that comprises the amino acid sequence set forth in SEQ ID NO: 22 (or a variant thereof),
an HCDR3 that comprises the amino acid sequence set forth in SEQ ID NO: 24 (or a variant thereof),
and a light chain variable region including
an LCDR1 that comprises the amino acid sequence set forth in SEQ ID NO: 28 (or a variant thereof),
an LCDR2 that comprises the amino acid sequence set forth in SEQ ID NO: 30 (or a variant thereof),
an LCDR3 that comprises the amino acid sequence set forth in SEQ ID NO: 32 (or a variant thereof);
(c)
a heavy chain variable region including
an HCDR1 that comprises the amino acid sequence set forth in SEQ ID NO: 36 (or a variant thereof),
an HCDR2 that comprises the amino acid sequence set forth in SEQ ID NO: 38 (or a variant thereof),
an HCDR3 that comprises the amino acid sequence set forth in SEQ ID NO: 40 (or a variant thereof),
and a light chain variable region including
an LCDR1 that comprises the amino acid sequence set forth in SEQ ID NO: 44 (or a variant thereof),
an LCDR2 that comprises the amino acid sequence set forth in SEQ ID NO: 46 (or a variant thereof),
an LCDR3 that comprises the amino acid sequence set forth in SEQ ID NO: 48 (or a variant thereof);
(d)
a heavy chain variable region including
an HCDR1 that comprises the amino acid sequence set forth in SEQ ID NO: 52 (or a variant thereof),
an HCDR2 that comprises the amino acid sequence set forth in SEQ ID NO: 54 (or a variant thereof),
an HCDR3 that comprises the amino acid sequence set forth in SEQ ID NO: 56 (or a variant thereof),
and a light chain variable region including
an LCDR1 that comprises the amino acid sequence set forth in SEQ ID NO: 60 (or a variant thereof),
an LCDR2 that comprises the amino acid sequence set forth in SEQ ID NO: 62 (or a variant thereof),
an LCDR3 that comprises the amino acid sequence set forth in SEQ ID NO: 64 (or a variant thereof);
(e)
a heavy chain variable region including
an HCDR1 that comprises the amino acid sequence set forth in SEQ ID NO: 68 (or a variant thereof),
an HCDR2 that comprises the amino acid sequence set forth in SEQ ID NO: 70 (or a variant thereof),
an HCDR3 that comprises the amino acid sequence set forth in SEQ ID NO: 72 (or a variant thereof),
and a light chain variable region including
an LCDR1 that comprises the amino acid sequence set forth in SEQ ID NO: 76 (or a variant thereof),
an LCDR2 that comprises the amino acid sequence set forth in SEQ ID NO: 78 (or a variant thereof),
an LCDR3 that comprises the amino acid sequence set forth in SEQ ID NO: 80 (or a variant thereof);
(f)
a heavy chain variable region including
an HCDR1 that comprises the amino acid sequence set forth in SEQ ID NO: 84 (or a variant thereof),
an HCDR2 that comprises the amino acid sequence set forth in SEQ ID NO: 86 (or a variant thereof),
an HCDR3 that comprises the amino acid sequence set forth in SEQ ID NO: 88 (or a variant thereof),
and a light chain variable region including
an LCDR1 that comprises the amino acid sequence set forth in SEQ ID NO: 92 (or a variant thereof),
an LCDR2 that comprises the amino acid sequence set forth in SEQ ID NO: 94 (or a variant thereof),
an LCDR3 that comprises the amino acid sequence set forth in SEQ ID NO: 96 (or a variant thereof);
(g)
a heavy chain variable region including
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region including
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
(h)
a heavy chain variable region including
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region including
an LCDR1 that comprises the amino acid sequence set forth in SEQ ID NO: 116 (or a variant thereof),
an LCDR2 that comprises the amino acid sequence set forth in SEQ ID NO: 118 (or a variant thereof),
an LCDR3 that comprises the amino acid sequence set forth in SEQ ID NO: 120 (or a variant thereof);
(i)
a heavy chain variable region including
an HCDR1 that comprises the amino acid sequence set forth in SEQ ID NO: 124 (or a variant thereof),
an HCDR2 that comprises the amino acid sequence set forth in SEQ ID NO: 126 (or a variant thereof),
an HCDR3 that comprises the amino acid sequence set forth in SEQ ID NO: 128 (or a variant thereof),
and a light chain variable region including
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
(i)
a heavy chain variable region including
an HCDR1 that comprises the amino acid sequence set forth in SEQ ID NO: 100 (or a variant thereof),
an HCDR2 that comprises the amino acid sequence set forth in SEQ ID NO: 102 (or a variant thereof),
an HCDR3 that comprises the amino acid sequence set forth in SEQ ID NO: 104 (or a variant thereof),
and a light chain variable region including
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
(k)
a heavy chain variable region including
an HCDR1 that comprises the amino acid sequence set forth in SEQ ID NO: 140 (or a variant thereof),
an HCDR2 that comprises the amino acid sequence set forth in SEQ ID NO: 142 (or a variant thereof),
an HCDR3 that comprises the amino acid sequence set forth in SEQ ID NO: 144 (or a variant thereof),
and a light chain variable region including
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
(l)
a heavy chain variable region including
an HCDR1 that comprises the amino acid sequence set forth in SEQ ID NO: 148 (or a variant thereof),
an HCDR2 that comprises the amino acid sequence set forth in SEQ ID NO: 150 (or a variant thereof),
an HCDR3 that comprises the amino acid sequence set forth in SEQ ID NO: 152 (or a variant thereof),
and a light chain variable region including
an LCDR1 that comprises the amino acid sequence set forth in SEQ ID NO: 108 (or a variant thereof),
an LCDR2 that comprises the amino acid sequence set forth in SEQ ID NO: 110 (or a variant thereof),
an LCDR3 that comprises the amino acid sequence set forth in SEQ ID NO: 112 (or a variant thereof);
(m)
a heavy chain variable region including
an HCDR1 that comprises the amino acid sequence set forth in SEQ ID NO: 124 (or a variant thereof),
an HCDR2 that comprises the amino acid sequence set forth in SEQ ID NO: 126 (or a variant thereof),
an HCDR3 that comprises the amino acid sequence set forth in SEQ ID NO: 128 (or a variant thereof),
and a light chain variable region including
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
(n)
a heavy chain variable region including
an HCDR1 that comprises the amino acid sequence set forth in SEQ ID NO: 148 (or a variant thereof),
an HCDR2 that comprises the amino acid sequence set forth in SEQ ID NO: 150 (or a variant thereof),
an HCDR3 that comprises the amino acid sequence set forth in SEQ ID NO: 152 (or a variant thereof),
and a light chain variable region including
an LCDR1 that comprises the amino acid sequence set forth in SEQ ID NO: 116 (or a variant thereof),
an LCDR2 that comprises the amino acid sequence set forth in SEQ ID NO: 118 (or a variant thereof),
an LCDR3 that comprises the amino acid sequence set forth in SEQ ID NO: 120 (or a variant thereof);
(o)
a heavy chain variable region including
an HCDR1 that comprises the amino acid sequence set forth in SEQ ID NO: 148 (or a variant thereof),
an HCDR2 that comprises the amino acid sequence set forth in SEQ ID NO: 150 (or a variant thereof),
an HCDR3 that comprises the amino acid sequence set forth in SEQ ID NO: 152 (or a variant thereof),
and a light chain variable region including
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
(p)
a heavy chain variable region including
an HCDR1 that comprises the amino acid sequence set forth in SEQ ID NO: 140 (or a variant thereof),
an HCDR2 that comprises the amino acid sequence set forth in SEQ ID NO: 142 (or a variant thereof),
an HCDR3 that comprises the amino acid sequence set forth in SEQ ID NO: 144 (or a variant thereof),
and a light chain variable region including
an LCDR1 that comprises the amino acid sequence set forth in SEQ ID NO: 132 (or a variant thereof),
an LCDR2 that comprises the amino acid sequence set forth in SEQ ID NO: 134 (or a variant thereof),
an LCDR3 that comprises the amino acid sequence set forth in SEQ ID NO: 136 (or a variant thereof);
(q)
a heavy chain variable region including
an HCDR1 that comprises the amino acid sequence set forth in SEQ ID NO: 156 (or a variant thereof),
an HCDR2 that comprises the amino acid sequence set forth in SEQ ID NO: 158 (or a variant thereof),
an HCDR3 that comprises the amino acid sequence set forth in SEQ ID NO: 160 (or a variant thereof),
and a light chain variable region including
an LCDR1 that comprises the amino acid sequence set forth in SEQ ID NO: 164 (or a variant thereof),
an LCDR2 that comprises the amino acid sequence set forth in SEQ ID NO: 166 (or a variant thereof),
an LCDR3 that comprises the amino acid sequence set forth in SEQ ID NO: 168 (or a variant thereof);
(r)
a heavy chain variable region including
an HCDR1 that comprises the amino acid sequence set forth in SEQ ID NO: 172 (or a variant thereof),
an HCDR2 that comprises the amino acid sequence set forth in SEQ ID NO: 174 (or a variant thereof),
an HCDR3 that comprises the amino acid sequence set forth in SEQ ID NO: 176 (or a variant thereof),
and a light chain variable region including
an LCDR1 that comprises the amino acid sequence set forth in SEQ ID NO: 180 (or a variant thereof),
an LCDR2 that comprises the amino acid sequence set forth in SEQ ID NO: 182 (or a variant thereof),
an LCDR3 that comprises the amino acid sequence set forth in SEQ ID NO: 184 (or a variant thereof);
(s)
a heavy chain variable region including
an HCDR1 that comprises the amino acid sequence set forth in SEQ ID NO: 188 (or a variant thereof),
an HCDR2 that comprises the amino acid sequence set forth in SEQ ID NO: 190 (or a variant thereof),
an HCDR3 that comprises the amino acid sequence set forth in SEQ ID NO: 192 (or a variant thereof),
and a light chain variable region including
an LCDR1 that comprises the amino acid sequence set forth in SEQ ID NO: 196 (or a variant thereof),
an LCDR2 that comprises the amino acid sequence set forth in SEQ ID NO: 198 (or a variant thereof),
an LCDR3 that comprises the amino acid sequence set forth in SEQ ID NO: 200 (or a variant thereof);
(t)
a heavy chain variable region including
an HCDR1 that comprises the amino acid sequence set forth in SEQ ID NO: 204 (or a variant thereof),
an HCDR2 that comprises the amino acid sequence set forth in SEQ ID NO: 206 (or a variant thereof),
an HCDR3 that comprises the amino acid sequence set forth in SEQ ID NO: 208 (or a variant thereof),
and a light chain variable region including
an LCDR1 that comprises the amino acid sequence set forth in SEQ ID NO: 212 (or a variant thereof),
an LCDR2 that comprises the amino acid sequence set forth in SEQ ID NO: 214 (or a variant thereof),
an LCDR3 that comprises the amino acid sequence set forth in SEQ ID NO: 216 (or a variant thereof);
(u)
a heavy chain variable region including
an HCDR1 that comprises the amino acid sequence set forth in SEQ ID NO: 220 (or a variant thereof),
an HCDR2 that comprises the amino acid sequence set forth in SEQ ID NO: 222 (or a variant thereof),
an HCDR3 that comprises the amino acid sequence set forth in SEQ ID NO: 224 (or a variant thereof),
and a light chain variable region including
an LCDR1 that comprises the amino acid sequence set forth in SEQ ID NO: 228 (or a variant thereof),
an LCDR2 that comprises the amino acid sequence set forth in SEQ ID NO: 230 (or a variant thereof),
an LCDR3 that comprises the amino acid sequence set forth in SEQ ID NO: 232 (or a variant thereof);
(v)
a heavy chain variable region including
an HCDR1 that comprises the amino acid sequence set forth in SEQ ID NO: 236 (or a variant thereof),
an HCDR2 that comprises the amino acid sequence set forth in SEQ ID NO: 238 (or a variant thereof),
an HCDR3 that comprises the amino acid sequence set forth in SEQ ID NO: 240 (or a variant thereof),
and a light chain variable region including
an LCDR1 that comprises the amino acid sequence set forth in SEQ ID NO: 244 (or a variant thereof),
an LCDR2 that comprises the amino acid sequence set forth in SEQ ID NO: 246 (or a variant thereof),
an LCDR3 that comprises the amino acid sequence set forth in SEQ ID NO: 248 (or a variant thereof);
(w)
a heavy chain variable region including
an HCDR1 that comprises the amino acid sequence set forth in SEQ ID NO: 252 (or a variant thereof),
an HCDR2 that comprises the amino acid sequence set forth in SEQ ID NO: 254 (or a variant thereof),
an HCDR3 that comprises the amino acid sequence set forth in SEQ ID NO: 256 (or a variant thereof),
and a light chain variable region including
an LCDR1 that comprises the amino acid sequence set forth in SEQ ID NO: 260 (or a variant thereof),
an LCDR2 that comprises the amino acid sequence set forth in SEQ ID NO: 262 (or a variant thereof),
an LCDR3 that comprises the amino acid sequence set forth in SEQ ID NO: 264 (or a variant thereof);
(x)
a heavy chain variable region including
an HCDR1 that comprises the amino acid sequence set forth in SEQ ID NO: 268 (or a variant thereof),
an HCDR2 that comprises the amino acid sequence set forth in SEQ ID NO: 270 (or a variant thereof),
an HCDR3 that comprises the amino acid sequence set forth in SEQ ID NO: 272 (or a variant thereof),
and a light chain variable region including
an LCDR1 that comprises the amino acid sequence set forth in SEQ ID NO: 260 (or a variant thereof),
an LCDR2 that comprises the amino acid sequence set forth in SEQ ID NO: 262 (or a variant thereof),
an LCDR3 that comprises the amino acid sequence set forth in SEQ ID NO: 264 (or a variant thereof);
(y)
a heavy chain variable region including
an HCDR1 that comprises the amino acid sequence set forth in SEQ ID NO: 276 (or a variant thereof),
an HCDR2 that comprises the amino acid sequence set forth in SEQ ID NO: 278 (or a variant thereof),
an HCDR3 that comprises the amino acid sequence set forth in SEQ ID NO: 280 (or a variant thereof),
and a light chain variable region including
an LCDR1 that comprises the amino acid sequence set forth in SEQ ID NO: 284 (or a variant thereof),
an LCDR2 that comprises the amino acid sequence set forth in SEQ ID NO: 286 (or a variant thereof),
an LCDR3 that comprises the amino acid sequence set forth in SEQ ID NO: 288 (or a variant thereof);
(z)
a heavy chain variable region including
an HCDR1 that comprises the amino acid sequence set forth in SEQ ID NO: 292 (or a variant thereof),
an HCDR2 that comprises the amino acid sequence set forth in SEQ ID NO: 294 (or a variant thereof),
an HCDR3 that comprises the amino acid sequence set forth in SEQ ID NO: 296 (or a variant thereof),
and a light chain variable region including
an LCDR1 that comprises the amino acid sequence set forth in SEQ ID NO: 300 (or a variant thereof),
an LCDR2 that comprises the amino acid sequence set forth in SEQ ID NO: 302 (or a variant thereof),
an LCDR3 that comprises the amino acid sequence set forth in SEQ ID NO: 304 (or a variant thereof);
(aa)
a heavy chain variable region including
an HCDR1 that comprises the amino acid sequence set forth in SEQ ID NO: 308 (or a variant thereof),
an HCDR2 that comprises the amino acid sequence set forth in SEQ ID NO: 310 (or a variant thereof),
an HCDR3 that comprises the amino acid sequence set forth in SEQ ID NO: 312 (or a variant thereof),
and a light chain variable region including
an LCDR1 that comprises the amino acid sequence set forth in SEQ ID NO: 316 (or a variant thereof),
an LCDR2 that comprises the amino acid sequence set forth in SEQ ID NO: 318 (or a variant thereof),
an LCDR3 that comprises the amino acid sequence set forth in SEQ ID NO: 320 (or a variant thereof);
(ab)
a heavy chain variable region including
an HCDR1 that comprises the amino acid sequence set forth in SEQ ID NO: 324 (or a variant thereof),
an HCDR2 that comprises the amino acid sequence set forth in SEQ ID NO: 326 (or a variant thereof),
an HCDR3 that comprises the amino acid sequence set forth in SEQ ID NO: 328 (or a variant thereof),
and a light chain variable region including
an LCDR1 that comprises the amino acid sequence set forth in SEQ ID NO: 332 (or a variant thereof),
an LCDR2 that comprises the amino acid sequence set forth in SEQ ID NO: 334 (or a variant thereof),
an LCDR3 that comprises the amino acid sequence set forth in SEQ ID NO: 336 (or a variant thereof);
and/or
(ac)
a heavy chain variable region including
an HCDR1 that comprises the amino acid sequence set forth in SEQ ID NO: 340 (or a variant thereof),
an HCDR2 that comprises the amino acid sequence set forth in SEQ ID NO: 342 (or a variant thereof),
an HCDR3 that comprises the amino acid sequence set forth in SEQ ID NO: 344 (or a variant thereof),
and a light chain variable region including
an LCDR1 that comprises the amino acid sequence set forth in SEQ ID NO: 348 (or a variant thereof),
an LCDR2 that comprises the amino acid sequence set forth in SEQ ID NO: 350 (or a variant thereof),
an LCDR3 that comprises the amino acid sequence set forth in SEQ ID NO: 352 (or a variant thereof).

In an embodiment of the disclosure, an antibody or antigen-binding fragment thereof that binds specifically to C5, which is in a co-formulation of the present disclosure comprises:
(i)
a heavy chain variable region that comprises the amino acid sequence set forth in SEQ ID NO: 2 (or a variant thereof),
and
a light chain variable region that comprises the amino acid sequence set forth in SEQ ID NO: 10 (or a variant thereof);
(ii)
a heavy chain variable region that comprises the amino acid sequence set forth in SEQ ID NO: 18 (or a variant thereof),
and
a light chain variable region that comprises the amino acid sequence set forth in SEQ ID NO: 26 (or a variant thereof);
(iii)
a heavy chain variable region that comprises the amino acid sequence set forth in SEQ ID NO: 34 (or a variant thereof),
and
a light chain variable region that comprises the amino acid sequence set forth in SEQ ID NO: 42 (or a variant thereof);
(iv)
a heavy chain variable region that comprises the amino acid sequence set forth in SEQ ID NO: 50 (or a variant thereof),
and
a light chain variable region that comprises the amino acid sequence set forth in SEQ ID NO: 58 (or a variant thereof);
(v)
a heavy chain variable region that comprises the amino acid sequence set forth in SEQ ID NO: 66 (or a variant thereof),
and
a light chain variable region that comprises the amino acid sequence set forth in SEQ ID NO: 74 (or a variant thereof);
(vi)
a heavy chain variable region that comprises the amino acid sequence set forth in SEQ ID NO: 82 (or a variant thereof),
and
a light chain variable region that comprises the amino acid sequence set forth in SEQ ID NO: 90 (or a variant thereof);
(vii)
a heavy chain variable region that comprises the amino acid sequence set forth in SEQ ID NO: 98 (or a variant thereof),
and
a light chain variable region that comprises the amino acid sequence set forth in SEQ ID NO: 106 (or a variant thereof);
(viii)
a heavy chain variable region that comprises the amino acid sequence set forth in SEQ ID NO: 98 (or a variant thereof),
and
a light chain variable region that comprises the amino acid sequence set forth in SEQ ID NO: 114 (or a variant thereof);
(ix)
a heavy chain variable region that comprises the amino acid sequence set forth in SEQ ID NO: 122 (or a variant thereof),
and
a light chain variable region that comprises the amino acid sequence set forth in SEQ ID NO: 106 (or a variant thereof);
(x)
a heavy chain variable region that comprises the amino acid sequence set forth in SEQ ID NO: 98 (or a variant thereof),
and
a light chain variable region that comprises the amino acid sequence set forth in SEQ ID NO: 130 (or a variant thereof);
(xi)
a heavy chain variable region that comprises the amino acid sequence set forth in SEQ ID NO: 138 (or a variant thereof),
and
a light chain variable region that comprises the amino acid sequence set forth in SEQ ID NO: 106 (or a variant thereof);
(xii)
a heavy chain variable region that comprises the amino acid sequence set forth in SEQ ID NO: 146 (or a variant thereof),
and
a light chain variable region that comprises the amino acid sequence set forth in SEQ ID NO: 106 (or a variant thereof);
(xiii)
a heavy chain variable region that comprises the amino acid sequence set forth in SEQ ID NO: 122 (or a variant thereof),
and
a light chain variable region that comprises the amino acid sequence set forth in SEQ ID NO: 130 (or a variant thereof);
(xiv)
a heavy chain variable region that comprises the amino acid sequence set forth in SEQ ID NO: 146 (or a variant thereof),
and
a light chain variable region that comprises the amino acid sequence set forth in SEQ ID NO: 114 (or a variant thereof);
(xv)
a heavy chain variable region that comprises the amino acid sequence set forth in SEQ ID NO: 146 (or a variant thereof),
and
a light chain variable region that comprises the amino acid sequence set forth in SEQ ID NO: 130 (or a variant thereof);
(xvi)
a heavy chain variable region that comprises the amino acid sequence set forth in SEQ ID NO: 138 (or a variant thereof),
and
a light chain variable region that comprises the amino acid sequence set forth in SEQ ID NO: 130 (or a variant thereof);
(xvii)
a heavy chain variable region that comprises the amino acid sequence set forth in SEQ ID NO: 154 (or a variant thereof),
and
a light chain variable region that comprises the amino acid sequence set forth in SEQ ID NO: 162 (or a variant thereof);
(xviii)
a heavy chain variable region that comprises the amino acid sequence set forth in SEQ ID NO: 170 (or a variant thereof),
and
a light chain variable region that comprises the amino acid sequence set forth in SEQ ID NO: 178 (or a variant thereof);
(xix)
a heavy chain variable region that comprises the amino acid sequence set forth in SEQ ID NO: 186 (or a variant thereof),
and
a light chain variable region that comprises the amino acid sequence set forth in SEQ ID NO: 194 (or a variant thereof);
(xx)
a heavy chain variable region that comprises the amino acid sequence set forth in SEQ ID NO: 202 (or a variant thereof),
and
a light chain variable region that comprises the amino acid sequence set forth in SEQ ID NO: 210 (or a variant thereof);
(xxi)
a heavy chain variable region that comprises the amino acid sequence set forth in SEQ ID NO: 218 (or a variant thereof),
and
a light chain variable region that comprises the amino acid sequence set forth in SEQ ID NO: 226 (or a variant thereof);
(xxii)
a heavy chain variable region that comprises the amino acid sequence set forth in SEQ ID NO: 234 (or a variant thereof),
and
a light chain variable region that comprises the amino acid sequence set forth in SEQ ID NO: 242 (or a variant thereof);
(xxiii)
a heavy chain variable region that comprises the amino acid sequence set forth in SEQ ID NO: 250 (or a variant thereof),
and
a light chain variable region that comprises the amino acid sequence set forth in SEQ ID NO: 258 (or a variant thereof);
(xxiv)
a heavy chain variable region that comprises the amino acid sequence set forth in SEQ ID NO: 266 (or a variant thereof),
and
a light chain variable region that comprises the amino acid sequence set forth in SEQ ID NO: 258 (or a variant thereof);
(xxv)
a heavy chain variable region that comprises the amino acid sequence set forth in SEQ ID NO: 274 (or a variant thereof),
and
a light chain variable region that comprises the amino acid sequence set forth in SEQ ID NO: 282 (or a variant thereof);
(xxvi)
a heavy chain variable region that comprises the amino acid sequence set forth in SEQ ID NO: 290 (or a variant thereof),
and
a light chain variable region that comprises the amino acid sequence set forth in SEQ ID NO: 298 (or a variant thereof);
(xxvii)
a heavy chain variable region that comprises the amino acid sequence set forth in SEQ ID NO: 306 (or a variant thereof),
and
a light chain variable region that comprises the amino acid sequence set forth in SEQ ID NO: 314 (or a variant thereof);
(xxviii)
a heavy chain variable region that comprises the amino acid sequence set forth in SEQ ID NO: 322 (or a variant thereof),
and
a light chain variable region that comprises the amino acid sequence set forth in SEQ ID NO: 330 (or a variant thereof);
and/or
(xxix)
a heavy chain variable region that comprises the amino acid sequence set forth in SEQ ID NO: 338 (or a variant thereof),
and
a light chain variable region that comprises the amino acid sequence set forth in SEQ ID NO: 346 (or a variant thereof).

In an embodiment of the disclosure, an antibody or antigen-binding fragment thereof that binds specifically to C5, which is in a co-formulation of the present disclosure comprises a heavy chain including the amino acid sequence:

(SEQ ID NO: 358)
QVQLQESGPGLVKPSETLSLTCTVSGDSVSSSYWTWIRQPPGKGLEWIGYIYYSGSSNYN
PSLKSRATISVDTSKNQFSLKLSSVTAADTAVYYCAREGNVDTTMIFDYWGQGTLVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGK;

and a light chain including the amino acid sequence:

(SEQ ID NO: 359)
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;

such an antibody may be referred to herein as Pozelimab or REGN3918 (variable regions and CDRs underscored). See Proposed INN: List 120, WHO Drug Information, Vol. 32, No. 4, 2018.

“H2M11683N”; “H2M11686N”; “H4H12159P”; “H4H12161P”; “H4H12163P”; “H4H12164P”; “H4H12166P”; “H4H12166P2”; “H4H12166P3”; “H4H12166P4”; “H4H12166P5”; “H4H12166P6”; “H4H12166P7”; “H4H12166P8”; “H4H12166P9”; “H4H12166P10”; “H4H12167P”; “H4H12168P”; “H4H12169P”; “H4H12170P”; “H4H12171P”; “H4H12175P”; “H4H12176P2”; “H4H12177P2”; “H4H12183P2”; “H2M11682N”; “H2M11684N”; “H2M11694N” or “H2M11695N”, unless otherwise stated, refer to anti-C5 antigen-binding proteins, e.g., antibodies and antigen-binding fragments thereof (including multispecific antigen-binding proteins), that bind specifically to C5, including an immunoglobulin heavy chain or variable region thereof (V_{H}) including the amino acid sequence specifically set forth herein corresponding to Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 2; 18; 34; 50; 66; 82; 98; 98; 122; 98; 138; 146; 122; 146; 146; 138; 154; 170; 186; 202; 218; 234; 250; 266; 274; 290; 306; 322 or 338) (or a variant thereof), and/or an immunoglobulin light chain or variable region thereof (V_{L}) including the amino acid sequence specifically set forth herein corresponding to Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 10; 26; 42; 58; 74; 90; 106; 114; 106; 130; 106; 106; 130; 114; 130; 130; 162; 178; 194; 210; 226; 242; 258; 258; 282; 298; 314; 330 or 346) (or a variant thereof); and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In an embodiment of the disclosure, the V_{H} is linked to an IgG constant heavy chain domain (e.g., lgG1 or lgG4 (e.g., lgG4 (S228P mutant))) and/or the V_{L} is linked to a lambda or kappa constant light chain domain.

An “anti-C5” antibody or antigen-binding fragment thereof or antibody or antigen-binding fragment thereof that “binds specifically” to C5 binds to human C5 with a Kp of at least 1 nM (i.e., 1 nM or a higher affinity), e.g., about 0.1 or 0.2 nM.

In an embodiment of the disclosed technology, an anti-C5 antibody or antigen-binding fragment thereof is missing the C-terminal Lysine from the heavy chain.

### Interfering RNA (iRNA)

The present invention includes methods and compositions including the use of C5 iRNAs such as Cemdisiran (e.g., Cemdisiran Na⁺ form, other salt form or free form), for example for treating or preventing MG.

In an embodiment of the invention, the C5 iRNA, e.g., Cemdisiran, is in an aqueous solution including water for Injection, US Pharmcopeia (USP) and sodium hydroxide and/or phosphoric acid to adjust the pH to ˜7.0.

The present disclosure provides a co-formulation that includes an anti-C5 antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; Crovalimab; Eculizumab, Tesidolumab, Mubodina or Ravulizumab; preferably, Pozelimab); and an iRNA which effects the RNA-induced silencing complex (RISC)-mediated cleavage of RNA transcripts of a C5 gene (C5 iRNA), e.g. Cemdisiran (e.g., Cemdisiran/Pozelimab). The C5 gene may be within a cell, e.g., a cell within a subject, such as a human. The present disclosure provides iRNA agents for inclusion in a co-formulation of the disclosure which effect the RNA-induced silencing complex (RISC)-mediated cleavage of RNA transcripts of a complement component C5 gene.

Dosages of Cemdisiran specifically cited herein include embodiments wherein an equivalent molar amount of the Cemdisiran free form or a corresponding salt form is administered.

As discussed in detail herein, the present invention also includes a method for treating or preventing a C5 associated disease or disorder, preferably MG, comprising administering a therapeutically effective amount of Cemdisiran, e.g., Na⁺ salt form or another salt form or free form.

The present invention also includes a method for treating or preventing a C5 associated disease or disorder, preferably MG, comprising administering about 567 or 566.6 mg (±57 mg) of the Cemdisiran free form or an equivalent molar amount of the free molecule but in a salt form, e.g., Na⁺ form, about every 12 weeks (±7 days).

Cemdisiran is a chemically synthesized double-stranded oligonucleotide glycoconjugate that is covalently linked to a ligand containing 3 GalNAc residues to facilitate targeted delivery to the liver. All nucleosides are modified with 2′-deoxy, 2′-methoxy, or 2′-fluoro groups and are connected through 3′ to 5′ phosphodiester linkages, thus forming the sugar-phosphate backbone of the oligonucleotide.

The sense strand (A-125167) contains 21 nucleotides and the antisense strand (A-125647) contains 25. The 3′-end of the sense strand is conjugated to a triantennary GalNAc moiety (referred to as L96) through a phosphodiester linkage.

The antisense strand (A-125647) contains four phosphorothioate linkages, two consecutive phosphorothioate linkages at the 3′ end and two at the 5′ end. The sense strand (A-125167) contains two phosphorothioate linkages at the 5′ end. The 21 nucleotides of the sense strand hybridize with the complementary 21 nucleotides of the antisense strand, thus forming 21 nucleotide base pairs duplex with a 4-base overhang at the 3′-end of the antisense strand. The bases involved in base pair formation are connected with a center dot (see chemical structure below). Cemdisiran is preferably in a salt form, e.g., the Na⁺ salt form, but the present disclosure includes embodiments including Cemdisiran in the free acid form as well as in other salt forms, e.g., Ca²⁺ or Mg²⁺ salts.

Cemdisiran, including salt forms thereof, may be associated with a lipid nanoparticle (LNP) delivery system, e.g., including lipids PEG-DMG (1,2-dimyristoyl-rac-glycero-3-[methoxy (polyethylene glycol)-2000]), PEG-DMPE (1,2-dimyristoyl-sn-glycero-3-phosphoethanolamine-N-[methoxy (polyethylene glycol)-2000]), DSPC (1,2-distearoyl-sn-glycero-3-phosphocholine) or DOPE (1,2-dioleoyl-sn-glycero-3-phosphoethanolamine). In an embodiment of the invention, the Cemdisiran is not associated with a LNP.

When expressing, herein, the concentration of RNAi in a composition, such as a co-formulation of the present disclosure, in terms of mass per volume (e.g., mg/ml), the RNAi is in a salt form or a free acid form. Preferably, when referring to Cemdisiran as such, the Cemdisiran is in salt form, preferably Na⁺ salt form. Na⁺ counter-ions are present due to the net-negatively charged ribonucleotide phosphate backbone. The quantity of Cemdisiran free acid form can be obtained by multiplying the Cemdisiran Na⁺ salt form concentration by 0.9443. For example, about 566.6 or 567 mg of a 600 mg quantity of Cemdisiran Na⁺ form is the Cemdisiran free form.

Some embodiments of the present invention refer to a quantity of Cemdisiran free form which is in a salt form, e.g., 567 mg of Cemdisiran free form which is in Na⁺ salt form. This would refer to a quantity of Cemdisiran Na+ form wherein the amount of free form component of Cemdisiran (not including the Na⁺) is 567 mg.

In addition, an amount of Cemdisiran salt form which is the molar equivalent to an amount of Cemdisiran Na⁺ form can be calculated based on the molecular weight of Cemdisiran Na⁺ form of the duplex molecule which is summarized below in Table C. For example, 600 mg of Cemdisiran Na⁺ salt is about 0.00003376143 moles (about 3.38×10⁻⁵ moles or 33.8 micromoles).

TABLE C
Cemdisiran Molecular Weight

| | ALN-62643 | A-125167 | A-125647 |
|---|---|---|---|
| | Duplex | (sense strand) | (antisense strand) |
| Molecular weight of the | 17,771.76 Da | 9144 Da | 8628 Da |
| sodium salt | | | |
| Molecular weight of the free | 16,782.56 Da | 8682 Da | 8100 Da |
| acid | | | |

Cemdisiran includes salt forms, preferably Na⁺ salt form; or another salt form or free form.

In an embodiment of the invention, the C5 iRNA is ALN-62643.

In an embodiment of the invention the C5 iRNA has the structure shown below wherein A-125167 is on top (5′-3′) and A-125647 is on bottom (3′-5′):

```
                                                                                                                                                                    3′
                    5′ Ams-Ams-Gf-Cm-Af-Am-Gf-Am-Uf-Af-Uf-Um-Uf-Um-Um-Af-Um-Af-Am-Um-Am-L96
                            • • • • • • • • • • • • • • • • • • • • •
dT-dT-Ums-Ums-Um-Um-Cm-Gf-Um-Uf-Cm-Uf-Am-Um Am-Af-Am-Af-Am-Um-Af-Um-Ufs-Afs-Um
3′ 5′
```

Af, Gf and Uf=2′-F ribonucleosides
Am, Cm and Um-2′-OMe ribonucleosides
dT=thymidine
S=phosphorothioate
L96 is

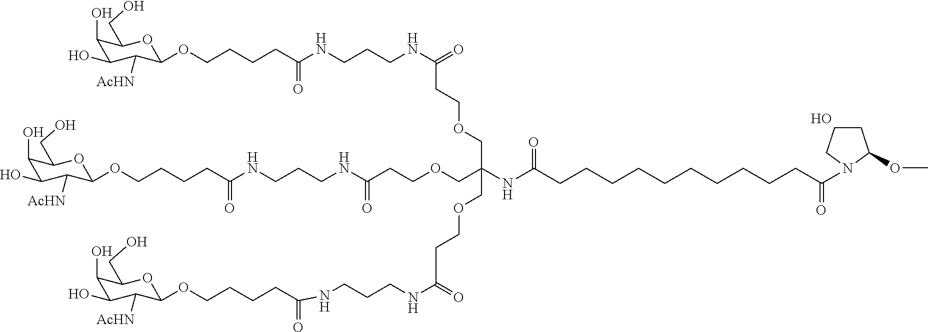

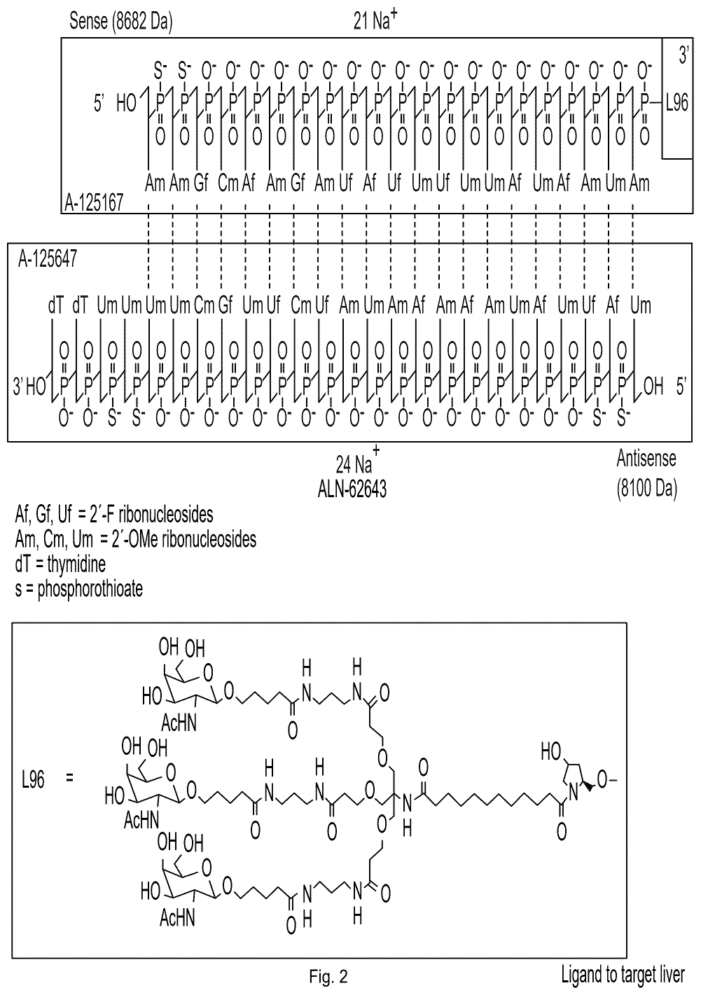

### See FIG. 2.

SEQ ID NO: 406 is 5′AAGCAAGAUAUUUUUAUAAUA3′ with the modified bases and phosporothioate linkages described above. SEQ ID NO: 369 is 5′UAUUAUAAAAAUAUCUUGCUUUUTT3′ with the modified bases and phosporothioate linkages described above.

The present invention includes methods of use a C5 iRNA comprising a double stranded RNA, which may include one or more overhanging bases, including nucleotide strands 5′AAGCAAGAUAUUUUUAUAAUA3′ (SEQ ID NO: 406) and 5′UAUUAUAAAAAUAUCUUGCUUUUTT3′ (SEQ ID NO: 369); wherein one more of the nucleoside bases is 2′-deoxy, 2′-methoxy and/or 2′-fluoro modified; which may include one more sugar ligands.

In an embodiment of the disclosure the C5 iRNA (e.g., dsRNA) is characterized by the strand structures:

(3′-5′) A=A=G-C-A-A-G-A-U-A-U-U-U-U-U-A-U-A-A-U-A-R1
• • • • • • • • • • • • • • • • • •
• • •
(5′-3′)Z-Z-U=U=U-U-C-G-U-U-C-U-A-U-A-A-A-A-A-U-A-U-U=A=U

[3′-5′ and 5′-3′ refers to polarity of the internucleotide bonds]
wherein,
X is 2′-deoxy-2′-fluoro
X is 2′-O-methyl
Z is thymidine

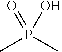

— is

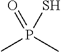

═ is
R1- is

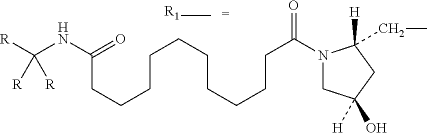

R— is

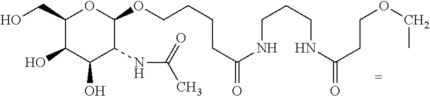 [SMILES: CCOCCC(=O)NCCCNC(=O)CCCCO[C@@H]1O[C@H](CO)[C@H](O)[C@H](O)[C@H]1NC(C)=O]

In an embodiment of the invention, the C5 iRNA is Cemdisiran. See International Nonproprietary Names for Pharmaceutical Substances (INN) (Proposed INN: List 114), WHO Drug Information, Vol. 29, No. 4, 2015; or CAS No. 1639267-24-5. See also “Abbreviations and Symbols for Nucleic Acids, Polynucleotides and their Constituents” Pure and Applied Chemistry, vol. 40, no. 3, 1974, pp. 277-331. See FDA UNII (Unique Ingredient Identifiers (UNIIs)) record S66Z65E10T or X0DZ4POQ4K.

The C5 iRNAs that can be included in co-formulations of the disclosure and/or used in methods for treating or preventing MG include an RNA strand (e.g., the antisense strand) having a region which is about 30 nucleotides or less in length, e.g., at least 15, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of a C5 gene.

In an embodiment of the disclosure, a C5 iRNA is a glycoconjugate that includes a double stranded RNA complementary to a region of C5 which is conjugated (e.g., by a linker) to a terminal mono-, or bi-, tri-antennary N-acetylgalactosamine (GalNAc) group, preferably triantennary N-acetylgalactosamine.

In an embodiment of the disclosure, an iRNA agent which may be included in a co-formulation of the present disclosure and/or used in methods for treating or preventing MG, includes a single stranded RNA that interacts with a target RNA sequence, e.g., a C5 target mRNA sequence, to direct the cleavage of the target RNA. Without wishing to be bound by theory, it is believed that long double stranded RNA introduced into cells is broken down into siRNA by a Type III endonuclease known as Dicer (Sharp et al. (2001) Genes Dev. 15:485). Dicer, a ribonuclease-Ill-like enzyme, processes the dsRNA into 19-23 base pair short interfering RNAs (siRNAs) with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a siRNA generated within a cell and which promotes the formation of a RISC complex to effect silencing of the target gene, i.e., a C5 gene. Accordingly, the term “siRNA” is also used herein to refer to an iRNA as described above.

In another embodiment, the iRNA agent which may be included in a co-formulation of the present disclosure and/or used in methods for treating or preventing MG may be a single-stranded siRNA that is introduced into a cell or organism to inhibit a target mRNA. Single-stranded iRNA agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an iRNA for use in the compositions, uses, and methods of the disclosure is a double-stranded RNA and is referred to herein as a “double stranded iRNA agent,” “double-stranded RNA (dsRNA) molecule,” “dsRNA agent,” or “dsRNA”. The term “dsRNA”, refers to a complex of ribonucleic acid molecules, having a duplex structure including two anti-parallel and substantially complementary nucleic acid strands, referred to as having “sense” and “antisense” orientations with respect to a target RNA, i.e., a C5 gene. In some embodiments of the disclosure, a double-stranded RNA (dsRNA) triggers the degradation of a target RNA, e.g., an mRNA, through a post-transcriptional gene-silencing mechanism referred to herein as RNA interference or RNAi.

In an embodiment of the disclosure, the iRNA is a double-stranded ribonucleic acid (dsRNA) wherein the dsRNA comprises a sense strand and an antisense strand, wherein the sense strand comprises nucleotides (e.g., at least 15 contiguous nucleotides) differing by no more than 3 nucleotides from the nucleotide sequence of C5 (open reading frame underscored):

(SEQ ID NO: 360)
tatatccgtg gtttcctgct acctccaacc atgggccttt tgggaatact ttgtttttta 60
atcttcctgg ggaaaacctg gggacaggag caaacatatg tcatttcagc accaaaaata 120
ttccgtgttg gagcatctga aaatattgtg attcaagttt atggatacac tgaagcattt 180
gatgcaacaa tctctattaa aagttatcct gataaaaaat ttagttactc ctcaggccat 240
gttcatttat cctcagagaa taaattccaa aactctgcaa tcttaacaat acaaccaaaa 300
caattgcctg gaggacaaaa cccagtttct tatgtgtatt tggaagttgt atcaaagcat 360
ttttcaaaat caaaaagaat gccaataacc tatgacaatg gatttctctt cattcataca 420
gacaaacctg tttatactcc agaccagtca gtaaaagtta gagtttattc gttgaatgac 480
gacttgaagc cagccaaaag agaaactgtc ttaactttca tagatcctga aggatcagaa 540
gttgacatgg tagaagaaat tgatcatatt ggaattatct cttttcctga cttcaagatt 600
ccgtctaatc ctagatatgg tatgtggacg atcaaggcta aatataaaga ggacttttca 660
acaactggaa ccgcatattt tgaagttaaa gaatatgtct tgccacattt ttctgtctca 720
atcgagccag aatataattt cattggttac aagaacttta agaattttga aattactata 780
aaagcaagat atttttataa taaagtagtc actgaggctg acgtttatat cacatttgga 840
ataagagaag acttaaaaga tgatcaaaaa gaaatgatgc aaacagcaat gcaaaacaca 900
atgttgataa atggaattgc tcaagtcaca tttgattctg aaacagcagt caaagaactg 960
tcatactaca gtttagaaga tttaaacaac aagtaccttt atattgctgt aacagtcata 1020
gagtctacag gtggattttc tgaagaggca gaaatacctg gcatcaaata tgtcctctct 1080
ccctacaaac tgaatttggt tgctactcct cttttcctga agcctgggat tccatatccc 1140
atcaaggtgc aggttaaaga ttcgcttgac cagttggtag gaggagtccc agtaacactg 1200
aatgcacaaa caattgatgt aaaccaagag acatctgact tggatccaag caaaagtgta 1260
acacgtgttg atgatggagt agcttccttt gtgcttaatc tcccatctgg agtgacggtg 1320
ctggagttta atgtcaaaac tgatgctcca gatcttccag aagaaaatca ggccagggaa 1380
ggttaccgag caatagcata ctcatctctc agccaaagtt acctttatat tgattggact 1440
gataaccata aggctttgct agtgggagaa catctgaata ttattgttac ccccaaaagc 1500
ccatatattg acaaaataac tcactataat tacttgattt tatccaaggg caaaattatc 1560
cactttggca cgagggagaa attttcagat gcatcttatc aaagtataaa cattccagta 1620
acacagaaca tggttccttc atcccgactt ctggtctatt acatcgtcac aggagaacag 1680
acagcagaat tagtgtctga ttcagtctgg ttaaatattg aagaaaaatg tggcaaccag 1740
ctccaggttc atctgtctcc tgatgcagat gcatattctc caggccaaac tgtgtctctt 1800
aatatggcaa ctggaatgga ttcctgggtg gcattagcag cagtggacag tgctgtgtat 1860
ggagtccaaa gaggagccaa aaagcccttg gaaagagtat ttcaattctt agagaagagt 1920
gatctgggct gtggggcagg tggtggcctc aacaatgcca atgtgttcca cctagctgga 1980
cttaccttcc tcactaatgc aaatgcagat gactcccaag aaaatgatga accttgtaaa 2040
gaaattctca ggccaagaag aacgctgcaa aagaagatag aagaaatagc tgctaaatat 2100
aaacattcag tagtgaagaa atgttgttac gatggagcct gcgttaataa tgatgaaacc 2160
tgtgagcagc gagctgcacg gattagttta gggccaagat gcatcaaagc tttcactgaa 2220
tgttgtgtcg tcgcaagcca gctccgtgct aatatctctc ataaagacat gcaattggga 2280
aggctacaca tgaagaccct gttaccagta agcaagccag aaattcggag ttattttcca 2340
gaaagctggt tgtgggaagt tcatcttgtt cccagaagaa aacagttgca gtttgcccta 2400
cctgattctc taaccacctg ggaaattcaa ggcgttggca tttcaaacac tggtatatgt 2460
gttgctgata ctgtcaaggc aaaggtgttc aaagatgtct tcctggaaat gaatatacca 2520
tattctgttg tacgaggaga acagatccaa ttgaaaggaa ctgtttacaa ctataggact 2580
tctgggatgc agttctgtgt taaaatgtct gctgtggagg gaatctgcac ttcggaaagc 2640
ccagtcattg atcatcaggg cacaaagtcc tccaaatgtg tgcgccagaa agtagagggc 2700
tcctccagtc acttggtgac attcactgtg cttcctctgg aaattggcct tcacaacatc 2760
aatttttcac tggagacttg gtttggaaaa gaaatcttag taaaaacatt acgagtggtg 2820
ccagaaggtg tcaaaaggga aagctattct ggtgttactt tggatcctag gggtatttat 2940
ggtaccatta gcagacgaaa ggagttccca tacaggatac ccttagattt ggtccccaaa 2940
acagaaatca aaaggatttt gagtgtaaaa ggactgcttg taggtgagat cttgtctgca 3000
gttctaagtc aggaaggcat caatatccta acccacctcc ccaaagggag tgcagaggcg 3060
gagctgatga gcgttgtccc agtattctat gtttttcact acctggaaac aggaaatcat 3120
tggaacattt ttcattctga cccattaatt gaaaagcaga aactgaagaa aaaattaaaa 3180
gaagggatgt tgagcattat gtcctacaga aatgctgact actcttacag tgtgtggaag 3240
ggtggaagtg ctagcacttg gttaacagct tttgctttaa gagtacttgg acaagtaaat 3300
aaatacgtag agcagaacca aaattcaatt tgtaattctt tattgtggct agttgagaat 3360
tatcaattag ataatggatc tttcaaggaa aattcacagt atcaaccaat aaaattacag 3420
ggtaccttgc ctgttgaagc ccgagagaac agcttatatc ttacagcctt tactgtgatt 3480
ggaattagaa aggctttcga tatatgcccc ctggtgaaaa tcgacacagc tctaattaaa 3540
gctgacaact ttctgcttga aaatacactg ccagcccaga gcacctttac attggccatt 3600
tctgcgtatg ctctttccct gggagataaa actcacccac agtttcgttc aattgtttca 3660
gctttgaaga gagaagcttt ggttaaaggt aatccaccca tttatcgttt ttggaaagac 3720
aatcttcagc ataaagacag ctctgtacct aacactggta cggcacgtat ggtagaaaca 3780
actgcctatg ctttactcac cagtctgaac ttgaaagata taaattatgt taacccagtc 3840
atcaaatggc tatcagaaga gcagaggtat ggaggtggct tttattcaac ccaggacaca 3900
atcaatgcca ttgagggcct gacggaatat tcactcctgg ttaaacaact ccgcttgagt 3960
atggacatcg atgtttctta caagcataaa ggtgccttac ataattataa aatgacagac 4020
aagaatttcc ttgggaggcc agtagaggtg cttctcaatg atgacctcat tgtcagtaca 4080
ggatttggca gtggcttggc tacagtacat gtaacaactg tagttcacaa aaccagtacc 4140
tctgaggaag tttgcagctt ttatttgaaa atcgatactc aggatattga agcatcccac 4200
tacagaggct acggaaactc tgattacaaa cgcatagtag catgtgccag ctacaagccc 4260
agcagggaag aatcatcatc tggatcctct catgcggtga tggacatctc cttgcctact 4320
ggaatcagtg caaatgaaga agacttaaaa gcccttgtgg aaggggtgga tcaactattc 4380
actgattacc aaatcaaaga tggacatgtt attctgcaac tgaattcgat tccctccagt 4440
gatttccttt gtgtacgatt ccggatattt gaactctttg aagttgggtt tctcagtcct 4500
gccactttca cagtgtacga ataccacaga ccagataaac agtgtaccat gttttatagc 4560
acttccaata tcaaaattca gaaagtctgt gaaggagccg cgtgcaagtg tgtagaagct 4620
gattgtgggc aaatgcagga agaattggat ctgacaatct ctgcagagac aagaaaacaa 4680
acagcatgta aaccagagat tgcatatgct tataaagtta gcatcacatc catcactgta 4740
gaaaatgttt ttgtcaagta caaggcaacc cttctggata tctacaaaac tggggaagct 4800
gttgctgaga aagactctga gattaccttc attaaaaagg taacctgtac taacgctgag 4860
ctggtaaaag gaagacagta cttaattatg ggtaaagaag ccctccagat aaaatacaat 4920
ttcagtttca ggtacatcta ccctttagat tccttgacct ggattgaata ctggcctaga 4980
gacacaacat gttcatcgtg tcaagcattt ttagctaatt tagatgaatt tgccgaagat 5040
atctttttaa atggatgcta aaattcctga agttcagctg catacagttt gcacttatgg 5100
actcctgttg ttgaagttcg tttttttgtt ttcttctttt tttaaacatt catagctggt 5160
cttatttgta aagctcactt tacttagaat tagtggcact tgcttttatt agagaatgat 5220
ttcaaatgct gtaactttct gaaataacat ggccttggag ggcatgaaga cagatactcc 5280
tccaaggtta ttggacaccg gaaacaataa attggaacac ctcctcaaac ctaccactca 5340
ggaatgtttg ctggggccga aagaacagtc cattgaaagg gagtattaca aaaacatggc 5400
ctttgcttga aagaaaatac caaggaacag gaaactgatc attaaagcct gagtttgctt 5460
tcaaaaaaaa aaaaaaaaaa 5480,

and the antisense strand comprises nucleotides (e.g., at least 15 contiguous nucleotides) differing by no more than 3 nucleotides from the nucleotide sequence of:

(SEQ ID NO: 361)
tttttttttt ttttttttga aagcaaactc aggctttaat gatcagtttc ctgttccttg 60
gtattttctt tcaagcaaag gccatgtttt tgtaatactc cctttcaatg gactgttctt 120
tcggccccag caaacattcc tgagtggtag gtttgaggag gtgttccaat ttattgtttc 180
cggtgtccaa taaccttgga ggagtatctg tcttcatgcc ctccaaggcc atgttatttc 240
agaaagttac agcatttgaa atcattctct aataaaagca agtgccacta attctaagta 300
aagtgagctt tacaaataag accagctatg aatgtttaaa aaaagaagaa aacaaaaaaa 360
cgaacttcaa caacaggagt ccataagtgc aaactgtatg cagctgaact tcaggaattt 420
tagcatccat ttaaaaagat atcttcggca aattcatcta aattagctaa aaatgcttga 480
cacgatgaac atgttgtgtc tctaggccag tattcaatcc aggtcaagga atctaaaggg 540
tagatgtacc tgaaactgaa attgtatttt atctggaggg cttctttacc cataattaag 600
tactgtcttc cttttaccag ctcagcgtta gtacaggtta cctttttaat gaaggtaatc 660
tcagagtctt tctcagcaac agcttcccca gttttgtaga tatccagaag ggttgccttg 720
tacttgacaa aaacattttc tacagtgatg gatgtgatgc taactttata agcatatgca 780
atctctggtt tacatgctgt ttgttttctt gtctctgcag agattgtcag atccaattct 840
tcctgcattt gcccacaatc agcttctaca cacttgcacg cggctccttc acagactttc 900
tgaattttga tattggaagt gctataaaac atggtacact gtttatctgg tctgtggtat 960
tcgtacactg tgaaagtggc aggactgaga aacccaactt caaagagttc aaatatccgg 1020
aatcgtacac aaaggaaatc actggaggga atcgaattca gttgcagaat aacatgtcca 1080
tctttgattt ggtaatcagt gaatagttga tccacccctt ccacaagggc ttttaagtct 1140
tcttcatttg cactgattcc agtaggcaag gagatgtcca tcaccgcatg agaggatcca 1200
gatgatgatt cttccctgct gggcttgtag ctggcacatg ctactatgcg tttgtaatca 1260
gagtttccgt agcctctgta gtgggatgct tcaatatcct gagtatcgat tttcaaataa 1320
aagctgcaaa cttcctcaga ggtactggtt ttgtgaacta cagttgttac atgtactgta 1380
gccaagccac tgccaaatcc tgtactgaca atgaggtcat cattgagaag cacctctact 1440
ggcctcccaa ggaaattctt gtctgtcatt ttataattat gtaaggcacc tttatgcttg 1500
taagaaacat cgatgtccat actcaagcgg agttgtttaa ccaggagtga atattccgtc 1560
aggccctcaa tggcattgat tgtgtcctgg gttgaataaa agccacctcc atacctctgc 1620
tcttctgata gccatttgat gactgggtta acataattta tatctttcaa gttcagactg 1680
gtgagtaaag cataggcagt tgtttctacc atacgtgccg taccagtgtt aggtacagag 1740
ctgtctttat gctgaagatt gtctttccaa aaacgataaa tgggtggatt acctttaacc 1800
aaagcttctc tcttcaaagc tgaaacaatt gaacgaaact gtgggtgagt tttatctccc 1860
agggaaagag catacgcaga aatggccaat gtaaaggtgc tctgggctgg cagtgtattt 1920
tcaagcagaa agttgtcagc tttaattaga gctgtgtcga ttttcaccag ggggcatata 1980
tcgaaagcct ttctaattcc aatcacagta aaggctgtaa gatataagct gttctctcgg 2040
gcttcaacag gcaaggtacc ctgtaatttt attggttgat actgtgaatt ttccttgaaa 2100
gatccattat ctaattgata attctcaact agccacaata aagaattaca aattgaattt 2160
tggttctgct ctacgtattt atttacttgt ccaagtactc ttaaagcaaa agctgttaac 2220
caagtgctag cacttccacc cttccacaca ctgtaagagt agtcagcatt tctgtaggac 2280
ataatgctca acatcccttc ttttaatttt ttcttcagtt tctgcttttc aattaatggg 2340
tcagaatgaa aaatgttcca atgatttcct gtttccaggt agtgaaaaac atagaatact 2400
gggacaacgc tcatcagctc cgcctctgca ctccctttgg ggaggtgggt taggatattg 2460
atgccttcct gacttagaac tgcagacaag atctcaccta caagcagtcc ttttacactc 2520
aaaatccttt tgatttctgt tttggggacc aaatctaagg gtatcctgta tgggaactcc 2580
tttcgtctgc taatggtacc ataaataccc ctaggatcca aagtaacacc agaatagctt 2640
tcccttttga caccttctgg caccactcgt aatgttttta ctaagatttc ttttccaaac 2700
caagtctcca gtgaaaaatt gatgttgtga aggccaattt ccagaggaag cacagtgaat 2760
gtcaccaagt gactggagga gccctctact ttctggcgca cacatttgga ggactttgtg 2820
ccctgatgat caatgactgg gctttccgaa gtgcagattc cctccacagc agacatttta 2880
acacagaact gcatcccaga agtcctatag ttgtaaacag ttcctttcaa ttggatctgt 2940
tctcctcgta caacagaata tggtatattc atttccagga agacatcttt gaacaccttt 3000
gccttgacag tatcagcaac acatatacca gtgtttgaaa tgccaacgcc ttgaatttcc 3060
caggtggtta gagaatcagg tagggcaaac tgcaactgtt ttcttctggg aacaagatga 3120
acttcccaca accagctttc tggaaaataa ctccgaattt ctggcttgct tactggtaac 3180
agggtcttca tgtgtagcct tcccaattgc atgtctttat gagagatatt agcacggagc 3240
tggcttgcga cgacacaaca ttcagtgaaa gctttgatgc atcttggccc taaactaatc 3300
cgtgcagctc gctgctcaca ggtttcatca ttattaacgc aggctccatc gtaacaacat 3360
ttcttcacta ctgaatgttt atatttagca gctatttctt ctatcttctt ttgcagcgtt 3420
cttcttggcc tgagaatttc tttacaaggt tcatcatttt cttgggagtc atctgcattt 3480
gcattagtga ggaaggtaag tccagctagg tggaacacat tggcattgtt gaggccacca 3540
cctgccccac agcccagatc actcttctct aagaattgaa atactctttc caagggcttt 3600
ttggctcctc tttggactcc atacacagca ctgtccactg ctgctaatgc cacccaggaa 3660
tccattccag ttgccatatt aagagacaca gtttggcctg gagaatatgc atctgcatca 3720
ggagacagat gaacctggag ctggttgcca catttttctt caatatttaa ccagactgaa 3780
tcagacacta attctgctgt ctgttctcct gtgacgatgt aatagaccag aagtcgggat 3840
gaaggaacca tgttctgtgt tactggaatg tttatacttt gataagatgc atctgaaaat 3900
ttctccctcg tgccaaagtg gataattttg cccttggata aaatcaagta attatagtga 3960
gttattttgt caatatatgg gcttttgggg gtaacaataa tattcagatg ttctcccact 4020
agcaaagcct tatggttatc agtccaatca atataaaggt aactttggct gagagatgag 4080
tatgctattg ctcggtaacc ttccctggcc tgattttctt ctggaagatc tggagcatca 4140
gttttgacat taaactccag caccgtcact ccagatggga gattaagcac aaaggaagct 4200
actccatcat caacacgtgt tacacttttg cttggatcca agtcagatgt ctcttggttt 4260
acatcaattg tttgtgcatt cagtgttact gggactcctc ctaccaactg gtcaagcgaa 4320
tctttaacct gcaccttgat gggatatgga atcccaggct tcaggaaaag aggagtagca 4380
accaaattca gtttgtaggg agagaggaca tatttgatgc caggtatttc tgcctcttca 4440
gaaaatccac ctgtagactc tatgactgtt acagcaatat aaaggtactt gttgtttaaa 4500
tcttctaaac tgtagtatga cagttctttg actgctgttt cagaatcaaa tgtgacttga 4560
gcaattccat ttatcaacat tgtgttttgc attgctgttt gcatcatttc tttttgatca 4620
tcttttaagt cttctcttat tccaaatgtg atataaacgt cagcctcagt gactacttta 4680
ttataaaaat atcttgcttt tatagtaatt tcaaaattct taaagttctt gtaaccaatg 4740
aaattatatt ctggctcgat tgagacagaa aaatgtggca agacatattc tttaacttca 4800
aaatatgcgg ttccagttgt tgaaaagtcc tctttatatt tagccttgat cgtccacata 4860
ccatatctag gattagacgg aatcttgaag tcaggaaaag agataattcc aatatgatca 4920
atttcttcta ccatgtcaac ttctgatcct tcaggatcta tgaaagttaa gacagtttct 4980
cttttggctg gcttcaagtc gtcattcaac gaataaactc taacttttac tgactggtct 5040
ggagtataaa caggtttgtc tgtatgaatg aagagaaatc cattgtcata ggttattggc 5100
attctttttg attttgaaaa atgctttgat acaacttcca aatacacata agaaactggg 5160
ttttgtcctc caggcaattg ttttggttgt attgttaaga ttgcagagtt ttggaattta 5220
ttctctgagg ataaatgaac atggcctgag gagtaactaa attttttatc aggataactt 5280
ttaatagaga ttgttgcatc aaatgcttca gtgtatccat aaacttgaat cacaatattt 5340
tcagatgctc caacacggaa tatttttggt gctgaaatga catatgtttg ctcctgtccc 5400
caggttttcc ccaggaagat taaaaaacaa agtattccca aaaggcccat ggttggaggt 5460
agcaggaaac cacggatata 5480.

The present disclosure includes an iRNA which can be used in methods for treating or preventing MG and/or included in a co-formulation of the present disclosure that is a double-stranded ribonucleic acid (dsRNA) agent (e.g., having a complementarity region of 19-23 nucleotides in length and/or having a strand length of no more than 30 nucleotides) that inhibits expression of complement component C5, wherein the dsRNA agent comprises a sense strand and an antisense strand, the antisense strand including a region of complementarity which comprises at least 17 contiguous nucleotides differing by no more than 3 nucleotides from the nucleotide sequence of 5′-UAUUAUAAAAAUAUCUUGCUUUU-3′ (SEQ ID NO: 364), wherein one or more of the dsRNA nucleotides are modified. The dsRNA agent may include at least one modified nucleotide e.g., with 2′-deoxy, 2′-methoxy, and/or 2′-fluoro groups; for example, where substantially all of the nucleotides of the sense strand and antisense strand are modified nucleotides. Moreover, the sense strand can be conjugated to a ligand attached at the 3′-terminus, e.g., terminally modified with a triantennary GalNAc moiety.

The modified nucleotides that may be included in a dsRNA which may be used in a co-formulation of the present invention or in a method for treating or preventing MG may include a 3′-terminal deoxy-thymine (dT) nucleotide, a 2′-O-methyl modified nucleotide, a 2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base including nucleotide, a nucleotide including a 5′-phosphorothioate group, and a terminal nucleotide linked to a cholesteryl derivative or a dodecanoic acid bisdecylamide group. The dsRNA may include a phosphorothioate and/or methylphosphonate internucleotide linkage.

A dsRNA is double stranded, but may include one or more overhangs, such as at the 3′ end of one or more strands (e.g., 2 or more nucleotides of overhang).

Double stranded RNAs of the present disclosure may include a ligand (e.g., a N-acetylgalactosamine (GalNAc) derivative,

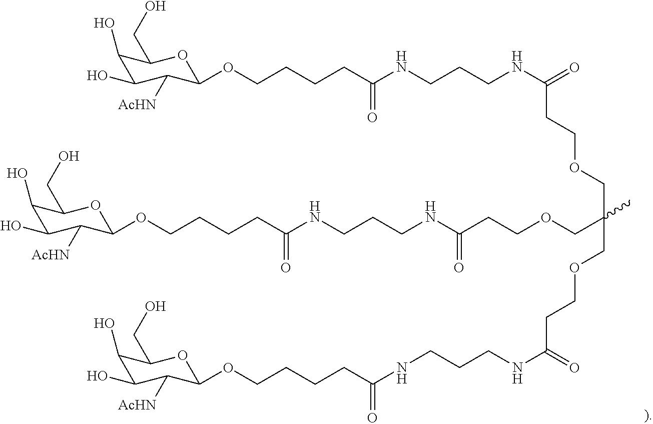 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In an embodiment of the disclosure, the ligand is conjugated to the 3′ end of the sense strand of the dsRNA.

In one aspect, the present disclosure provides a double-stranded ribonucleic acid (dsRNA) agent that inhibits expression of complement component C5 which can be included in a co-formulation of the present disclosure and/or used in methods for treating or preventing MG, wherein the dsRNA agent comprises a sense strand and an antisense strand, wherein the sense strand comprises the nucleotide sequence 5′-AAGCAAGAUAUUUUUAUAAUA-3′ (SEQ ID NO: 365) and wherein the antisense strand comprises the nucleotide sequence 5′-UAUUAUAAAAAUAUCUUGCUUUU-3′ (SEQ ID NO: 364), e.g., wherein one or more of the dsRNA nucleotides are modified; e.g., with 2′-deoxy, 2′-methoxy, and/or 2′-fluoro groups and/or terminally modified with a triantennary GalNAc moiety. In one embodiment, the dsRNA agent comprises at least one modified nucleotide, as described herein.

In one aspect, the present disclosure provides a double stranded iRNA agent, which can be included in a co-formulation of the present disclosure and/or used in methods for treating or preventing MG, that inhibits expression of complement component C5 wherein the double stranded iRNA agent comprises a sense strand and an antisense strand forming a double-stranded region, wherein the sense strand comprises at least 15 contiguous nucleotides differing by no more than 3 nucleotides from the nucleotide sequence of SEQ ID NO: 365 and the antisense strand comprises at least 15 contiguous nucleotides differing by no more than 3 nucleotides from the nucleotide sequence of SEQ ID NO: 364, wherein substantially all of the nucleotides of the sense strand and substantially all of the nucleotides of the antisense strand are modified nucleotides, and wherein the sense strand is conjugated to a ligand attached at the 3′-terminus. In one embodiment, the dsRNA agent comprises at least one modified nucleotide, as described herein.

In one embodiment, substantially all of the nucleotides of the sense strand are modified nucleotides selected from the group consisting of a 2′-O-methyl modification, a 2′-fluoro modification and a 3′-terminal deoxy-thymine (dT) nucleotide. In another embodiment, substantially all of the nucleotides of the antisense strand are modified nucleotides selected from the group consisting of a 2′-O-methyl modification, a 2′-fluoro modification and a 3′-terminal deoxy-thymine (dT) nucleotide. In another embodiment, the modified nucleotides are a short sequence of deoxy-thymine (dT) nucleotides. In another embodiment, the sense strand comprises two phosphorothioate internucleotide linkages at the 5′-terminus. In one embodiment, the antisense strand comprises two phosphorothioate internucleotide linkages at the 5′-terminus and two phosphorothioate internucleotide linkages at the 3′-terminus. In yet another embodiment, the sense strand is conjugated to one or more GalNAc derivatives attached through a branched bivalent or trivalent linker at the 3′-terminus.

In one embodiment, at least one of the modified nucleotides is selected from the group consisting of a 3′-terminal deoxy-thymine (dT) nucleotide, a 2′-O-methyl modified nucleotide, a 2′-fluoro modified nucleotide, a 2′-deoxy-modified nucleotide, a locked nucleotide, a basic nucleotide, a 2′-amino-modified nucleotide, a 2′-alkyl-modified nucleotide, a morpholino nucleotide, a phosphoramidate, a non-natural base including nucleotide, a nucleotide 20 including a 5′-phosphorothioate group, and a terminal nucleotide linked to a cholesteryl derivative or a dodecanoic acid bisdecylamide group.

In another embodiment, the modified nucleotides comprise a short sequence of 3′-terminal deoxy-thymine (dT) nucleotides (e.g., 2).

In one embodiment, the region of complementarity is at least 17 nucleotides in length. In another embodiment, the region of complementarity is between 19 and 21 nucleotides in length. In one embodiment, the region of complementarity is 19 nucleotides in length. In one embodiment, each strand is no more than 30 nucleotides in length. In one embodiment, at least one strand comprises a 3′ overhang of at least 1 nucleotide. In another embodiment, at least one strand comprises a 3′ overhang of at least 2 nucleotides. In one embodiment, the dsRNA agent further comprises a ligand. In one embodiment, the ligand is conjugated to the 3′ end of the sense strand of the dsRNA agent. In one embodiment, the ligand is an N-acetylgalactosamine (GalNAc) derivative. In one embodiment, the ligand is

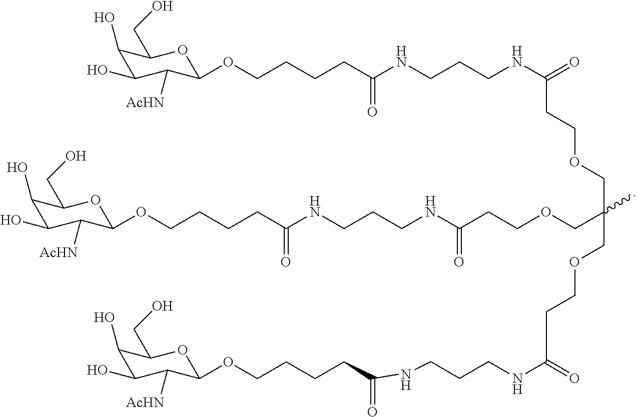

In one embodiment, the dsRNA agent is conjugated to the ligand as shown in the following schematic

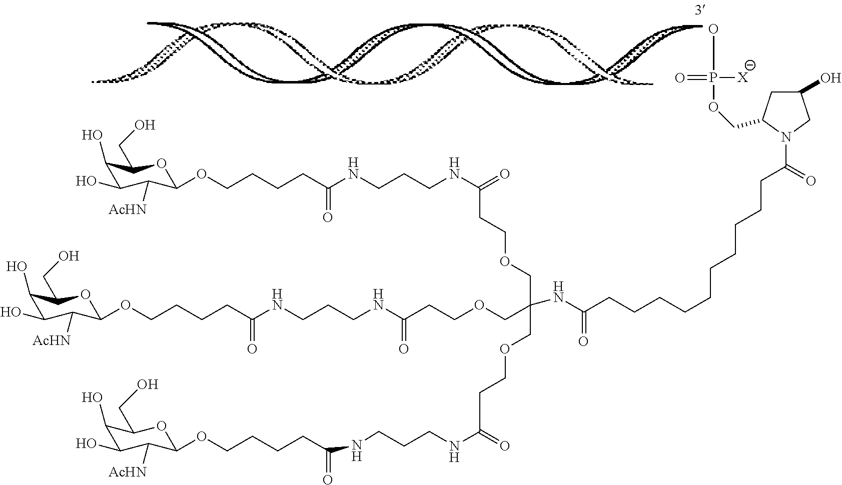

and, wherein X is O or S. In one embodiment, the X is O.

In an embodiment of the present disclosure, the C5 iRNA includes an RNA strand that is complementary to an mRNA transcribed from the C5 gene sense strand DNA sequence AAGCAAGATATTTTTATAATA (SEQ ID NO: 407), for example, wherein the iRNA is a dsRNA that includes another hybridized RNA strand.

In another aspect, the present disclosure provides a double-stranded ribonucleic acid (dsRNA) agent that inhibits expression of complement component C5, wherein the dsRNA agent comprises a sense strand and an antisense strand, wherein the sense strand comprises the nucleotide sequence 5′AAGCAAGAUAUUUUUAUAAUA3′ (SEQ ID NO: 366) and wherein the antisense strand comprises the nucleotide sequence 5′UAUUAUAAAAAUAUCUUGCUUUUdTdT3′ (SEQ ID NO: 367).

In another aspect, the present disclosure provides a double-stranded ribonucleic acid (dsRNA) agent that inhibits expression of complement component C5, wherein the dsRNA agent comprises a sense strand and an antisense strand, wherein the sense strand comprises the nucleotide sequence 5′AAGCAAGAUAUUUUUAUAAUA3′ with the modified bases and phosporothioate linkages described above (SEQ ID NO: 406) (i.e., asasGfcAfaGfaUfAfUfuUfuuAfuAfaua), and wherein the antisense strand comprises the nucleotide sequence 5′UAUUAUAAAAAUAUCUUGCUUUUTT5′ with the modified bases and phosporothioate linkages described above (SEQ ID NO: 369) (i.e., usAfsUfuAfuaAfaAfauaUfcUfuGfcuususudTdT), wherein the 3′-end of the sense strand is conjugated to L96.

In an embodiment of the disclosure, the sense or antisense strands of a dsRNA that can be included in a formulation of the present disclosure comprises sequences selected from the group consisting of A-118320, A-118321, A-118316, A-118317, A-118332, A-118333, A-118396, A-118397, A-118386, A-118387, A-118312, A-118313, A-118324, A-118325, A-119324, A-119325, A-119332, A-119333, A-119328, A-119329, A-119322, A-119323, A-119324, A-119325, A-119334, A-119335, A-119330, A-119331, A-119326, A-119327, A-125167, A-125173, A-125647, A-125157, A-125173, and A-125127. In one embodiment, the dsRNA agent comprises at least one modified nucleotide.

TABLE D
Sense and Anti-sense RNA strands of C5 dsRNAs
(unmodified and modified strands shown)

| | |
|---|---|
| A-118320 | aagcaagaua uuuuuauaau a |
| (SEQ ID NO: 370) | AfaGfcAfaGfaUfAfUfuUfuUfaUfaAfuAfL96 |
| sense strand | |
| A-118321 | uauuauaaaa auaucuugcu uuu |
| anti-sense strand | uAfuUfaUfaAfaAfauaUfcUfuGfcUfusUfsu |
| (SEQ ID NO: 371) | |
| A-118316 | gacaaaauaa cucacuauaa u |
| sense strand | GfaCfaAfaAfuAfAfCfuCfaCfuAfuAfaUfL96 |
| (SEQ ID NO: 372) | |
| A-118317 | auuauaguga guuauuuugu caa |
| anti-sense strand | aUfuAfuAfg UfgAfguuAfuUfuUfgUfcsAfsa |
| (SEQ ID NO: 373) | |
| A-118332 | aggauuuuga guguaaaagg a |
| sense strand | AfgGfaUfuUfuGf AfGfuGfuAfaAfaGfgAfL96 |
| (SEQ ID NO: 374) | |
| A-118333 | uccuuuuaca cucaaaaucc uuu |
| anti-sense strand | |
| (SEQ ID NO: 375) | |
| A-118396 | guuccggaua uuugaacuuu u |
| sense strand | GfuUfcCfgGfaUfAfUfuUfgAfaCfuUfuUfL96 |
| (SEQ ID NO: 376) | |
| A-118397 | aaaaguucaa auauccggaa ccg |
| anti-sense strand | aAfaAfgUfuCfaAfauaUfcCfgGfaAfcsCfsg |
| (SEQ ID NO: 377) | |
| A-118386 | cagaucaaac acaauuucag u |
| sense strand | CfaGfaUfcAfaAfCfAfcAfaUfuUfcAfgUfL96 |
| (SEQ ID NO: 378) | |
| A-118387 | acugaaauug uguuugaucu gca |
| anti-sense strand | aCfuGfaAfaUfuGfuguUfuGfaUfcUfgsCfsa |
| (SEQ ID NO: 379) | |
| A-118312 | ugacaaaaua acucacuaua a |
| sense strand | UfgAfcAfaAfaUfAfAfcUfcAfcUfaUfaAfL96 |
| (SEQ ID NO: 380) | |
| A-118313 | uuauagugag uuauuuuguc aau |
| anti-sense strand | uUfaUfaGfuGfaGfuuaUfuUfuGfuCfasAfsu |
| (SEQ ID NO: 381) | |
| A-118324 | auuuaaacaa caaguaccuu u |
| sense strand | AfuUfuAfaAfcAfAfCfaAfgUfaCfcUfuUfL96 |
| (SEQ ID NO: 382) | |
| A-118325 | aaagguacuu guuguuuaaa ucu |
| anti-sense strand | AfuUfuAfaAfcAfAfCfaAfgUfaCfcUfuUfL96 |
| (SEQ ID NO: 383) | |
| A-119324 | gacaaaauaa cucacuauaa u |
| sense strand | AfuUfuAfaAfcAfAfCfaAfgUfaCfcUfuUfL96 |
| (SEQ ID NO: 384) | |
| A-119325 | auuauaguga guuauuuugu caa |
| anti-sense strand | asUfsuAfuAfgUfgAfguuAfuUfuUfgUfcsasa |
| (SEQ ID NO: 385) | |
| A-119332 | cagaucaaac acaauuucag u |
| sense strand | CfsasGfaUfcAfaAfCfAfcAfaUfuUfcAfgUfL96 |
| (SEQ ID NO: 386) | |
| A-119333 | acugaaauug uguuugaucu gca |
| anti-sense strand | asCfsuGfaAfaUfuGfuguUfuGfaUfcUfgscsa |
| (SEQ ID NO: 387) | |
| A-119328 | auuuaaacaa caaguaccuu u |
| sense strand | AfsusUfuAfaAfcAfAfCfaAfgUfaCfcUfuUfL96 |
| (SEQ ID NO: 388) | |
| A-119329 | aaagguacuu guuguuuaaa ucu |
| anti-sense strand | asAfsaGfg UfaCfuUfguuGfuUfuAfaAfusesu |
| (SEQ ID NO: 389) | |
| A-119322 | ugacaaaaua acucacuaua a |
| sense strand | UfsgsAfcAfaAfaUfAfAfcUfcAfcUfaUfaAfL96 |
| (SEQ ID NO: 390) | |
| A-119323 | uuauagugag uuauuuuguc aau |
| anti-sense strand | |
| (SEQ ID NO: 391) | |
| A-119324 | gacaaaauaa cucacuauaa u |
| sense strand | GfsasCfaAfaAfuAfAfCfuCfaCfuAfuAfaUfL96 |
| (SEQ ID NO: 392) | |
| A-119325 | auuauagugaguuauuuugucaa |
| anti-sense strand | asUfsuAfuAfgUfgAfguuAfuUfuUfgUfcsasa |
| (SEQ ID NO: 393) | |
| A-119334 | guuccggaua uuugaacuuu u |
| sense strand | GfsusUfcCfgGfaUfAfUfuUfgAfaCfuUfuUfL96 |
| (SEQ ID NO: 394) | |
| A-119335 | aaaaguucaa auauccggaa ccg |
| anti-sense strand | asAfsaAfgUfuCfaAfauaUfcCfgGfaAfesesg |
| (SEQ ID NO: 395) | |
| A-119330 | ugcagaucaa acacaauuuc a |
| sense strand | UfsgsCfaGfaUfcAfAfAfcAfcAfaUfuUfcAfL96 |
| (SEQ ID NO: 396) | |
| A-119331 | ugaaauugug uuugaucugc aga |
| anti-sense strand | usGfsaAfaUfuGfuGfuuuGfaUfcUfgCfasgsa |
| (SEQ ID NO: 397) | |
| A-119326 | aagcaagaua uuuuuauaau a |
| sense strand | AfsasGfcAfaGfaUfAfUfuUfuUfaUfaAfuAfL96 |
| (SEQ ID NO: 398) | |
| A-119327 | uauuauaaaa auaucuugcu uuu |
| anti-sense strand | usAfsuUfaUfaAfaAfauaUfcUfuGfcUfususu |
| (SEQ ID NO: 399) | |
| A-125167 | aagcaagaua uuuuuauaau a |
| sense strand | asasGfcAfaGfaUfAfUfuUfuuAfuAfauaL96 |
| (SEQ ID NO: 400) | |
| A-125173 | uauuauaaaa auaucuugcu uuutt |
| anti-sense strand | usAfsUfuAfuAfAfaAfauaUfcUfuGfcuususudTdT |
| (SEQ ID NO: 401) | |
| A-125647 | uauuauaaaa auaucuugcu uuutt |
| anti-sense stand | usAfsUfuAfuaAfaAfauaUfcUfuGfcuususudTdT |
| (SEQ ID NO: 402) | |
| A-125157 | aagcaagaua uuuuuauaau a |
| sense strand | asasGfcAfaGfaUfAfUfuUfuuAfuaAfuaL96 |
| (SEQ ID NO: 403) | |
| A-125173 | uauuauaaaa auaucuugcu uuutt |
| anti-sense strand | usAfsUfuAfuAfAfaAfauaUfcUfuGfcuususudTdT |
| (SEQ ID NO: 404) | |
| A-125127 | aagcaagaua uuuuuauaau a |
| sense strand | asasgcaagaUfaUfuuuuauaauaL96 |
| (SEQ ID NO: 405) | |

wherein,
A = Adenosine-3′-phosphate
Af = 2′-fluoroadenosine-3′-phosphate
Afs = 2′-fluoroadenosine-3′-phosphorothioate
As = adenosine-3′-phosphorothioate
C = cytidine-3′-phosphate
Cf = 2′-fluorocytidine-3′-phosphate
Cfs = 2′-fluorocytidine-3′-phosphorothioate
Cs = cytidine-3′-phosphorothioate
G = guanosine-3′-phosphate
Gf = 2′-fluoroguanosine-3′-phosphate
Gfs-2′-fluoroguanosine-3′-phosphorothioate
Gs-guanosine-3′-phosphorothioate
T = 5′-methyluridine-3′-phosphate
Tf = 2′-fluoro-5-methyluridine-3′-phosphate
Tfs = 2′-fluoro-5-methyluridine-3′-phosphorothioate
Ts-5-methyluridine-3′-phosphorothioate
U = Uridine-3′-phosphate
Uf = 2′-fluorouridine-3′-phosphate
Ufs = 2′-fluorouridine-3′-phosphorothioate
Us = uridine-3′-phosphorothioate
N = any nucleotide (G, A, C, T or U)
a = 2′-0-methyladenosine-3′-phosphate
as = 2′-0-methyladenosine-3′-phosphorothioate
c = 2′-0-methylcytidine-3′-phosphate
cs = 2′-0-methylcytidine-3′-phosphorothioate
g = 2′-0-methylguanosine-3′-phosphate
gs = 2′-0-methylguanosine-3′-phosphorothioate
t = 2′-0-methyl-5-methyluridine-3′-phosphate
ts = 2′-0-methyl-5-methyluridine-3′-phosphorothioate
u = 2′-0-methyluridine-3′-phosphate
us = 2′-0-methyluridine-3′-phosphorothioate
s = phosphorothioate linkage
L96 = N-[tris(GalNAc-alkyl)-amidodecanoyl)]-4-hydroxyprolinol
Hyp - (GalNAc-alkyl)₃
(dt) = deoxy-thymine

In an embodiment of the disclosure, the dsRNA comprises the two following pairs of strands:
A-118320 & A-118321;
A-118316 & A-118317;
A-118332 & A-118333;
A-118396 & A-118397;
A-118386 & A-118387;
A-118312 & A-118313;
A-118324 & A-118325;
A-119324 & A-119325;
A-119332 & A-119333;
A-119328 & A-119329;
A-119322 & A-119323;
A-119324 & A-119325;
A-119334 & A-119335;
A-119330 & A-119331;
A-119326 & A-119327;
A-125167 & A-125173 or A-125647;
A-125157 & A-125173 or A-125647;
A-125127 & A-125173 or A-125647; and/or
A-125167 & A-125647.

In an embodiment of the disclosure, the C5 iRNA (e.g., Cemdisiran) comprises one or more galactosamines, e.g., 3, for example, represented by the structure:

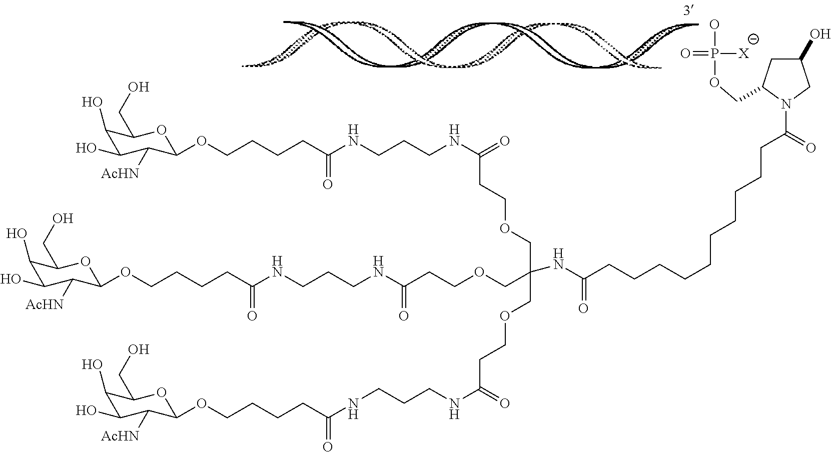

wherein the wavy double helix-like structure represents the RNA portion of the molecule and X is O or X is S; e.g.,

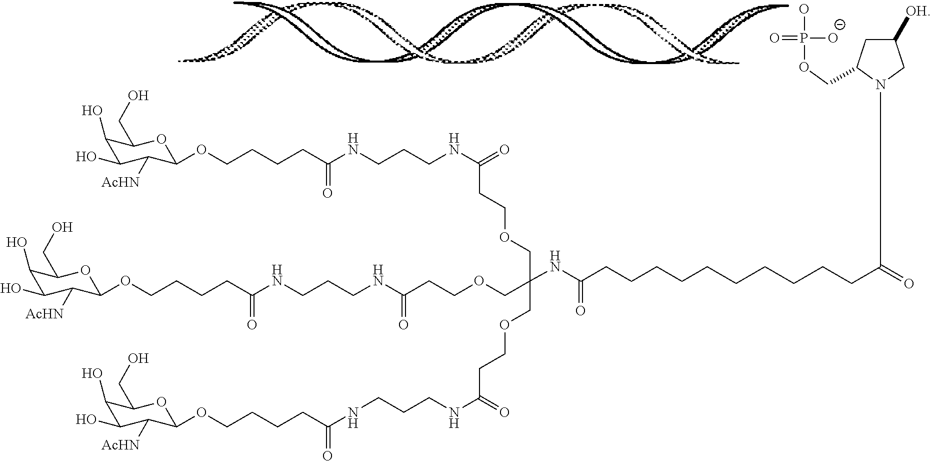 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2COP(=O)([O-])O)OC(CO)C(O)C1O]

However, in an embodiment of the disclosure, a co-formulation of the present disclosure further comprises degradation products represented by one or more of the following structures (wavy line represents double stranded RNA structure):

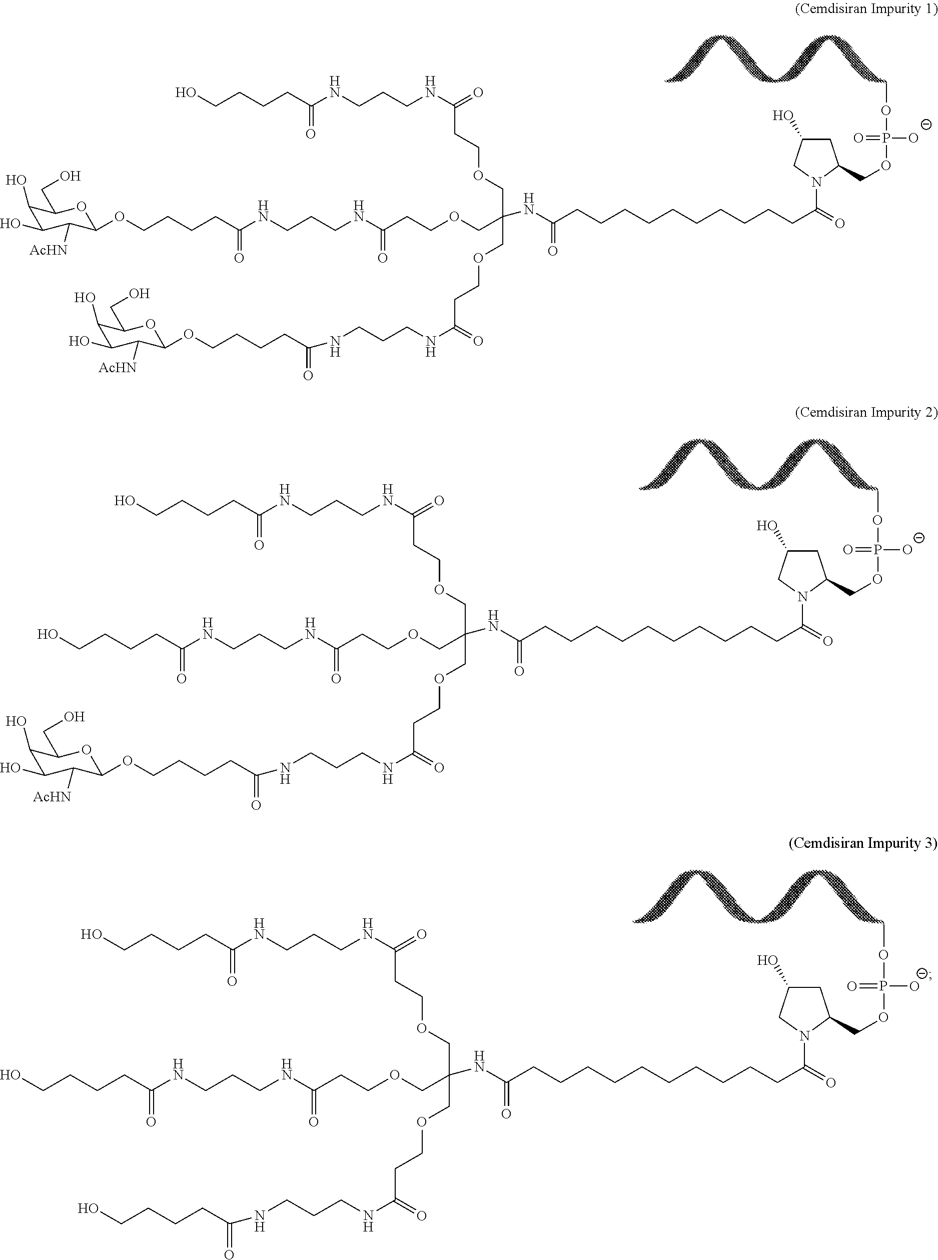 [SMILES: COP(=O)([O-])OC[C@@H]1C[C@@H](O)CN1C(=O)CCCCCCCCCCC(=O)NC(COCCC(=O)NCCCNC(=O)CCCCO)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O ; COP(=O)([O-])OC[C@@H]1C[C@@H](O)CN1C(=O)CCCCCCCCCCC(=O)NC(COCCC(=O)NCCCNC(=O)CCCCO)(COCCC(=O)NCCCNC(=O)CCCCO)COCCC(=O)NCCCNC(=O)CCCCOC1OC(CO)C(O)C(O)C1NC(C)=O ; COP(=O)([O-])OC[C@@H]1C[C@@H](O)CN1C(=O)CCCCCCCCCCC(=O)NC(COCCC(=O)NCCCNC(=O)CCCCO)(COCCC(=O)NCCCNC(=O)CCCCO)COCCC(=O)NCCCNC(=O)CCCCO]

wherein 1, 2 or 3 of the terminal N-acetylgalactosamines (GalNAc) are missing.

iRNAs of the present disclosure can be chemically linked, by the RNA portion of the molecule, to one or more ligands, moieties or conjugates that enhance the activity, cellular distribution or cellular uptake of the iRNA. Such moieties include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

A ligand can be a carbohydrate. A carbohydrate conjugated RNA is advantageous for the in vivo delivery of nucleic acids. As used herein, “carbohydrate” ligand refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the disclosure is a monosaccharide. In one embodiment, the monosaccharide is an N-acetylgalactosamine, such as

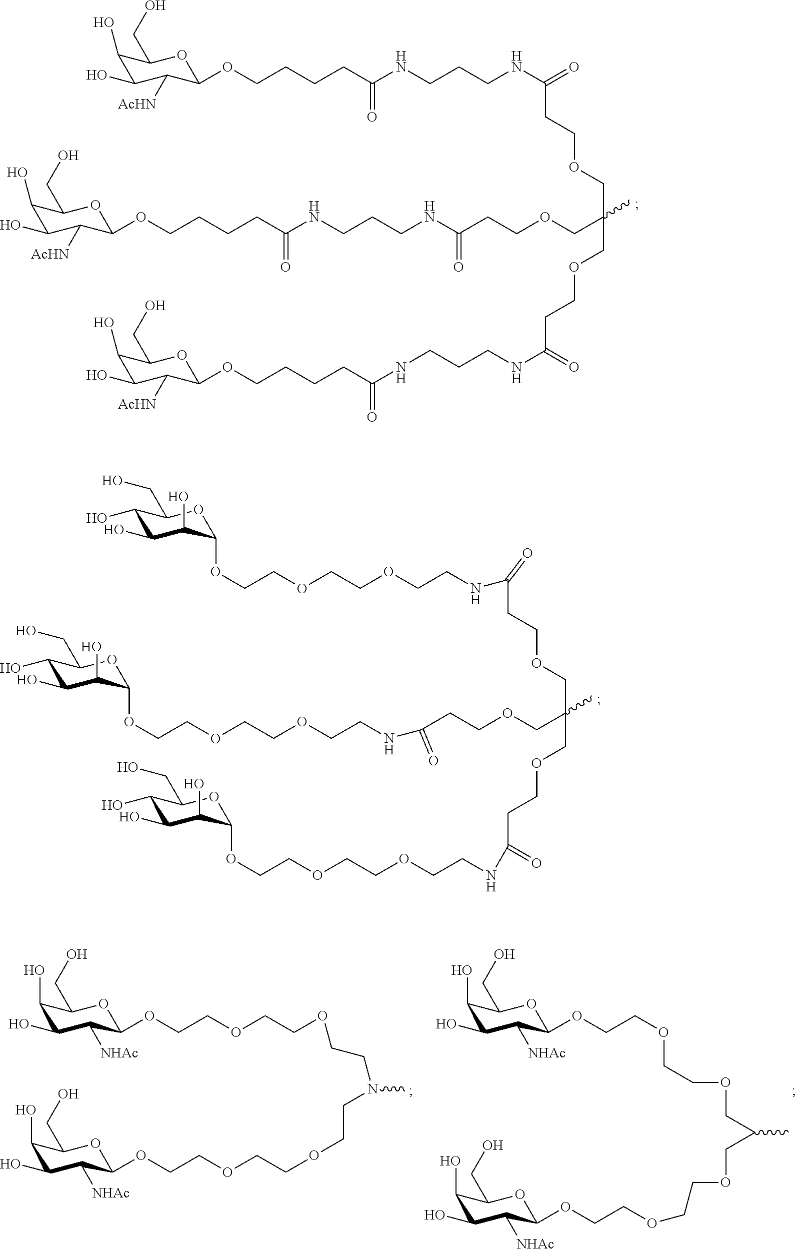

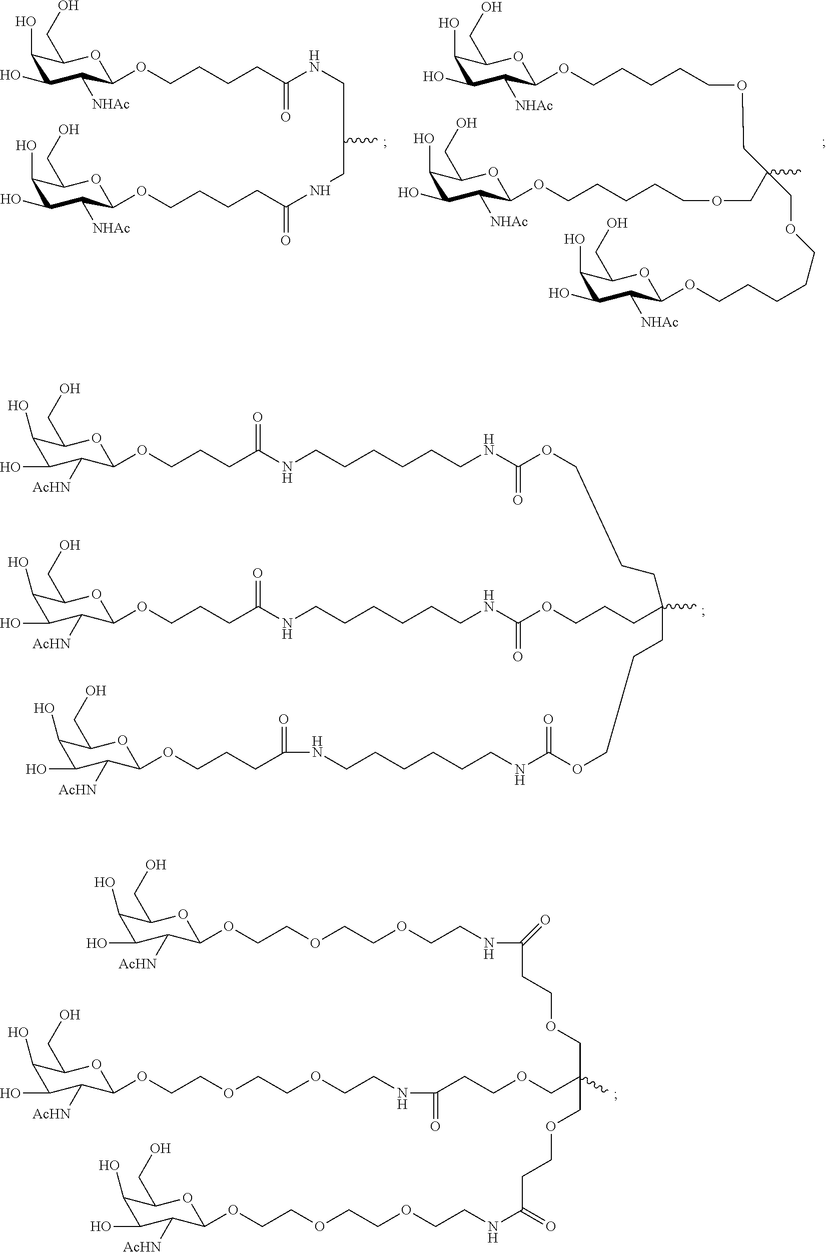

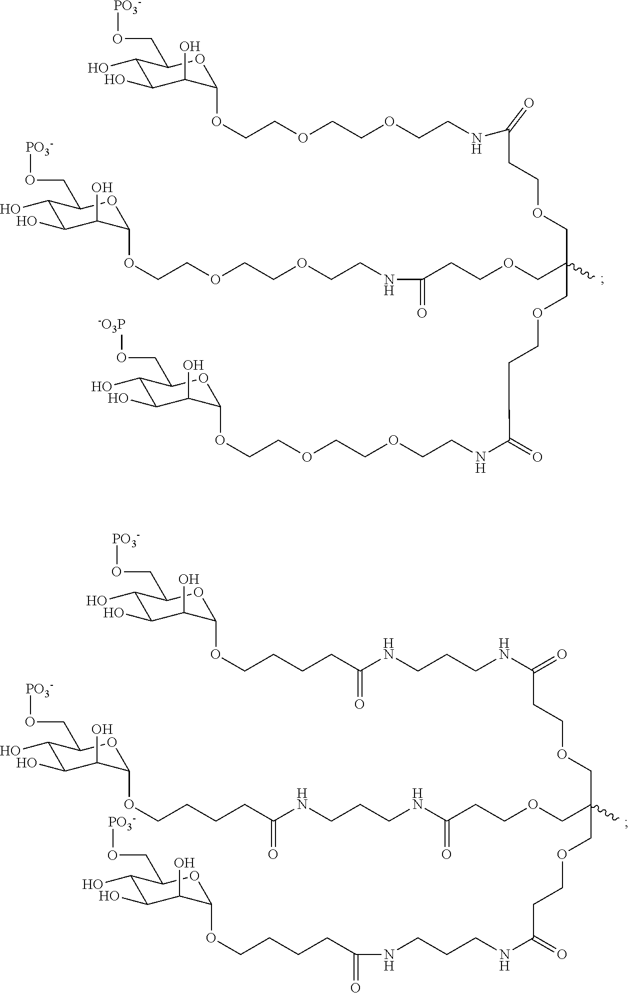

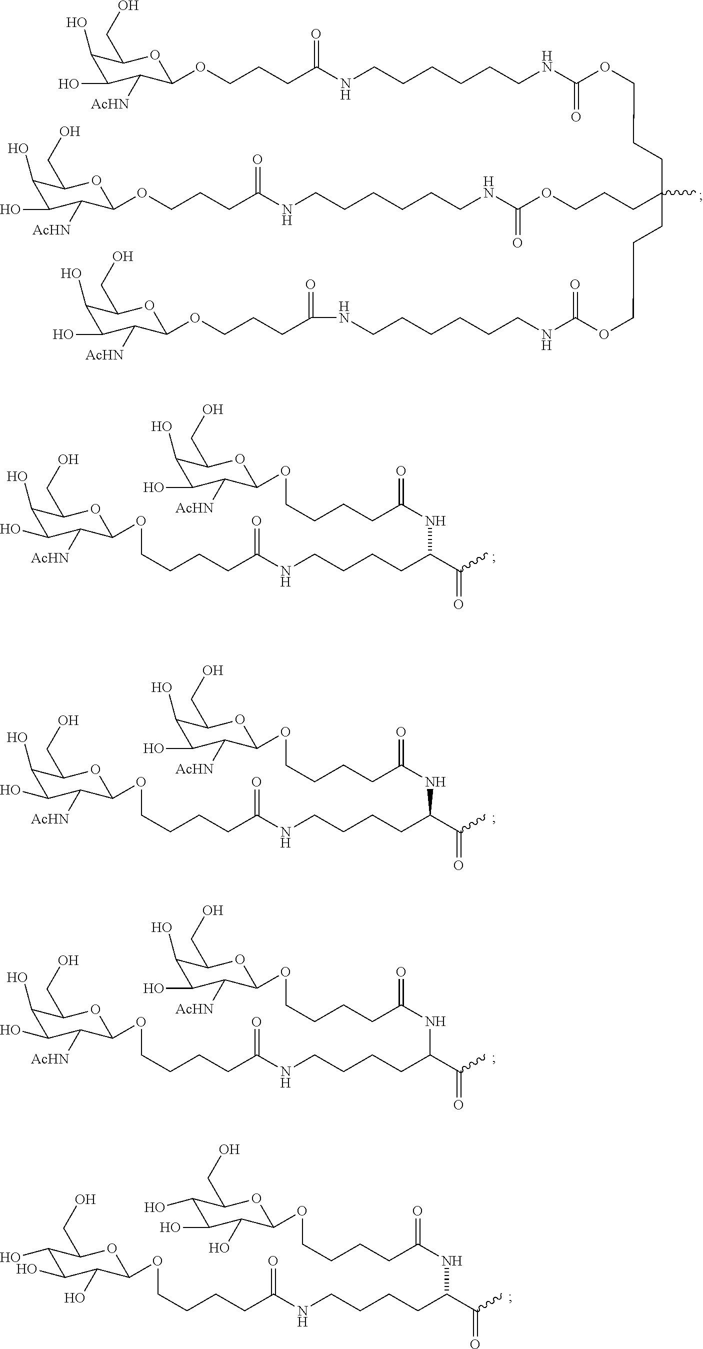

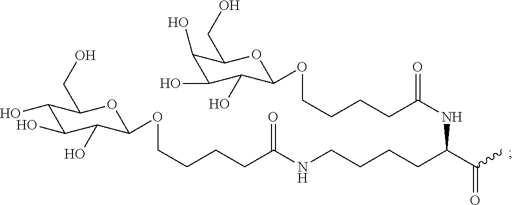

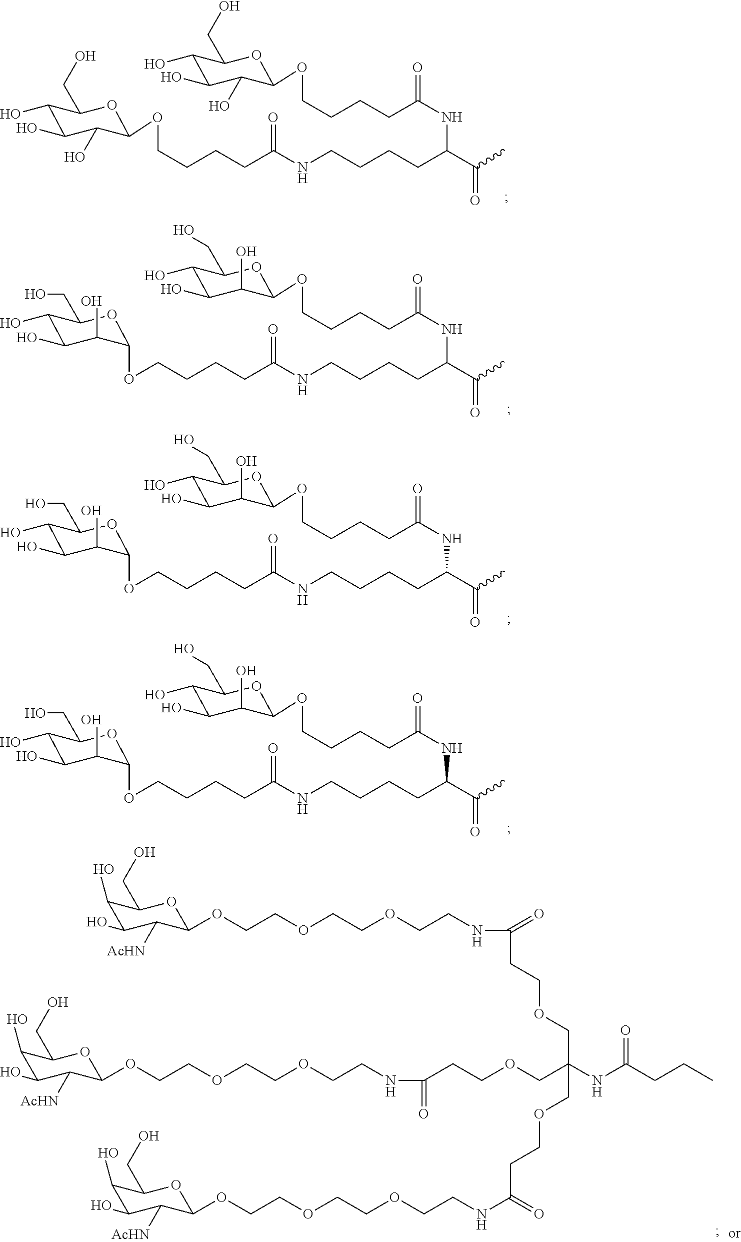

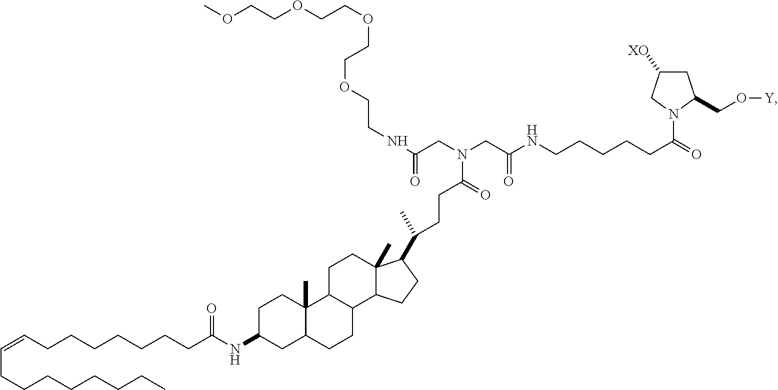

when one of X or Y is an oligonucleotide, the other is a hydrogen.

In an embodiment of the invention, the iRNA, e.g., Cemdisiran, is in a lipid nanoparticle.

In some embodiments, the conjugate or ligand described herein can be attached to an iRNA oligonucleotide with various linkers that can be cleavable or non-cleavable. The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-16, or 8-16 atoms. Linkers may comprise redox cleavable linking groups, phosphate-based cleavable linking groups, acid cleavable linking groups, ester-based linking groups and/or peptide-based cleaving groups.

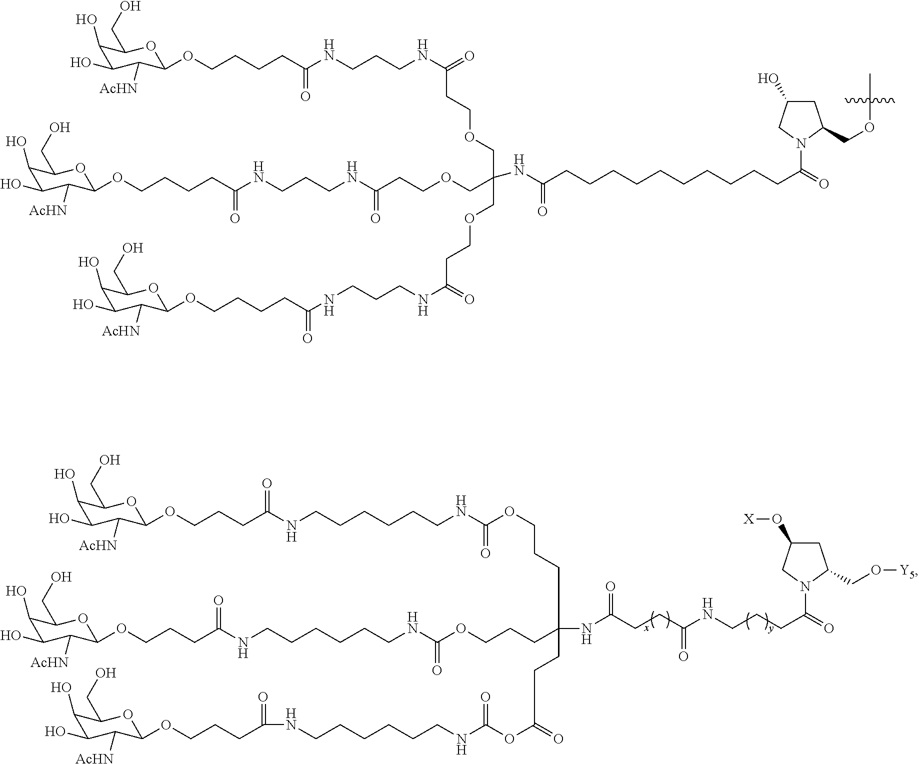

wherein x=1-30 and y=1-15;

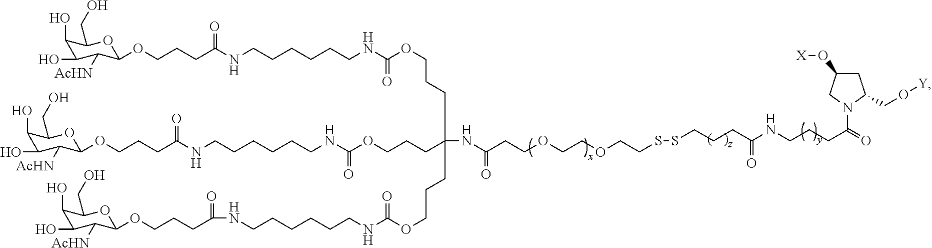

wherein y=1-30 and y=1-15;

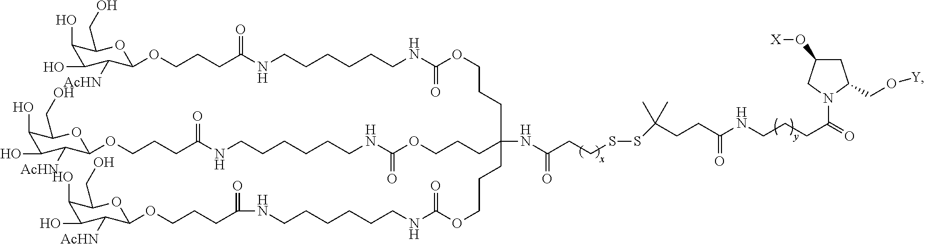

wherein x=0-30 and y=1-15;

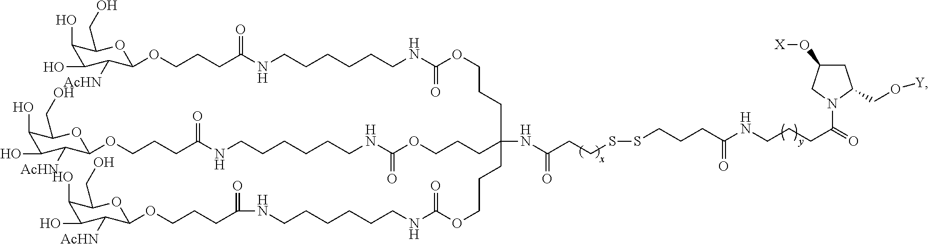

wherein x=0-30, y=1-15 and z=1-20;

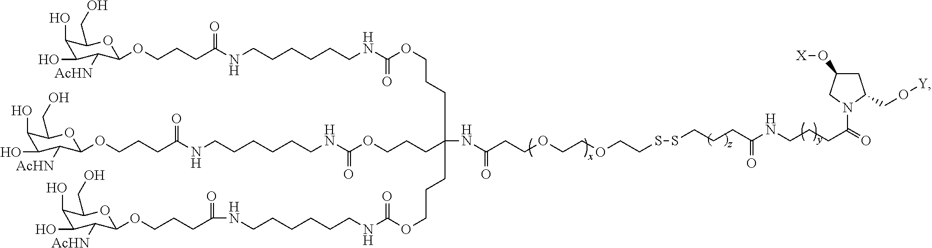

wherein x=1-30, y=1-15 and z=1-20;

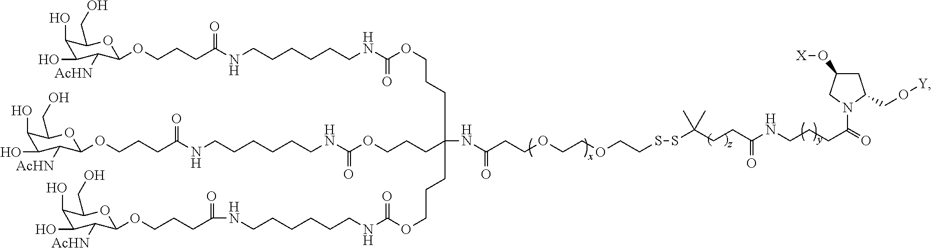

wherein x=1-30, y=1-15 and z=1-20; when one of X or Y is an oligonucleotide, the other is a hydrogen. In certain embodiments of the compositions and methods of the disclosure, a ligand is one or more GalNAc (N-acetylgalactosamine) derivatives attached through a bivalent or trivalent branched linker.

### Co-Formulations

The present disclosure provides pharmaceutical, preferably aqueous, co-formulations and combinations that include a pharmaceutically acceptable carrier and the components:
(i) an anti-C5 antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; Crovalimab; Eculizumab, Tesidolumab, Mubodina or Ravulizumab; preferably, Pozelimab); and
(ii) a C5 iRNA, preferably a glycoconjugate, such as Cemdisiran. Methods of treating or preventing a C5-associated disease or disorder, e.g., MG, including the step of administering a therapeutically effective amount of a co-formulation or combination as described herein, e.g., wherein 200 mg of the antibody or fragment and 200 mg of the C5 iRNA are administered on or more times, e.g., about once every 4 weeks.

A co-formulation may be designated in the form: antibody/iRNA; for example, “Pozelimab/Cemdisiran” or “Cemdisiran/Pozelimab” denotes a co-formulation of the present disclosure including Pozelimab and Cemdisiran.

A co-formulation or pharmaceutical co-formulation, as used herein, refers to a formulation including an anti-C5 antigen-binding protein (e.g., antibody or antigen-binding fragment thereof), a C5 iRNA and a pharmaceutically acceptable carrier. A pharmaceutically acceptable carrier includes, for example, one or more excipients. In an embodiment of the disclosure, a co-formulation of the present disclosure is aqueous, i.e., includes water.

Pharmaceutical formulations including anti-C5 antigen-binding proteins may be prepared by admixing the antigen-binding protein with one or more excipients (see, e.g., Hardman et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

The present invention provides a method for making a co-formulation including combining a C5 iRNA (e.g., Cemdisiran or the Na⁺ salt thereof; e.g., wherein the C5 iRNA is reconstituted with water from a lyophilized composition thereof); an antibody or antigen-binding fragment thereof that binds specifically to C5 (e.g., Pozelimab); a buffer (e.g., Histidine); a viscosity reducer (e.g., L-arginine); a stabilizer (e.g., sucrose); and a non-ionic surfactant (e.g., polysorbate 80), and, optionally, adjusting the co-formulation pH to greater than or less than about 6 (e.g., about 6.5±0.2); and, optionally sterile filtering the co-formulation.

The present disclosure provides methods for making a co-formulation of the present disclosure including combining an RNAi (e.g., Cemdisiran) and the antibody or antigen-binding fragment thereof (e.g., Pozelimab) (e.g., that includes detectable quantities of beta-hexosaminidase contaminant), and (i) adding GalNAc to the co-formulation and/or (ii) adjusting the pH of the co-formulation to about or below about 6 (e.g., within not less than 0.5). In an embodiment of the disclosure, other excipients are also combined, e.g., buffer, viscosity reducer, stabilizer and/or surfactant. Co-formulations (e.g., Cemdisiran/Pozelimab) produced by such methods are part of the present disclosure. In an embodiment of the disclosure, the antibody or fragment which is combined with the other components is initially in a lot that includes beta-hexosaminidase (also referred to herein as “beta-hex”) contaminant and is diluted by a factor of 0.25, 0.5 or 0.75 when incorporated into the co-formulation.

Various viscosity reducers are known in the art for use with co-formulations. viscosity reducer are agents that can reduce the viscosity of a formulation. Viscosity reducers may also function as tonicifiers that modulate the osmolality of the formulation. Such viscosity reducer include an adipic acid; an amino acid or salt thereof; (D- or L-) arginine; L-arginine HCl; (D- or L-) alanine; benzenesulfonic acid; caffeine; a dicarboxylic acid; an ester of citric acid; (D- or L-) glutamate; Glycine; (D- or L-) histidine; an inorganic salt; L-Ornithine; (D- or L-) lysine; Proline; (D- or L-) phenylalanine; (D- or L-) serine; NaCl; pyridoxamine; pyridoxine; thiamine phosphoric acid ester chloride dihydrate; triethyl citrate; (D- or L-) valine; and/or a xanthine. In an embodiment of the disclosure, the amino acid is an L-amino acid such as L-arginine. L-arginine acts both as a tonicifier as well as a stabilizer and viscosity reducer. Arginine HCl can decrease Cemdisiran degradation and allow for a near isotonic solution.

Stabilizers include agents, such as sugars or polyols, that aid in the reduction of degradation, for example, of antibodies or antigen-binding fragments, e.g., aggregation. Polyols are sugar alcohols having multiple hydroxyl groups. Stabilizers include a sugar or polyol, e.g., trehalose, sorbitol, mannitol, taurine, propane sulfonic acid, L-proline, sucrose, glycerol, threitol, maltitol, and/or polyethylene glycol (PEG; such as PEG3350).

Non-ionic surfactants contain molecules with head groups that are uncharged. Non-ionic surfactants include a non-ionic surfactant including a polyoxyethylene moiety; a sorbitan; a polyoxyethylene glycol alkyl ether, such as octaethylene glycol monododecyl ether; pentaethylene glycol monododecyl ether; polyoxypropylene glycol alkyl ether; glucoside alkyl ether, such as decyl glucoside, lauryl glucoside, octyl glucoside; polyoxyethylene glycol octylphenol ether, such as triton X-100; polyoxyethylene glycol alkylphenol ether, such as nonoxynol-9; glycerol alkyl ester, such as glyceryl laurate; polyoxyethylene glycol sorbitan alkyl ester, such as polysorbate; sorbitan alkyl ester, such as spans; cocamide MEA, cocamide DEA, dodecyldimethylamine oxide; block copolymer of polyethylene glycol and polypropylene glycol, such as poloxamer; and polyethoxylated tallow amine (POEA); poloxamer 188, polyethylene glycol 3350, a polyethylene glycol (e.g., PEG3350) or a polysorbate such as polysorbate 80 (PS80) or polysorbate 20 (PS20). In an embodiment of the disclosure, the non-ionic detergent is polysorbate-20 (PS20), polysorbate-80 (PS80).

A buffer is a mixture of a weak acid and its conjugate base or vice versa which resists changes in its pH and therefore keeps the pH at a nearly constant value. Various buffers may be used in the co-formulations of the present disclosure, for example, histidine-based buffer, phosphate buffer or citrate buffer. A histidine-based buffer is a buffer including histidine. Examples of histidine buffers include histidine chloride, histidine hydrochloride, histidine acetate, histidine phosphate, and histidine sulphate.

The present disclosure encompasses co-formulations having any of the specifically recited components, e.g., at the specifically recited concentrations, but wherein the pH of the co-formulation is about 6.5.

In an embodiment of the disclosure, the co-formulation contains the impurity, beta-hexosaminidase, e.g., in a quantity of about 0.04 to about 0.17 micrograms/ml, e.g., when pH of the co-formulation is less than or greater than about 6 (e.g., by at least 0.5), e.g., 6.5.

In an embodiment of the invention, the anti-C5 antibody or antigen-binding fragment thereof (e.g., Pozelimab) and the C5 iRNA (e.g., Cemdisiran Na⁺ form or another salt form or free acid form) are formulated in separate aqueous formulations, for example, wherein the Pozelimab is formulated at about 200 mg/mL in about 20 mM histidine (e.g., histidine-based buffer), about 100 mM L-arginine (e.g., L-arginine hydrochloride), about 2% sugar or polyol (e.g., sucrose) (w/v), about 0.15% (w/v) nonionic surfactant (e.g., polysorbate 80), pH about 5.8; and/or wherein the Cemdisiran is formulated at about 189 mg/mL (of the free acid form) in water. In an embodiment of the invention, the antibody or fragment is taken from a vial (e.g., a glass vial or pre-filled syringe or autoinjector) containing about 200 mg (±20 mg) of the antibody or fragment and/or the C5 iRNA is taken from a vial containing about 200 mg (±20 mg) or the molar equivalent thereof or about 10-13 (±2) micromoles of the C5 iRNA.

For example, the disclosure includes an aqueous pharmaceutical co-formulation (e.g., Cemdisiran/Pozelimab) including:
One or more C5 iRNAs, for example as set forth herein (e.g., Cemdisiran, preferably the Na⁺ form), for example, at a concentration of about 20-100, 20, 25, 30, 35, 40, 45, 50, 55, 60, 65, 70, 75, 80, 85, 90, 95, 100 mg/ml (or higher, e.g., 110, 115, 120, 130, 140, 150, 155, 160, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 20-50, 20-200, 20-300, 20-400, 100-300, or 100-400 mg/ml mg/ml); wherein the free acid form content can be determined by multiplying the Na⁺ form concentration by 0.9443;
One or more anti-C5 antibodies or antigen-binding fragments thereof (e.g., Pozelimab), for example, at a concentration from about 90 to about 275 mg/ml (e.g., about 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 211, 220, 242, 274 mg/ml) or at least about 150 mg/ml, at least about 175 mg/ml, at least about 200 mg/ml, at least about 211 mg/ml, at least about 220 mg/ml, at least about 242 mg/ml or at least about 274 mg/ml, 100-275, 100-300, 150-275, or 150-300);
A viscosity reducer such as L-arginine (e.g., L-arginine HCl) (e.g., at a concentration from about 40-140 mM, e.g., about 50 mM or 90 mM) (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139 or 140 mM, 30-150, 40-100 or 50-150);
A stabilizer, such as a sugar or polyol (e.g., at a concentration from about 0.8 to about 3.6% (w/v), for example about 1%) (e.g., 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6% (w/v)), 0.5-4.0, 1-2, 1-3, or 2-4% (w/v));
A non-ionic surfactant, such as polysorbate 80 (PS80) or polysorbate 20 (PS20) (e.g., at a concentration of about 0.025 to about 0.2% (w/v), e.g., about 0.075% (w/v) (e.g., 0.025, 0.05, 0.075, 0.1, 0.125, 0.15, 0.175, 0.2% (w/v)) or 0.02-0.25, 0.025-0.1, 0.025-0.15, or 0.03-0.2% (w/v);
A buffer such as a histidine-based buffer (e.g., at a concentration from about 10 to about 50 mM, for example about 30 mM e.g., 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50 mM; or 10-30, 10-40, 20-40, or 20-50 mM)); and having
A pH of about 5.5 to about 7.0, e.g., about 6.5; or within not less than 0.5 of pH 6.0 or 5.5-6, 5.5-6.5, 6-6.5, 6-7, 0-5.5, or 6.5-14.

In an embodiment of the disclosure, a co-formulation (e.g., Cemdisiran/Pozelimab) comprises (e.g., for example, with detectable quantities of beta-hexosaminidase as discussed herein):
A double stranded C5 iRNA that is conjugated to a triantennary GalNAc moiety; and
an anti-C5 antibody or antigen-binding fragment thereof that was expressed and isolated from a mammalian host cell that includes beta-hexosaminidase,
a pH above or below (by at least 0.5) 6.0;
a C5 iRNA (e.g., that is conjugated to a triantennary GalNAc moiety),
an anti-C5 antibody or antigen-binding fragment thereof (e.g., that was expressed and isolated from a mammalian host cell that includes beta-hexosaminidase),
a buffer,
a viscosity reducer,
a stabilizer, and
a non-ionic surfactant;
a C5 iRNA (e.g., that is conjugated to a triantennary GalNAc moiety),
an anti-C5 antibody or antigen-binding fragment thereof (e.g., that was expressed and isolated from a mammalian host cell that includes beta-hexosaminidase),
Histidine-based buffer,
L-arginine,
a stabilizer, and
a non-ionic surfactant;
a C5 iRNA (e.g., that is conjugated to a triantennary GalNAc moiety),
An anti-C5 antibody or antigen-binding fragment thereof (e.g., that was expressed and isolated from a mammalian host cell that includes beta-hexosaminidase),
Histidine-based buffer,
L-arginine,
a sugar or polyol, and
a non-ionic surfactant;
Cemdisiran,
Pozelimab (e.g., that was expressed and isolated from a mammalian host cell that includes beta-hexosaminidase),
Histidine-based buffer,
L-arginine,
a stabilizer, and
a non-ionic surfactant,
pH about 6.5;
Cemdisiran,
Pozelimab (e.g., that was expressed and isolated from a mammalian host cell that includes beta-hexosaminidase),
Histidine-based buffer,
L-arginine,
sucrose, and
polysorbate 80,
pH about 6.5;
100 (±10) mg/mL C5 iRNA such as Cemdisiran (Na⁺ form),
100 (±10) mg/ml anti-C5 antibody or antigen-binding fragment thereof such as Pozelimab (e.g., that was expressed and isolated from a mammalian host cell that includes beta-hexosaminidase),
50 (±10) mM viscosity reducer such as L-arginine (e.g., L-arginine HCl),
10 (±2) mM buffer such as histidine-based buffer,
1.0 (±0.2) % stabilizer such as sucrose,
0.075 (±0.00375) % non-ionic surfactant such as PS80,
pH 6.5;
75 (±7.5) mg/ml C5 iRNA such as Cemdisiran (Na⁺ form),
150 (±15) mg/ml anti-C5 antibody or antigen-binding fragment thereof such as Pozelimab (e.g., that was expressed and isolated from a mammalian host cell that includes beta-hexosaminidase),
75 (±15) mM viscosity reducer such as L-arginine (e.g., L-arginine HCl),
15 (±3) mM buffer such as histidine-based buffer,
1.5 (±0.3) % stabilizer such as sucrose,
0.1125 (±0.056) % non-ionic surfactant such as PS80,
pH 6.5;
50 (±5) mg/mL C5 iRNA such as Cemdisiran (Na⁺ form),
100 (±10) mg/ml anti-C5 antibody or antigen-binding fragment thereof such as Pozelimab (e.g., that was expressed and isolated from a mammalian host cell that includes beta-hexosaminidase),
75 mM (±15) viscosity reducer such as L-arginine (e.g., L-arginine HCl),
15 (±3) mM buffer such as histidine-based buffer,
1.5 (±0.3) % stabilizer such as sucrose,
0.1125 (±0.056) % non-ionic surfactant such as PS80;
pH 6.5;
50 (±5) mg/mL C5 iRNA such as Cemdisiran (Na⁺ form),
100 (±10) mg/ml anti-C5 antibody or antigen-binding fragment thereof such as Pozelimab (e.g., that was expressed and isolated from a mammalian host cell that includes beta-hexosaminidase),
75 (±15) mM viscosity reducer such as L-arginine (e.g., L-arginine HCl),
35 (±7) mM buffer such as histidine-based buffer,
1.5 (±0.3) % stabilizer such as sucrose,
0.1125 (±0.056) % non-ionic surfactant such as PS80, pH 6.5;
100 (±10) mg/ml C5 iRNA such as Cemdisiran (Na⁺ form),
100 (±10) mg/ml anti-C5 antibody or antigen-binding fragment thereof such as Pozelimab (e.g., that was expressed and isolated from a mammalian host cell that includes beta-hexosaminidase),
50 (±10) mM viscosity reducer such as L-arginine (e.g., L-arginine HCl),
30 (±6) mM buffer such as histidine-based buffer,
1 (±0.2) % stabilizer such as sucrose,
0.075 (±0.00375) % non-ionic surfactant such as PS80,
pH 6.5;
50 (±5) mg/mL C5 iRNA such as Cemdisiran (Na⁺ form),
100 (±10) mg/mL anti-C5 antibody or antigen-binding fragment thereof such as Pozelimab (e.g., that was expressed and isolated from a mammalian host cell that includes beta-hexosaminidase),
90 (±18) mM viscosity reducer such as L-arginine (e.g., L-arginine HCl),
30 (±6) mM buffer such as histidine-based buffer,
1 (±0.2) % stabilizer such as sucrose,
0.075 (±0.00375) % non-ionic surfactant such as PS80,
pH 6.5;
100 mg/mL Cemdisiran (Na⁺ form),
100 mg/mL Pozelimab,
50 mM L-arginine (e.g., L-arginine HCl),
30 mM histidine-based buffer,
1% (w/v) sucrose,
0.075% (w/v) PS80,
pH 6.5;
50 mg/mL Cemdisiran (Na⁺ form),
100 mg/mL Pozelimab,
90 mM L-arginine (e.g., L-arginine HCl),
30 mM histidine-based buffer,
1% (w/v) sucrose,
0.075% (w/v) PS80,
pH 6.5;
100 mg/mL Cemdisiran (Na⁺ form),
100 mg/mL Pozelimab,
50 mM L-arginine (e.g., L-arginine HCl),
10 mM histidine-based buffer,
1.0% sucrose,
0.075% PS80,
pH 6.5;
75 mg/mL Cemdisiran (Na⁺ form),
150 mg/mL Pozelimab,
75 mM L-arginine (e.g., L-arginine HCl),
15 mM histidine-based buffer,
1.5% sucrose,
0.1125% PS80,
pH 6.5;
50 mg/mL Cemdisiran (Na⁺ form),
100 mg/mL Pozelimab,
75 mM L-arginine (e.g., L-arginine HCl),
15 mM histidine-based buffer,
1.5% sucrose,
0.1125% PS80;
pH 6.5;
50 mg/mL Cemdisiran (Na⁺ form),
100 mg/mL Pozelimab,
75 mM L-arginine (e.g., L-arginine HCl),
35 mM histidine-based buffer,
1.5% sucrose,
0.1125% PS80,
pH 6.5;
100 mg/mL Cemdisiran (Na⁺ form),
100 mg/mL Pozelimab,
50 mM L-arginine (e.g., L-arginine HCl),
30 mM histidine-based buffer,
1% sucrose,
0.075% PS80,
pH 6.5;
47.2 mg/mL Cemdisiran (Free Acid Form (FAF)) molecule, which may be any salt form, such as Na⁺,
100 mg/mL Pozelimab,
30 mM histidine,
90 mM L-arginine,
1% (w/v) sucrose,
0.075% (w/v) polysorbate 80 (e.g., super refined grade (SR)),
pH 6.5
50 mg/mL Cemdisiran (Na⁺ form),
100 mg/mL Pozelimab,
90 mM L-arginine (e.g., L-arginine HCl),
30 mM buffer such as histidine-based buffer,
1% stabilizer such as sucrose,
0.075% PS80,
pH 6.5;
optionally, any of the co-formulations set forth herein further comprises GalNAc or GlcNAc, e.g., about 5% (w/v). The present invention includes methods for treating or preventing a C5-associated disease or disorder, e.g., MG, including administering 200 mg of the antibody or fragment and 200 mg of the C5 iRNA one or more time, e.g., about once every 4 weeks.

In an embodiment of the disclosure, a co-formulation of the present disclosure includes the antibody and iRNA and is in association with a further therapeutic agent, such as, for example, an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and/or an anti-thrombotic agent.

The present invention includes method for treating or preventing a C5-associated disease or disorder, e.g., MG, by administering 200 mg of anti-C5 antibody or antigen-binding fragment thereof in association with 200 mg of a C5 iRNA about once every 4 weeks.

The term “in association with” indicates that multiple components (e.g., Pozelimab and Cemdisiran) can be formulated into a single composition, e.g., for simultaneous delivery, or formulated separately into two or more compositions (e.g., a kit including each component, for example, wherein the further therapeutic agent is in a separate formulation). Components administered in association with each another can be administered to a subject at the same time or at a different time than when the other component is administered or in a given sequence of one component and then the other or the reverse thereof; for example, each administration may be given simultaneously (e.g., together in a single composition or essentially simultaneously during the same administration session) or non-simultaneously at one or more intervals over a given period of time. Moreover, the separate components administered in association with each other may be administered to a subject by the same or by a different route.

In an embodiment of the invention, the further therapeutic agent that may be administered in association with an anti-C5 antibody or antigen binding fragment and a C5 iRNA may be one or more of: an anticholinesterase agent, an immunosuppressive drug, a C5 complement inhibitor, a neonatal Fc receptor blocker, prednisone, tacrolimus, rituximab, eculizumab, ravulizumab, efgartigimod, rozanolixizumab, pyridostigmine, plasmapheresis (PLEX) and/or intravenous IgG.

The present disclosure includes co-formulations described herein wherein the concentration of the antibody and/or iRNA is ±10% the value shown; the concentration of surfactant is ±50% the value shown; and/or any of the other excipient concentrations (e.g., viscosity reducer, buffer, stabilizer) or pH are ±20% the value shown.

In an embodiment of the disclosure, a co-formulation of the present disclosure,
comprises about 0.04 to 0.17 micrograms/ml of beta-hexosaminidase (e.g., about 0.04; 0.05; 0.06; 0.06; 0.0605; 0.061, 0.0605; 0.0605; 0.062, 0.063; 0.07; 0.07; 0.075; 0.0765; 0.078; 0.08; 0.14; 0.141; 0.15; 0.1525; 0.166; or 0.17 micrograms/ml; or 0.02-0.2, 0.05-0.15, or 0.1-0.15 (or no more than such an amount));
is a clear to slightly opalescent liquid;
is essentially free of visible particulates;
has a colorless to yellow color;
is characterized by a ratio of Cemdisiran concentration:Pozelimab concentration of about 1:1, 1:2, 1:3, 1:4, 1:5 or 2:3, e.g., wherein the viscosity if less than about 20 cP or less than about 30 cP at 20° C.;
has a Viscosity <30 cP (at 20° C.) (e.g., about 6, 10, 20 or 30 cP, such as 6-30, 6-25, or 6-20 cP));
has an osmolality of 266-706 (e.g., about 266; 276; 286; 296; 306; 316; 326; 334; 336; 346; 356; 366; 376; 386; 396; 406; 416; 426; 436; 446; 456; 466; 476; 486; 496; 506; 516; 526; 536; 546; 556; 566; 576; 586; 596; 606; 616; 626; 636; 646; 656; 666; 676; 686; 696; or 706; or 270-700, 300-500, 300-600, 400-600, 400-700, or 500-700) mOsm/kg;
has a density of about 1.1 or 1.061 or 1.05-1.1 or 1.075-1.1 g/ml;
has a pH of about 6.5±0.2;
Has a pH higher than 6 which results in a significant reduction in Cemdisiran degradation at 40° C., 25° C., and 2-8° C. relative to pH 6;
exhibits a Cemdisiran Purity (%) by dIPRP of about 90.5% at t=0; 91.1% after 1 month storage at 2-8° C.; 90.8% after 3 months storage at 2-8° C.; 90% after 6 months storage at 2-8° C.; 88.8% after 9 months storage at 2-8° C.; 88.7% after 12 months storage at 2-8° C.; 89% after 18 months storage at 2-8° C.; 89.4% after 24 months storage at 2-8° C.; and/or 87.4% after 36 months storage at 2-8° C., e.g., wherein the co-formulation was liquid (aqueous) during storage and includes 100 mg/ml Cemdisiran and 100 mg/ml Pozelimab (e.g., at pH 6.0); for example wherein the co-formulation includes about 60.5 ng/ml beta-hex;
exhibits a Cemdisiran Purity (%) by dIPRP of about 90.8% at t=0; 90.6% after 1 month storage at 2-8° C.; 90.5% after 3 months storage at 2-8° C.; 89.4% after 6 months storage at 2-8° C.; 88.3% after 9 months storage at 2-8° C.; 87.8% after 12 months storage at 2-8° C.; 87.8% after 18 months storage at 2-8° C.; and/or 87.4% after 24 months storage at 2-8° C. and/or about 85.4% after 36 months storage at 2-8° C.; e.g., wherein the co-formulation was liquid (aqueous) during storage and includes 75 mg/ml Cemdisiran and 150 mg/ml Pozelimab (e.g., at pH 6.0); for example wherein the co-formulation includes about 60.5 ng/ml beta-hex;
exhibits a Cemdisiran Single Strand Purity (%) by dIPRP of about 90.5% at t=0; 90.2% after 1 month storage at 25° C. and 60% RH (relative humidity); 87.8% after 3 months storage at 25° C. and 60% RH; 85.1% after 6 months storage at 25° C. and 60% RH; 90% after 0.5 months storage at 40° C. and 75% RH; 88.9% after 1 month storage at 40° C. and 75% RH; 85.8% after 3 months storage at 40° C. and 75% RH; e.g., wherein the co-formulation was liquid (aqueous) during storage and includes 100 mg/ml Cemdisiran and 100 mg/ml Pozelimab (e.g., at pH 6.0); for example wherein the co-formulation includes about 60.5 ng/ml beta-hex;
exhibits a Cemdisiran Single Strand Purity (%) by dIPRP of about 91.4% after about 48 hours agitation, and/or about 90.7% after about 4 freeze-thaw cycles e.g., wherein the co-formulation was liquid (aqueous) and includes 100 mg/ml Cemdisiran and 100 mg/ml Pozelimab (e.g., at pH 6.0);
exhibits a Cemdisiran Purity (%) by dIPRP of about 90.8% at t=0; 88.8% after 1 month storage at 25° C. and 60% RH; 85.9% after 3 months storage at 25° C. and 60% RH; 82.3% after 6 months storage at 25° C. and 60% RH; 88.9% after 0.5 months storage at 40° C. and 75% RH; 87.3% after 1 month storage at 40° C. and 75% RH; 82.3% after 3 months storage at 40° C. and 75% RH; e.g., wherein the co-formulation was liquid (aqueous) during storage and includes 75 mg/ml Cemdisiran and 150 mg/ml Pozelimab (e.g., at pH 6.0); for example wherein the co-formulation includes about 60.5 or 91 ng/ml beta-hex;
exhibits a Cemdisiran Purity (%) by dIPRP of about 90.7% after about 48 hours agitation, and/or about 91.2% after about 4 freeze-thaw cycles e.g., wherein the co-formulation was liquid (aqueous) during storage and includes 75 mg/ml Cemdisiran and 150 mg/ml Pozelimab (e.g., at pH 6.0);
exhibits no more (e.g., within 5%) increase in detectable high molecular weight species (HMW species) after 48 hours of agitation on an orbital shaker at 250 rpm in the presence of 0.025% non-ionic surfactant (e.g., polysorbate 80) than in the presence of 0.050, 0.075, 0.100, 0.125, 0.150, 0.175 or 0.200% (w/v); e.g., wherein the co-formulation includes 50 or 100 mg/mL Cemdisiran and 100 mg/ml Pozelimab, for example, 100 mg/mL Cemdisiran, 100 mg/mL Pozelimab, 50 mM arginine HCl, 30 mM histidine, 1% sucrose, X % PS80, pH 6.5, or 50 mg/mL Cemdisiran, 100 mg/mL Pozelimab, 90 mM arginine HCl, 30 mM histidine, 1% sucrose, X % PS80, pH 6.5 (wherein X is about 0.025% to about 0.2% (w/v), e.g., 0.075%);
exhibits a Cemdisiran purity (%) by dIPRP of about 90.9% at t=0; about 90.1% after 1 month of storage at 25° C., 60% RH; about 90.9% after 3 months of storage at 25° C., 60% RH; about 90.4% after 6 months of storage at 25° C., 60% RH; about 89.9% after 0.5 months of storage at 40° C., 75% RH; about 89.7% after 1 month of storage at 40° C., 75% RH; and/or about 89.5% after 3 months of storage at 40° C., 75% RH; e.g., wherein the co-formulation was liquid (aqueous) during storage and contained 50 mg/ml Cemdisiran and 100 mg/ml Pozelimab, pH6 with 5% GlcNAc; e.g., wherein the co-formulation includes about 78 ng/ml beta-hex;
exhibits a Cemdisiran purity (%) by dIPRP of about 90.8% at t=0; about 90.2% after 1 month of storage at 25° C., 60% RH; about 90.8% after 3 months of storage at 25° C., 60% RH; about 90.3% after 6 months of storage at 25° C., 60% RH; about 89.5% after 0.5 months of storage at 40° C., 75% RH; about 89.6% after 1 month of storage at 40° C., 75% RH; and/or about 89.1% after 3 months of storage at 40° C., 75% RH; e.g., wherein the co-formulation was liquid (aqueous) during storage and contained 50 mg/ml Cemdisiran and 100 mg/ml Pozelimab, pH6 with 5% GalNAc; e.g., wherein the co-formulation includes about 78 ng/ml beta-hex;
exhibits a Cemdisiran purity (%) by dIPRP of about 90.5% at t=0; about 89.9% after 1 month of storage at 25° C., 60% RH; about 90.8% after 3 months of storage at 25° C., 60% RH; about 90.4% after 6 months of storage at 25° C., 60% RH; about 90.1% after 0.5 months of storage at 40° C., 75% RH; about 89.6% after 1 month of storage at 40° C., 75% RH; and/or about 89.9% after 3 months of storage at 40° C., 75% RH; e.g., wherein the co-formulation was liquid (aqueous) during storage and contained 100 mg/ml Cemdisiran and 100 mg/ml Pozelimab, pH6 with 5% GlcNAc; e.g., wherein the co-formulation includes about 78 ng/ml beta-hex; and/or
exhibits a Cemdisiran purity (%) by dIPRP of about 91.1% at t=0; about 90% after 1 month of storage at 25° C., 60% RH; about 91% after 3 months of storage at 25° C., 60% RH; about 90.7% after 6 months of storage at 25° C., 60% RH; about 90% after 0.5 months of storage at 40° C., 75% RH; about 89.7% after 1 month of storage at 40° C., 75% RH; and/or about 89.9% after 3 months of storage at 40° C., 75% RH; e.g., wherein the co-formulation was liquid (aqueous) during storage and contained 100 mg/ml Cemdisiran and 100 mg/ml Pozelimab, pH 6 with 5% GalNAc; e.g., wherein the co-formulation includes about 78 ng/ml beta-hex.

### Dosing Regimens of Anti-C5 and C5 iRNA

The present disclosure includes methods that comprise administering, to a subject in need thereof, with a disease or disorder or condition associated with C5 (e.g., MG, for example, wherein the subject is anti-acetylcholine receptor (AChR) and/or anti-low-density lipoprotein receptor-related protein 4 (LRP4) antibody positive):
a C5 iRNA such as Cemdisiran (optionally, wherein an additional C5 inhibitor is not administered); or
an anti-C5 antibody or antigen-binding fragment thereof in combination with a C5 iRNA (e.g., in the form of a co-formulation including both the antibody or fragment and the iRNA, e.g., as set forth herein; or separate injections of the antibody or fragment and iRNA),
   at a dosing amount and frequency that achieves a safe and effective therapeutic response (combination therapy of the present disclosure).

The present disclosure encompasses methods for administering a C5 iRNA (but, optionally, not an anti-C5 antibody or antigen-binding fragment thereof or other C5 inhibitor) including introducing the C5 iRNA into the body of a subject, e.g., by injection, such as by subcutaneous injection or intravenous infusion, for example, under a schedule according to any of the dosing regimens discussed herein, e.g.,
about 567 mg (±57 mg) of C5 iRNA, 32-35 or 33.8 (±4) micromoles of C5 iRNA or 600 mg (±60 mg) of C5 iRNA which is in salt for such as Nat form (e.g., wherein the C5 iRNA is Cemdisiran Na⁺ form of another salt form or the free form), preferably subcutaneously, about every 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year;
   or
about 200 mg (±20 mg) of the anti-C5 antibody or antigen-binding fragment thereof (e.g., Pozelimab) and about 189 mg (±19 mg) of C5 iRNA, 11-13 (±2) micromoles of C5 iRNA or 200 mg (±20 mg) of C5 iRNA which is in salt for such as Na⁺ form (e.g., wherein the C5 iRNA is Cemdisiran Nat form of another salt form or the free form), which may be administered in a single co-formulation or by two separate administrations of the antibody or fragment and the C5 iRNA, about once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days), preferably subcutaneously.
   In an embodiment of the invention, the Cemdisiran Na⁺ form is in an aqueous pharmaceutical formulation comprising 200 mg/ml Cemdisiran Na⁺ form. In an embodiment of the invention, the 600 mg of Cemdisiran is in an aqueous pharmaceutical formulation and 3 ml of the formulation is administered to achieve a full dose, e.g., in two doses of 1.5 ml each or 3 injections of 1 ml each or a single injection with a full dose.

In some embodiments, the present disclosure relates to the administration of one or more doses of an anti-C5 antibody or antigen-binding fragment thereof (e.g., Pozelimab) in combination with one or more doses of a C5 iRNA (e.g., Cemdisiran). In an embodiment of the invention, the administration is in a co-formulation of the present disclosure (as discussed herein), e.g., 100:100 or 50:100 (Cemdisiran mg/ml:Pozelimab mg/ml)), for example in an injection volume of about 2 ml.

Generally, herein, a co-formulation including Cemdisiran and Pozelimab may be referred to in the following format: 100:100. In such notation, when referring to such a co-formulation, the first number indicates the mg/ml of Cemdisiran and the second number indicates the mg/ml of Pozelimab.

Thus, the present disclosure provides a method for administering to a subject, e.g., who is in need thereof, e.g., who is suffering from a C5-associated disease or disorder (for example, MG, for example, wherein the subject is anti-acetylcholine receptor (AChR) and/or anti-low-density lipoprotein receptor-related protein 4 (LRP4) antibody positive) including introducing into the body of the subject, e.g., parenterally, a C5 iRNA (e.g., Cemdisiran) and/or a combination of C5 iRNA (e.g., Cemdisiran) and anti-C5 antibody or antigen-binding fragment thereof (e.g., Pozelimab) according to the following:
One or more doses of about 600 mg (±60 mg) C5 iRNA Na⁺ form or a molar equivalent amount of another salt form or the free form;
One or more doses about 32-25 (±4) (e.g., 32, 33, 33.8, 34 or 35) micromoles C5 iRNA, e.g., Na⁺ form or another salt form or the free form; or
One or more doses about 567 mg (±57 mg) of C5 iRNA free form which is in Nat salt form or another salt form;
One or more doses of about 600 mg (±60 mg) Cemdisiran Na⁺ form or a molar equivalent amount of another salt form or the free form;
One or more doses of about 32-25 (±4) (e.g., 32, 33, 33.8, 34 or 35) micromoles Cemdisiran, e.g., Na⁺ form or another salt form or the free form;
One or more doses of about 567 mg (±57 mg) of Cemdisiran free form which is in Na⁺ salt form or another salt form;
about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year;
about 32-25 (±4) (e.g., 32, 33, 33.8, 34 or 35) micromoles Cemdisiran, e.g., Na⁺ form or another salt form or the free form, about every 84 days (±1, 2, 3, 4, 5, 6 or 7 days), or about every 12 weeks (+1, 2, 3, 4, 5, 6 or 7 days), or about every 3 months (±1, 2, 3, 4, 5, 6 or 7 days) or about every ¼ year; or
about 567 mg (±57 mg) of Cemdisiran free form which is in Na⁺ salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year;
one or more doses of about 200 mg (±20 mg) of the anti-C5 antibody or antigen-binding fragment thereof (e.g., Pozelimab) subcutaneously, in combination with about 200 mg (±20 mg) C5 iRNA which is in Na⁺ salt form or the molar equivalent of another salt form or the free form, subcutaneously, e.g., once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days);
one or more does of about 200 mg (±20 mg) of the anti-C5 antibody or antigen-binding fragment thereof (e.g., Pozelimab), in combination with about 10-13 (±2) micromoles of the C5 iRNA (e.g., Cemdisiran Na⁺ form of another salt form or the free form), subcutaneously, e.g., once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days); and/or
one or more doses of about 200 mg (±20 mg) of the anti-C5 antibody or antigen-binding fragment thereof (e.g., Pozelimab) in combination with about 189 mg of the C5 iRNA free form which is in a salt form, e.g., Cemdisiran free form which is in Nat salt form or another salt form, subcutaneously, e.g., once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days);
   for example, wherein a combination including the antibody or fragment and the C5 iRNA is administered in a co-formulation that includes the antibody or fragment and the C5 iRNA; or in two separate formulations, one including the antibody or fragment and the other including the C5 iRNA.

Thus, the present disclosure provides a method for treating or preventing a C5-associated disease or disorder (for example, MG, for example, wherein the subject is anti-acetylcholine receptor (AChR) and/or anti-low-density lipoprotein receptor-related protein 4 (LRP4) antibody positive) in a subject in need thereof including administering to the subject a C5 iRNA according to the following:
One or more doses of about 600 mg (±60 mg) C5 iRNA Na⁺ form or a molar equivalent amount of another salt form or the free form;
One or more doses about 32-35 (±4) (e.g., 32, 33, 33.8, 34 or 35) micromoles C5 iRNA, e.g., Na⁺ form or another salt form or the free form; or
One or more doses about 567 mg (±57 mg) of the C5 iRNA free form which is in Na⁺ salt form or another salt form,
   for example, wherein the doses are administered once every about 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year. In an embodiment of the invention, the C5 iRNA is in an aqueous formulation comprising 200 mg/ml C5 iRNA and 3 ml of the formulation is administered to achieve a full dose.

Thus, the present disclosure provides a method for treating or preventing a C5-associated disease or disorder (for example, MG, for example, wherein the subject is anti-acetylcholine receptor (AChR) and/or anti-low-density lipoprotein receptor-related protein 4 (LRP4) antibody positive) in a subject in need thereof including administering to the subject Cemdisiran according to the following:
One or more doses of about 600 mg (±60 mg) Cemdisiran Na⁺ form or a molar equivalent amount of another salt form or the free form;
One or more doses about 32-35 (±4) (e.g., 32, 33, 33.8, 34 or 35) micromoles Cemdisiran, e.g., Na⁺ form or a molar equivalent amount of another salt form or the free form; or
One or more doses about 567 mg (±57 mg) of the Cemdisiran free form which is in Na⁺ salt form or another salt form.
   In an embodiment of the invention, no further C5 inhibitor, such as an anti-C5 antibody or antigen-binding fragment thereof, is administered. In an embodiment of the invention, the Cemdisiran Na⁺ form is in an aqueous formulation comprising about 200 mg/ml Cemdisiran Na⁺ form, about 10-13 micromoles/ml of Cemdisiran (Na⁺ form or other salt form or free form); or about 189 mg/ml Cemdisiran free form which is in Nat form or other salt form; and about 3 ml of the formulation is administered to achieve a full dose. Such a dosing regimen may be referred to herein as Cemdisiran monotherapy.

Thus, the present disclosure provides a method for treating or preventing a C5-associated disease or disorder (for example, MG, for example, wherein the subject is anti-acetylcholine receptor (AChR) and/or anti-low-density lipoprotein receptor-related protein 4 (LRP4) antibody positive) in a subject in need thereof including administering to the subject Cemdisiran according to the following:
about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year;
about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year;
   or
about 567 mg (±57 mg) of Cemdisiran free form which is in Na⁺ salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year.
   In an embodiment of the invention, no further C5 inhibitor, such as an anti-C5 antibody or antigen-binding fragment thereof, is administered. In an embodiment of the invention, the subject receives at least two doses of the C5 iRNA. In an embodiment of the invention, the Cemdisiran Na⁺ form is in an aqueous formulation comprising about 200 mg/ml Cemdisiran Na⁺ form, about 10-13 micromoles/ml of Cemdisiran (Na⁺ form or other salt form or free form); or about 189 mg/ml Cemdisiran free form which is in Nat form or other salt form; and about 3 ml of the formulation is administered to achieve a full dose.

In an embodiment of the invention, a full dose of C5 iRNA is delivered in three doses of ⅓ of a dose each or two doses of half a dose each.

Thus, the present disclosure provides a method for treating or preventing a C5-associated disease or disorder (for example, MG, for example, wherein the subject is anti-acetylcholine receptor (AChR) and/or anti-low-density lipoprotein receptor-related protein 4 (LRP4) antibody positive) in a subject in need thereof including administering to the subject an anti-C5 antibody or antigen-binding fragment thereof (“the anti-C5 Ab”) and a C5 iRNA according to the following:
(i) one or more doses of about 200 mg (±20 mg) of the anti-C5 antibody or antigen-binding fragment thereof (e.g., Pozelimab) subcutaneously, in combination with about 200 mg (±20 mg) or 10-13 (±2) micromoles of the C5 iRNA (e.g., Cemdisiran Na⁺ form or a molar equivalent amount of another salt form or the free form) subcutaneously;
(ii) one or more does of about 200 mg (±20 mg) of the anti-C5 antibody or antigen-binding fragment thereof (e.g., Pozelimab), in combination with about 200 mg (±20 mg) or 10-13 (±2) micromoles of the C5 iRNA (e.g., Cemdisiran Na⁺ form or a molar equivalent amount of another salt form or the free form); or
(iii) one or more doses of about 200 mg (±20 mg) of the anti-C5 antibody or antigen-binding fragment thereof (e.g., Pozelimab) in combination with about 189 mg of C5 iRNA, e.g., Cemdisiran free form which is in Na⁺ salt form or another salt form;
   administered subcutaneously, for example, about every 4 weeks (±7 days), preferably in a single injection of a co-formulation that includes both the anti-C5 Ab and the C5 iRNA, or in two separate injections of the antibody or fragment and the C5 iRNA.

In an embodiment of the disclosure, the subject is administered, concurrently,
about 200 mg (±20 mg) of the anti-C5 antibody or antigen-binding fragment thereof (e.g., Pozelimab) subcutaneously about once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days); and
about 200 mg (±20 mg) C5 iRNA, e.g., Cemdisiran Na⁺ form or a molar equivalent amount of another salt form or the free form) or 10-13 (±2) micromoles of the C5 iRNA, e.g., Cemdisiran Na⁺ form or another salt form or free form, subcutaneously about once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days).
   This dosing regimen may be referred to herein as “Pozelimab Q4W and Cemdisiran”, “Pozelimab and Cemdisiran Q4W”, or “Pozelimab 200 mg (±20 mg) SC Q4W+Cemdisiran 200 mg (±20 mg) SC Q4W”).

Generally, an initial dose or doses that are non-recurring may be referred to as “loading” doses and subsequent doses that are recurring may be referred to as “maintenance” doses.

In an embodiment of the disclosed technology, a loading dose is administered.

In an embodiment of the disclosed technology, no loading dose is administered.

In an embodiment of the disclosed technology, the anti-C5 antibody or antigen-binding fragment thereof (e.g., Pozelimab) and/or the C5 iRNA (e.g., Cemdisiran) is administered intravenously.

In an embodiment of the disclosed technology, the anti-C5 antibody or antigen-binding fragment thereof (e.g., Pozelimab) and/or the C5 iRNA (e.g., Cemdisiran) is not administered intravenously.

A dosing regimen including monthly doses of both anti-C5 antibody or antigen-binding fragment thereof (e.g., Pozelimab) and C5 iRNA (e.g., Cemdisiran), may be referred to as the q4w or Q4W combination regimen.

A dosing regimen including monthly doses of anti-C5 antibody or antigen-binding fragment thereof (e.g., Pozelimab) may be referred to as the q4w or Q4W anti-C5 regimen.

A dosing regimen including doses every 3 months of C5 iRNA (e.g., Cemdisiran) may be referred to as the q12w or Q12W C5 iRNA regimen.

In an embodiment of the invention, q4w refers to doses about every 4 weeks, about every month, about every 30 days, about 3 times per quarter or about 12 times per year.

The term “4 weeks”, in an embodiment of the disclosed technology, refers to about 28, 29 or 30 days (±1, 2, 3, 4, 5, 6 or 7 days) or about 4 calendar weeks (±1, 2, 3, 4, 5, 6 or 7 days).

The term “2 weeks”, in an embodiment of the disclosed technology, refers to about 14 days (±1, 2, 3, 4, 5, 6 or 7 days) or about 2 calendar weeks (±1, 2, 3, 4, 5, 6 or 7 days).

The term “one month”, in an embodiment of the disclosed technology, refers to approximately a calendar month (±1, 2, 3, 4, 5, 6 or 7 days); 28, 29 or 30 days (±1, 2, 3, 4, 5, 6 or 7 days) or 4 weeks (±1, 2, 3, 4, 5, 6 or 7 days).

The term “3 months”, in an embodiment of the disclosed technology, refers to a period of approximately 3 calendar months (±1, 2, 3, 4, 5, 6 or 7 days), e.g., a period of from January 1 to March 31 or April 1; about 12 weeks (±1, 2, 3, 4, 5, 6 or 7 days); or about 84 days (±1, 2, 3, 4, 5, 6 or 7 days).

The term “12 weeks”, in an embodiment of the disclosed technology, refers to approximately 12 calendar weeks (±1, 2, 3, 4, 5, 6 or 7 days); or about 84 days (±1, 2, 3, 4, 5, 6 or 7 days) which may include 77, 78, 79, 80, 81, 82, 83, 84, 85, 86, 87, 88, 89, 90 or 91 days.

The term “¼ year”, in an embodiment of the disclosed technology, refers to a period of approximately 4 times per year (e.g., 365 days or a calendar year) or about 3 months (±1, 2, 3, 4, 5, 6 or 7 days).

The term 600 mg (±60 mg) includes about 600 mg (±50 mg), 600 mg (±40 mg), 600 mg (±30 mg), 600 mg (±20 mg), 600 mg (±10 mg), 600 mg (±10 mg), 600 mg (±1 mg) 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg or 660 mg.

Anti-C5 antibody or antigen-binding fragment thereof 200 mg (±20 mg) SC Q4W refers to administration of about 200 mg (±20 mg) of the antibody or fragment (e.g., Pozelimab) subcutaneously about every 4 weeks (e.g., 28 days (±1, 2, 3, 4, 5, 6 or 7 days)).

C5 iRNA 200 mg (±20 mg) SC Q4W refers to administration of about 200 mg (±20 mg) of the iRNA (e.g., Cemdisiran Na⁺ form or a molar equivalent amount of another salt form or the free form) subcutaneously about every 4 weeks (e.g., 28 days (±1, 2, 3, 4, 5, 6 or 7 days)).

C5 iRNA about 600 mg (±60 mg) or about 32-35 or 33.8 (±4) micromoles SC Q12W refers to administration of about 600 mg (±60 mg) or about 32-35 or 33.8 (±4) micromoles of the iRNA (e.g., Cemdisiran Na⁺ form or a molar equivalent amount of another salt form or the free form) subcutaneously about every 3 months, 12 weeks or 84 days (+1, 2, 3, 4, 5, 6 or 7 days) or about 4 times per year.

As set forth herein, any dosing episode (e.g., which involves multiple doses of drugs), may be followed by a 30 minute to 2-hour observation period after the last administration or for however long, in the judgment of the treating physician, no adverse events are likely to occur acutely. Typically, on a given day when a subject receives an intravenous dose and one or more subcutaneous doses, the intravenous dose is given first; however, the scope of the present disclosure includes embodiments wherein the doses are given in any order, e.g., SC then IV then SC.

In an embodiment of the invention, Cemdisiran is provided as three separate vials containing about 1 ml of solution including Cemdisiran. Administration can include one or more of the steps:
removing the vials from refrigeration and allowing the vials to sit for at least 15 minutes at room temperature 20° C. to 25° C. (68° F. to 77° F.) before use.
inspecting the solution visually for particulate matter and discoloration prior to administration and discarding if the solution is cloudy, discolored or contains particulate matter. Cemdisiran should be a clear, colorless to yellow solution that is free from visible particulates.
gently swirling the vials in an upright position. Shaking should be avoided since this may result in foaming.
using a withdrawal needle (21 gauge (G) needle with Luer-Lok) to withdraw 3 mL of the Cemdisiran solution.
dividing the dose equally into two syringes (each containing 1.5 mL) or 3 syringes (each containing 1 ml) or a single syringe with a full dose.
changing the needle on each syringe to an injection needle fulfilling the following criteria: 25 G to 27 G and ½ or ⅝-inch stainless steel needle with Luer-Lok.
administering the subcutaneous injections consecutively, each at a different injection site, into the thigh, back of the upper arm, or abdomen, on opposite sides or at least 2 cm apart. Injections should not be performed into moles, scars, or areas where the skin is tender, bruised, red, hard, or not intact.
observing the subject for 30 minutes following completion of the first visit.
administering the subcutaneous injection within 4 hours of preparation.

In an embodiment of the disclosure, a subject receiving the combination therapy of anti-C5 Ab and C5 iRNA achieves or achieves and maintains while receiving the therapy one or more of the following:
Improvement in Myasthenia Gravis-Activities of Daily Living (MG-ADL) total score from baseline to week 24;
Improvement in Quantitative Myasthenia Gravis (QMG) score from baseline to week 24;
≥3-point improvement from baseline to week 24 on the MG-ADL;
≥5-point improvement from baseline to week 24 on the QMG;
At least a 2-point MG-ADL improvement on 2 or more consecutive assessments on the MG-ADL spanning 4 or more weeks during the double-blind treatment period (DBTP);
Minimal symptom expression (MSE), defined by a score of 0 to 1 on the MG-ADL at week 24;
Improvement in the Myasthenia Gravis Composite (MGC) total score from baseline to week 24;
Improvement in Myasthenia Gravis Quality of Life (MG-QOL15r) total score from baseline to week 24;
Improvement point threshold of ≥2, 4, 5, 6, 7, 8, 9, or 10 on MG-ADL at week 24;
Improvement point threshold of ≥3, 4, 6, 7, 8, 9, or 10 on QMG at week 24;
Reduced incidence and severity of treatment-related adverse events (TEAEs), serious adverse events (SAEs), and adverse events of special interest (AESIs) in comparison to placebo through week 24;
Improvement from baseline in MG-ADL total score by visit;
Improvement from baseline in QMG total score by visit;
Reduced time to achievement of a 3-point improvement from baseline on the MG-ADL in comparison to placebo;
Reduced time to achievement of a 5-point improvement from baseline on the QMG in comparison to placebo;
Improvement from baseline in the physical performance measures of hand grip strength, supine leg lift, supine head lift, onset of dysarthria, and all timed items of the QMG;
Improvement from baseline in individual domains (ocular, bulbar, respiratory, and gross motor/limb impairment) of the MG-ADL;
Improvement from baseline in sentinel domains (ocular, facial, bulbar, gross motor, axial, and respiratory) of the QMG;
Improvement from baseline in continuous variable measures from the MGC;
Improvement from baseline in timed step count, cadence, and other exploratory measures using a wearable device;
Increased total MG-ADL score and secondary efficacy endpoints in comparison to placebo through week 48 and through week 120;
Improvement in patient-reported fatigue as measured by the Neuro-QoL-Fatigue from baseline to week 24;
Improvement from baseline in patient-reported health status as measured by the EuroQol-5 Dimensions-5 Levels (EQ-5D-5L);
Reduced number of myasthenic crises (including impending crises), days of hospitalization due to MG, and/or procedures related to MG from time periods prior to randomization;
Improvement from baseline and percent change from baseline in alternative pathway hemolytic activity assay (AH50) and total C5 over time;
Reduced use of rescue therapy from baseline to week 24;
Increase in corticosteroid dosage from baseline to week 24;
Decrease in corticosteroid dosage from baseline to week 24;
Reduced incidence and severity of adverse events through the end of study in comparison to placebo;
Improvement from baseline in Modified MGFA Post-Intervention Status (MG-PIS) at week 24, week 48, and week 120;
A percent change in serum CH50 by week 1, week 2, week 4, week 12 or week 24, after treatment initiation with Pozelimab monotherapy (200 mg, q4w) of about −18.49, −9.19, −7.06, −6 or −7.31, U/ml, respectively;
A percent change in serum CH50 by week 1, week 2, week 4, week 12 or week 24 after treatment initiation with Pozelimab (PZ) and Cemdisiran (CMD) combination (200 mg PZ+200 CMD, q4w) of about −63.31, −83.53, −84.62, −98.91 or −99.57, U/ml, respectively;
A percent change in serum CH50 by week 1, week 2, week 4, week 12 or week 24 after treatment initiation with Cemdisiran monotherapy (about 600 mg, q12w; preferably, about 600 mg Cemdisiran Na⁺ form; or the molar equivalent of Cemdisiran free form or of another salt form thereof) of about −32.62, −55.75, −76.77, −73.53 or −76.61, U/ml, respectively;
A change in MG-ADL total score by week 2, 4, 8, 12, 16, 20 or 24 after treatment initiation with Pozelimab monotherapy (200 mg, q4w) of about −1.31, −1.98, −2.59, −2.66, −2.80, −2.59 or −2.77, respectively;
A change in MG-ADL total score by week 2, 4, 8, 12, 16, 20 or 24 after treatment initiation with Pozelimab (PZ)+Cemdisiran (CMD) combination (200 mg PZ+200 mg CMD, q4w) of about −2.56, −3.08, −3.38, −3.76, −3.75, −3.91 or −3.96, respectively;
A change in MG-ADL total score by week 2, 4, 8, 12, 16, 20 or 24 after treatment initiation with Cemdisiran monotherapy (about 600 mg, q12w; preferably, about 600 mg Cemdisiran Na⁺ form; or the molar equivalent of Cemdisiran free form or of another salt form thereof) of about −2.50, −3.22, −3.96, −4.31, −4.14, −4.59 or −4.52, respectively;
An absolute MD-ADL score, by week, 2, 4, 8, 12, 16, 20 or 24 after treatment initiation with Pozelimab (200 mg, q4w), of about 8.50, 7.87, 7.08, 7.13, 6.94, 7.15 or 6.92, respectively;
An absolute MD-ADL score, by week, 2, 4, 8, 12, 16, 20 or 24 after treatment initiation with Pozelimab (PZ)+Cemdisiran (CMD) (200 mg PZ+200 mg CMD, q4w), of about 6.91, 6.25, 5.84, 5.45, 5.33, 5.22 or 5.08, respectively;
An absolute MD-ADL score, by week, 2, 4, 8, 12, 16, 20 or 24 after treatment initiation with Cemdisiran (about 600 mg, q12w; preferably, about 600 mg Cemdisiran Na⁺ form; or the molar equivalent of Cemdisiran free form or of another salt form thereof), of about 6.52, 5.81, 5.08, 4.70, 4.81, 4.44 or 4.53, respectively;
A change in QMG total score by week 2, 4, 8, 12, 16, 20 or 24 after treatment initiation with Pozelimab monotherapy (200 mg, q4w) of about −0.83, −0.73, −1.58, −1.78, −1.44, −1.77 or −1.55, respectively;
A change in QMG total score by week 2, 4, 8, 12, 16, 20 or 24 after treatment initiation with Pozelimab (PZ) and Cemdisiran (CMD) combination (200 mg PZ+200 CMD, q4w) of about −2.11, −2.77, −2.56, −3.07, −3.27, −3.18 or −3.32, respectively;
A change in QMG total score by week 2, 4, 8, 12, 16, 20 or 24 after treatment initiation with Cemdisiran monotherapy (about 600 mg, q12w; preferably, about 600 mg Cemdisiran Na⁺ form; or the molar equivalent of Cemdisiran free form or of another salt form thereof) of about −1.95, −2.60, −3.91, −3.94, −4.11, −3.92 or −4.24, respectively;
An absolute MD-ADL score, by week, 2, 4, 8, 12, 16, 20 or 24 after treatment initiation with Pozelimab monotherapy (200 mg, q4w), of about 15.33, 15.54, 14.38, 14.41, 14.47, 14.19 or 14.31, respectively;
An absolute MD-ADL score, by week, 2, 4, 8, 12, 16, 20 or 24 after treatment initiation with Pozelimab (PZ)+Cemdisiran (CMD) (200 mg PZ+200 mg CMD, q4w) (200 mg, q4w), of about 13.84, 12.85, 12.94, 12.33, 11.97, 11.98 or 11.94, respectively;
An absolute MD-ADL score, by week, 2, 4, 8, 12, 16, 20 or 24 after treatment initiation with Cemdisiran monotherapy (about 600 mg, q12w; preferably, about 600 mg Cemdisiran Na⁺ form; or the molar equivalent of Cemdisiran free form or of another salt form thereof), of about 13.64, 12.98, 11.69, 11.64, 11.45, 11.76 or 11.44, respectively;
A change in MGC score by week 2, 4, 8, 12, 16, 20 or 24 after treatment initiation with Pozelimab (PZ)+Cemdisiran (CMD) combination (200 mg PZ+200 mg CMD, q4w) of about −4.31, −4.77, −5.49, −5.89, −5.85, −6.01 or −6.04, respectively;
A change in MGC score by week 2, 4, 8, 12, 16, 20 or 24 after treatment initiation with Cemdisiran monotherapy (about 600 mg, q12w; preferably, about 600 mg Cemdisiran Na⁺ form; or the molar equivalent of Cemdisiran free form or of another salt form thereof) of about −4.49, −5.22, −6.55, −7.55, −7.57, −7.69 or −7.78, respectively;
A change in MGC score by week 2, 4, 8, 12, 16, 20 or 24 after treatment initiation with Pozelimab (PZ) monotherapy (200 mg, q4w) of about −1.64, −2.13, −3.34, −3.47, −3.30, −3.57 or −3.19, respectively;
A change in MG-QoL15r score by week 2, 4, 8, 12, 16, 20 or 24 after treatment initiation with Pozelimab (PZ)+Cemdisiran (CMD) combination (200 mg PZ+200 mg CMD, q4w) of about −2.44, −2.86, −4.00, −3.79, −3.86, −3.91 or −4.25, respectively;
A change in MG-QoL15r score by week 2, 4, 8, 12, 16, 20 or 24 after treatment initiation with Cemdisiran monotherapy (about 600 mg, q12w; preferably, about 600 mg Cemdisiran Na⁺ form; or the molar equivalent of Cemdisiran free form or of another salt form thereof) of about −1.43, −2.67, −4.08, −4.31, −4.52, −4.65 or −4.68, respectively; and/or
A change in MG-QoL15r score by week 2, 4, 8, 12, 16, 20 or 24 after treatment initiation with Pozelimab (PZ) monotherapy (200 mg, q4w) of about −1.65, −1.26, −0.92, −1.76, −1.49, −0.70 or −1.58, respectively.

Achieving a given score or score improvement (e.g., in MG-ADL score; QMG score, MGC score, MG-QOL15r score, Neuro-QoL-Fatigue score, or EQ-5D-5L score) includes embodiments wherein the subject's conditions are measured, for example, according to the relevant questionnaire or scale; and embodiments wherein, though not measured with the questionnaire or scale, the subject's condition would achieve such a score or improvement if measured with the questionnaire or scale.

The present invention includes a method for achieving or maintaining any one or more of
1. Improvement in Myasthenia Gravis-Activities of Daily Living (MG-ADL) total score e.g., from baseline to week 24;
2. Improvement in Quantitative Myasthenia Gravis (QMG) score, e.g., from baseline to week 24;
3. ≥3-point improvement, e.g., from baseline to week 24, on the MG-ADL;
4. ≥5-point improvement, e.g., from baseline to week 24, on the QMG;
5. At least a 2-point MG-ADL improvement on 2 or more consecutive assessments on the MG-ADL spanning 4 or more weeks during the double-blind treatment period (DBTP);
6. Minimal symptom expression (MSE), defined by a score of 0 to 1 on the MG-ADL, e.g., at week 24;
7. Improvement in the Myasthenia Gravis Composite (MGC) total score, e.g., from baseline to week 24;
8. Improvement in Myasthenia Gravis Quality of Life (MG-QOL15r) total score, e.g., from baseline to week 24;
9. Improvement point threshold of ≥2, 4, 5, 6, 7, 8, 9, or 10 on MG-ADL, e.g., at week 24;
10 Improvement point threshold of ≥3, 4, 6, 7, 8, 9, or 10 on QMG, e.g., at week 24;
11. Improvement from baseline in MG-ADL total score by visit;
12. Improvement from baseline in QMG total score by visit;
13. Improvement from baseline in the physical performance measures of hand grip strength, supine leg lift, supine head lift, onset of dysarthria, and all timed items of the QMG;
14. Improvement from baseline in individual domains (ocular, bulbar, respiratory, and gross motor/limb impairment) of the MG-ADL;
15 Improvement from baseline in sentinel domains (ocular, facial, bulbar, gross motor, axial, and respiratory) of the QMG;
16. Improvement from baseline in continuous variable measures from the MGC;
17. Improvement from baseline in timed step count, cadence, and other exploratory measures using a wearable device;
18. Improvement in patient-reported fatigue as measured by the Neuro-QoL-Fatigue, e.g., from baseline to week 24;
19 Improvement from baseline in patient-reported health status as measured by the EuroQol-5 Dimensions-5 Levels (EQ-5D-5L);
20. Reduced number of myasthenic crises (including impending crises), days of hospitalization due to MG, and/or procedures related to MG from time periods prior to treatment initiation;
21. Improvement from baseline and percent change from baseline in alternative pathway hemolytic activity assay (AH50) and total C5 over time;
22. Reduced use of rescue therapy, e.g., from baseline to week 24;
23 Increase in corticosteroid dosage, e.g., from baseline to week 24;
24. Decrease in corticosteroid dosage, e.g., from baseline to week 24;
25. Improvement from baseline in Modified MGFA Post-Intervention Status (MG-PIS), e.g., at week 24, week 48, and week 120;
26. A percent change in serum CH50 by week 1, week 2, week 4, week 12 or week 24, after treatment initiation of about −18.49, −9.19, −7.06, −6 or −7.31 U/ml;
27. A percent change in serum CH50 by week 1, week 2, week 4, week 12 or week 24 after treatment initiation of about −63.31, −83.53, −84.62, −98.91 or −99.57 U/ml;
28. A percent change in serum CH50 by week 1, week 2, week 4, week 12 or week 24 after treatment initiation of about −32.62, −55.75, −76.77, −73.53 or −76.61 U/ml;
29. A change in MG-ADL total score by week 2, 4, 8, 12, 16, 20 or 24 of about −1.31, −1.98, −2.59, −2.66, −2.80, −2.59 or −2.77;
30. A change in MG-ADL total score by week 2, 4, 8, 12, 16, 20 or 24 of about −2.56, −3.08, −3.38, −3.76, −3.75, −3.91 or −3.96;
31. A change in MG-ADL total score by week 2, 4, 8, 12, 16, 20 or 24 of about −2.50, −3.22, −3.96, −4.31, −4.14, −4.59 or −4.52;
32. An absolute MD-ADL score, by week, 2, 4, 8, 12, 16, 20 or 24 of about 8.50, 7.87, 7.08, 7.13, 6.94, 7.15 or 6.92;
33. An absolute MD-ADL score, by week, 2, 4, 8, 12, 16, 20 or 24 of about 6.91, 6.25, 5.84, 5.45, 5.33, 5.22 or 5.08;
34. An absolute MD-ADL score, by week, 2, 4, 8, 12, 16, 20 or 24 of about 6.52, 5.81, 5.08, 4.70, 4.81, 4.44 or 4.53;
35. A change in QMG total score by week 2, 4, 8, 12, 16, 20 or 24 of about −0.83, −0.73, −1.58, −1.78, −1.44, −1.77 or −1.55; 36. A change in QMG total score by week 2, 4, 8, 12, 16, 20 or 24 of about −2.11, −2.77, −2.56, −3.07, −3.27, −3.18 or −3.32;
37. A change in QMG total score by week 2, 4, 8, 12, 16, 20 or 24 of about −1.95, −2.60, −3.91, −3.94, −4.11, −3.92 or −4.24;
38. An absolute MD-ADL score, by week, 2, 4, 8, 12, 16, 20 or 24 of about 15.33, 15.54, 14.38, 14.41, 14.47, 14.19 or 14.31;
39. An absolute MD-ADL score, by week, 2, 4, 8, 12, 16, 20 or 24 of about 13.84, 12.85, 12.94, 12.33, 11.97, 11.98 or 11.94;
40. An absolute MD-ADL score, by week, 2, 4, 8, 12, 16, 20 or 24 of about 13.64, 12.98, 11.69, 11.64, 11.45, 11.76 or 11.44;
41. A change in MGC score by week 2, 4, 8, 12, 16, 20 or 24 of about −4.31, −4.77, −5.49, −5.89, −5.85, −6.01 or −6.04;
42. A change in MGC score by week 2, 4, 8, 12, 16, 20 or 24 of about −4.49, −5.22, −6.55, −7.55, −7.57, −7.69 or −7.78;
43. A change in MGC score by week 2, 4, 8, 12, 16, 20 or 24 of about −1.64, −2.13, −3.34, −3.47, −3.30, −3.57 or −3.19;
44. A change in MG-QoL15r score by week 2, 4, 8, 12, 16, 20 or 24 of about −2.44, −2.86, −4.00, −3.79, −3.86, −3.91 or −4.25;
45. A change in MG-QoL15r score by week 2, 4, 8, 12, 16, 20 or 24 of about −1.43, −2.67, −4.08, −4.31, −4.52, −4.65 or −4.68; and/or
46. A change in MG-QoL15r score by week 2, 4, 8, 12, 16, 20 or 24 of about −1.65, −1.26, −0.92, −1.76, −1.49, −0.70 or −1.58;
in a subject in need thereof having MG (for example, wherein the subject is anti-acetylcholine receptor (AChR) and/or anti-low-density lipoprotein receptor-related protein 4 (LRP4) antibody positive) including administering, to the subject,
   one or more 200 mg (±20 mg) doses of the anti-C5 antibody or antigen-binding fragment, e.g., about every 4 weeks (±7 days); and one or more 200 mg (±20 mg) doses of the C5 iRNA, e.g., about every 4 weeks (±7 days); preferably, wherein the antibody or fragment is Pozelimab and the C5 iRNA is Cemdisiran Na⁺ form or another salt form or the free form;
   about 600 mg (±60 mg) Cemdisiran Na⁺ form or a molar equivalent amount of another salt form or the free form every about 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year;
   about 32-25 (±4) (e.g., 32, 33, 33.8, 34 or 35) micromoles Cemdisiran, e.g., Na⁺ form or another salt form or the free form, every about 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year; or
   about 567 mg of Cemdisiran free form which is in Na⁺ salt form or another salt form every about 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year;
preferably, wherein each administration is by SC injection.

As used herein, the expression “in combination with” means that the anti-C5 antibody or antigen-binding fragment thereof is administered before, after, or concurrently with the C5 iRNA. This expression includes sequential or concurrent administration of the anti-C5 antibody or antigen-binding fragment thereof and a C5 iRNA. Thus, a method of treatment as set forth herein including administering an anti-C5 antibody or antigen-binding fragment thereof (e.g., 200 mg Pozelimab) in combination with a C5 iRNA (e.g., 200 mg Cemdisiran), to a subject in need thereof, may be referred to as a combination therapy of the present invention.

In some embodiments, when the anti-C5 antibody or antigen-binding fragment thereof is administered “before” the C5 iRNA, the anti-C5 antibody or antigen-binding fragment thereof may be administered about 4 weeks (±7 days), about 3 weeks (±7 days), about 2 weeks (±7 days), about 1 week (±7 days), about 150 hours, about 100 hours, about 72 hours, about 60 hours, about 48 hours, about 36 hours, about 24 hours prior to the administration of the C5 iRNA.

In some embodiments, when the anti-C5 antibody or antigen-binding fragment thereof is administered “after” the C5 iRNA, the anti-C5 antibody or antigen-binding fragment thereof may be administered about 24 hours, about 36 hours, about 48 hours, about 60 hours, about 72 hours, about 1 week (±7 days), about 2 weeks (±7 days), about 3 weeks (±7 days), about 4 weeks (±7 days) after the administration of the C5 iRNA.

As used herein, “concurrent” administration means that the anti-C5 antibody or antigen-binding fragment thereof (e.g., Pozelimab) and a C5 iRNA (e.g., Cemdisiran) are administered to the subject in a single dosage form (e.g., co-formulated) or in separate dosage forms administered to the subject during the same treatment episode, preferably within about 1 or 2 hours or 30 minutes or less of each other (i.e., before, after, or at the same time), such as about 15 minutes or less, or about 5 minutes or less. If administered in separate dosage forms, each dosage form may be administered via the same route (e.g., both administered intravenously, subcutaneously, etc.); or, alternatively, each dosage form may be administered via a different route. In any event, administering the components in a single dosage from, in separate dosage forms by the same route, or in separate dosage forms by different routes are all considered “concurrent” administration” for purposes of the present disclosure. In an embodiment of the disclosure, concurrent subcutaneous doses of anti-C5 antibody or antigen-binding fragment thereof and C5 iRNA are administered by injection into separate body areas, e.g., separate arms.

The co-formulations and/or combination therapy of the present disclosure (e.g., Cemdisiran/Pozelimab) are useful for treating or preventing a disease or disorder or condition associated with C5 that includes the step of administering a therapeutically effective amount of anti-C5 antibody or antigen-binding fragment thereof and a C5 iRNA, preferably in a co-formulation, e.g., by parenteral route, e.g., intramuscular (IM), subcutaneous (SC), or intravenous (IV) injection/infusion. Preferably, about 200 mg (±20 mg) of the antibody, preferably Pozelimab, is administered about every 4 weeks (±7 days) and about 200 mg (±20 mg) iRNA, preferably Cemdisiran Na⁺ form or a molar equivalent amount of another salt form or the free form, is administered about every 4 weeks (±7 days). Preferably, the antibody or fragment and the iRNA is administered by subcutaneous injection (SC).

In an embodiment of the invention, any dose mentioned herein (e.g., of Pozelimab or Cemdisiran) is administered ±1, ±2, ±3, ±4, ±5, ±6 or ±7 days, for example, q4w (±7 days).

In an embodiment of the invention, a subject receiving a dosing regimen as set forth herein, may receive one or more doses of anti-C5 antibody or antigen-binding fragment and/or C5 iRNA that are in addition to the regular doses required by the regimen.

In an embodiment of the invention, a subject receiving a dosing regimen as set forth herein, may skip one or more of the regular doses of anti-C5 antibody or antigen-binding fragment and/or C5 iRNA required by the regimen.

In an embodiment of the invention, the subject receives a dosing regimen of the present invention for at least one dose, at least 2 doses, 1 year, 2 years, 5 years or 10 years; or more.

The present invention includes methods for treating or preventing atypical hemolytic uremic syndrome (aHUS) in a subject in need thereof comprising administering, to the subject
(a) one or more about 200 mg (±20 mg) doses of the anti-C5 antibody or antigen-binding fragment (e.g., Pozelimab) (e.g., about every 4 weeks (±7 days)); and one or more about 200 mg (±20 mg) doses of the C5 iRNA (e.g., Cemdisiran Na⁺ form or a molar equivalent amount of another salt form or the free form) (e.g., about every 4 weeks (±7 days));
or
(b) about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; or about 567 mg (+57 mg) of Cemdisiran free form which is in Nat salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year.
   In an embodiment of the invention, the antibody or fragment and the iRNA are administered in a single co-formulation; in another embodiment, the antibody and iRNA are in separate formulations. Signs and symptoms of aHUS include, but are not limited to, platelet activation, hemolysis, systemic thrombotic microangiopathy (formation of blood clots in small blood vessels throughout the body) leading to stroke, heart attack, kidney failure and/or death, end-stage renal disease, permanent renal damage, abdominal pain, confusion, edema, fatigue, nausea/vomiting, diarrhea, and microangiopathic anemia. Thus, the present disclosure includes methods for treating or preventing aHUS, in a subject in need thereof, including the steps of administering a therapeutically effective amount of co-formulation of the present disclosure to the subject (e.g., by SC, IM or IV injection).

The present invention includes methods for treating or preventing paroxysmal nocturnal hemoglobinuria (PNH) in a subject in need thereof comprising administering, to the subject
(a) one or more about 200 mg (±20 mg) doses of the anti-C5 antibody or antigen-binding fragment (e.g., Pozelimab) (e.g., about every 4 weeks (±7 days)); and one or more about 200 mg (±20 mg) doses of the C5 iRNA (e.g., Cemdisiran Na⁺ form or a molar equivalent amount of another salt form or the free form) (e.g., about every 4 weeks (±7 days)); or
(b) about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; or about 567 mg (+57 mg) of Cemdisiran free form which is in Na⁺ salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year.
   In an embodiment of the invention, the antibody or fragment and the iRNA are administered in a single co-formulation; in another embodiment, the antibody and iRNA are in separate formulations. Signs and symptoms of PNH include, but are not limited to, destruction of red blood cells, thrombosis (including deep vein thrombosis, pulmonary embolism), intravascular hemolytic anemia, red discoloration of urine, symptoms of anemia such as tiredness, shortness of breath, and palpitations, abdominal pain and difficulty swallowing. Thus, the present disclosure includes methods for treating or preventing PNH, in a subject in need thereof, including the steps of administering a therapeutically effective amount of co-formulation of the present disclosure to the subject (e.g., by SC, IM or IV injection).

The present invention includes methods for treating or preventing CHAPLE disease (CD55 deficiency with hyperactivation of complement, angiopathic thrombosis and protein-losing enteropathy) in a subject in need thereof comprising administering, to the subject
(a) one or more about 200 mg (±20 mg) doses of the anti-C5 antibody or antigen-binding fragment (e.g., Pozelimab) (e.g., about every 4 weeks (±7 days)); and one or more about 200 mg (±20 mg) doses of the C5 iRNA (e.g., Cemdisiran Na⁺ form or a molar equivalent amount of another salt form or the free form) (e.g., about every 4 weeks (±7 days));
or
(b) about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; or about 567 mg (+57 mg) of Cemdisiran free form which is in Nat salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year.
   In an embodiment of the invention, the antibody or fragment and the iRNA are administered in a single co-formulation; in another embodiment, the antibody and iRNA are in separate formulations. CHAPLE disease is characterized by symptoms such as inflammatory bowel disease, protein losing enteropathy (which can be associated with hypoalbuminemia), hypogammaglobulinemia, intestinal lymphangiectasia, and/or thrombotic events. Thus, the present disclosure includes methods for treating or preventing CHAPLE, in a subject in need thereof, including the steps of administering a therapeutically effective amount of co-formulation of the present disclosure to the subject (e.g., by SC, IM or IV injection).

The present invention includes methods for treating or preventing (including reducing or eliminating signs or symptoms thereof, or reducing complement activation associated therewith) a disease or disorder or condition associated with C5 (e.g., MG) in a subject in need thereof comprising administering, to the subject,
(a) one or more about 200 mg (±20 mg) doses of the anti-C5 antibody or antigen-binding fragment (e.g., Pozelimab) (e.g., about every 4 weeks (±7 days)); and one or more about 200 mg (±20 mg) doses of the C5 iRNA (e.g., Cemdisiran Na⁺ form or a molar equivalent amount of another salt form or the free form) (e.g., about every 4 weeks (±7 days));
or
(b) about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; or about 567 mg (±57 mg) of Cemdisiran free form which is in Na⁺ salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year.
   In an embodiment of the invention, the antibody or fragment and the iRNA are administered in a single co-formulation; in another embodiment, the antibody and iRNA are in separate formulations. Thus, the present disclosure includes methods for treating or preventing any of such disorders, in a subject in need thereof, including the steps of administering a therapeutically effective amount of co-formulation of the present disclosure to the subject (e.g., by SC, IM or IV injection).

The diseases or disorders or conditions associated with C5 which may be treated or for which signs and symptoms may be reduced or eliminated or for which complement activation may be reduced by administering
(a) one or more about 200 mg (±20 mg) doses of the anti-C5 antibody or antigen-binding fragment (e.g., Pozelimab) (e.g., about every 4 weeks (±7 days)); and one or more about 200 mg (±20 mg) doses of the C5 iRNA (e.g., Cemdisiran Na⁺ form or a molar equivalent amount of another salt form or the free form) (e.g., about every 4 weeks (±7 days));
or
(b) about 600 mg (±60 mg) Cemdisiran Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; about 32-25 (±4) (e.g., 32, 33, 33.8, 34 or 35) micromoles Cemdisiran, e.g., Na⁺ form or a molar equivalent amount of another salt form or the free form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; or about 567 mg (±57 mg) of Cemdisiran free form which is in Na⁺ salt form or another salt form about every 84 days (±1, 2, 3, 4, 5, 6 or 7 days), or about every 12 weeks (±1, 2, 3, 4, 5, 6 or 7 days), or about every 3 months (±1, 2, 3, 4, 5, 6 or 7 days) or about every ¼ year; include, for example, adult respiratory distress syndrome; Alport's syndrome; Alzheimer's disease; atherosclerosis; bullous pemphigoid; C3 glomerulopathy; capillary leak syndrome; CHAPLE disease/syndrome (CD55 deficiency with hyperactivation of complement, angiopathic thrombosis and PLE); complement activation due to a burn; complement activation due to obesity; complement activation due to frostbite; complement activation due to sepsis; Crohn's disease; diabetes; diabetic nephropathy; epilepsy; glomerulopathy; Guillain-Barre Syndrome; hemolytic anemia; hyperacute allograft rejection; an infectious disease; inherited CD59 deficiency; interleukin-2 induced toxicity during IL-2 therapy; lupus nephritis; membranoproliferative glomerulonephritis; membranoproliferative nephritis; mesenteric artery reperfusion after aortic reconstruction; multiple sclerosis; myasthenia gravis; myocardial infarction; neuromyelitis optica; Parkinson's disease; progressive kidney failure; psoriasis; a post-ischemic reperfusion condition; a renal ischemia; a renal ischemia-reperfusion injury; rheumatoid arthritis; schizophrenia; SLE nephritis; stroke; systemic lupus erythematosus (SLE); traumatic brain injury; vasculitis; xenograft rejection, a lung disease or disorder such as dyspnea, hemoptysis, ARDS, asthma, chronic obstructive pulmonary disease (COPD), emphysema, pulmonary embolisms and infarcts, pneumonia, a fibrogenic dust disease, injury due to inert dusts and minerals (e.g., silicon, coal dust, beryllium, and asbestos), pulmonary fibrosis, an organic dust disease, a chemical injury (due to irritant gasses and chemicals, e.g., chlorine, phosgene, sulfur dioxide, hydrogen sulfide, nitrogen dioxide, ammonia, and hydrochloric acid), a smoke injury, a thermal injury (e.g., burn or freeze), allergy, bronchoconstriction, hypersensitivity pneumonitis, a parasitic disease, Goodpasture's Syndrome, pulmonary vasculitis, hereditary angioedema, immune complex-associated inflammation, an ocular disease such as age-related macular degeneration (AMD), diabetic macular edema (DME), diabetic retinopathy, ocular angiogenesis (ocular neovascularization affecting choroidal, corneal, or retinal tissue), geographic atrophy (GA), uveitis and/or neuromyelitis optica.

In an embodiment of the invention, the subject was receiving crovalimab, ronzanolixizumab, efgartigimod, belimumab, avacincaptad pegol and/or zilucoplan for treatment of MG prior to initiation of treatment with a method according to the present invention.

In an embodiment of the invention, the subject was receiving Eculizumab for treatment of MG, e.g.,
(a) 900 mg Eculizumab (e.g., intravenously (IV)) weekly for the first 4 weeks, followed by
(b) 1200 mg Eculizumab (e.g., intravenously (IV)) for the fifth dose 1 week later, then
(c) 1200 mg Eculizumab (e.g., intravenously (IV)) every 2 weeks thereafter (q2w);
and then switches to a treatment regimen according to the present invention.

In an embodiment of the invention, the subject was receiving Ravulizumab for treatment of MG, e.g.,
(a) For 40 to less than 60 kg bodyweight (BW), 2,400 mg loading dose and 3,000 mg doses every 8 weeks (e.g., intravenously (IV) or subcutaneously (SC));
(b) For 60 to less than 100 mg BW, 2,700 mg loading dose and 3,300 mg doses every 8 weeks (e.g., intravenously (IV) or subcutaneously (SC)); or
(c) For 100 mg or greater BW, 3,000 mg loading dose and 3,600 every 8 weeks (e.g., intravenously (IV) or subcutaneously (SC)).

“Treat” or “treating” refers to reducing or eliminating one or more signs or symptoms thereof in a subject e.g., reducing complement activation associated therewith.

A therapeutically effective dose or amount of C5 iRNA is about 567 mg (±57 mg) of C5 iRNA, 32-35 or 33.8 (±4) micromoles of C5 iRNA or 600 mg (±60 mg) of C5 iRNA which is in salt for such as Nat form (e.g., wherein the C5 iRNA is Cemdisiran Na⁺ form of another salt form or the free form, preferably subcutaneously, about every 84 days (±1, 2, 3, 4, 5, 6 or 7 days), about 12 weeks (±1, 2, 3, 4, 5, 6 or 7 days), about 3 months (±1, 2, 3, 4, 5, 6 or 7 days) or about ¼ year; or of anti-C5 antibody or antigen-binding fragment thereof and C5 iRNA is about 200 mg (±20 mg) of the anti-C5 antibody or antigen-binding fragment thereof (e.g., Pozelimab) and about 189 mg (±19 mg) of C5 iRNA, 11-13 (±2) micromoles of C5 iRNA or 200 mg (±20 mg) of C5 iRNA which is in salt for such as Na⁺ form (e.g., wherein the C5 iRNA is Cemdisiran Nat form of another salt form or the free form), which may be administered in a single co-formulation or by two separate administrations of the antibody or fragment and the C5 iRNA, about once every 28, 29 or 30 days (±1, 2, 3, 4, 5, 6 or 7 days), once a month (±1, 2, 3, 4, 5, 6 or 7 days) or once every 4 weeks (±1, 2, 3, 4, 5, 6 or 7 days), preferably subcutaneously.

A subject or patient—used interchangeably herein-refers to a mammal, preferably, a human. In an embodiment of the disclosure, the subject suffers from a disease or disorder or condition associated with C5, such as MG. In an embodiment of the present disclosure, a subject has a confirmed diagnosis (e.g., seropositive for anti-AChR or anti-LRP4 antibodies) of MG and/or has signs and/or symptoms of MG (as defined by MG-ADL score ≥6 and MGFA Class II to IVa at screening). In an embodiment of the present disclosure, the subject or patient does not have any one or more of the following characteristics:
An antibody profile that is only positive for MuSK (MuSK positivity is based on a documented prior history of positive serologic test for antibodies to MuSK or a positive result during screening);
History of thymectomy within 12 months prior to screening or planned during the study;
History of malignant thymoma, or history of cancer within the past 5 years, except for adequately treated basal cell skin cancer, squamous cell skin cancer, or in situ cervical cancer;
Myasthenic crisis or MGFA Class V within past 1 month;
Any systemic bacterial or other infection that was clinically significant in the opinion of a physician and had not been treated with appropriate therapy within the past 2 weeks;
Unresolved meningococcal infection;
Meningococcal vaccination within past 5 years;
Known contraindication to meningococcal vaccines (group ACWY conjugate and group B vaccines);
Requirement for antibiotics for meningococcal prophylaxis and have a contraindication, warning, or precaution precluding the use of penicillin class and penicillin-alternative antibiotics planned to be used for prophylaxis, or a history of intolerance leading to the discontinuation of these antibiotics;
Positive hepatitis B surface antigen or hepatitis C virus RNA during screening;
History of HIV infection or a positive test at screening per local requirements;
An estimated glomerular filtration rate (eGFR)<30 mL/min/1.73 m² (according to the Chronic Kidney Disease Epidemiology Collaboration creatinine equation, with one re-test allowed;
Documented history of liver cirrhosis or known liver disease with current evidence of ongoing impaired liver function (e.g., screening international normalized ratio [INR]>1.5×ULN that is considered hepatic in origin);
Screening serum ALT levels >3×ULN and/or total bilirubin >2×ULN (unless bilirubin elevation is due to suspected Gilbert's syndrome), with one re-test allowed;
Receiving azathioprine;
Receiving other intratympanic steroid therapy (ISTs) (e.g., systemic corticosteroids, mycophenolate mofetil, methotrexate, cyclosporine, tacrolimus, or cyclophosphamide);
Receiving cholinesterase inhibitors;
Undergoing ongoing treatment with IVIG or PLEX/other comparable therapeutic apheresis (TA) procedure or treatment within past 4 weeks;
Use of rituximab within past 6 months;
Use of complement inhibitor therapy within past 6 months or neonatal Fc receptor (FcRn) inhibitors within past 4 months or 5 half-lives, whichever is longer;
Muscular weakness predominantly due to conditions other than MG, such as myopathies, concurrent infections, medications, or hospitalization/bedrest immobilization;
Hypersensitivity to Pozelimab or Cemdisiran or to one of its excipients;
Functional or anatomic asplenia; and/or
Any life-time suicidal actions.
   In an embodiment of the present disclosure, the subject or patient has any one or more of the following characteristics:
Male or female patients ≥18 years of age;
Diagnosis of MG based on medical history and supported by previous evaluations. Examples of supportive evaluations include any one of the following:
   History of abnormal neuromuscular transmission by repetitive nerve stimulation/single-fiber electromyography
   History of positive anticholinesterase test
   Positive response (improvement in signs) with anticholinesterase treatment, as assessed by the treating physician;
History of positive serologic test or a positive result of anti-AChR antibodies or anti-LRP4 antibodies;
MGFA Clinical Classification Class II to IVa;
MG-ADL score ≥6;
Currently receiving an acetylcholinesterase inhibitor; and/or
Currently receiving an IST for MG (ISTs include, but are not limited to, systemic corticosteroids, azathioprine, mycophenolate mofetil, methotrexate, cyclosporine, tacrolimus, or cyclophosphamide).

In an embodiment of the invention, the subject is monitored for: Meningococcal infection; Serious infection; Sepsis; Embryofetal toxicity; Immunogenicity; Anti-drug antibody formation; Any clinical Consequences of large Drug-Target-Drug immune complexes in subjects who switch from Eculizumab or Ravulizumab to Pozelimab; Liver transaminase elevations; and/or Injection site reaction. In an embodiment of the invention, treatment is terminated or suspended of any one of the foregoing is observed.

### Myasthenia Gravis (MG)

In some embodiments, the disclosed co-formulations and/or combination therapy of the present disclosure (e.g., Cemdisiran/Pozelimab) are useful in treating or preventing myasthenia gravis (MG) of any type or class (e.g., generalized and/or ocular). Two types of myasthenia gravis are generalized myasthenia gravis and ocular myasthenia gravis. Generalized MG causes weakness in many muscle groups. Ocular myasthenia gravis affects the muscles that move the eyes and eyelids.

Myasthenia gravis is an autoimmune disorder affecting the neuromuscular junction. It manifests as a generalized muscle weakness which can involve the respiratory muscles and can lead to a myasthenic crisis, which is a medical emergency. A classic presentation of MG is fluctuating weakness that is more prominent in the afternoon. It usually involves muscles of the eyes, throat, and extremities. The reduced transmission of electrical impulses across the neuromuscular junction due to the formation of autoantibodies against the specific postsynaptic membrane proteins consequently causes muscle weakness. A wide variety of conditions can precipitate MG, such as infections, immunization, surgeries, and drugs. Myasthenia gravis causes a significant number of complications. These include myasthenic crisis, an acute respiratory paralysis that requires intensive care, as well as adverse events due to long term medication treatment like opportunistic infections and lymphoproliferative malignancies. In an embodiment of the invention, a subject has MG and one or more of such characteristics, e.g., generalized muscle weakness.

The presence of autoantibodies directed against the muscle nicotinic acetylcholine receptor (AChR) is a common cause of myasthenia gravis (MG). These antibodies damage the postsynaptic membrane of the neuromuscular junction and cause muscle weakness by depleting AChRs and thus impairing synaptic transmission.

Antibodies (Abs) specific for the low-density lipoprotein receptor-related protein 4 (LRP4) occur in some patients with myasthenia gravis (MG). LRP4 forms a multiprotein complex with MuSK and facilitates AChR clustering through the agrin/LRP4/MuSK/Dok7/rapsyn pathway.

Commonly implicated proteins in the NMJ against which autoantibodies are produced include
the nicotinic acetylcholine receptors (n-AChRs),
muscle-specific kinase (MuSK), and
lipoprotein-related protein 4 (LPR4).

The Agrin-LRP4-MuSK protein complex is essential for the formation and maintenance of NMJ, including the distribution and clustering of the AChR.

Classes of MG include, depending on the type of clinical features and the type of antibodies involved, include
(i) Early-onset MG characterized by an age at onset of less than 50 years with thymic hyperplasia;
(ii) Late-onset MG characterized by an age at onset of greater than 50 years with thymic atrophy;
(iii) Thymoma-associated MG;
(iv) MG with anti-MuSK antibodies;
(v) Ocular MG characterized by symptoms from periocular muscles; and
(vi) MG with no detectable AChR and MuSK antibodies.
Gilhus et al., Myasthenia gravis: a review of available treatment approaches. Autoimmune Dis. 2011; 2011:847393.

Clinical classification divides MG into 5 main classes based on the clinical features and the disease severity. Each class carries different prognoses or responses to therapy:
Class I: Involves any ocular muscle weakness, including weakness of eye closure. All other muscle groups are normal;
Class II: Involves mild weakness of muscles other than ocular muscles. Ocular muscle weakness of any severity may be present;
Class IIa: Involves predominant weakness of the limb, axial muscles, or both. It may also involve the oropharyngeal muscles to a lesser extent;
Class IIb: Involves mostly oropharyngeal, respiratory muscles, or both. It can have the involvement of limb, axial muscles, or both to a lesser extent;
Class III: Involves muscles other than ocular muscles moderately. Ocular muscle weakness of any severity can be present;
Class IIIa: involves the limb, axial muscles, or both predominantly. Oropharyngeal muscles can be involved to a lesser degree;
Class IIIb: Involves oropharyngeal, respiratory muscles, or both predominantly. The limb, axial muscles, or both can have lesser or equal involvement;
Class IV: Involves severe weakness of affected muscles. Ocular muscle weakness of any severity can be present;
Class IVa: Involves limb, axial muscles, or both predominantly. Oropharyngeal muscles can be involved to a lesser degree;
Class IVb: Involves oropharyngeal, respiratory muscles, or both predominantly. The limb, axial muscles, or both can have lesser or equal involvement. It also includes patients requiring feeding tubes without intubation; and
Class V: Involves intubation with or without mechanical ventilation, except when employed during routine postoperative management.
   (Beloor Suresh A, Asuncion R M D. Myasthenia Gravis. [Updated 2023 Aug. 8]. In: StatPearls [Internet]. Treasure Island (FL): StatPearls Publishing; 2025 January. Available from: https://www.ncbi.nlm.nih.gov/books/NBK559331/)

Diagnosis of MG can be done on the basis of laboratory investigations and procedures. Serologic Tests for anti-AChR Abs is very specific, and confirms the diagnosis in patients with classical clinical findings. It is present in four-fifths of patients with generalized MG and only in half of the patients with pure ocular MG (Bindu et al., Ann Indian Acad Neurol. 2008 October; 11 (4): 242-4). Other patients will demonstrate anti-MuSK antibodies, both anti-AChR and anti-MuSK; anti-LRP4 antibodies; or anti-striated muscle antibodies. The present invention includes treating MG in patients having any one or more of these antibodies.

Electrophysiologic tests (e.g., RNS or SFEMG) can be performed on patients who are seronegative for antibody testing. Tests for MG are the repetitive nerve stimulation (RNS) test and single-fiber electromyography (SFEMG). Both the tests assess for conduction delays in the NMJ. Routine nerve conduction studies are usually performed to determine the functioning of the nerves and muscles before undertaking these tests. The repeated nerve stimulation (RNS) test is done by stimulating the nerve at 2-3 Hz. RNS depletes the ACh in the NMJ, and produces a low excitatory postsynaptic potential (EPSP). A 10% or more decrease in the EPSP between the first and fifth stimulus is diagnostic of MG. Single-fiber electromyography (SFEMG) records action potential (AP) from individual muscle fibers, and thus allows to record the AP simultaneously from two muscle fibers innervated by a single motor neuron. The difference between the time of onset of these two action potentials is called the “jitter.” In MG, the “jitter” will increase because of the reduced NMJ transmission.

The Edrophonium (Tensilon) test is a short-acting acetylcholinesterase inhibitor that increases the availability of ACh in the NMJ. This is particularly useful for ocular MG, where electrophysiologic testing cannot be performed. It is administered intravenously, and the patient is observed for improvement in the symptoms of ptosis or diplopia.

The Ice-pack test, may be used when edrophonium testing is contraindicated. This test requires an ice-pack placed over the eye for 2-5 minutes. Then, an assessment for any improvement in ptosis is done. This test cannot be used for the evaluation of extraocular muscles.

Imaging such as Chest computed tomography (CT) or Magnetic resonance imaging (MRI) should be performed in patients diagnosed with MG to assess for thymoma. In cases presenting with pure ocular MG, MRI of orbits and the brain can be performed to evaluate for any localized mass lesions.

Myasthenia gravis commonly coexists with other autoimmune disorders, and testing for anti-nuclear (ANA) antibodies, rheumatoid factor (RF), and baseline thyroid functions can be indicative of MG.

The present invention includes methods of treatment that include a step of diagnosing MG in the subject before or during such treatment, e.g., wherein diagnosis is performed using RNS, SFEMG, Tensilon, Ice-pack, imaging tests. For example, the present invention includes a method for treating or preventing a MG in a subject in need thereof comprising administering, to the subject one or more 200 mg (±20 mg) doses of anti-C5 antibody or antigen-binding fragment; and one or more 200 mg (±20 mg) doses of C5 iRNA; wherein prior to or during said treatment, MG was diagnosed in the subject by repeated nerve stimulation (RNS) test comprising the step of stimulating a nerve at 2-3 Hz, thereby producing a low excitatory postsynaptic potential (EPSP), wherein a 10% or more decrease in the EPSP between the first and fifth stimulus is diagnostic of MG and wherein, if the subject was diagnosed as having MG, then initiating or continuing said treatment.

### Devices

The present disclosure also provides an injection device including the co-formulations of the present disclosure (e.g., Cemdisiran/Pozelimab) or C5 iRNA monotherapy formulations of the present invention, e.g., including Cemdisiran (e.g., but not including Pozelimab). An injection device is a device that introduces a substance into the body of a patient via a parenteral route, e.g., intramuscular, subcutaneous, or intravenous. For example, an injection device may be a syringe (e.g., pre-filled or auto-injector) which, for example, includes a cylinder or barrel for holding fluid to be injected (e.g., the formulation or co-formulation), a needle for piecing skin and/or blood vessels for injection of the fluid; and a plunger for pushing the fluid out of the cylinder and through the needle bore. In an embodiment of the disclosure, an injection device that comprises a formulation or co-formulation is suitable for subcutaneous, or intravenous (IV) injection. Such a device includes a formulation or co-formulation in a cannula or trocar/needle which may be attached to a tube which may be attached to a bag or reservoir for holding fluid (e.g., saline; or lactated ringer solution including NaCl, sodium lactate, KCl, CaCl₂) and optionally including glucose) introduced into the body of the patient through the cannula or trocar/needle.

A prefilled syringe is a single-use device that is provided containing a quantity of a drug product, e.g., Cemdisiran, where the user is not required to fill the device with the drug product.

The formulation or co-formulation can, in an embodiment of the disclosure, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line).

In an embodiment of the disclosure, an injection device is an autoinjector; a jet injector, an on-body subcutaneous injection device or an external infusion pump.

Autoinjectors are medical devices convenient for the self-administration of injections by patients and for easy administration by healthcare professionals in emergency situations. Although they vary in design and application, autoinjectors are typically built around a spring-loaded pre-filled syringe containing a pre-determined amount of pharmaceutical formulation. Autoinjectors are usually single-use and disposable.

A jet injector uses a high-pressure narrow jet of liquid which penetrate the epidermis to introduce a co-formulation to a patient's body. External infusion pumps are medical devices that deliver the co-formulation into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention includes embodiments herein wherein any quantities (e.g., mg) or concentrations (e.g., mg/ml or mM) that are specifically mentioned include such quantities or concentrations ±about 10%. Intervals between doses and other time periods specifically mentioned herein also includes such intervals ±1, 2, 3, 4, 5, 6 or 7 days.

### EXAMPLES

These examples are intended to exemplify the present disclosure are not a limitation thereof. Compositions and methods, including dosing regimens, set forth in the Examples form part of the present disclosure. For example, the present invention includes any of the dosing methods set forth in Example 1.

### Example 1: Study of Pozelimab and Cemdisiran Combination Therapy in Patients with Symptomatic Generalized Myasthenia Gravis (gMG) (NIMBLE; R3918-MG-2018)

This example describes a multinational, randomized, double-blind, placebo-controlled study in patients with symptomatic generalized myasthenia gravis (gMG) who have anti-acetylcholine receptor (anti-AChR) antibodies or anti-lipoprotein receptor-related protein 4 (anti-LRP4) antibodies. The study includes a double-blind, placebo-controlled treatment period (DBTP), a double-blind extension treatment period (ETP), a long-term open-label treatment period (OLTP), and a follow-up period (FUP). Patients will continue on their regional standard of care treatment for gMG during screening and during the study.

During the main placebo-controlled DBTP, the study evaluates Pozelimab (200 mg Q4W)+Cemdisiran (200 mg Q4W) combination (combo or combination arm) versus placebo on top of patients' existing stable background treatment regimens, or without such treatments based on documented reason by the investigator. In addition, a Cemdisiran monotherapy arm (600 mg (±60 mg) Q12W) (cemdi or cemdisiran arm) and a Pozelimab monotherapy arm (200 mg Q4W) are included in order to evaluate the contribution of the components in the combination therapy and the contribution of each therapy for optimal clinical response.

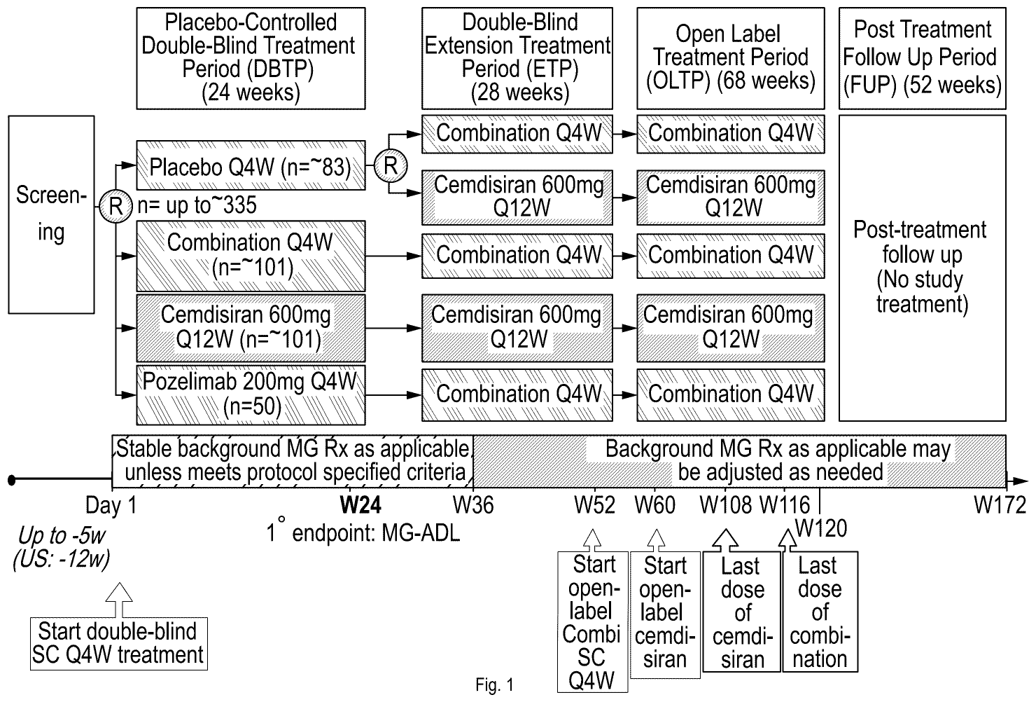

The study consists of the following periods (FIG. 1): up to 5-week screening period; a 24-week DBTP; a 28-week double-blind ETP at week 24; a 68-week OLTP at week 52 to week 120; and a FUP.

Pozelimab drug product or placebo is provided as a liquid formulation for SC administration. Placebo for Pozelimab is provided in an aqueous buffered solution. Cemdisiran drug product or placebo will be provided as a liquid formulation for SC administration. Placebo for Cemdisiran is 0.9% saline.

### INTRODUCTION

Cemdisiran is a synthetic small interfering ribonucleic acid (siRNA) targeting C5 messenger RNA (mRNA) that is covalently linked to a triantennary N-acetylgalactosamine (GalNAc) ligand. Cemdisiran is designed to suppress liver production of C5 protein, when administered via subcutaneous (SC) injection. C5 is encoded by a single gene and is expressed and secreted predominantly by hepatocytes. Through the RNA interference pathway, Cemdisiran leads to the degradation of C5 mRNA by RNases, thereby reducing C5 protein production, leading to reduced levels of circulating C5 protein.

Although Pozelimab alone at 800 mg SC weekly has been shown to reduce complement activity to undetectable levels, the dosing regimen for Pozelimab at 800 mg requires weekly 4 mL SC injections. The high concentration and frequency of dosing is not ideal for a chronic condition that is expected to require long-term treatment. While Cemdisiran does not directly block C5 but rather inhibits the expression and secretion of C5 in the liver, thereby substantially lowering levels of C5 in circulation of up to approximately 99%, Cemdisiran as a single agent remains insufficient in fully inhibiting the complement pathway. However, by taking advantage of the complementary mechanism of action of these two agents, the combination of the greatly reduced production of C5 from Cemdisiran with the high binding affinity of Pozelimab can provide the needed level of C5 blockade to manage the symptoms of gMG over a notably longer period of time.

Modeling of PK/PD based on observed data from both Pozelimab and Cemdisiran first-in-human (FIH) studies in healthy subject and patients with PNH suggest that, by combining Cemdisiran and Pozelimab, the dose of Pozelimab may be reduced and the interval for maintenance dosing increased from once weekly, when used at the more intensive regimen as a single agent, to monthly, when used in combination with Cemdisiran. As disclosed herein, the combination of Pozelimab and Cemdisiran is expected to result in the need for less Pozelimab, leading to a reduced SC injection volume, reduced dosing frequency, and a window of drug administration that is wider. In patients requiring chronic and long-term administration as in gMG, this should lead to improved compliance and quality of life compared to Pozelimab 800 mg SC weekly, while still providing for maximal inhibition of C5 activity in a greater percentage of patients than eculizumab therapy. The combination treatment is projected to achieve the pharmacologic objective of complete and sustained inhibition of C5 throughout the dosing interval.

As disclosed herein, it is expected that complete inhibition of the terminal complement pathway may be necessary to achieve optimal clinical response in patients with AChR antibody positive or LRP-4 positive gMG. Dose-dependent improvement in efficacy as well as the degree of inhibition of complement suggest that maximal complement inhibition may be necessary to provide rapid and pronounced disease suppression (Howard et al., JAMA Neurol, 77 (5): 582-592 (2020)). Because some patients with gMG who received eculizumab had incomplete inhibition of the complement pathway, there continues to be an unmet need to achieve complete inhibition of complement in nearly all patients. Additionally, SC delivery of treatment targeting C5 is advantageous to chronic IV treatment, given logistical issues with scheduled infusions (especially during a pandemic).

The concept that complete inhibition of the complement pathway is necessary in order to achieve optimal clinical response in patients with gMG is supported by preliminary clinical data (Howard, 2020); however, this concept is not yet fully validated. The present study will augment understanding of the relationship between the degree of C5 inhibition and clinical response by including a Cemdisiran monotherapy arm expected to have partial inhibition of complement. Patients may derive benefit with sub-maximal inhibition of complement as would be expected with Cemdisiran monotherapy. Zilucoplan, a peptide inhibitor of C5, evaluated 2 doses in a phase 2 study with the high dose having greater inhibition of complement relative to the low dose (Howard, 2020). Both doses did not achieve complete inhibition of complement yet showed improvements in clinical efficacy parameters, such as MG-ADL and QMG, over placebo with an overall trend favoring the high dose over the low dose.

### Treatments

During the DBTP and ETP, patients will receive double-dummy, double-blind study treatment.

### Study Drug: Pozelimab

### Dose/Route/Schedule:

200 mg SC Q4W for combination regimen.
200 mg SC Q4W for the Pozelimab monotherapy regimen until the end of the DBTP

### Placebo: Placebo for Pozelimab

### Route/Schedule:

SC Q4W for placebo regimen until the end of the DBTP.
SC Q4W for Cemdisiran monotherapy regimen until the end of the ETP

### Study Drug: Cemdisiran

### Dose/Route/Schedule:

200 mg SC Q4W in combination regimen
600 mg SC Q12W in Cemdisiran monotherapy regimen

### Placebo: Placebo for Cemdisiran

### Route/Schedule:

SC Q4W for placebo regimen and Pozelimab monotherapy regimen until the end-of-DBTP.
SC Q12W for combination regimen until the end-of-ETP.
SC Q4W for the Cemdisiran monotherapy regimen except on days where active Cemdisiran dosing occurs until the end-of-ETP

### Screening Period

Patients are randomized as soon as eligibility requirements are met (approximately 4 weeks), but the screening period may be as long as 5 weeks for patients who may need additional time (e.g., pending a laboratory result for determining eligibility). Eligible patients require evidence of immunization against N. meningitidis within the past 5 years, unless a shorter duration is recommended per current regional vaccination guidelines for vaccination use with complement inhibitors or local practice. For patients requiring vaccination during the study, administration should occur preferably at least 2 weeks prior to initiation of study treatment. If vaccination occurs less than 2 weeks from initiation of study treatment, then the patient must receive antibiotic prophylaxis for at least 2 weeks from the date of vaccination. During screening, patients who have not been vaccinated against Streptococcus pneumoniae and Haemophilus influenza type B may receive these vaccinations during the screening period.

### Placebo-Controlled Double-Blind Treatment Period (Day 1 to Week 24)

Before every visit in the DBTP, ETP and OLTP, patients are reminded to withhold cholinesterase inhibitors for at least 10 hours prior to their visit, to be resumed after visit assessments are complete.

On day 1, after all baseline assessments have been obtained, eligible patients are randomized in a 1:1:1:1 ratio to receive one of the following double-blind, double-dummy study treatments:
Placebo regimen: Pozelimab placebo SC+Cemdisiran placebo SC Q4W starting from day 1 until week 20, inclusive
Combination regimen: Pozelimab 200 mg SC+Cemdisiran 200 mg SC Q4W starting from day 1 until week 20, inclusive
Cemdisiran monotherapy regimen: Pozelimab placebo SC+Cemdisiran 600 mg (±60 mg) SC on day 1 and week 12 (to maintain the blind, patients in the Cemdisiran arm will receive Pozelimab placebo SC+Cemdisiran placebo SC on weeks 4, 8, 16, and 20).
Pozelimab monotherapy regimen: Pozelimab 200 mg SC+Cemdisiran placebo SC Q4W starting from day 1 until week 20, inclusive (once the number of patients randomized to the Pozelimab arm reaches approximately 40 patients, then further enrollment of patients, if applicable, will continue into the remaining 3 arms)

Patients receiving treatment with a cholinesterase inhibitor, systemically administered corticosteroid, or other immunosuppressive treatments are to maintain the dose and schedule of these medications until week 36, unless there is a compelling medical need for adjustment or protocol requirement (e.g., the withholding of cholinesterase inhibitors prior to study visits).

Study procedures include MG-ADL, QMG, MGC, CGIS/CGIC, Modified MGFA-PIS: Change in Status, MG-QOL15r, Neuro-QOL-Fatigue, PGIS/PGIC, and EQ-5D-5L, metrics related to hospitalization and myasthenic crises, corticosteroid use, body weight, and routine safety assessments (ECG, vital signs, physical examination). Treatment-emergent adverse events (TEAEs) and concomitant medications/procedures are monitored throughout the study. The last doses for the DBTP will be given at week 20. Assessments for the primary analysis will be completed at week 24.

### Double-Blind Extension Treatment Period (Week 24 to Week 52)

After completing all of the week 24 DBTP assessments, including the assessment of the primary efficacy endpoint, the patients will enter into the 28-week double-blind, double-dummy ETP at week 24. Patients who were randomized to placebo during the DBTP will be re-randomized in a 1:1 ratio to the combination regimen or the Cemdisiran monotherapy regimen. Patients who were randomized to combination treatment or Cemdisiran monotherapy treatment during the DBTP will continue the same study treatment in the ETP. Patients who were randomized to Pozelimab monotherapy treatment during the DBTP will transition to the combination regimen. The first dose of Cemdisiran 200 mg SC will be given at week 24.

### Open-Label Treatment Period (Week 52 to Week 120)

After completing the week 52 ETP assessments, the patients will enter into the 68-week OLTP at week 52. Patients who have been on the combination regimen during the ETP will continue the same study treatment in the OLTP and receive treatment Q4W starting on week 52 with the last dose administered on week 116. Patients who have been on Cemdisiran regimen during the ETP will continue the same study treatment in the OLTP and receive treatment Q12W starting on week 60 with the last dose administered on week 108.

Blood samples will be taken for measurement of drug concentrations and anti-drug antibodies (ADA). Patients will return at week 120 for OLTP safety and efficacy assessments.

### Post-Treatment Follow-Up Period (52 Weeks)

After patients discontinue study treatment, patients will participate in follow ups until 52 weeks after the end of the OLTP. Selected safety, efficacy, and laboratory assessments will be performed to assess MG signs and symptoms after study treatment is discontinued. Starting from 12 weeks after the last dose, patients can initiate prohibited medications such as anti-C5 therapy for the treatment of MG.

Study assessments and procedures are presented by study period and visit in Table 1-1 for DBTP, Table 1-2 for ETP and OLTP, and Table 1-3 for the post-treatment FUP (follow-up period).

TABLE 1-1
A. Schedule of Events for the Placebo-Controlled Double-Blind Treatment Period

| | Screening | | Placebo-Controlled Double- | | | | | | | | |
|---|---|---|---|---|---|---|---|---|---|---|---|
| | Per | | Blind Treatment Period | | | | | | | | |
| | Sc | Sc | B | | | | | | | | Visit |
| | rn | rn | L | Vi | Vi | Vi | Vi | Vi | Vi | Vi | 11a²⁵ |
| Study Procedure^{1,2} | [symbol] | [symbol] | [symbol] | [symbol] | [symbol] | [symbol] | [symbol] | [symbol] | [symbol] | [symbol] | [symbol] |
| Week | Up to | −2 | 0 | 1 | 2 | 4 | 8 | 12 | 16 | 20 | 24 |
| Day | Up to | −14 | 1 | 8 | 15 | 29 | 57 | 85 | 11 | 14 | 169 |
| Window (day) | — | ±7 | — | ± [symbol] | ± [symbol] | ±5 | ± [symbol] | ± [symbol] | ± [symbol] | ± [symbol] | ±5 |
| Screening/Baseline | | | | | | | | | | | |
| Inclusion/Exclusion | x | x | x | | | | | | | | |
| Informed consent (main ICF and ICFs for | x | | | | | | | | | | |
| Medical and surgical history⁴ | x | | | | | | | | | | |
| MGFA Classification | x | | | | | | | | | | |
| Prior medications | x | | | | | | | | | | |
| Demographics | x | | | | | | | | | | |
| Height | x | | | | | | | | | | |
| Tuberculosis history and optional assessment | x | | | | | | | | | | |
| Hepatitis B and C panel⁷ | x | | | | | | | | | | |
| Sample to assess serology (anti-AChR, anti- | x | | | | | | | | | | |
| LRP4 and anti-MuSK antibodies) | | | | | | | | | | | |
| FSH (if needed for post-menopausal status) | x | | | | | | | | | | |
| Vaccination against Streptococcus | x | | | | | | | | | | |
| pneumoniae and Haemophilus influenza type | | | | | | | | | | | |
| Vaccination/revaccinate for meningococcal | x | | | | | | | | | | |
| Risk assessment for Neisseria gonorrhea⁹ | x | | | | | | | | | | |
| Randomization (through IRT) | | | x | | | | | | | | |
| Study Treatment | | | | | | | | | | | |
| IRT (dispense for dosing) | | | x | | | x | x | x | x | x | See |
| Administer SC dose (pozelimab/placebo) | | | x [symbol] | | | x | x | x | x | x | Table |
| Administer SC dose (cemdisiran/placebo) | | | x [symbol] | | | x | x | x | x | x | 2 |
| Daily antibiotics prophylaxis (per | | (x⁸ | 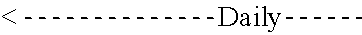 | | | | | | | | |
| Patient safety card for Neisseria meningitidis | | x | x | x | x | x | x | x | x | x | |

B. Schedule of Events for the Placebo-Controlled Double-Blind Treatment Period

| | Screening | | Placebo-Controlled Double- | | | | | | | | |
|---|---|---|---|---|---|---|---|---|---|---|---|
| | Period | | Blind Treatment Period | | | | | | | | |
| | | | | | | | | | | | Visit |
| | | | | | | | | | | | 11a²⁵ |
| | Scrn | Scrn | BL | | | | | | | | (pre- |
| | Visit | Visit | Visit | Visit | Visit | Visit | Visit | Visit | Visit | Visit | dose |
| Study Procedure^{1, 2} | 1 | 2 | 3 | 4 | 5 | 6 | 7 | 8 | 9 | 10 | procedures) |
| Week | Up to −5 | −2 | 0 | 1 | 2 | 4 | 8 | 12 | 16 | 20 | 24 |
| Day | Up to −36 | −14 | 1 | 8 | 15 | 29 | 57 | 85 | 113 | 141 | 169 |
| Window (day) | — | ±7 | — | ±2 | ±5 | ±5 | ±5 | +5 | ±5 | ±5 | ±5 |
| Study Assessments¹ | | | | | | | | | | | |
| MG-ADL¹³ | x | x | x | | x | x | x | x | x | x | x |
| OMG^{13,14,15} | | x | x | | x | x | x | x | x | x | x |
| MGC¹³ | | x | x | | x | x | x | x | x | x | x |
| CGIC (4 scales: MG body function, MG | | | | | | | | x | | | x |
| disease, ocular/bulbar weakness, and | | | | | | | | | | | |
| generalized weakness) | | | | | | | | | | | |
| CGIS (4 scales: MG body function, MG | | | x | | | | | x | | | x |
| disease, ocular/bulbar weakness, and | | | | | | | | | | | |
| generalized weakness) | | | | | | | | | | | |
| MG-PIS | | | | | | | | | | | x |
| Patient-Reported Assessments¹ | | | | | | | | | | | |
| MG-QOL15r | | | x | | x | x | x | x | x | x | x |
| Neuro-QOL-Fatigue | | | x | | x | x | x | x | x | x | x |
| EQ-5D-5L | | | x | | | | | x | | | x |
| PGIC (2 scales: MG functioning and MG | | | | | | | | x | | | x |
| disease) | | | | | | | | | | | |
| PGIS (2 scales: MG functioning and MG | | | x | | | | | x | | | x |
| disease) | | | | | | | | | | | |
| Safety | | | | | | | | | | | |
| Electrocardiogram | | x | | | x | | | | | | x |
| Vital signs | x | x | x | x | x | x | x | x | x | x | x |
| Body weight | x | | | | | x | | | | | x |
| Physical examination | x | | | | | | x | | | | x |
| Adverse events | x | x | x | x | x | x | x | x | x | x | x |
| Columbia-Suicide Severity Rating Scale (C- | x^{16a} | x^{16b} | x^{16b} | x^{16b} | x^{16b} | x^{16b} | x^{16b} | x^{16b} | x^{16b} | x^{16b} | x^{16b} |
| SSRS) | | | | | | | | | | | |
| Concomitant meds/treatment | x | x | x | x | x | x | x | x | x | x | x |
| MG crises and MG-related hospitalizations | x | x | x | x | x | x | x | x | x | x | x |

C. Schedule of Events for the Placebo-Controlled Double-Blind Treatment
Period

| | Screening | | Placebo-Controlled Double-Blind | | | | | | | | |
|---|---|---|---|---|---|---|---|---|---|---|---|
| | Period | | Treatment Period | | | | | | | | |
| | | | | | | | | | | | Visit |
| | | | | | | | | | | | 11a²⁵ |
| | Scrn | Scrn | BL | | | | | | | | (pre- |
| | Visit | Visit | Visit | Visit | Visit | Visit | Visit | Visit | Visit | Visit | dose |
| Study Procedure^{1, 2} | 1 | 2 | 3 | 4 | 5 | 6 | 7 | 8 | 9 | 10 | procedures) |
| Week | Up to −5 | −2 | 0 | 1 | 2 | 4 | 8 | 12 | 16 | 20 | 24 |
| Day | Up to −36 | −14 | 1 | 8 | 15 | 29 | 57 | 85 | 113 | 141 | 169 |
| Window (day) | — | ±7 | —- | ±2 | ±5 | ±5 | ±5 | ±5 | ±5 | ±5 | ±5 |
| Laboratory Testing | | | | | | | | | | | |
| Hematology | x | | x | | x | x | x | x | x | x | x |
| Blood chemistry | x | | x | | x | x | x | x | x | x | x |
| INR | x | | | | | | | x | | | x |
| Immunoglobulin G (IgG) | | | x | | | x | | x | | | |
| Pregnancy test (WOCBP | x | x | x | | | x | x | x | x | x | x |
| only)¹⁷ | | | | | | | | | | | |
| Urinalysis | x | x | x | | | x | | x | | x | x |
| Pharmacokinetics and Immunogenicity Sampling | | | | | | | | | | | |
| Serum pozelimab conc. | | | x | x | x | x | x | x | x | x | x |
| sample¹⁸ | | | | | | | | | | | |
| Plasma cemdisiran and | | | x | | | | | x | | | x |
| metabolites conc. sample | | | | | | | | | | | |
| (pre-dose and post- dose)¹⁹ | | | | | | | | | | | |
| Plasma total C5 sample²⁰ | x | | x | x | x | x | x | x | x | x | x |
| Serum pozelimab ADA | | | x | | | | | x | | | x |
| sample for | | | | | | | | | | | |
| immunogenicity²⁰ | | | | | | | | | | | |
| Serum cemdisiran ADA | | | x | | | | | x | | | x |
| sample for | | | | | | | | | | | |
| immunogenicity²⁰ | | | | | | | | | | | |
| Biomarkers | | | | | | | | | | | |
| Complement hemolytic | x | | x | x | x | x | | x | | | x |
| assay (serum CH50)²¹ | | | | | | | | | | | |
| Complement hemolytic | | | x | x | x | x | | x | | | x |
| assay (serum AH50))²¹ | | | | | | | | | | | |
| CCP Wieslab²¹ | | | x | x | x | x | | | | | x |
| CAP Wieslab²¹ | | | x | x | x | x | | x | | | x |
| Anti-LRP4 (ELISA) | x | | | | | | | | | | |
| Sub-Studies | | | | | | | | | | | |
| Provide/retrieve wearable | x^{22a} | x^{22b} | | | | | | | | x^{22a} | ×^{22b} |
| device (if possible) | | | | | | | | | | | |
| Serum and plasma for FBR | | | x | x | | x | | x | | | x |
| (optional FBR sub-study)²³ | | | | | | | | | | | |
| Whole blood sample for | | | x | | | | | | | | |
| DNA isolation (optional | | | | | | | | | | | |
| genomics sub-study)²⁴ | | | | | | | | | | | |
| Whole Blood sample for | | | x | | | | | x | | | x |
| RNA isolation (optional | | | | | | | | | | | |
| genomics sub-study) | | | | | | | | | | | |

Footnotes for Table 1-1
¹Before screening visits and every visit in the DBTP, study staff should contact the patient by phone with a reminder to withhold cholinesterase inhibitors for at least 10 hours prior to their visit, to be resumed after visit assessments are complete. A consistent sequence of procedures should be followed across visits, such as: MG-QOL15r, Neuro-QOL-Fatigue, PGIC, PGIS, MG-ADL, QMG, MGC, CGIC, CGIS, EQ-5D-5L, ECG, vital signs and other safety assessments, blood collection, study treatment administration, followed by any postdose safety assessments. The C-SSRS can be done at any time after the MGC, but before study treatment administration. Sites should follow specific details described in the study manual.
²Study procedures should be performed at the study clinic for visits in the DBTP. In exceptional cases (e.g., pandemic-related travel restrictions), remote and home visits are allowed, but QMG and MGC can only be performed on site.
³Includes informed consent forms (ICFs) for the main study as well as ICFs for wearable device sub-study (if possible), optional future biomedical research (FBR) sub-study, and optional pharmacogenomics sub-study.
⁴Medical and surgical history: Duration of MG disease should be captured as accurately as possible. Surgical history, including thymectomy, should be captured. If applicable include detailed schedule of procedures such as IVIG and PLEx. Any hospitalizations due to MG in the prior 2 years should be recorded. Additional disease characteristics will be recorded.
⁵Prior medications: if applicable include detailed IST, complement inhibitor, cholinesterase inhibitors, and N. meningitidis vaccination and other vaccination history.
⁶Tuberculosis history and optional assessment. Optional screening by tuberculin skin test or T-cell interferon-gamma release assay may be performed according to local practice or guidelines at the discretion of the investigator. T-cell interferon gamma release assay may be done by the central lab, as needed.
⁷The hepatitis B panel includes hepatitis B surface antigen (HBsAg), hepatitis B core antibodies (anti-HBC), and hepatitis B surface antibodies (anti-HBs). Hepatitis C panel includes hepatitis C antibody and hepatitis C virus RNA.
⁸Patients must be vaccinated for N. meningitidis or revaccinated if >5 years since last vaccination, or a shorter duration as required by current national vaccination guidelines. If a patient receives meningococcal vaccination that precedes initiation of study treatment by less than 2 weeks, the patient must receive antibiotic prophylaxis for a minimum of 2 weeks from the date of vaccination.
⁹A risk factor assessment for N. gonorrhea is recommended, and counseling is advised for at-risk patients.
¹⁰Study treatment administration: On day 1, the patient should be monitored for at least 30 minutes after the SC injections.
¹¹Investigators may consider the use of daily oral antibiotic prophylaxis to mitigate the risk of infections, starting on the first day of dosing and continuing until 52 weeks after the last dose of study treatment.
¹²Patient safety card: provide the patient safety card for N. meningitidis infection to the patient on day 1 or any other visit when needed. Site should review the instructions on the safety card with the patient at each visit.
¹³The MG-ADL, QMG, and MGC should be performed by the same clinician evaluator across visits.
¹⁴The QMG should be done at approximately the same time of day at each visit. Patients should be reminded to withhold their acetylcholinesterase inhibitor (e.g., pyridostigmine) for at least10 hours prior to visits when the QMG is administered.
¹⁵During the course of the QMG, clinicians will record continuous variable results from select measures (hand grip strength, supine leg lift, supine head lift, onset of dysarthria) on a separate sheet.
¹⁶
ªUse the ″Baseline/Screening″ version of C-SSRS for visit 1.
^{b}For all other visits, use the ″Since Last Visit″ version of the C-SSRS. If there is a positive response of suicide attempt or suicidal ideation, the investigator should immediately evaluate the patient and determine the need for an urgent referral to a specialist. The investigator in conjunction with the specialist should determine if the patient should be discontinued from the study as well as the appropriate management for the patient's safety should be initiated.
¹⁷Pregnancy testing for WOCBP only: A serum pregnancy test will be performed at the first visit, and urine pregnancy tests will be performed at subsequent visits.
¹⁸Blood samples for drug concentration of pozelimab: obtain blood samples prior to any SC study treatment administration.
¹⁹Blood samples for concentrations of cemdisiran and its metabolites will be collected prior to any SC study treatment administration (pre-dose at 0 hour) and once at 1 to 4 hours postdose at each time point specified.
²⁰After randomization on day 1, blood samples for measurement of total C5, anti-pozelimab, and anti-cemdisiran antibodies will be collected before the administration of study treatment (pre-dose at 0 hr). In the event of suspected SAEs, such as anaphylaxis or hypersensitivity, additional samples for drug concentration and ADA may be collected at or near the onset and the resolution of the event.
²¹After randomization on day 1, blood samples for measurement of CH50, AH50, CCP Wieslab, and CAP Wieslab will be collected prior to study treatment administration.
²²
ªWearable devices (if possible and allowed by local regulation) will be used to collect exploratory data. At the specified time points, patients will be provided with a wearable device and be directed to wear the device for a continuous period while performing daily activities. Detailed instructions will be provided separately to each patient.
^{b}At the specified time points, the wearable device will be collected/retrieved for analysis.
²³Future biomedical research, serum and plasma (optional): Samples should be collected as permitted by patient consent and local regulatory policies. They may be stored for up to 15 years or as permitted by local regulatory policies, whichever is shorter, for exploratory research.
²⁴Whole blood samples (optional) for DNA extraction should be collected on day 1 (predose) but can be collected at a later study visit during the DBTP.
²⁵Study treatment administration and post-dose assessments at week 24 are described in Table 1-2. At week 24, after all DBTP assessments are made, patients progress to the ETP and double-blind SC are administered to all patients. [symbol]
indicates data missing or illegible when filed

TABLE 1-2
A. Schedule of Events for Double-Blind Extension and Open-Label
Treatment Periods

| | Double-Blind Extension Treatment | | | | | | | | | | | | | | | |
|---|---|---|---|---|---|---|---|---|---|---|---|---|---|---|---|---|
| | Period | | | | | | | | Open-Label Treatment Period | | | | | | | |
| | Visit 11b | | | | | | | | | | | | | | | |
| | (dosing | | | | | | | | | | | | | | | |
| | and | | | | | | | | | | | | | | | |
| | post- | | | | | | | | | | | | | | | |
| Study | dose | Visit | Visit | Visit | Visit | Visit | Visit | Visit | Visit | Visit | Visit | Visit | Visit | Visit | Visit | Visit |
| Procedure¹ | procedures) | 12 | 13 | 14 | 15 | 16 | 17 | 18a | 18b | 19 | 20 | 21 | 22 | 23 | 24 | 25 |
| Week | 24 | 28 | 32 | 36 | 40 | 44 | 48 | 52 | 52 | 60 | 72 | 84 | 96 | 108 | 116 | 120 |
| Day | (Cont. from | 197 | 225 | 253 | 281 | 309 | 337 | 365 | 365 | 421 | 505 | 589 | 673 | 757 | 813 | 841 |
| Window (day) | Table 1) | ±5 | ±5 | ±5 | ±5 | ±5 | ±5 | ±5 | ±5 | ±5 | ±5 | ±5 | ±5 | ±5 | ±5 | ±5 |
| Clinic visits² | | | x | | x | | x | | | x | | x | | x | | x |
| Randomization | x | | | | | | | | | | | | | | | |
| (through IRT) | | | | | | | | | | | | | | | | |
| Treatment | | | | | | | | | | | | | | | | |
| IRT (dispense | x | x | x | x | x | x | x | | x | x | x | x | x | x | x | x |
| for dosing)³ | | | | | | | | | | | | | | | | |
| Administer SC | x⁴ | x | x | x | x | x | x | | | | | | | | | |
| dose | | | | | | | | | | | | | | | | |
| (pozelimab/ | | | | | | | | | | | | | | | | |
| placebo) | | | | | | | | | | | | | | | | |
| Administer SC | x⁴ | x | x | x | x | x | x | | | | | | | | | |
| dose | | | | | | | | | | | | | | | | |
| (cemdisiran/ | | | | | | | | | | | | | | | | |
| placebo) | | | | | | | | | | | | | | | | |
| Administer open-label pozelimab SC | | | | | | | | | x | 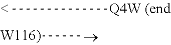 | | | | | | |
| Q4W (patients | | | | | | | | | | | | | | | | |
| assigned to | | | | | | | | | | | | | | | | |
| combination | | | | | | | | | | | | | | | | |
| treatment)⁵ | | | | | | | | | | | | | | | | |
| Administer open-label cemdisiran SC | | | | | | | | | x | 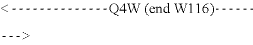 | | | | | | |
| Q4W(patients | | | | | | | | | | | | | | | | |
| assigned to | | | | | | | | | | | | | | | | |
| combination | | | | | | | | | | | | | | | | |
| treatment)⁵ | | | | | | | | | | | | | | | | |
| Administer open-label cemdisiran 600 mg SC Q12W (patients assigned to cemdisiran | | | | | | | | | | 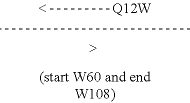 | | | | | | |
| monotherapy | | | | | | | | | | | | | | | | |
| treatment) | | | | | | | | | | | | | | | | |
| Daily | | 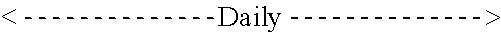 | | | | | | | | | | | | | | x |
| antibiotics | | | | | | | | | | | | | | | | |
| prophylaxis | | | | | | | | | | | | | | | | |
| (per | | | | | | | | | | | | | | | | |
| investigator)⁶ | | | | | | | | | | | | | | | | |
| Patient safety | x | x | x | x | x | x | x | x | | x | x | x | x | x | x | x |
| card for | | | | | | | | | | | | | | | | |
| Neisseria | | | | | | | | | | | | | | | | |
| meningitidis⁷ | | | | | | | | | | | | | | | | |
| SC injection training (as needed) | | | | | | | | | 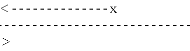 | | | | | | | |
| Patient diary⁸ | | | | | | | | | x | x | x | x | x | x | x | |
| Study Assessments¹ | | | | | | | | | | | | | | | | |
| MG-ADL⁹ | | x | x | x | | | x | | | x | | x | | x | | x |
| OMG^{9,10,11} | | | x | x | | | x | | | x | | x | | x | | x |
| MGC⁹ | | | | x | | | x | | | x | | x | | x | | x |
| CGIC | | | | | | | x | | | | | | | | | x |
| CGIS | | | | | | | x | | | | | | | | | x |
| MGFA PIS | | | | | | | x | | | | | | | | | x |

B. Schedule of Events for Double-Blind Extension and Open-Label
Treatment Periods

| | Double-Blind Extension Treatment | | | | | | | | | | | | | | | |
|---|---|---|---|---|---|---|---|---|---|---|---|---|---|---|---|---|
| | Period | | | | | | | | Open-Label Treatment Period | | | | | | | |
| | Visit 11b | | | | | | | | | | | | | | | |
| | (dosing | | | | | | | | | | | | | | | |
| | and | | | | | | | | | | | | | | | |
| | post- | | | | | | | | | | | | | | | |
| Study | dose | Visit | Visit | Visit | Visit | Visit | Visit | Visit | Visit | Visit | Visit | Visit | Visit | Visit | Visit | Visit |
| Procedure¹ | procedures) | 12 | 13 | 14 | 15 | 16 | 17 | 18a | 18b | 19 | 20 | 21 | 22 | 23 | 24 | 25 |
| Week | 24 | 28 | 32 | 36 | 40 | 44 | 48 | 52 | 52 | 60 | 72 | 84 | 96 | 108 | 116 | 120 |
| Day | (Cont. from | 197 | 225 | 253 | 281 | 309 | 337 | 365 | 365 | 421 | 505 | 589 | 673 | 757 | 813 | 841 |
| Window (day) | Table 1) | ±5 | ±5 | ±5 | ±5 | ±5 | ±5 | ±5 | ±5 | ±5 | ±5 | ±5 | ±5 | ±5 | ±5 | ±5 |
| Patient-Reported | | | | | | | | | | | | | | | | |
| Assessments¹ | | | | | | | | | | | | | | | | |
| MG-QOL15r | | x | | x | | | x | | | x | | x | | x | | x |
| Neuro-QOL | | x | | x | | | x | | | x | | x | | x | | x |
| Fatigue | | | | | | | | | | | | | | | | |
| EQ-5D-5L | | | | x | | | x | | | x | | | | | | x |
| PGIC | | | | | | | x | | | | | | | | | x |
| PGIS | | | | | | | x | | | | | | | | | |
| Safety | | | | | | | | | | | | | | | | |
| Vital signs | | | x | x | x | x | x | x | | x | x | x | x | x | x | x |
| Body weight | | | | | | | | x | | | | | | | | x |
| Physical | | | | | | | | x | | | | | | | | x |
| examination | | | | | | | | | | | | | | | | |
| Electrocardio | | | | | | | | x | | | | | | | | x |
| gram | | | | | | | | | | | | | | | | |
| Adverse | | x | x | x | x | x | x | x | | x | x | x | x | x | x | x |
| events | | | | | | | | | | | | | | | | |
| Columbia- | x | x | x | x | x | x | x | x | x | x | x | x | x | x | x | x |
| Suicide | | | | | | | | | | | | | | | | |
| Severity | | | | | | | | | | | | | | | | |
| Rating | | | | | | | | | | | | | | | | |
| Scale | | | | | | | | | | | | | | | | |
| (C-SSRS)¹² | | | | | | | | | | | | | | | | |
| Concomitant | | x | x | x | x | x | x | x | | x | x | x | x | x | x | x |
| meds/treatment | | | | | | | | | | | | | | | | |
| MG crises | | x | x | x | x | x | x | x | | x | x | x | x | x | x | x |
| and MG- | | | | | | | | | | | | | | | | |
| related | | | | | | | | | | | | | | | | |
| hospitalizations | | | | | | | | | | | | | | | | |
| Laboratory Testing | | | | | | | | | | | | | | | | |
| Hematology | | | x | x | x | x | x | x | | x | x | x | x | x | x | x |
| Blood | | | x | x | x | x | x | x | | x | x | x | x | x | x | x |
| chemistry | | | | | | | | | | | | | | | | |
| INR | | | | | x | | x | | | | | | | | | |
| Pregnancy test (WOCBP)¹³ | | | | | x | x | x | x | | 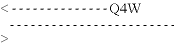 | | | | | | x |
| Urinalysis | | | | x | | | | x | | | x | | x | | | x |

C. Schedule of Events for Double-Blind Extension and Open-Label
Treatment Periods

| | Double-Blind Extension Treatment | | | | | | | | | | | | | | | |
|---|---|---|---|---|---|---|---|---|---|---|---|---|---|---|---|---|
| | Period | | | | | | | | Open-Label Treatment Period | | | | | | | |
| | Visit 11b | | | | | | | | | | | | | | | |
| | (dosing | | | | | | | | | | | | | | | |
| | and | | | | | | | | | | | | | | | |
| | post- | | | | | | | | | | | | | | | |
| Study | dose | Visit | Visit | Visit | Visit | Visit | Visit | Visit | Visit | Visit | Visit | Visit | Visit | Visit | Visit | Visit |
| Procedure¹ | procedures) | 12 | 13 | 14 | 15 | 16 | 17 | 18a | 18b | 19 | 20 | 21 | 22 | 23 | 24 | 25 |
| Week | 24 | 28 | 32 | 36 | 40 | 44 | 48 | 52 | 52 | 60 | 72 | 84 | 96 | 108 | 116 | 120 |
| Day | (Cont. from | 197 | 225 | 253 | 281 | 309 | 337 | 365 | 365 | 421 | 505 | 589 | 673 | 757 | 813 | 841 |
| Window (day) | Table 1) | ±5 | ±5 | ±5 | ±5 | ±5 | ±5 | ±5 | ±5 | ±5 | ±5 | ±5 | ±5 | ±5 | ±5 | ±5 |
| Pharmacokinetics and | | | | | | | | | | | | | | | | |
| Immunogenicity Sampling | | | | | | | | | | | | | | | | |
| Serum | | x | | x | | | x | | | x | x | x | x | x | x | x |
| pozelimab | | | | | | | | | | | | | | | | |
| conc. sample¹⁴ | | | | | | | | | | | | | | | | |
| Plasma | x | | | x | | | x | | | | | | | | | |
| cemdisiran | | | | | | | | | | | | | | | | |
| and | | | | | | | | | | | | | | | | |
| metabolites | | | | | | | | | | | | | | | | |
| conc. sample | | | | | | | | | | | | | | | | |
| (pre-dose and | | | | | | | | | | | | | | | | |
| post-dose)¹⁵ | | | | | | | | | | | | | | | | |
| Plasma total | | x | | x | | | x | | | x | x | x | x | x | x | x |
| C5 sample¹⁶ | | | | | | | | | | | | | | | | |
| Serum | | | | x | — | | x | | | | x | | x | | | x |
| pozelimab | | | | | | | | | | | | | | | | |
| ADA sample | | | | | | | | | | | | | | | | |
| for | | | | | | | | | | | | | | | | |
| immunogenicit¹⁶ | | | | | | | | | | | | | | | | |
| Serum | | | | x | | | x | | | | x | | x | | | x |
| cemdisiran | | | | | | | | | | | | | | | | |
| ADA sample | | | | | | | | | | | | | | | | |
| for | | | | | | | | | | | | | | | | |
| immunogenicity¹⁶ | | | | | | | | | | | | | | | | |
| Biomarkers | | | | | | | | | | | | | | | | |
| Complement | | x | | x | | | x | | | | | x | | | x | x |
| hemolytic | | | | | | | | | | | | | | | | |
| assay | | | | | | | | | | | | | | | | |
| (serum | | | | | | | | | | | | | | | | |
| CH50)¹⁷ | | | | | | | | | | | | | | | | |
| Complement | | x | | x | | | x | | | | | x | | | x | x |
| hemolytic | | | | | | | | | | | | | | | | |
| assay | | | | | | | | | | | | | | | | |
| (serum | | | | | | | | | | | | | | | | |
| AH50)¹⁷ | | | | | | | | | | | | | | | | |
| CCP Wieslab¹⁷ | | x | | x | | | x | | | | | x | | | x | x |
| CAP Wieslab¹⁷ | | x | | x | | | x | | | | | x | | | x | x |
| Sub-studies | | | | | | | | | | | | | | | | |
| Provide/ | | | | | | | x^{18a} | x^{18b} | | | | | | | | |
| retrieve | | | | | | | | | | | | | | | | |
| wearable | | | | | | | | | | | | | | | | |
| device (if | | | | | | | | | | | | | | | | |
| possible) | | | | | | | | | | | | | | | | |
| Serum and | | x | | x | | | x | | | | | x | | | x | x |
| plasma for | | | | | | | | | | | | | | | | |
| FBR (optional | | | | | | | | | | | | | | | | |
| sub- study) 19 | | | | | | | | | | | | | | | | |

Footnotes for Table 1-2
¹Before every visit in the ETP and OLTP, study staff should contact the patient by phone with a reminder to withhold cholinesterase inhibitors for at least 10 hours prior to their visit, to be resumed after visit assessments are complete. A consistent sequence of procedures should be followed across visits, such as: MG-QOL15r, Neuro-QOL-Fatigue, PGIC, PGIS, MG-ADL, QMG, MGC, CGIC, CGIS, EQ-5D-5L, ECG, vital signs and other safety assessments, blood collection, study treatment administration, followed by any postdose safety assessments. The C-SSRS can be done at any time after the MGC but before study treatment administration. Sites should follow specific details described in the study manual.
²These visits should be conducted at the clinical site. Other visits that are not indicated to be at the clinical site are still expected to be conducted at the clinical site, but in exceptional cases (eg, pandemic-related travel restrictions), remote and home visits are allowed. Please note that QMG and MGC can only be performed on site due to constraints of home health visits and remote video assessments. If the option of home nursing visits is available for the site's region and if approved by the sponsor, other visits during the ETP and OLTP may be conducted at the clinical site or at another preferred location such as patient's home.
³IRT dispensations will occur every 4 to 12 weeks depending on whether the patient is receiving dose administration at the clinic, or administration by patient/caregiver at the patient's preferred location (e.g., patient's home). At week 24, after all assessments are completed for the DBTP (Table 1-1), sites will be required to call IRT for re-randomization.
⁴Study treatment administration: At week 24, the first dose of study treatment is administered as a double-blind double-dummy SC, and patients should be monitored for at least 30 minutes post-administration since patients initially assigned to placebo will receive either combination or cemdisiran monotherapy for the first time.
⁵Patients will receive a telephone call from the study site when dosing is administered at home. Care must be taken to coordinate dosing for visits where a post-dose sample is collected to measure the concentration of cemdisiran and its metabolites.
⁶Investigators may consider the use of daily oral antibiotic prophylaxis to mitigate the risk of infections, starting on the first day of dosing and continuing until 52 weeks after discontinuation of study treatment.
⁷Patient safety card: provide the patient safety card for N. meningitidis infection to the patient on day 1 or any other visit when needed. Site should review the instructions on the safety card with the patient at each visit.
⁸A patient diary will be provided at week 52 for patients to record study treatment administration during the OLTP. The diary will be reviewed at each visit for treatment compliance.
⁹The MG-ADL, QMG, and MGC should be performed by the same clinician evaluator across visits.
¹⁰The QMG should be done at approximately the same time of day at each visit. Patients should be reminded to withhold their acetylcholinesterase inhibitor (e.g., pyridostigmine) for at least 10 hours prior to visits when the QMG is administered.
¹¹During the course of the QMG, clinicians will record continuous variable results from select measures (hand grip strength, supine leg lift, supine head lift, onset of dysarthria) on a separate sheet.
¹²Use the ″Since Last Visit″ version of the C-SSRS for visits in the ETP and OLTP. If there is a positive response of suicide attempt or suicidal ideation, the investigator should immediately evaluate the patient and determine the need for an urgent referral to a specialist. The investigator in conjunction with the specialist should determine if the patient should be discontinued from the study and the appropriate management for the patient's safety should be initiated.
¹³Pregnancy testing: Monthly urine pregnancy testing will be conducted for WOCBP only. If performed via at-home testing kits, patients should be reminded to call the study staff if their pregnancy test result is positive.
¹⁴Blood samples for drug concentration of pozelimab should be collected prior to any SC study treatment administration.
¹⁵Blood samples for concentrations of cemdisiran and its metabolites will be collected prior to any SC study treatment administration (pre-dose at 0 hr) and once at 1 to 4 hours postdose at each time point specified.
¹⁶Blood samples for measurement of total C5, anti-pozelimab and anti-cemdisiran antibodies will be collected before the administration of study treatment (pre-dose at 0 hr). In the event of suspected SAEs, such as anaphylaxis or hypersensitivity, additional PK and ADA samples may be collected at or near the onset and the resolution of the event.
¹⁷Blood samples for measurement of CH50, AH50, CCP Wieslab, and CAP Wieslab will be collected prior to study treatment administration.
¹⁸
ªWearable devices (if possible and allowed by local regulation) will be used to collect exploratory data. At the specified time points, patients will be provided with a wearable device and directed to wear the device for a continuous period while performing daily activities. Detailed instructions will be provided separately to each patient.
^{b}At the specified time points, the wearable device will be collected/retrieved for analysis.
¹⁹Future research serum and plasma (optional): Library samples should be collected, as permitted by patient consent and local regulatory policies. They may be stored for up to 15 years or as permitted by local regulatory policies, whichever is shorter, for exploratory research.

TABLE 1-3
Schedule of Events for Post-Treatment Follow-Up Period

| | | | | | Phone | |
|---|---|---|---|---|---|---|
| | | | | | visit | Phone visit |
| Visit¹ | FU-0 | FU-1 | FU-2 | FU-3 | FU-4 | FU-5 |
| Week (after end of OLTP | 4 | 8 | 16 | 26 | 38 | 52 |
| or last dose of study | | | | | | |
| treatment, as applicable) | | | | | | |
| Window (day) | ±10 | ±10 | ±10 | ±10 | ±10 | ±10 |
| Efficacy Assessment | | | | | | |
| MG-ADL | | x | | x | | |
| Safety Assessments | | | | | | |
| Daily antibiotics prophylaxis (per | 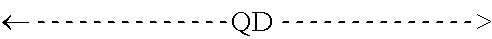 | | | | | |
| investigator) | | | | | | |
| Vital signs | x | x | x | x | | |
| Physical examination | | | | x | | |
| Concomitant | x | x | x | x | x | x |
| meds/treatment | | | | | | |
| Adverse event reporting | x | x | x | x | x | x |
| Columbia-Suicide | x | x | x | x | | |
| Severity Rating Scale | | | | | | |
| (C-SSRS)³ | | | | | | |
| Pregnancy reporting | x | x | x | x | x | x |
| Laboratory Testing | | | | | | |
| Hematology | x | x | x | x | | |
| Blood chemistry | x | x | x | x | | |
| Monthly urine pregnancy test (WOCBP)⁴ | 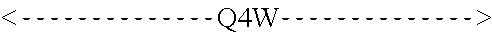 | | | | | |
| Serum pozelimab conc. | | | | x | | |
| sample | | | | | | |
| Serum pozelimab ADA | | | | x | | |
| sample for immunogenicity | | | | | | |
| Serum cemdisiran ADA | | | | x | | |
| sample for immunogenicity | | | | | | |
| Plasma total C5 sample | | | | x | | |

Footnotes for Table 1-3
¹Patients who discontinue study treatment during the DBTP will be asked to remain in the study until week 24 and follow the original schedule of events as applicable. After the week 24 visit, their entry point into the follow-up schedule will depend on the number of weeks that have elapsed since their last dose (ie, a patient who is 20 weeks after their final dose of study treatment at the week 24 visit will enter into the follow-up period at visit FU-3 [26 weeks after last dose])
²Investigators may consider the use of daily oral antibiotic prophylaxis to mitigate the risk of infections, starting on the first day of dosing and continuing until 52 weeks after discontinuation of study treatment.
³Use the ″Since Last Visit″ version of the C-SSRS at all visits in the FU period. If there is a positive response of suicide attempt or suicidal ideation, the investigator should immediately evaluate the patient and determine the need for an urgent referral to a specialist. The investigator in conjunction with the specialist should determine if the patient should be discontinued from the study as well as the appropriate management for the patient's safety should be initiated.
⁴Pregnancy testing: Monthly urine pregnancy testing will be conducted for WOCBP only. If performed via at-home testing kits, patients should be reminded to call the study staff if their pregnancy test result is positive.

### Study Patient Population

Eligible patients consist of adult men and women with a confirmed clinical and serologic diagnosis (seropositive for anti-AChR or anti-LRP4 antibodies) of gMG having signs and symptoms of MG (as defined by MG-ADL score ≥6 and MGFA Class II to IVa at screening). Method of treatment assignment and stratification factors are described herein:

Approximately 235 patients will be randomized in a 1:1:1:1 ratio to receive either combination, Cemdisiran, Pozelimab, or placebo according to a central randomization scheme provided by IRT to the designated study pharmacist or qualified designee. Once the number of patients randomized to the Pozelimab arm reaches approximately 40 patients, then further enrollment of patients, if applicable, will continue in a 1:1:1 ratio into the remaining 3 arms.

Randomization will be stratified according to the values for: (1) MGFA Clinical Classification Class II at screening (yes/no); (2) Treatment with IST on day 1 (none/treatment with all IST for <1 year/treatment with any IST for >1 year); (3) Region at screening (Japan/Rest-of-world).

Regarding the second stratification factor, treatment with IST on day 1, the following is noted: (i) if patient has no ongoing use of IST on day 1, then the patient should be assigned “none;” (ii) if the patient has ongoing use of one or more IST on day 1 with all IST having a duration of ≤1 year, then the patient should be assigned “treatment with all IST for ≤1 year;” (iii) if the patient has ongoing use of IST on day 1 with any one or more IST having a duration of >1 year, then the patient is assigned “treatment with any IST for >1 year.”

At week 24, patients randomized to the placebo group are re-randomized 1:1 to receive combination or Cemdisiran. Patients randomized to combination or Cemdisiran treatment during the DBTP remain in their treatment group in the ETP. Patients randomized to the Pozelimab arm during the DBTP receive combination treatment in the ETP.

At week 52, patients on combination during the ETP continue combination and patients on Cemdisiran during the ETP continue Cemdisiran. See Table 1-4.

TABLE 1-4
Treatment Period Summary

| Treatment group in DBTP | Treatment group in ETP | Treatment group in OLTP |
|---|---|---|
| Placebo | Re-randomize 1:1 to: | Continue treatment from ETP |
| | Combination | Combination |
| | Cemdisiran | Cemdisiran |
| Combination | Combination | Combination |
| Cemdisiran | Cemdisiran | Cemdisiran |
| Pozelimab | Combination | Combination |

DBTP = double-blind treatment period
ETP = extension treatment period
OLTP = open-label treatment period

### Inclusion Criteria

A patient must meet the following criteria to be eligible for inclusion in the study:
1. Male or female patients ≥18 years of age at screening (or ≥ legal age of adulthood based on local regulations, whichever is older)
2. Patient with documented diagnosis of MG based on medical history and supported by previous evaluations. Examples of supportive evaluations include any one of the following:
   History of abnormal neuromuscular transmission by repetitive nerve stimulation/single-fiber electromyography
   History of positive anticholinesterase test
   Positive response (improvement in signs) with anticholinesterase treatment, as assessed by the treating physician
3. Documented prior history of positive serologic test or a positive result during screening of anti-AChR antibodies or anti-LRP4 antibodies. One retest is allowed.
4. MGFA Clinical Classification Class II to IVa at screening
5. MG-ADL score ≥6 at screening (either week-5 or week-2) and baseline (day 1). Ocular items should not contribute more than 50% of MG-ADL total score
6. Currently receiving an acetylcholinesterase inhibitor or documented reason for not using acetylcholinesterase inhibitor therapy per investigator
7. Currently receiving an IST for MG, or documented reason why the patient is not taking an IST per investigator (ISTs include, but are not limited to, systemic corticosteroids, azathioprine, mycophenolate mofetil, methotrexate, cyclosporine, tacrolimus, or cyclophosphamide.)
8. If currently receiving an IST, not anticipated to have IST dosage changed before randomization or during DBTP. Patients who require time to stabilize their treatment regimen may be rescreened.
9. Patient agrees to participate in the study and sign the study consent
10. Willing and able to comply with clinic visits and study-related procedures
11. Able to understand and complete study-related questionnaires

### Exclusion Criteria

A patient who meets any of the following criteria will be excluded from the study:
1. Patients with antibody profile that is only positive for MuSK (MuSK positivity is based on a documented prior history of positive serologic test for antibodies to MuSK or a positive result during screening)
2. History of thymectomy within 12 months prior to screening or planned during the study
3. History of malignant thymoma (patients with stage 1 may be enrolled), or history of cancer within the past 5 years, except for adequately treated basal cell skin cancer, squamous cell skin cancer, or in situ cervical cancer
4. Myasthenic crisis or MGFA Class V within 1 month of screening
5. Any systemic bacterial or other infection that was clinically significant in the opinion of the investigator and had not been treated with appropriate therapy within 2 weeks of screening or during the screening period
6. Unresolved meningococcal infection
7. No documented meningococcal vaccination within 5 years prior to screening visit unless vaccination will be administered during the screening period and prior to initiation of study treatment
8. Known contraindication to meningococcal vaccines (group ACWY conjugate and group B vaccines). Refer to the most recent local product information for each vaccine, if approved, for the current list of contraindications
9. Patients who require antibiotics for meningococcal prophylaxis and have a contraindication, warning, or precaution precluding the use of penicillin class and penicillin-alternative antibiotics planned to be used for prophylaxis, or a history of intolerance leading to the discontinuation of these antibiotics
10. Positive hepatitis B surface antigen or hepatitis C virus RNA during screening. NOTE: Cases with unclear interpretation should be discussed with the medical monitor
11. History of HIV infection or a positive test at screening per local requirements
12. Patients with an estimated glomerular filtration rate (eGFR)<30 mL/min/1.73 m2 (according to the Chronic Kidney Disease Epidemiology Collaboration creatinine equation (Levey, 2009)) at screening visit. One retest is allowed
13. Documented history of liver cirrhosis or known liver disease with current evidence of ongoing impaired liver function (e.g., screening international normalized ratio [INR]>1.5×ULN that is considered hepatic in origin)
14. Patients with screening serum ALT levels >3×ULN and/or total bilirubin >2×ULN (unless bilirubin elevation is due to suspected Gilbert's syndrome). One retest is allowed
15. Patients receiving azathioprine who have not been on azathioprine for at least 6 months prior to screening, of which a stable dosing regimen is maintained for at least 2 months immediately preceding screening. Once the patient is stable on his/her background therapy for the specified amount of time, the patient may enter the screening period
16. Patients receiving other ISTs (e.g., systemic corticosteroids, mycophenolate mofetil, methotrexate, cyclosporine, tacrolimus, or cyclophosphamide) who have not been on the IST for at least 3 months prior to screening, of which a stable dosing regimen is maintained for at least 4 weeks immediately preceding screening
17. Patients receiving cholinesterase inhibitors who have not been on a stable dosing regimen for at least 2 weeks prior to screening
18. Ongoing treatment with IVIG or PLEX/other comparable therapeutic apheresis (TA) procedure or treatment within 4 weeks prior to randomization (day 1)
19. Use of rituximab within 6 months prior to screening
20. Use of complement inhibitor therapy within 6 months of screening or neonatal Fc receptor (FcRn) inhibitors within 4 months or 5 half-lives of screening, whichever is longer, or the use of either of these classes of medications at any time during the screening period. Exception is patients with a documented C5 variant R885H/C, who may be screened 1 month after the last dose of eculizumab or ravulizumab
21. Muscular weakness predominantly due to conditions other than MG, such as myopathies, concurrent infections, medications, or hospitalization/bedrest immobilization
22. Participation in any other investigational drug study or exposure to other investigational agent, device, or procedures within 30 days prior to screening
23. Hypersensitivity to Pozelimab or Cemdisiran or to one of its excipients
24. Any medical condition or clinically significant abnormality that, in the opinion of the investigator, might interfere with the patient's participation in the study, pose any added risk for the patient, or confound the assessment of efficacy (such as instability of MG signs and symptoms or high doses of prednisone that are not expected to be maintained during the DBTP in the study)
25. Patients with functional or anatomic asplenia
26. Any life-time suicidal actions (per investigator judgement i.e., actual attempt, interrupted attempt, aborted attempt, or preparatory acts or behavior)
27. Any suicidal ideation of type 4 or 5 in the Columbia Suicide Severity Rating Scale (C-SSRS) in the past 1 year (i.e., active suicidal thought with intent but without specific plan, or active suicidal thought with plan and intent)
28. Pregnant or breastfeeding women
29. WOCBP* who are unwilling to practice highly effective contraception prior to the first dose of study treatment, during the study, and for at least 52 weeks after the last dose of study treatment. Highly effective contraceptive measures include: (a) stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening; (b) intrauterine device; intrauterine hormone-releasing system; (c) bilateral tubal ligation; (d) vasectomized partner (provided that the male vasectomized partner is the sole sexual partner of the WOCBP study participant and that the vasectomized partner has obtained medical assessment of surgical success for the procedure); and/or (e) sexual abstinence†‡
   * WOCBP are defined as women who are fertile following menarche until becoming postmenopausal, unless permanently sterile. Permanent sterilization methods include hysterectomy, bilateral salpingectomy, and bilateral oophorectomy.
   A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance. Pregnancy testing and contraception are not required for women with documented hysterectomy or tubal ligation.
   † Sexual abstinence is considered a highly effective method only if defined as refraining from heterosexual intercourse during the entire period of risk associated with the study treatment. The reliability of sexual abstinence needs to be evaluated in relation to the duration of the clinical trial and the preferred and usual lifestyle of the subject.
   ‡ Periodic abstinence (calendar, symptothermal, post-ovulation methods), withdrawal (coitus interruptus), spermicides only, and lactational amenorrhea method are not acceptable methods of contraception. Female condom and male condom should not be used together.
30. Considered by the investigator or any sub-investigator as inappropriate for this study for any reason, e.g., (a) deemed unable to meet specific protocol requirements, such as scheduled visits; (b) deemed unable to administer or tolerate need for chronic injections; (c) presence of any other conditions (e.g., geographic, social) actual or anticipated, that the investigator feels would restrict or limit the patient's participation for the duration of the study; (d) part of a vulnerable population such as the institutionalized.
31. Members of the clinical site study team and/or his/her immediate family unless prior approval granted by the sponsor.

### Prohibited Medications and Procedures

The following medications and procedures are prohibited during the DBTP, ETP, OLTP, and the first 4 weeks after the last dose in the follow-up period:
Within 24 hours prior to each clinic visit when blood is drawn, the patient should not consume any alcohol. Patients should also refrain from strenuous exercise within 24 hours prior to each clinic visit when blood is drawn, if possible
Patients should be asked to withhold cholinesterase inhibitors for at least 10 hours prior to each visit during the screening period, DBTP, ETP, and OLTP, to be resumed after visit assessments are complete.
Any other complement inhibitor therapy (Anti-C5 therapy should not be started until 12 weeks after the last dose in the follow-up period. Consideration should be given to the projected long-acting effects of Cemdisiran.)
Anti-CD20 medications, e.g., rituximab
Anti-FcRn medications
Androgen deprivation therapy.
Testing with complement assays during the DBTP and ETP other than those specified in the protocol

### Permitted Medications and Procedures

The following medications and procedures are permitted, under the following conditions:
Throughout the DBTP, patients should maintain stable background treatments for gMG such as cholinesterase inhibitor, systemic corticosteroids, or other immunosuppressive treatments with no adjustments (i.e., initiation, discontinuation, or alteration of the dosing regimen). Allowed exceptions include the use of rescue therapy, the withholding of cholinesterase inhibitors for at least 10 hours prior to a study visit, and changes that are clinically indicated for safety or tolerability.
During the first 12 weeks of the ETP, it should be attempted to maintain a stable regimen of cholinesterase inhibitor, systemic corticosteroid therapy and other immunosuppressive therapies. If possible, adjustments to these medications should be reserved until after week 36.
Rescue therapy is allowed and includes: PLEX, IVIG, other comparable TA procedures or techniques, any medication required to treat an AE, including systemic corticosteroids
Meningococcal vaccination
Oral antibiotic prophylaxis for meningococcal infection
COVID-19 vaccination and influenza vaccination
Vaccination against Streptococcus pneumoniae and Haemophilus influenza type B
Oral contraceptives and hormone-replacement therapy may continue
Acetaminophen/paracetamol, aspirin, or ibuprofen
Vitamin and electrolyte supplementation.
Any other medication required for the treatment of patient's background medical conditions

### Objectives

A primary objective of the study is to evaluate the effect of Pozelimab+Cemdisiran on daily functioning that is impacted by signs and symptoms in patients with symptomatic gMG.

Secondary objectives of the study include: (a) to evaluate the effect of Pozelimab+Cemdisiran (i.e., combination) and Cemdisiran monotherapy on: clinician-assessed signs of MG and muscle strength; daily functioning that is impacted by signs and symptoms in patients with symptomatic gMG (Cemdisiran monotherapy only); proportion of patients with improvements in daily function that is impacted by signs and symptoms of MG; proportion of patients that have improvements in clinician-assessed signs of MG and muscle strength; health related quality of life; proportion of patients with minimal MG symptoms; patient- and clinician-reported signs and symptoms of MG; (b) to evaluate the safety and tolerability of Pozelimab+Cemdisiran and Cemdisiran monotherapy; (c) to assess the concentration of total Pozelimab in serum; (d) to assess the concentration of total C5 in plasma; (e) to assess the concentrations of Cemdisiran and its metabolites in plasma; (f) to assess the immunogenicity of Pozelimab; (g) to assess the immunogenicity of Cemdisiran; and (h) to study the effect of Pozelimab+Cemdisiran and Cemdisiran monotherapy on complement activation.

### Endpoints

A primary endpoint of the study is change in MG-ADL total score from baseline to week 24. A secondary endpoint is the change from baseline in QMG score at week 24. Other secondary endpoints include:
Proportion of patients responding on the MG-ADL, with a responder defined as one with a ≥3-point improvement from baseline to week 24
Proportion of patients responding on the QMG, with a responder defined as one with a ≥5-point improvement from baseline to week 24.
Proportion of patients with consistent response on the MG-ADL, defined as patients with at least a 2-point MG-ADL improvement on 2 or more consecutive assessments spanning 4 or more weeks during the DBTP
Proportion of patients with minimal symptom expression (MSE, defined by a score of 0 to 1 on the MG-ADL) at week 24.
Change from baseline in the MGC total score at week 24.
Change from baseline in MG-QOL15r total score at week 24.
Proportion of patients with improvement point thresholds of ≥2, 4, 5, 6, 7, 8, 9, or 10 on MG-ADL at week 24
Proportion of patients with improvement point thresholds of ≥3, 4, 6, 7, 8, 9, or 10 on QMG at week 24
Incidence and severity of treatment-emergent adverse events (TEAEs), serious adverse events (SAEs), and adverse events of special interest (AESIs) in patients treated with Pozelimab+Cemdisiran or placebo through week 24
Concentrations of total Pozelimab in serum at nominal time-points
Concentrations of Cemdisiran and its metabolites in plasma at nominal time points
Incidence of treatment-emergent anti-drug antibodies (ADAs) to Pozelimab after repeated doses over time
Incidence of treatment-emergent ADAs to Cemdisiran after repeated doses over time
Change and percent change in CH50 over time

### Assessments

Myasthenia Gravis-Activities of Daily Living (MG-ADL): a primary endpoint is the change from baseline in MG-ADL total score at week 24. The MG-ADL is a clinician-administered, patient-reported outcome measure assessing daily functions impacted by gMG symptoms (Wolfe et al., Neurology, 52 (7): 1487-1489 (1999)). The MG-ADL includes 8 items: 2 items assessing ocular, 3 assessing bulbar, 1 assessing respiratory, and 2 assessing extremity dysfunction. Each item is graded from 0 to 3. The total MG-ADL score ranges from 0 to 24 points, with higher scores indicating greater functional impairment and disability. The recall period is the previous 7 days. The MG-ADL will be conducted by the same trained and certified clinical evaluator at each assessment throughout the study, as best as possible. A 2-point reduction in MG-ADL total score is considered a minimally clinically meaningful improvement (Muppidi, Ann N Y Acad Sci, 1274:114-119 (2012)). An alternative threshold for clinically meaningful reduction in MG-ADL with intervention is 3 points, which was used as a responder definition in the REGAIN trial (Howard et al., Lancet Neurol, 16 (12): 976-986 (2017)).

Minimal symptom expression (MSE) is defined as achievement of an MG-ADL total score of 0 or 1 (range 0 to 24 points). The MSE has been used in a phase 3 study with eculizumab as an assessment of therapeutic effectiveness in this patient population (Howard et al., JAMA Neurol, 77 (5): 582-592 (2020); Vissing et al., J Neurol, 267 (7): 1991-2001 (2020)).

Quantitative Myasthenia Gravis (QMG) is a 13-item performance test that assesses the patient's current signs and symptoms severity of MG through the evaluation of muscle weakness and body function impairment (Soliris® Eculizumab (Prescribing information)) (Barohn et al, Ann N Y Acad Sci, 841:769-772 (1998)). It is administered by a trained and certified evaluator/clinician with the same evaluator at each assessment throughout the study, as best as possible, and evaluates muscle strength, function, and fatiguability based on quantitative testing of specific muscle groups: ocular (2 items), facial (1 item), bulbar (2 items), gross motor (6 items), axial (1 item), and respiratory (1 item). Each item is graded on a scale of 0 (none) to 3 (severe), and QMG total scores range from 0 to 39, with higher scores representing greater impairment. The QMG score is a validated objective measure recommended for use in studies for the treatment of MG by the Task Force of the Medical Scientific Advisory Board of the MGFA (Bedlack et al., Neurology, 64 (11): 1968-1970 (2005); Jaretzki et al., Ann Thorac Surg, 70 (1): 327-334 (2000)). A 3-point reduction from the baseline total score indicates clinically significant improvement (Katzberg et al., Muscle Nerve, 49 (5): 661-665 (2014)).

Myasthenia Gravis Composite (MGC) consists of 10 items that measure symptoms and signs of MG, including ptosis (upward gaze), double-vision on lateral gaze, eye closure, talking ability, chewing ability, swallowing ability, breathing ability, neck flexion or extension, shoulder abduction, and hip flexion (Burns et al, Muscle Nerve, 38 (6): 1553-1562 (2008)). Each item has 4 response options based on level of impact (normal, mild, moderate, or severe) and is rated by a trained and certified evaluator/clinician with the same evaluator at each assessment throughout the study, as best as possible, based on examination of the patient as well as patient-reported history. Each response option of each item is weighted. MGC score ranges from 0 to 50, with higher score indicating higher impairment. The MGC has been used in intervention studies in the MG population, and the test items are frequently abnormal in patients with active disease, respond to clinical changes and have been chosen to be meaningful to the physician and the patient. A 3-point reduction from the baseline total score indicates clinically significant improvement (Burns et al., Neurology, 74 (18): 1434-1440 (2010)).

Myasthenia Gravis Quality of Life (MG-QOL15r) is a self-administered, validated patient-reported outcome (PRO) instrument consisting of 15 items that measures the patient's quality of life as related to MG, including physical, social, and psychological domains (Howard et al., Lancet Neurol, 16 (12): 976-986 (2017)). Each item is scored on a 3-response option scale ranging from “not at all” to “very much”, and the total score ranges from 0 to 30 points. A higher score represents greater impairment.

Quality of Life in Neurological Disorders: Fatigue (Neuro-QoL-Fatigue) is a self-administered questionnaire consisting of 8 items (Tran et al., Muscle Nerve, 58 (2): 197-203 (2018)). It is a short form subset of the larger Neuro-QoL instrument family initially developed for adult patients with disorders such as multiple sclerosis, Parkinson's disease, stroke, epilepsy, and muscular dystrophy (Cook et al., Qual Life Res, 24 (3): 575-589 (2015)). The Neuro-QoL is recommended as a supplemental assessment in MG by the National Institute of Neurological Disorders and Stroke (NINDS, 2020), and the Neuro-QoL-Fatigue 8-item short form is validated for MG (Tran et al., Muscle Nerve, 58 (2): 197-203 (2018)). Each of the 8 items in the subscale is scored on a 5-point Likert scale ranging from 1=“never” to 5=“always”, with a recall period of 7 days. The total score ranges from 8 to 40, with a higher score indicating greater fatigue.

EuroQol-5 Dimensions-5 Levels (EQ-5D-5L) is a self-administered, generic, standardized health status measure consisting of 6 questions (EuroQol, EQ-5D-3L User Guide, v.6.0, EuroQol Research Foundation (2018)). The EQ-5D-5L descriptive system assesses 5 dimensions of health: mobility, self-care, usual activities, pain/discomfort, and anxiety/depression. Each dimension is rated on a 5-level scale: no problems, slight problems, moderate problems, severe problems, and ‘unable to’. Recall period is “Today”. The EQ visual analog scale component is a vertical, visual analog scale used by patients to rate their health today, and ranges from 0 (worst health you can imagine) to 100 (best health you can imagine).

Patient and Clinician Global Measures are separate, single item measures developed for this study. The answers on the Patient Global Impression of Severity (PGIS) and Patient Global Impression of Change (PGIC) items serve as “anchors” to help interpret the mean change in disease-specific PRO measures over time and to establish thresholds for a meaningful change. The items about daily activity impairment and overall severity will serve as anchors for the MG-ADL.

Patient Global Impression of Severity (PGIS)—MG Functioning is a single-item, self-administered PRO question in which the patient is asked to pick the response that best describes the severity of their impairment when doing their usual daily activities over the past 7 days. At study visits, patients will be asked to select their response on a 4-point Likert scale ranging from none, mild, moderate, and severe.

Patient Global Impression of Severity (PGIS)—MG Disease is a single-item, self-administered PRO question in which the patient is asked to pick the response that best describes the severity of their MG disease over the past 7 days. At study visits, patients will be asked to select their response on a 4-point Likert scale ranging from none, mild, moderate, and severe.

Patient Global Impression of Change (PGIC)—MG Functioning is a single-item, self-administered PRO question in which the patient is asked to choose the response that best describes the overall change of their impairment when doing their usual daily activities since having started the study treatment. At study visits, patients will be asked to rate the change on a 7-point Likert scale ranging from ‘a lot better’ to ‘no change’ to ‘a lot worse’.

Patient Global Impression of Change (PGIC)—MG Disease is a single-item, self-administered PRO question in which the patient is asked to choose the response that best describes the overall change of their MG disease since having started the study treatment. At study visits, patients will be asked to rate the change on a 7-point Likert scale ranging from ‘a lot better’ to ‘no change’ to ‘a lot worse’.

Clinician Global Impression of Severity (CGIS) MG Body Function is a single-item, clinician-rated measure to assess the clinician's impression of the severity of the weakness of the patient's body and muscle functions. Clinicians will rate their assessment on a 4-point Likert scale from none, mild, moderate, and severe.

Clinician Global Impression of Severity (CGIS) MG Disease is a single-item, clinician-rated measure to assess the clinician's impression of the severity of the patient's MG disease. Clinicians will rate their assessment on a 4-point Likert scale from none, mild, moderate, and severe.

Clinician Global Impression of Severity (CGIS) Ocular/Bulbar Weakness is a single-item clinician-rated measure to assess the clinician's impression of severity of the weakness of the patient's ocular and bulbar function due to MG. Clinicians will rate their assessment on a 4-point Likert scale is from none, mild, moderate, and severe.

Clinician Global Impression of Severity (CGIS) Generalized Weakness is a single-item clinician-rated measure to assess the clinician's overall impression of the severity of the patient's generalized muscle weakness (excluding ocular and bulbar functions) due to MG. Clinicians will rate their assessment on a 4-point Likert scale is from none, mild, moderate, and severe.

Clinician Global Impression of Change (CGIC) Body Function is a single-item clinician-rated measure to assess the clinician's impression of the overall change in the weakness of their patient's body and muscle functions since the patient started taking the study medication. Clinicians will rate their assessment on a 7-point Likert scale from ‘a lot better’ to ‘no change’ to ‘a lot worse’.

Clinician Global Impression of Change (CGIC) MG Disease is a single-item clinician-rated measure to assess the clinician's impression of the overall change in the patient's MG disease since the patient started taking the study medication. Clinicians will rate their assessment on a 7-point Likert scale from ‘a lot better’ to ‘no change’ to ‘a lot worse’.

Clinician Global Impression of Change (CGIC) Ocular/Bulbar Weakness is a single-item clinician-rated measure to assess the clinician's impression of the overall change in the weakness of the patient's ocular/bulbar function due to MG since the patient started taking the study medication. Clinicians will rate their assessment on a 7-point Likert scale from ‘a lot better’ to ‘no change’ to ‘a lot worse’.

Clinician Global Impression of Change (CGIC) Generalized Weakness is a single-item clinician-rated measure to assess the clinician's impression of the overall change in the patient's generalized muscle weakness (excluding ocular and bulbar functions) due to MG since the patient started taking the study medication. Clinicians will rate their assessment on a 7-point Likert scale from ‘a lot better’ to ‘no change’ to ‘a lot worse’.

Medication Usage. Corticosteroids are standard treatments for symptoms of MG. A change in the dosage of corticosteroids may be an indicator of disease worsening (an increase from baseline) or improvement (a decrease from baseline). Corticosteroid dose will be recorded at each visit.

Modified MGFA Postintervention Status (MG-PIS): Change in Status is performed by the clinician to assess the clinical state of patients following the initiation of treatment for MG (Jaretzki et al., Ann Thorac Surg, 70 (1): 327-334 (2000)). The MG-PIS defines criteria for complete stable remission, pharmacologic remission, and minimal manifestations (with subcategories). It also assesses change in status based on pre-specified definitions for 5 categories: ‘improved’, ‘unchanged’, ‘worse’, ‘exacerbation’, and ‘died of MG’.

### Results

Cemdisiran, an siRNA that reduces circulating levels of complement factor 5 (C5), as monotherapy in this trial (NIMBLE), was associated with an average of 74% inhibition of complement activity. The trial also assessed a combination of Cemdisiran and Pozelimab, an anti-C5 antibody; this combination (“cemdi-poze”), which resulted in nearly 99% inhibition of complement activity, also met the primary and key secondary endpoints, though Cemdisiran monotherapy was numerically better across these endpoints.

The results of this clinical trial demonstrated that, in myasthenia gravis, robust efficacy can be achieved without complete complement blockade, whereas in other diseases such as paroxysmal nocturnal hemoglobinuria (PNH), complete inhibition is likely to be necessary. This was surprising, in part, because, in a Phase 2 trial using Zilucoplan (a C5 inhibitor) to treat MG that evaluated 2 doses, the higher dose of 0.3 mg/kg demonstrated greater CH50 reduction (95%) than the lower dose of 0.1 mg/kg (82%), and also showed slightly better efficacy as measured by change in MG-ADL score from baseline (−2.3 vs −2.2). See Table 1-8. This suggested that, to treat MG, one would need to maximize complement inhibition in order to achieve maximal efficacy. See Howard et al., Clinical Effects of the Self-administered Subcutaneous Complement Inhibitor Zilucoplan in Patients With Moderate to Severe Generalized Myasthenia Gravis: Results of a Phase 2 Randomized, Double-Blind, Placebo-Controlled, Multicenter Clinical Trial. JAMA Neurol. 2020 May 1; 77 (5): 582-592). This aspect of Cemdisiran monotherapy is favorable since achieving a high degree of efficacy in treating MG without full complement inhibition may lead to a favorable safety profile due to the lowered risk of infection.

NIMBLE evaluated adults with symptomatic gMG who had antibodies to the acetylcholine receptor (anti-AChR) and may be receiving stable doses of standard of care immunosuppressants based on the investigator's discretion. Patients were randomized to receive subcutaneous administrations of: Cemdisiran (600 mg) every 12 weeks, cemdi-poze (Cemdisiran 200 mg and pozelimab 200 mg) every 4 weeks, or placebo every 4 weeks. The primary endpoint assessed total score changes from baseline to week 24 in the Myasthenia Gravis Activities of Daily Living (MG-ADL) total score, a patient-reported questionnaire that measures daily functions impacted by gMG, such as talking, eating, breathing, vision and mobility. The key secondary endpoint assessed total score changes from baseline in the Quantitative Myasthenia Gravis (QMG) total score, a physician-administered assessment evaluating vision, speaking/swallowing, breathing and limb function.

Cemdisiran monotherapy, dosed subcutaneously every three months, met the primary and key secondary endpoints, showing a 2.3-point placebo-adjusted improvement in Myasthenia Gravis Activities of Daily Living (MG-ADL) total score.

Both Cemdisiran and cemdi-poze demonstrated improvements in activities of daily functioning at week 24, with Cemdisiran showing numerically better results across all gMG-specific outcomes. In the MG-ADL and QMG, greater reductions in total scores indicated greater improvement in disease symptoms and better treatment effect. These results are summarized in Table 1-5.

TABLE 1-5
Summary of Trial Results

| | Cemdisiran | Cemdi-Poze | Placebo |
|---|---|---|---|
| | (n = 64) | (n = 67) | (n = 59) |
| Patients completing | 100% | 96% | 90% |
| trial treatment | | | |

Primary endpoint: Change from baseline in MG-ADL total score

| | | | |
|---|---|---|---|
| Placebo-adjusted | −2.30 (p = 0.0005) | −1.74 (p = 0.0086) | |
| treatment difference | | | |
| Mean change from | −4.52 | −3.96 | −2.22 |
| baseline^{†} | | | |

Key secondary endpoint: Change from baseline in QMG total score

| | | | |
|---|---|---|---|
| Placebo-adjusted | −2.77 (p = 0.0015) | −1.86 (p = 0.0348) | |
| treatment difference | | | |
| Mean change from | −4.24 | −3.32 | −1.46 |
| baseline^{†} | | | |

Other secondary endpoints

| | | | |
|---|---|---|---|
| MG-ADL: Patients | 76.6% | 65.7% | 44.1% |
| achieving ≥3-point | Relative risk: | Relative risk: | |
| reduction | 1.84 (p = 0.0001)^{†} | 1.51 (p = 0.0135)^{†} | |
| QMG: Patients | 48.4% | 35.8% | 19% |
| achieving ≥5-point | Relative risk: | Relative risk: | |
| reduction | 2.67 (p = 0.0006)^{†} | 1.93 (p = 0.0367)^{†} | |

^{†}Means and relative risks were adjusted per the amount immunosuppressant treatment and the Myasthenia Gravis Foundation of America Clinical Classification determined at screening. Means were also adjusted for baseline MG-ADL or QMG score.

There were no meningococcal infections in any patient. There were no treatment discontinuations due to adverse events through week 24 in the Cemdisiran arm.

Across all arms, treatment-emergent adverse events (TEAEs) occurred in 69% of patients treated with Cemdisiran, 81% with cemdi-poze, and 77% with placebo. The most common TEAEs observed in ≥5% of patients receiving Cemdisiran, cemdi-poze or placebo were: worsening of MG (1%, 5%, 17%), upper respiratory tract infection (12%, 8%, 11%), urinary tract infection (5%, 6%, 3%), nasopharyngitis (5%, 3%, 4%), headache (5%, 11%, 10%), rash (5%, 3%, 1%), injection site reaction (4%, 8%, 1%), diarrhea (3%, 14%, 7%), arthralgia (1%, 6%, 1%), pain in extremity (1%, 5%, 1%), cough (1%, 5%, 1%), and pruritus (0%, 5%, 0%). Serious TEAEs occurred in 3% of patients treated with Cemdisiran, 9% with cemdi-poze and 14% with placebo. There were no deaths during the 24-week placebo-controlled portion of the trial. During the extension period, one death due to pneumonia occurred in the Cemdisiran arm, and one death due to septic shock occurred in the combination arm; both deaths occurred in patients who were on concomitant immunosuppressive therapies.

All endpoints in approved product labels were significant (MG-ADL, QMG, 3 pt MG-ADL responders, 5 pt QMG Responders) for both combination therapy (Pozelimab+Cemdisiran SC Q4W) and Cemdisiran monotherapy (SC Q12W) versus placebo; though Cemdisiran monotherapy was evaluated after the combination therapy in our hierarchy, the monotherapy was numerically better than the combination and all three approved C5 inhibitors (e.g., Soliris [Eculizumab], Ultomiris [Ravulizumab], ZILBRYSQ [Zilucoplan]) in the key primary endpoint (Change from baseline in MG-ADL total score). Remarkably, robust efficacy was achieved with Cemdisiran monotherapy with incomplete complement inhibition (CH50).

### Baseline

Baseline disease characteristics and demographics of the arms are summarized in Table 1-5 and Table 1-6. Baseline disease characteristics and demographics were generally balanced between groups.

[symbol]

BLE 1-5
BaselineDiseaseChA-MyastheniaGravis Foundationof America [symbol]
-ElectronicData Capture
TT-Primary
ImmunosuppressiveTherapy
ITT = modifiedintentionto treat

[symbol]

| | Placebo | | Combination | | Cemdisiran | | Pozelimab | | Total | |
|---|---|---|---|---|---|---|---|---|---|---|
| | (N = 59) | | (N = 67) | | (N = 64) | | (N = 49) | | N = 239 | |
| Duration of MG at screening (years) | | | | | | | | | | |
| n | 59 | | 67 | | 64 | | 49 | | 239 | |
| Mean (SD) | 8.16 | (9.21) | 6.10 | (7.14) | 8.10 | (8.69) | 8.50 | (7.56) | 7.64 | (8.20) |
| Median | 4.93 | | 4.08 | | 3.26 | | 7.68 | | 4.88 | |
| Q1:Q3 | 1.4:11.1 | | 1.8:8.1 | | 1.8:12.4 | | 2.6:11.3 | | 1.8:10.8 | |
| Min:Max | 0.1:42.0 | | 0.1:38.2 | | 0.1:47.2 | | 0.2:34.8 | | 0.1:47.2 | |
| Prior history of Myasthenic Crisis/Impending Crisis, n (%) | | | | | | | | | | |
| None | 39 | (66.1%) | 47 | (70.1%) | 39 | (60.9%) | 30 | (61.2%) | 155 | (64.9%) |
| 1 | 12 | (20.3%) | 13 | (19.4%) | 14 | (21.9%) | 14 | (28.6%) | 53 | (22.2%) |
| 2 | 5 | (8.5%) | 4 | (6.0%) | 7 | (10.9%) | 3 | (6.1%) | 19 | (7.9%) |
| 3 | 1 | (1.7%) | 3 | (4.5%) | 2 | (9.1%) | 2 | (4.1%) | 8 | (3.3%) |
| More than 3 | 2 | (3.4%) | 0 | | 2 | (3.1%) | 0 | | 4 | (1.7%) |
| MGFA Classification at Screening per EDC, n (%) | | | | | | | | | | |
| IIA | 12 | (20.3%) | 18 | (26.9%) | 12 | (18.8%) | 12 | (24.5%) | 54 | (22.6%) |
| IIB | 12 | (20.3%) | 10 | (14.9%) | 14 | (21.9%) | 5 | (10.2%) | 41 | (17.2%) |
| IIIA | 18 | (30.5%) | 23 | (34.3%) | 23 | (35.9%) | 16 | (32.7%) | 80 | (33.5%) |
| IIIB | 14 | (23.7%) | 15 | (22.4%) | 13 | (20.3%) | 13 | (26.5%) | 55 | (23.0%) |
| IVA | 3 | (5.1%) | 1 | (1.5%) | 2 | (3.1%) | 3 | (6.1%) | 9 | (3.8%) |
| Treatment with IST on day 1 per EDC, n (%) | | | | | | | | | | |
| All <= 1 Year | 10 | (16.9%) | 8 | (11.9%) | 6 | (9.4%) | 7 | (14.3%) | 31 | (13.0%) |
| Any IST >1 Year | 33 | (55.9%) | 43 | (64.2%) | 43 | (67.2%) | 11 | (63.3%) | 150 | (62.8%) |
| None/Discontinued | 16 | (27.1%) | 16 | (23.9%) | 15 | (23.4%) | 11 | (22.4%) | 58 | (24.3%) |

[symbol]

indicates data missing or illegible when filed

1-6. Baseline Demographics

| | | | | |
|---|---|---|---|---|
| Placebo | Pozelimab | Combination | Cemdisiran | Total |
| (N = 59) | (N = 49) | (N = 67) | (N = 64) | (N = 239) |

Documented prior history of positive nerologic
test for anti-AChR antibodies, n (%)

| Yes | 55 | (93.2%) | 48 | (98.0%) | 63 | (94.0%) | 60 | (93.8%) | 226 | (94.6%) |
|---|---|---|---|---|---|---|---|---|---|---|
| No | 4 | (6.8%) | 1 | (2.0%) | 4 | (6.0%) | 4 | (6.3%) | 13 | (5.4%) |
| Documented prior history of positive nerologic | | | | | | | | | | |
| test for anti-LRP4 antibodies, n (%) | | | | | | | | | | |
| Yes | 9 | (13.6%) | 7 | (14.3%) | 6 | (9.0%) | 5 | (7.8%) | 26 | (10.9%) |
| No | 31 | (86.4%) | 42 | (85.7%) | 61 | (91.0%) | 59 | (92.2%) | 213 | (89.1%) |
| Documented prior history of nerologic test for | | | | | | | | | | |
| anti-AChR antibodies and anti-LRP4 antibodies, n (%) | | | | | | | | | | |
| Both negative | 1 | (1.7%) | 0 | | 0 | | 1 | (1.6%) | 2 | (0.8%) |
| Both positive | 5 | (8.5%) | 6 | (12.2%) | 2 | (3.0%) | 2 | (3.1%) | 15 | (6.3%) |
| Only positive for anti-AChR antibodies | 30 | (84.7%) | 42 | (85.7%) | 61 | (91.0%) | 58 | (90.6%) | 211 | (88.3%) |
| Only positive for anti-LRP4 antibodies | 3 | (5.1%) | 1 | (2.0%) | 4 | (6.0%) | 3 | (4.7%) | 11 | (4.6%) |
| Documented prior history of positive nerologic | | | | | | | | | | |
| test for anti-MuSK antibodies, n (%) | | | | | | | | | | |
| Yes | 0 | | 0 | | 2 | (3.0%) | 0 | | 2 | (0.8%) |
| No | 59 | (100%) | 49 | (100%) | 65 | (97.0%) | 64 | (100%) | 237 | (99.2%) |
| Study Baseline MG-ADL total score | | | | | | | | | | |
| n | 59 | | 49 | | 67 | | 64 | | 239 | |
| Mean (SD) | 9.4 | (2.6) | 9.8 | (3.4) | 9.4 | (2.9) | 8.9 | (2.4) | 9.3 | (2.8) |
| Median | 9.0 | | 9.0 | | 9.0 | | 8.5 | | 9.0 | |
| Q1:Q3 | 7:11 | | 7:12 | | 7:11 | | 7:10 | | 7:11 | |
| Min:Max | 6:17 | | 6:20 | | 2:17 | | 6:17 | | 2:20 | |
| Study Baseline QMG total score | | | | | | | | | | |
| n | 59 | | 49 | | 67 | | 64 | | 239 | |
| Mean (SD) | 15.5 | (6.2) | 16.1 | (6.1) | 15.6 | (5.5) | 15.5 | (4.9) | 15.7 | (5.6) |

| Median | 16.0 | 15.0 | 15.0 | 15.0 | 15.0 |
|---|---|---|---|---|---|
| Q1:Q3 | 11:19 | 12:19 | 12:20 | 12:18 | 12:19 |
| Min:Max | 4:32 | 4:37 | 3:29 | 6:30 | 3:37 |

### Efficacy

Both treatment arms achieved statistical significance for key endpoints with Cemdisiran monotherapy (cemdi) showing numerically better MG-ADL than approved C5 inhibitors. See Table 1-7.

TABLE 1-7
Summary of Endpoints in Treatment Arms vs. Placebo;
Compared to Treatment Effect in other C5 Inhibitors
Cemdisiran monotherapy was numerically better than Combination
(Cemdisiran and Pozelimab); No difference was observed for
Pozelimab monotherapy versus Placebo

| Combination versus Placebo | | | Cemdisiran versus Placebo | | | |
|---|---|---|---|---|---|---|
| Significant improvement (more | | | Substantial improvement (more | | | |
| reduction) observed in primary | | | reduction) observed in primary | | | Treatment effect in |
| and secondary endpoints for | | | and secondary endpoints | | | other C5 |
| combination versus placebo | | | for Cemdisiran versus placebo | | | inhibitors** |
| Primary endpoint: Change from baseline in MG-ADL total score at week 24 | | | | | | Zilucoplan: −2.1 |
| [LS means (SE)] | | | | | | Eculizumab: −1.9 |
| | | | | | | Ravulizumab: −1.6 |
| Combination = −3.96 | Placebo = −2.22 | Trt Diff* = −1.74 | Cemdisiran = −4.52 | Placebo = −2.22 | Trt Diff = −2.30 | |
| (0.45) | (0.49) | (p-value = | (0.45) | (0.49) | (p-value = | |
| Baseline = 9.39 | Baseline = | 0.0086) | Baseline = 8.88 | Baseline = | 0.0005) | |
| | 9.41 | | | 9.41 | | |
| Key secondary endpoint: Change from baseline in QMG total score at week 24 | | | | | | Zilucoplan: −2.9 |
| [LS means (SE)] | | | | | | Eculizumab: −3.0 |
| | | | | | | Ravulizumab: −2.0 |
| Combination = −3.32 | Placebo= −1.46 | Trt Diff = −1.86 | Cemdisiran = −4.24 | Placebo= −1.46 | Trt Diff= −2.77 | |
| (0.58) | (0.66) | (p-value = | (0.57) | (0.66) | (p-value = | |
| Baseline = | Baseline = | 0.0348) | Baseline = | Baseline = | 0.0015) | |
| 15.63 | 15.49 | | 15.53 | 15.49 | | |
| Secondary endpoint: Achievement of 3-point reduction (improvement) in MG- | | | | | | Zilucoplan: 1.50\hat{ } |
| ADL total score from baseline at week 24 [Proportion] | | | | | | Eculizumab:1.50\hat{ } |
| | | | | | | Ravulizumab: 1.60 |
| Combination = | Placebo = | Relative | Cemdisiran = | Placebo= | Relative | |
| 65.7% | 44.1% | risk = 1.51 | 76.6% | 44.1% | risk = 1.84 | |
| | | (p-value = | | | (p-value = | |
| | | 0.0135) | | | 0.0001) | |
| Secondary endpoint: Achievement of 5-point reduction (improvement) in | | | | | | Zilucoplan: 1.80\hat{ } |
| QMG total score from baseline at week 24 (Proportion) | | | | | | Eculizumab: 2.37\hat{ } |
| | | | | | | Ravulizumab: 2.70 |
| Combination = | Placebo = | Relative | Cemdisiran = | Placebo= | Relative | |
| 35.8% | 19% | risk = 1.93 | 48.4% | 19% | risk = 2.67 | |
| | | (p-value = | | | (p-value = | |
| | | 0.0367) | | | 0.0006) | |

Zilucoplan: SC QD; Eculizumab: IV Q2 W; Ravulizumab: IV Q8 W
*Trt Diff = Treatment Difference
\hat{ }relative risk were calculated by using percents shown on the plot in the publication

Cemdisiran monotherapy (Cemdisiran) achieved the greatest MG-ADL response compared to the three approved C5 inhibitors. See Table 1-8.

TABLE 1-8
Comparison between MG-ADL Scores in Treatment Arms and that of Other
C5 Inhibitors

| | | | | | | | Percent of | | | | | |
|---|---|---|---|---|---|---|---|---|---|---|---|---|
| | | | | | | | Improvement | | | | | |
| | | | | LS* Mean of Change | | | from | | Absolute | | % | |
| | | Baseline | | from Baseline | | | Baselinee | | Score | | Responders** | |
| Treatment | Endpoint | Active | Placebo | Active | Placebo | Difference | Active | Placebo | Active | Placebo | Active | Placebo |
| Cemdisiran | MG-ADL | 8.9 | 9.4 | −4.52 | −2.22 | −2.30 | 50.8% | 23.6% | 4.53 | 6.68 | 76.6% | 44.1% |
| | at Week | | | | | | | | | | | |
| | 24 | | | | | | | | | | | |
| Poze + Cemdi | MG-ADL | 9.4 | 9.4 | −3.96 | −2.22 | −1.74 | 42.1% | 23.6% | 5.08 | 6.68 | 65.7% | 44.1% |
| | at Week | | | | | | | | | | | |
| | 24 | | | | | | | | | | | |
| Ravulizumab | MG-ADL | 9.1 | 8.9 | −3.1 | −1.4 | −1.6 | 34.1% | 15.7% | 6.0 | 7.5 | 57% | 34% |
| | at Week | | | | | | | | | | | |
| | 26 | | | | | | | | | | | |
| Eculizumab | MG-ADL | 10.5 | 9.9 | −4.2 | −2.3 | −1.9 | 40.0% | 23.2% | 6.3 | 7.6 | 60% | 40% |
| | at Week | | | | | | | | | | | |
| | 26 | | | | | | | | | | | |
| Zilucoplan | MG-ADL | 10.3 | 10.9 | −4.4 | −2.3 | −2.1 | 42.7% | 21.1% | 5.9 | 8.6 | 73% | 46% |
| | at Week | | | | | | | | | | | |
| | 12 | | | | | | | | | | | |
| Cemdisiran | QMG at | 15.0 | 15.5 | −4.24 | −1.46 | −2.77 | 28.3% | 9.4% | 11.5 | 13.3 | 48.4% | 19% |
| | Week | | | | | | | | | | | |
| | 24 | | | | | | | | | | | |
| Poze + Cemdi | QMG at | 15.6 | 15.5 | −3.32 | −1.46 | −1.86 | 21.3% | 9.4% | 12.0 | 13.3 | 35.8% | 19% |
| | Week | | | | | | | | | | | |
| | 24 | | | | | | | | | | | |
| Ravulizumab | QMG at | 14.8 | 14.5 | −2.8 | −0.8 | −2.0 | 18.9% | 5.5% | 12.0 | 13.7 | 30% | 11% |
| | Week | | | | | | | | | | | |
| | 26 | | | | | | | | | | | |
| Eculizumab | QMG at | 17.3 | 16.9 | −4.6 | −1.6 | −3.0 | 26.6% | 9.5% | 12.7 | 15.3 | 45% | 19% |
| | Week | | | | | | | | | | | |
| | 26 | | | | | | | | | | | |
| Zilucoplan | QMG at | 18.7 | 19.4 | −6.2 | −3.3 | −2.9 | 33.2% | 17.0% | 12.5 | 16.1 | 58% | 33% |
| | Week | | | | | | | | | | | |
| | 12 | | | | | | | | | | | |

*LS: Least Square
**≥3 points improvement for MG-ADL, ≥5 points for QMG
Data Source: USPIs

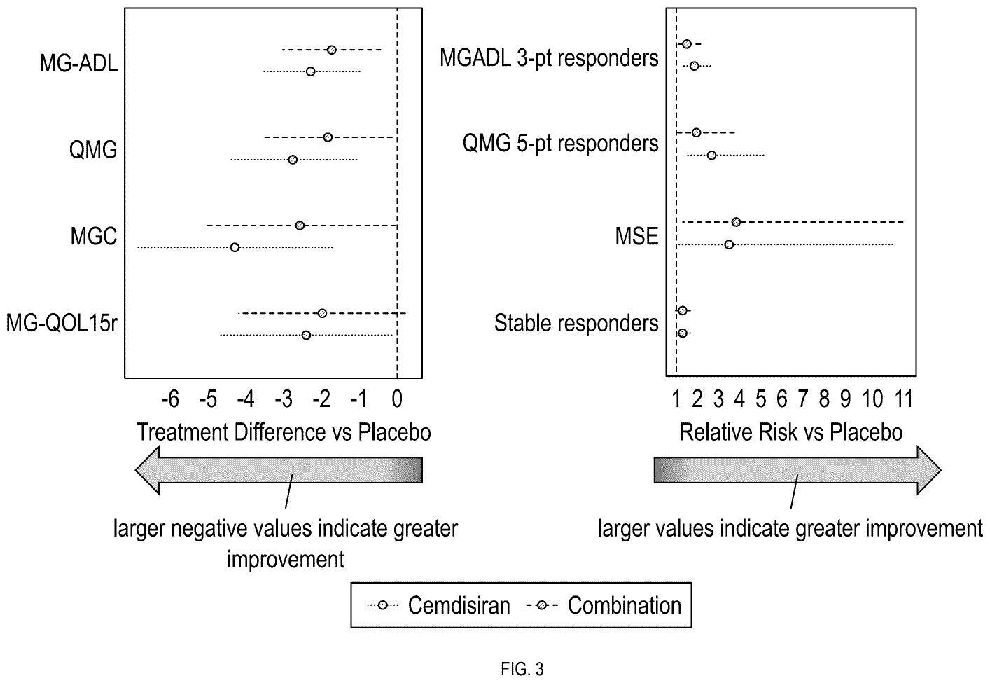

FIG. 3 sets forth the data demonstrating consistent efficacy across all primary and secondary endpoints which favors Cemdisiran monotherapy.

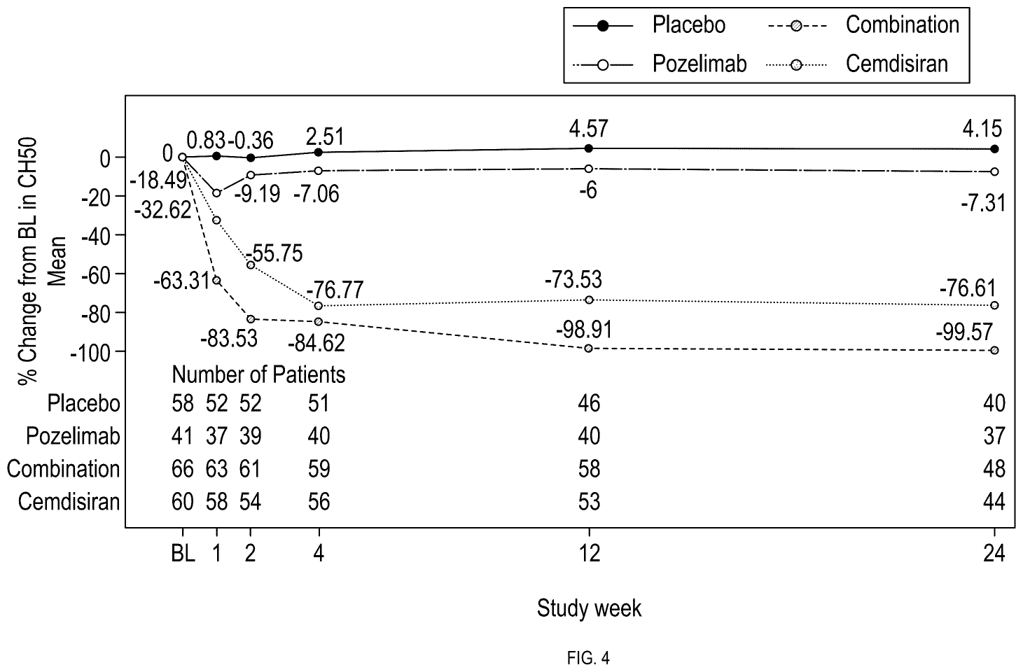

A greater pharmacodynamic CH50 inhibition in the Combination arm did not translate to greater efficacy. See Table 1-9 and 1-9A and FIG. 4.

TABLE 1-9
Observed Mean (SD) of Percent Change from Baseline CH50 (serum)

| Treatment | Baseline | Week 1 | Week 2 | Week 4 | Week 12 | Week 24 |
|---|---|---|---|---|---|---|
| Placebo | 313.16 | 0.83 | −0.36 | 2.51 | 4.57 | 4.15 |
| | U/mL | (13.74) | (12.90) | (12.89) | (16.26) | (25.67) |
| | (59.43) | | | | | |
| Pozelimab | 313.71 | −18.49 | −9.19 | −7.06 | −6.00 | −7.31 |
| | U/mL | (16.13) | (17.82) | (13.34) | (20.46) | (25.12) |
| | (55.08) | | | | | |
| Combination | 314.33 | −63.31 | −83.53 | −84.62 | −98.91 | −99.57 |
| | U/mL | (25.51) | (18.00) | (21.64) | (4.63) | (2.36) |
| | (79.09) | | | | | |
| Cemdisiran | 315.83 | −32.62 | −55.75 | −76.77 | −73.53 | −76.61 |
| | U/mL | (13.32) | (15.22) | (14.62) | (16.24) | (15.86) |
| | (59.87) | | | | | |

TABLE 1-9A
Summary and MMRM Statistical Analysis of the Change of Total Complement
Hemolysis Activity Assay (CH50) During the Double-Blind Treatment
Period (DBTP) - Modified Intent-to-Treat Analysis Set (mITT)

| | Combination | | Cemdisiran | | Pozelimab | | Placebo | |
|---|---|---|---|---|---|---|---|---|
| CH50 (U/mL) | (N = 75) | | (N = 75) | | (N = 49) | | (N = 66) | |
| Study Baseline | | | | | | | | |
| n | 66 | | 60 | | 41 | | 58 | |
| Mean (SD) | 314.33 | (79.09) | 315.83 | (59.87) | 313.71 | (55.08) | 313.16 | (59.43) |
| Median | 315.50 | | 313.50 | | 309.00 | | 314.00 | |
| Q1:Q3 | 257.0:364.0 | | 280.5:347.0 | | 282.0:351.0 | | 266.0:344.0 | |
| Min:Max | 80.0:485.0 | | 194.0:485.0 | | 170.0:445.0 | | 216.0:481.0 | |
| 95% CI for Mean | (294.89, 333.78) | | (300.37, 331.30) | | (296.32, 331.09) | | (297.53, 328.78) | |
| Week 1 | | | | | | | | |
| n | 63 | | 60 | | 37 | | 52 | |
| Mean (SD) | 118.70 | (91.39) | 209.02 | (54.84) | 256.19 | (70.07) | 313.29 | (64.92) |
| Median | 98.00 | | 199.50 | | 263.00 | | 310.00 | |
| Q1:Q3 | 58.0:157.0 | | 174.0:244.0 | | 200.0:305.0 | | 259.0:360.5 | |
| Min:Max | 0.0:481.0 | | 37.0:334.0 | | 123.0:420.0 | | 205.0:474.0 | |
| 95% CI for Mean | (95.68, 141.72) | | (194.85, 223.18) | | (232.83, 279.55) | | (295.22, 331.36) | |
| Change from Study | | | | | | | | |
| Baseline | | | | | | | | |
| n | 63 | | 58 | | 37 | | 52 | |
| Mean (SD) | −196.57 | (86.87) | −102.78 | (48.45) | −58.41 | (58.07) | 1.27 | (40.17) |
| Median | −210.00 | | −98.50 | | −46.00 | | −5.50 | |
| Q1:Q3 | −249.0:−149.0 | | −118.0:−71.0 | | −82.0:−26.0 | | −26.5:24.5 | |
| Min:Max | −402.0:171.0 | | −282.0:−29.0 | | −271.0:40.0 | | −82.0:131.0 | |
| 95% CI for Mean | (−218.45, −174.69) | | (−115.52, −90.04) | | (−77.77, −39.05) | | (−9.91, 12.45) | |
| Week 1 | | | | | | | | |
| Percent Change from | | | | | | | | |
| Study Baseline | | | | | | | | |
| n | 63 | | 58 | | 37 | | 52 | |
| Mean (SD) | −63.31 | (25.51) | −32.62 | (13.32) | −18.49 | (16.13) | 0.83 | (13.74) |
| Median | −67.15 | | −30.53 | | −16.53 | | −2.23 | |
| Q1:Q3 | −76.5:−51.8 | | −39.1:−25.2 | | −27.2:−7.5 | | −8.4:7.0 | |
| Min:Max | −100.0:55.2 | | −85.5:−9.6 | | −60.9:10.5 | | −25.8:49.2 | |
| 95% CI for Mean | (−69.73, −56.88) | | (−36.12, −29.12) | | (−23.87, −13.11) | | (−2.99, 4.66) | |
| Week 2 | | | | | | | | |
| n | 61 | | 56 | | 39 | | 53 | |
| Mean (SD) | 57.20 | (67.44) | 137.20 | (51.66) | 284.05 | (66.97) | 304.40 | (57.05) |
| Median | 35.00 | | 133.50 | | 289.00 | | 304.00 | |
| Q1:Q3 | 5.0:78.0 | | 107.0:161.0 | | 232.0:325.0 | | 261.0:336.0 | |
| Min:Max | 0.0:285.0 | | 25.0:317.0 | | 144.0:482.0 | | 192.0:485.0 | |
| 95% CI for Mean | (39.92, 74.47) | | (123.36, 151.03) | | (262.34, 305.76) | | (288.67, 320.12) | |
| Change from Study | | | | | | | | |
| Baseline | | | | | | | | |
| n | 61 | | 54 | | 39 | | 52 | |
| Mean (SD) | −262.84 | (74.40) | −174.06 | (58.91) | −31.05 | (58.20) | −3.62 | (36.83) |
| Median | −272.00 | | −179.50 | | −26.00 | | −6.50 | |
| Q1:Q3 | −313.0:−221.0 | | −205.0:−147.0 | | −68.0:−5.0 | | −29.5:11.5 | |
| Min:Max | −418.0:−79.0 | | −343.0:5.0 | | −158.0:109.0 | | −89.0:103.0 | |
| 95% CI for Mean | (−281.89, −243.78) | | (−190.13, −157.98) | | (−49.92, −12.19) | | (−13.87, 6.64) | |
| Week 2 | | | | | | | | |
| Percent Change from | | | | | | | | |
| Study Baseline | | | | | | | | |
| n | 61 | | 54 | | 39 | | 52 | |
| Mean (SD) | −83.53 | (18.00) | −55.75 | (15.22) | −9.19 | (17.82) | −0.36 | (12.90) |
| Median | −87.01 | | −57.63 | | −8.38 | | −2.55 | |
| Q1:Q3 | −98.4:−76.7 | | −66.6:−46.8 | | −22.4:−1.6 | | −8.5:4.1 | |
| Min:Max | −100.0:−21.7 | | −90.1:1.8 | | −46.1:29.2 | | −19.8:47.7 | |
| 95% CI for Mean | (−88.14, −78.92) | | (−59.91, −51.60) | | (−14.97, −3.41) | | (−3.96, 3.23) | |
| Week 4 | | | | | | | | |
| n | 59 | | 59 | | 40 | | 52 | |
| Mean (SD) | 52.15 | (78.20) | 74.00 | (50.35) | 290.20 | (57.35) | 311.15 | (61.61) |
| Median | 19.00 | | 69.00 | | 291.50 | | 312.50 | |
| Q1:Q3 | 0.0:81.0 | | 37.0:106.0 | | 255.0:333.0 | | 267.5:356.0 | |
| Min:Max | 0.0:389.0 | | 0.0:219.0 | | 155.0:398.0 | | 170.0:460.0 | |
| 95% CI for Mean | (31.77, 72.53) | | (60.88, 87.12) | | (271.86, 308.54) | | (294.00, 328.30) | |
| Change from Study | | | | | | | | |
| Baseline | | | | | | | | |
| n | 59 | | 56 | | 40 | | 51 | |
| Mean (SD) | −262.44 | (91.45) | −240.13 | (56.30) | −24.08 | (43.45) | 6.73 | (38.16) |
| Median | −269.00 | | −237.50 | | −15.00 | | 1.00 | |
| Q1:Q3 | −334.0:−216.0 | | −272.5:−199.0 | | −44.5:2.5 | | −18.0:30.0 | |
| Min:Max | −447.0:25.0 | | −420.0:−131.0 | | −140.0:47.0 | | −87.0:122.0 | |
| 95% CI for Mean | (−286.27, −238.61) | | (−255.20, −225.05) | | (−37.97, −10.18) | | (−4.01, 17.46) | |
| Week 4 | | | | | | | | |
| Percent Change from | | | | | | | | |
| Study Baseline | | | | | | | | |
| n | 59 | | 56 | | 40 | | 51 | |
| Mean (SD) | −84.62 | (21.64) | −76.77 | (14.62) | −7.06 | (13.34) | 2.51 | (12.89) |
| Median | −94.57 | | −77.48 | | −5.33 | | 0.42 | |
| Q1:Q3 | −100.0:−76.3 | | −87.9:−65.8 | | −12.8:1.0 | | −5.6:10.6 | |
| Min:Max | −100.0:6.9 | | −100.0:−38.9 | | −39.2:19.3 | | −25.9:37.4 | |
| 95% CI for Mean | (−90.26, −78.98) | | (−80.69, −72.86) | | (−11.32, −2.79) | | (−1.11, 6.14) | |
| Week 12 | | | | | | | | |
| n | 58 | | 56 | | 40 | | 47 | |
| Mean (SD) | 4.26 | (18.82) | 81.96 | (55.56) | 294.25 | (84.11) | 318.79 | (69.91) |
| Median | 0.00 | | 80.50 | | 291.00 | | 317.00 | |
| Q1:Q3 | 0.0:0.0 | | 39.0:118.0 | | 252.0:346.0 | | 262.0:361.0 | |
| Min:Max | 0.0:103.0 | | 2.0:228.0 | | 1.0:485.0 | | 207.0:485.0 | |
| 95% CI for Mean | (−0.69, 9.21) | | (67.09, 96.84) | | (267.35, 321.15) | | (298.26, 339.31) | |
| Change from Study | | | | | | | | |
| Baseline | | | | | | | | |
| n | 58 | | 53 | | 40 | | 46 | |
| Mean (SD) | −311.45 | (80.49) | −225.04 | (55.99) | −17.98 | (61.70) | 12.93 | (50.65) |
| Median | −313.00 | | −225.00 | | −15.50 | | 11.00 | |
| Q1:Q3 | −359.0:−256.0 | | −258.0:−184.0 | | −37.0:10.5 | | −23.0:38.0 | |
| Min:Max | −485.0:−80.0 | | −343.0:−112.0 | | −277.0:105.0 | | −90.0:155.0 | |
| 95% CI for Mean | (−332.61, −290.28) | | (−240.47, −209.61) | | (−37.71, 1.76) | | (−2.11, 27.98) | |
| Week 12 | | | | | | | | |
| Percent Change from | | | | | | | | |
| Study Baseline | | | | | | | | |
| n | 58 | | 53 | | 40 | | 46 | |
| Mean (SD) | −98.91 | (4.63) | −73.53 | (16.24) | −6.00 | (20.46) | 4.57 | (16.26) |
| Median | −100.00 | | −75.16 | | −5.07 | | 3.30 | |
| Q1:Q3 | −100.0:−100.0 | | −88.3:−64.5 | | −11.9:3.3 | | −7.1:11.2 | |
| Min:Max | −100.0:−73.3 | | −99.0:−32.9 | | −99.6:27.6 | | −22.2:47.0 | |
| 95% CI for Mean | (−100.13, −97.70) | | (−78.01, −69.06) | | (−12.55, 0.54) | | (−0.26, 9.39) | |
| Week 24 | | | | | | | | |
| n | 48 | | 46 | | 37 | | 41 | |
| Mean (SD) | 1.85 | (10.48) | 71.72 | (53.68) | 286.73 | (91.24) | 320.44 | (88.23) |
| Median | 0.00 | | 71.00 | | 300.00 | | 324.00 | |
| Q1:Q3 | 0.0:0.0 | | 17.0:112.0 | | 244.0:341.0 | | 268.0:358.0 | |
| Min:Max | 0.0:71.0 | | 0.0:197.0 | | 0.0:433.0 | | 0.0:485.0 | |
| 95% CI for Mean | (−1.19, 4.90) | | (55.78, 87.66) | | (256.31, 317.15) | | (292.59, 348.29) | |
| Change from Study | | | | | | | | |
| Baseline | | | | | | | | |
| n | 48 | | 44 | | 37 | | 40 | |
| Mean (SD) | −301.77 | (74.32) | −230.66 | (52.92) | −23.65 | (79.75) | 8.95 | (83.41) |
| Median | −311.50 | | −223.50 | | −3.00 | | 7.50 | |
| Q1:Q3 | −355.5:−256.5 | | −273.5:−192.5 | | −39.0:16.0 | | −18.5:39.5 | |
| Min:Max | −471.0:−80.0 | | −330.0:−140.0 | | −346.0:108.0 | | −336.0:169.0 | |
| 95% CI for Mean | (−323.35, −280.19) | | (−246.75, −214.57) | | (−50.24, 2.94) | | (−17.73, 35.63) | |
| Week 24 | | | | | | | | |
| Percent Change from | | | | | | | | |
| Study Baseline | | | | | | | | |
| n | 48 | | 44 | | 37 | | 40 | |
| Mean (SD) | −99.57 | (2.36) | −76.61 | (15.86) | −7.31 | (25.12) | 4.15 | (25.67) |

| Median | −100 | −77.29 | −1.34 | 2.07 |
|---|---|---|---|---|
| Q1:Q3 | −100.0:−100.0 | −89.5:−65.8 | −12.9:5.8 | −5.5:17.2 |
| Min:Max | −100.0:−84.2 | −100.0:−42.0 | −100.0:35.0 | −100.0:57.9 |
| 95% CI for Mean | (−100.25, −98.88) | (−81.43, −71.79) | (−15.68, 1.06) | (−4.06, 12.36) |

Max = maximum;
Min = minimum;
Q1 = first quartile;
Q3 = third quartile;
SD = standard deviation

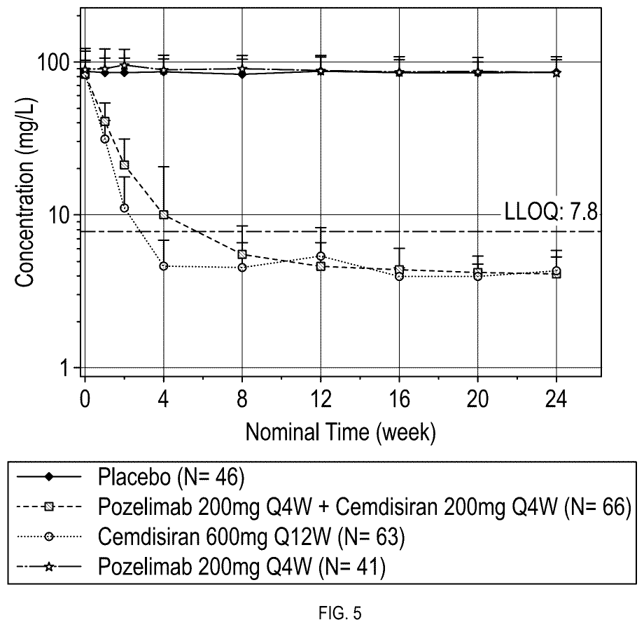

A decrease in total C5 concentration in plasma to below LOQ by week 4 in the Cemdisiran arm vs. week 8 in the combination arm was observed. The delay in attainment of LOQ in the combination arm was because of slow clearance of the C5:Pozelimab complex. Concentrations of Total C5 were maintained below LOQ through week 24 in both arms. See Table 1-10 and FIG. 5.

TABLE 1-10
Summary of Percent Change from Baseline in
Total C5 in plasma by Nominal Time and Treatment
Group in Participants with gMG (TTAS)

| | | | | | | | | Pozelimab |
|---|---|---|---|---|---|---|---|---|
| Sampling | | Placebo | Combination | | Cemdisiran | | | (N = |
| Time Post | | (N = 46) | (N = 66) | | (N = 63) | | | 41) |
| First Dose | | Mean | | Mean | | Mean | | Mean |
| (week) | n | (SD) | n | (SD) | n | (SD) | n | (SD) |
| 1 | 44 | −1.20 | 66 | −50.6 | 61 | −61.7 | 41 | 0.170 |
| | | (17.0) | | (15.0) | | (10.5) | | (13.6) |
| 2 | 45 | −2.02 | 64 | −74.5 | 61 | −88.2 | 40 | 6.34 |
| | | (14.6) | | (12.3) | | (9.08) | | (18.9) |
| 4 | 45 | 0.740 | 65 | −90.0 | 59 | −98.6 | 40 | −1.23 |
| | | (15.7) | | (14.4) | | (4.07) | | (16.3) |
| 8 | 45 | −3.07 | 65 | −97.1 | 60 | −98.8 | 39 | 0.938 |
| | | (21.5) | | (5.47) | | (3.40) | | (15.9) |
| 12 | 45 | 1.51 | 60 | −98.8 | 59 | −97.6 | 40 | −1.04 |
| | | (20.9) | | (3.53) | | (5.07) | | (25.6) |
| 16 | 44 | 0.713 | 57 | −99.1 | 55 | −99.8 | 39 | −1.45 |
| | | (17.5) | | (3.02) | | (1.23) | | (26.7) |
| 20 | 45 | 0.198 | 57 | −99.4 | 55 | −99.8 | 40 | 0.256 |
| | | (21.0) | | (2.38) | | (1.53) | | (24.4) |
| 24 | 42 | 1.74 | 53 | −99.6 | 51 | −99.1 | 40 | −1.97 |
| | | (19.2) | | (1.83) | | (3.20) | | (31.3) |

n = Number of participants contributing to the statistical results;
TTAS = Total target analysis set (participants received at least one dose of active drug and with at least one post-dose C5 measurement);
SD = Standard deviation
Note:
Below LLOQ value is set to 0.

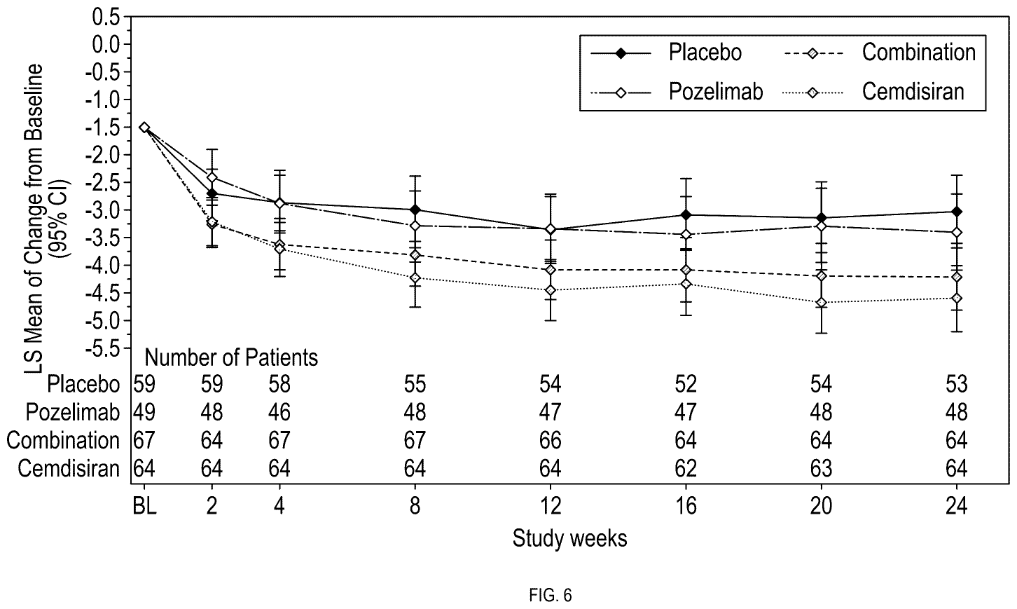

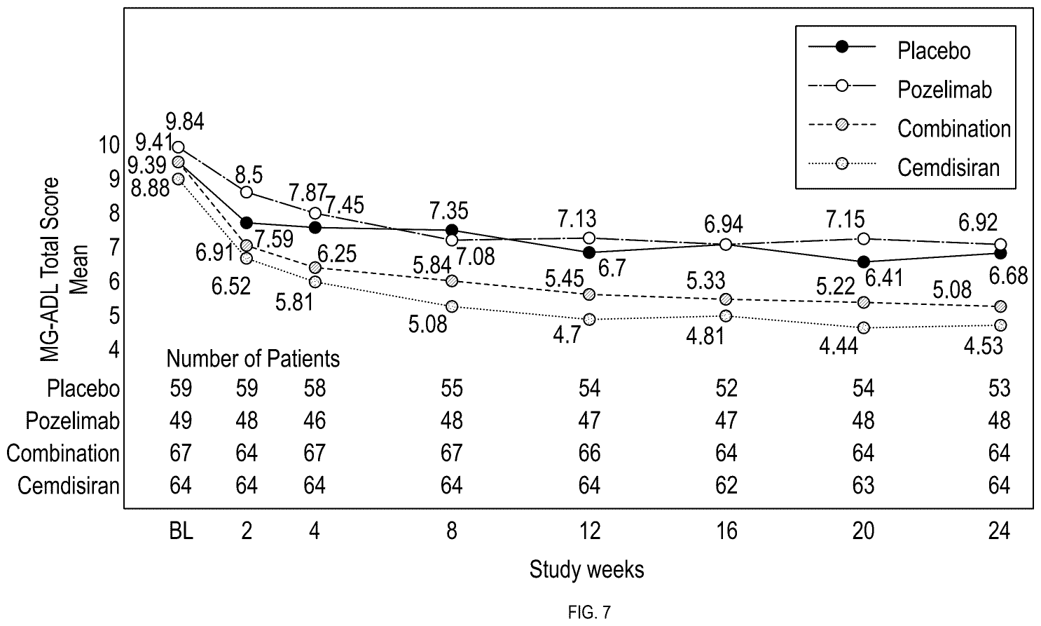

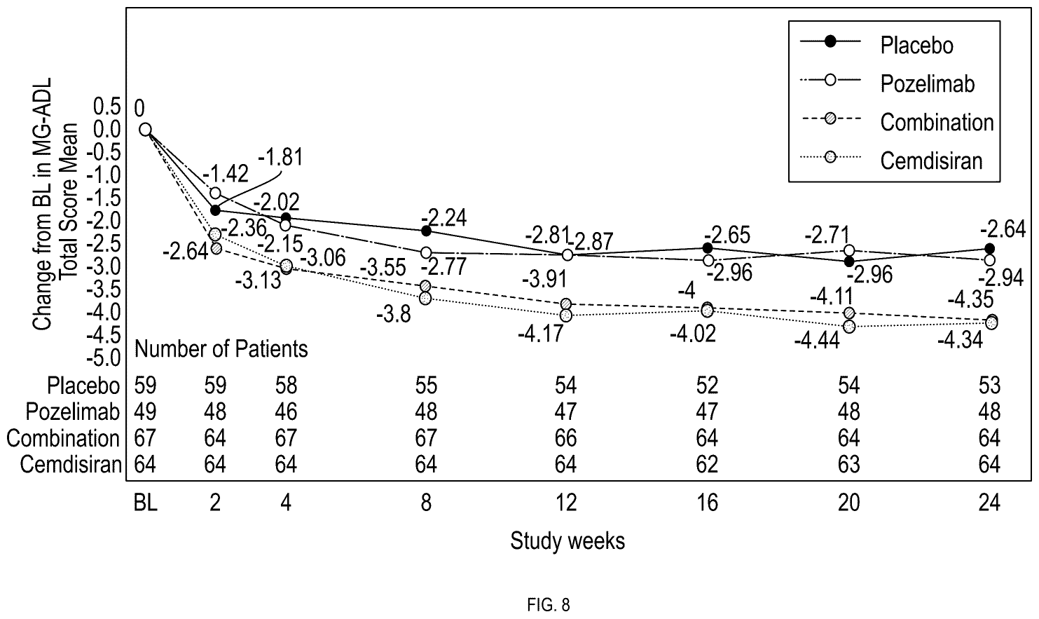

Cemdisiran monotherapy (Cemdisiran) was numerically better than the combination of Pozelimab and Cemdisiran (Combination) with respect to primary endpoint: least square mean of change from baseline in MG-ADL (Myasthenia Gravis-Activities of Daily Living) total score. See summaries of the least square mean change from baseline (Table 1-11; FIG. 6); mean absolute value over time (Table 1-12; FIG. 7) and observed mean change from baseline (Table 1-13; FIG. 8) in MG-ADL score. Tables 1-13 (A to H) contains the summary and MMRM statistical analysis of the change of MG-ADL total score during the double-blind treatment period (DBTP) modified intent-to-treat analysis set (mITT).

TABLE 1-11
Least Square Mean (SE) of Change from Baseline in MG-
ADL Total Score & LS Mean Difference vs Placebo (SE)

| | | Least Square Mean (SE) of Change from Baseline in | | | | | | | |
|---|---|---|---|---|---|---|---|---|---|
| | Mean | MG-ADL Total Score | | | | | | | LS Mean Difference |
| | (SD) at | Week | Week | Week | Week | Week | Week | Week | vs Placebo (SE) |
| Treatment | Baseline | 2 | 4 | 8 | 12 | 16 | 20 | 24 | Week 24 |
| Placebo | 9.41 | −1.75 | −1.98 | −2.15 | −2.69 | −2.27 | −2.38 | −2.22 | |
| | (2.59) | (0.34) | (0.38) | (0.43) | (0.44) | (0.48) | (0.48) | (0.49) | |
| Pozelimab | 9.84 | −1.31 | −1.98 | −2.59 | −2.66 | −2.80 | −2.59 | −2.77 | −0.55 |
| | (3.39) | (0.38) | (0.42) | (0.47) | (0.48) | (0.51) | (0.50) | (0.52) | (0.71); |
| | | | | | | | | | p-value = 0.4375 |
| Combination | 9.39 | −2.56 | −3.08 | −3.38 | −3.76 | −3.75 | −3.91 | −3.96 | −1.74 |
| | (2.90) | (0.32) | (0.36) | (0.41) | (0.40) | (0.44) | (0.43) | (0.45) | (0.66); |
| | | | | | | | | | p-value = 0.0086 |
| Cemdisiran | 8.88 | −2.50 | −3.22 | −3.96 | −4.31 | −4.14 | −4.59 | −4.52 | −2.30 |
| | (2.37) | (0.33) | (0.37) | (0.41) | (0.41) | (0.44) | (0.43) | (0.45) | (0.66); |
| | | | | | | | | | p-value = 0.0005 |

Note:
The number of subjects per arm is based on the raw data prior to addressing intercurrent events.

TABLE 1-12
Mean (SD) Absolute Values of MG-ADL Total Score

| Treatment | Baseline | Week 2 | Week 4 | Week 8 | Week 12 | Week 16 | Week 20 | Week 24 |
|---|---|---|---|---|---|---|---|---|
| Placebo | 9.41(2.59) | 7.59(3.08) | 7.45(3.48) | 7.35(3.45) | 6.70(3.24) | 6.94(3.79) | 6.41(3.57) | 6.68(3.47) |
| Pozelimab | 9.84(3.39) | 8.50(3.53) | 7.87(3.90) | 7.08(4.41) | 7.13(4.39) | 6.94(4.74) | 7.15(4.67) | 6.92(4.85) |
| Combination | 9.39(2.90) | 6.91(3.54) | 6.25(3.53) | 5.84(3.55) | 5.45(4.04) | 5.33(3.77) | 5.22(4.15) | 5.08(4.01) |
| Cemdisiran | 8.88(2.37) | 6.52(3.43) | 5.81(3.51) | 5.08(3.73) | 4.70(3.63) | 4.81(3.87) | 4.44(3.68) | 4.53(3.83) |

TABLE 1-13
Observed Mean (SD) Change from Baseline in MG-ADL Total Score

| Treatment | Week 2 | Week 4 | Week 8 | Week 12 | Week 16 | Week 20 | Week 24 |
|---|---|---|---|---|---|---|---|
| Placebo | −1.81(2.27) | −2.02(2.55) | −2.24(2.84) | −2.87(2.91) | −2.65(3.16) | −2.96(2.85) | −2.64(2.99) |
| Pozelimab | −1.42(2.71) | −2.15(3.13) | −2.77(3.68) | −2.81(3.33) | −2.96(3.82) | −2.71(3.59) | −2.94(3.99) |
| Combination | −2.64(3.04) | −3.13(3.45) | −3.55(3.57) | −3.91(3.72) | −4.00(3.4) | −4.11(3.68) | −4.25(3.46) |
| Cemdisiran | −2.36(2.7) | −3.06(2.98) | −3.80(3.16) | −4.17(3.12) | −4.02(3.34) | −4.44(3.14) | −4.34(3.31) |

TABLE 1-13A
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Study Baseline | | | | | | | | |
| n | 67 | | 64 | | 49 | | 59 | |
| Mean (SD) | 9.39 | (2.90) | 8.88 | (2.37) | 9.84 | (3.39) | 9.41 | (2.59) |

| Median | 9.00 | | 8.50 | | 9.00 | | 9.00 | |
|---|---|---|---|---|---|---|---|---|
| Q1:Q3 | 7.0:11.0 | | 7.0:10.0 | | 7.0:12.0 | | 7.0:11.0 | |
| Min:Max | 2.0:17.0 | | 6.0:17.0 | | 6.0:20.0 | | 6.0:17.0 | |
| 95% CI for Mean | (8.68, 10.10) | | (8.28, 9.47) | | (8.86, 10.81) | | (8.73, 10.08) | |
| Week 2 | | | | | | | | |
| n | 64 | | 64 | | 48 | | 59 | |
| Mean (SD) | 6.91 | (3.54) | 6.52 | (3.43) | 8.50 | (3.53) | 7.59 | (3.08) |

| Median | 6.50 | 6.50 | 8.00 | 8.00 |
|---|---|---|---|---|
| Q1:Q3 | 5.0:9.0 | 4.5:9.0 | 7.0:10.0 | 6.0:10.0 |
| Min:Max | 0.0:16.0 | 0.0:16.0 | 3.0:21.0 | 1.0:16.0 |
| 95% CI for Mean | (6.02, 7.79) | (5.66, 7.37) | (7.47, 9.53) | (6.79, 8.40) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13B
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 2 | | | | | | | | |
| Change from Study | | | | | | | | |
| Baseline | | | | | | | | |
| n | 64 | | 64 | | 48 | | 59 | |
| Mean (SD) | −2.64 | (3.04) | −2.36 | (2.70) | −1.42 | (2.71) | −1.81 | (2.27) |
| Median | −3.00 | | −2.00 | | −1.00 | | −2.00 | |
| Q1:Q3 | −4.0:−0.5 | | −3.0:−0.5 | | −3.0:0.0 | | −3.0:0.0 | |
| Min:Max | −10.0:5.0 | | −11.0:3.0 | | −14.0:3.0 | | −9.0:2.0 | |
| 95% CI for Mean | (−3.40, −1.88) | | (−3.03, −1.68) | | (−2.20, −0.63) | | (−2.41, −1.22) | |
| LS Mean (SE) | −2.56 | (0.32) | −2.50 | (0.33) | −1.31 | (0.38) | −1.75 | (0.34) |
| 95% CI | (−3.19, −1.92) | | (−3.14, −1.85) | | (−2.05, −0.57) | | (−2.42, −1.09) | |
| Week 4 | | | | | | | | |
| n | 67 | | 64 | | 46 | | 58 | |
| Mean (SD) | 6.25 | (3.53) | 5.81 | (3.51) | 7.87 | (3.90) | 7.45 | (3.48) |

| Median | 6.00 | 5.50 | 8.00 | 8.00 |
|---|---|---|---|---|
| Q1:Q3 | 4.0:9.0 | 3.0:8.0 | 5.0:10.0 | 4.0:10.0 |
| Min:Max | 0.0:13.0 | 0.0:15.0 | 1.0:21.0 | 1.0:15.0 |
| 95% CI for Mean | (5.39, 7.11) | (4.94, 6.69) | (6.71, 9.03) | (6.53, 8.36) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13C
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 4 | | | | | | | | |
| Change from Study | | | | | | | | |
| Baseline | | | | | | | | |
| n | 67 | | 64 | | 46 | | 58 | |
| Mean (SD) | −3.13 | (3.45) | −3.06 | (2.98) | −2.15 | (3.13) | −2.02 | (2.55) |
| Median | −3.00 | | −3.00 | | −2.00 | | −1.00 | |
| Q1:Q3 | −5.0:−1.0 | | −4.0:−1.0 | | −3.0:0.0 | | −4.0:0.0 | |
| Min:Max | −13.0:3.0 | | −13.0:2.0 | | −13.0:4.0 | | −9.0:3.0 | |
| 95% CI for Mean | (−3.98, −2.29) | | (−3.81, −2.32) | | (−3.08, −1.22) | | (−2.69, −1.35) | |
| LS Mean (SE) | −3.08 | (0.36) | −3.22 | (0.37) | −1.98 | (0.42) | −1.98 | (0.38) |
| 95% CI | (−3.79, −2.38) | | (−3.94, −2.50) | | (−2.81, −1.15) | | (−2.73, −1.23) | |
| Week 8 | | | | | | | | |
| n | 67 | | 64 | | 48 | | 55 | |
| Mean (SD) | 5.84 | (3.55) | 5.08 | (3.73) | 7.08 | (4.41) | 7.35 | (3.45) |

| Median | 6.00 | 4.50 | 6.00 | 7.00 |
|---|---|---|---|---|
| Q1:Q3 | 2.0:8.0 | 2.0:7.5 | 4.0:10.0 | 5.0:10.0 |
| Min:Max | 0.0:14.0 | 0.0:17.0 | 0.0:21.0 | 1.0:14.0 |
| 95% CI for Mean | (4.97, 6.70) | (4.15, 6.01) | (5.80, 8.36) | (6.41, 8.28) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13D
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 8 | | | | | | | | |
| Change from Study | | | | | | | | |
| Baseline | | | | | | | | |
| n | 67 | | 64 | | 48 | | 55 | |
| Mean (SD) | −3.55 | (3.57) | −3.80 | (3.16) | −2.77 | (3.68) | −2.24 | (2.84) |
| Median | −4.00 | | −4.00 | | −2.00 | | −2.00 | |
| Q1:Q3 | −6.0:−1.0 | | −6.0:−1.0 | | −4.5:−0.5 | | −4.0:0.0 | |
| Min:Max | −14.0:4.0 | | −12.0:2.0 | | −16.0:4.0 | | −9.0:3.0 | |
| 95% CI for Mean | (−4.42, −2.68) | | (−4.59, −3.01) | | (−3.84, −1.70) | | (−3.00, −1.47) | |
| LS Mean (SE) | −3.38 | (0.41) | −3.96 | (0.41) | −2.59 | (0.47) | −2.15 | (0.43) |
| 95% CI | (−4.18, −2.59) | | (−4.77, −3.16) | | (−3.52, −1.66) | | (−3.01, −1.30) | |
| Week 12 | | | | | | | | |
| n | 66 | | 64 | | 47 | | 54 | |
| Mean (SD) | 5.45 | (4.04) | 4.70 | (3.63) | 7.13 | (4.39) | 6.70 | (3.24) |

| | | | | |
|---|---|---|---|---|
| Median | 5.00 | 4.00 | 6.00 | 7.00 |
| Q1:Q3 | 2.0:7.0 | 2.0:7.0 | 5.0:10.0 | 5.0:9.0 |
| Min:Max | 0.0:17.0 | 0.0:16.0 | 0.0:21.0 | 0.0:14.0 |
| 95% CI for Mean | (4.46, 6.45) | (3.80, 5.61) | (5.84, 8.42) | (5.82, 7.59) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13E
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 12 | | | | | | | | |
| Change from Study | | | | | | | | |
| Baseline | | | | | | | | |
| n | 66 | | 64 | | 47 | | 54 | |
| Mean (SD) | −3.91 | (3.72) | −4.17 | (3.12) | −2.81 | (3.33) | −2.87 | (2.91) |
| Median | −4.00 | | −4.00 | | −2.00 | | −3.00 | |
| Q1:Q3 | −6.0:−1.0 | | −6.0:−2.0 | | −6.0:0.0 | | −5.0:−1.0 | |
| Min:Max | −14.0:4.0 | | −13.0:3.0 | | −11.0:3.0 | | −12.0:3.0 | |
| 95% CI for Mean | (−4.82, −2.99) | | (−4.95, −3.39) | | (−3.79, −1.83) | | (−3.66, −2.08) | |
| LS Mean (SE) | −3.76 | (0.40) | −4.31 | (0.41) | −2.66 | (0.48) | −2.69 | (0.44) |
| 95% CI | (−4.55, −2.97) | | (−5.12, −3.50) | | (−3.59, −1.72) | | (−3.55, −1.82) | |
| Week 16 | | | | | | | | |
| n | 64 | | 62 | | 47 | | 52 | |
| Mean (SD) | 5.33 | (3.77) | 4.81 | (3.87) | 6.94 | (4.74) | 6.94 | (3.79) |

| Median | 5.00 | 4.00 | 6.00 | 7.00 |
|---|---|---|---|---|
| Q1:Q3 | 2.0:7.0 | 2.0:8.0 | 3.0:10.0 | 4.5:9.0 |
| Min:Max | 0.0:18.0 | 0.0:17.0 | 0.0:21.0 | 0.0:17.0 |
| 95% CI for Mean | (4.39, 6.27) | (3.82, 5.79) | (5.54, 8.33) | (5.89, 8.00) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13F
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 16 | | | | | | | | |
| Change from Study | | | | | | | | |
| Baseline | | | | | | | | |
| n | 64 | | 62 | | 47 | | 52 | |
| Mean (SD) | −4.00 | (3.40) | −4.02 | (3.34) | −2.96 | (3.82) | −2.65 | (3.16) |
| Median | −4.00 | | −4.50 | | −2.00 | | −3.00 | |
| Q1:Q3 | −6.0:−2.0 | | −6.0:−2.0 | | −5.0:0.0 | | −5.0:−0.5 | |
| Min:Max | −13.0:4.0 | | −13.0:3.0 | | −16.0:5.0 | | −11.0:3.0 | |
| 95% CI for Mean | (−4.85, −3.15) | | (−4.86, −3.17) | | (−4.08, −1.84) | | (−3.53, −1.78) | |
| LS Mean (SE) | −3.75 | (0.44) | −4.14 | (0.44) | −2.80 | (0.51) | −2.27 | (0.48) |
| 95% CI | (−4.62, −2.89) | | (−5.00, −3.27) | | (−3.79, −1.81) | | (−3.20, −1.34) | |
| Week 20 | | | | | | | | |
| n | 64 | | 63 | | 48 | | 54 | |
| Mean (SD) | 5.22 | (4.15) | 4.44 | (3.68) | 7.15 | (4.67) | 6.41 | (3.57) |

| Median | 5.00 | 3.00 | 6.00 | 7.00 |
|---|---|---|---|---|
| Q1:Q3 | 2.0:7.5 | 1.0:7.0 | 4.5:9.0 | 4.0:8.0 |
| Min:Max | 0.0:18.0 | 0.0:16.0 | 0.0:20.0 | 0.0:14.0 |
| 95% CI for Mean | (4.18, 6.26) | (3.52, 5.37) | (5.79, 8.50) | (5.43, 7.38) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13G
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 20 | | | | | | | | |
| Change from Study | | | | | | | | |
| Baseline | | | | | | | | |
| n | 64 | | 63 | | 48 | | 54 | |
| Mean (SD) | −4.11 | (3.68) | −4.44 | (3.14) | −2.71 | (3.59) | −2.96 | (2.85) |
| Median | −4.50 | | −5.00 | | −2.00 | | −3.00 | |
| Q1:Q3 | −6.0:−2.5 | | −6.0:−2.0 | | −4.0:0.0 | | −5.0:−1.0 | |
| Min:Max | −14.0:4.0 | | −13.0:2.0 | | −13.0:5.0 | | −10.0:3.0 | |
| 95% CI for Mean | (−5.03, −3.19) | | (−5.24, −3.65) | | (−3.75, −1.67) | | (−3.74, −2.18) | |
| LS Mean (SE) | −3.91 | (0.43) | −4.59 | (0.43) | −2.59 | (0.50) | −2.38 | (0.48) |
| 95% CI | (−4.76, −3.07) | | (−5.43, −3.75) | | (−3.58, −1.61) | | (−3.31, −1.44) | |
| Week 24 | | | | | | | | |
| n | 64 | | 64 | | 48 | | 53 | |
| Mean (SD) | 5.08 | (4.01) | 4.53 | (3.83) | 6.92 | (4.85) | 6.68 | (3.47) |

| Median | 4.00 | 3.00 | 6.00 | 8.00 |
|---|---|---|---|---|
| Q1:Q3 | 2.0:8.0 | 2.0:7.5 | 3.5:10.0 | 4.0:9.0 |
| Min:Max | 0.0:15.0 | 0.0:16.0 | 0.0:21.0 | 0.0:14.0 |
| 95% CI for Mean | (4.08, 6.08) | (3.58, 5.49) | (5.51, 8.33) | (5.72, 7.64) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

TABLE 1-13H
Primary Endpoint: Summary and MMRM Statistical Analysis of the
Change of MG-ADL Total Score During the Double-Blind Treatment
Period (DBTP) Modified Intent-to-Treat Analysis Set (mITT)

| MG-ADL Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 24 | | | | | | | | |
| Change from Study | | | | | | | | |
| Baseline | | | | | | | | |
| n | 64 | | 64 | | 48 | | 53 | |
| Mean (SD) | −4.25 | (3.46) | −4.34 | (3.31) | −2.94 | (3.99) | −2.64 | (2.99) |
| Median | −4.50 | | −5.00 | | −3.00 | | −3.00 | |
| Q1:Q3 | −6.5:−2.0 | | −6.0:−3.0 | | −5.5:−1.0 | | −5.0:0.0 | |
| Min:Max | −14.0:2.0 | | −12.0:4.0 | | −15.0:6.0 | | −10.0:3.0 | |
| 95% CI for Mean | (−5.11, −3.39) | | (−5.17, −3.52) | | (−4.09, −1.78) | | (−3.47, −1.82) | |
| LS Mean (SE) | −3.96 | (0.45) | −4.52 | (0.45) | −2.77 | (0.52) | −2.22 | (0.49) |

| 95% CI | (−4.84, −3.09) | | (−5.40, −3.64) | | (−3.78, −1.76) | | (−3.18, −1.26) |
|---|---|---|---|---|---|---|---|
| Difference:Active | | | | | | | |
| vs. Placebo | | | | | | | |
| LS Mean (SE) | −1.74 | (0.66) | −2.30 | (0.66) | −0.55 | (0.71) | |

| 95% CI | (−3.04, −0.44) | (−3.60, −1.00) | (−1.94, 0.84) |
|---|---|---|---|
| p-value | 0.0086 | 0.0005 | 0.4375 |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-ADL total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-ADL total score is included as a covariate in the model. The descriptive summary statistics are calculated prior to multiple imputation for ICEs.

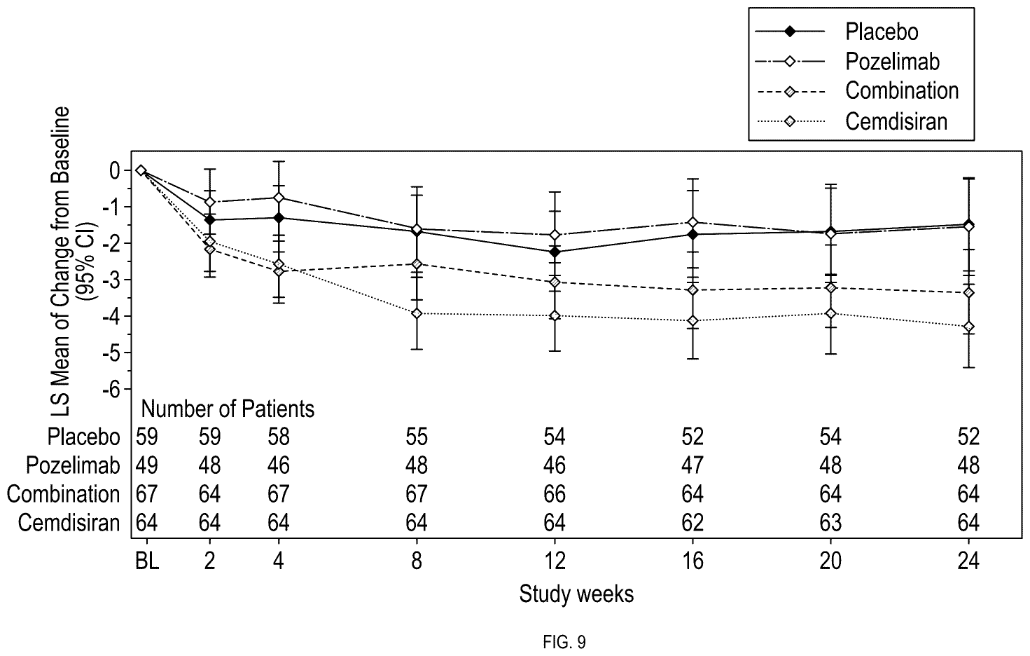

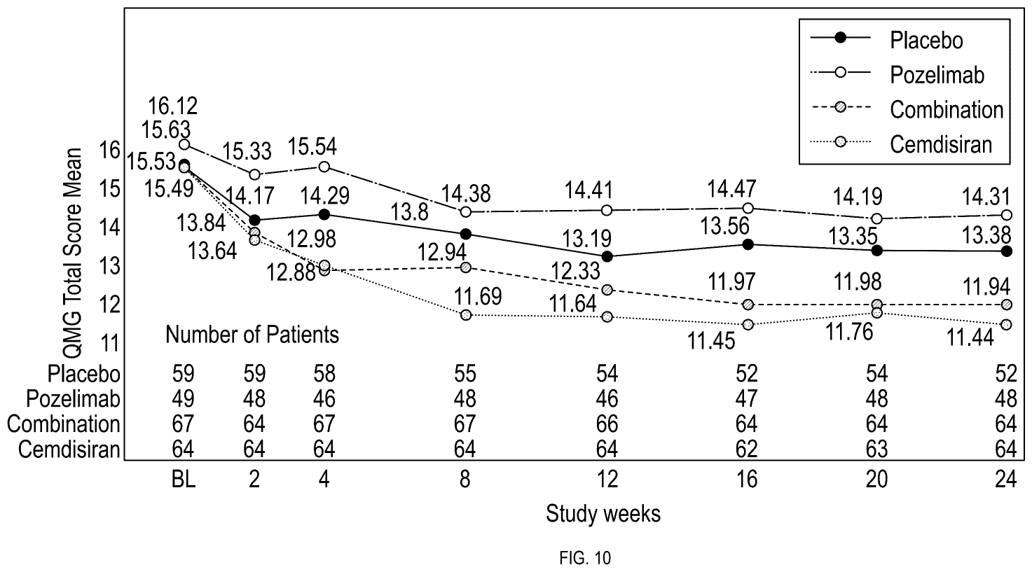

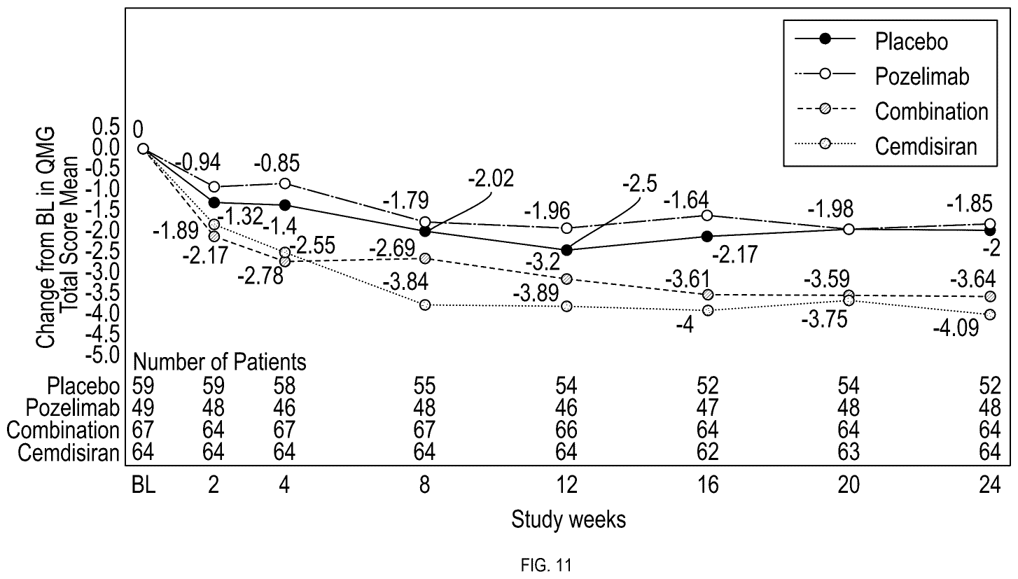

Cemdisiran monotherapy (Cemdisiran) was numerically better than Combination (combination of Pozelimab and Cemdisiran) as shown by the key secondary endpoint: least square mean of change from baseline in QMG (Quantitative Myasthenia Gravis) total score. See summaries of the least square mean change from baseline (Table 1-14; FIG. 9); mean QMG total score over time (Table 1-15; FIG. 10) and observed mean change from baseline (Table 1-16; FIG. 11) in QMG score. Tables 1-16 A-H contain the summary and MMRM statistical analysis of the change of QMG total score during the double-blind treatment period (DBTP) modified intent-to-treat analysis set (mITT)

TABLE 1-14
Least Square Mean (SE) of Change from Baseline in QMG
Total Score & LS Mean Difference vs Placebo (SE)

| | | Least Square Mean (SE) of Change from Baseline in | | | | | | | |
|---|---|---|---|---|---|---|---|---|---|
| | Mean | QMG Total Score | | | | | | | LS Mean Difference |
| | (SD) at | Week | Week | Week | Week | Week | Week | Week | vs Placebo (SE) |
| Treatment | Baseline | 2 | 4 | 8 | 12 | 16 | 20 | 24 | Week 24 |
| Placebo | 15.49 | −1.34 | −1.31 | −1.69 | −2.22 | −1.73 | −1.62 | −1.46 | |
| | (6.21) | (0.41) | (0.46) | (0.54) | (0.56) | (0.60) | (0.64) | (0.66) | |
| Pozelimab | 16.12 | −0.83 | −0.73 | −1.58 | −1.78 | −1.44 | −1.77 | −1.55 | −0.09 (0.93); |
| | (6.14) | (0.45) | (0.51) | (0.60) | (0.61) | (0.62) | (0.67) | (0.67) | p-value = 0.9265 |
| Combination | 15.63 | −2.11 | −2.77 | −2.56 | −3.07 | −3.27 | −3.18 | −3.32 | −1.86 (0.88); |
| | (5.50) | (0.40) | (0.44) | (0.51) | (0.51) | (0.54) | (0.58) | (0.58) | p-value = 0.0348 |
| Cemdisiran | 15.53 | −1.95 | −2.60 | −3.91 | −3.94 | −4.11 | −3.92 | −4.24 | −2.77 (0.87); |
| | (4.94) | (0.39) | (0.44) | (0.51) | (0.52) | (0.53) | (0.57) | (0.57) | p-value = 0.0015 |

Note:
The number of subjects per arm is based on the raw data prior to addressing intercurrent events

TABLE 1-15
Mean (SD) Absolute Values of QMG Total Score

| Treatment | Baseline | Week 2 | Week 4 | Week 8 | Week 12 | Week 16 | Week 20 | Week 24 |
|---|---|---|---|---|---|---|---|---|
| Placebo | 15.49(6.21) | 14.17(6.07) | 14.29(6.70) | 13.80(6.00) | 13.19(6.63) | 13.56(6.87) | 13.35(6.51) | 13.38(6.61) |
| Pozelimab | 16.12(6.14) | 15.33(6.07) | 15.54(6.42) | 14.38(6.53) | 14.41(6.59) | 14.47(7.22) | 14.19(6.75) | 14.31(6.78) |
| Combinatio | 15.63(5.50) | 13.84(4.62) | 12.85(5.12) | 12.94(5.54) | 12.33(5.82) | 11.97(5.20) | 11.98(5.23) | 11.94(5.53) |
| n | | | | | | | | |
| Cemdisiran | 15.53(4.94) | 13.64(5.02) | 12.98(5.39) | 11.69(5.53) | 11.64(5.64) | 11.45(5.41) | 11.76(6.27) | 11.44(5.69) |

TABLE 1-16
Mean (SD) of Change from Baseline in QMG Total Score

| Treatment | Week 2 | Week 4 | Week 8 | Week 12 | Week 16 | Week 20 | Week 24 |
|---|---|---|---|---|---|---|---|
| Placebo | −1.32(3.09) | −1.4(3.69) | −2.02(3.93) | −2.5(4.02) | −2.17(4.27) | −1.98(4.25) | −2.00(4.63) |
| Pozelimab | −0.94(3.44) | −0.85(3.25) | −1.79(3.79) | −1.96(4.05) | −1.64(4.18) | −1.98(4.35) | −1.85(4.24) |
| Combination | −2.17(3.35) | −2.78(3.56) | −2.69(4.27) | −3.2(4.35) | −3.61(3.79) | −3.59(4.32) | −3.64(4.31) |
| Cemdisiran | −1.89(3.36) | −2.55(3.93) | −3.84(4.57) | −3.89(4.13) | −4.00(4.37) | −3.75(4.77) | −4.09(4.85) |

TABLE 1-16A
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Study Baseline | | | | | | | | |
| n | 67 | | 64 | | 49 | | 59 | |
| Mean (SD) | 15.63 | (5.50) | 15.53 | (4.94) | 16.12 | (6.14) | 15.49 | (6.21) |
| Median | 15.00 | | 15.00 | | 15.00 | | 16.00 | |
| Q1:Q3 | 12.0:20.0 | | 12.0:18.0 | | 12.0:19.0 | | 11.0:19.0 | |
| Min:Max | 3.0:29.0 | | 6.0:30.0 | | 4.0:37.0 | | 4.0:32.0 | |
| 95% CI for Mean | (14.28, 16.97) | | (14.30, 16.77) | | (14.36, 17.89) | | (13.87, 17.11) | |
| Week 2 | | | | | | | | |
| n | 64 | | 64 | | 48 | | 59 | |
| Mean (SD) | 13.84 | (4.62) | 13.64 | (5.02) | 15.33 | (6.07) | 14.17 | (6.07) |

| Median | 13.00 | 13.50 | 14.00 | 14.00 |
|---|---|---|---|---|
| Q1:Q3 | 10.0:17.0 | 11.0:16.5 | 11.0:20.0 | 9.0:19.0 |
| Min:Max | 5.0:25.0 | 3.0:30.0 | 1.0:33.0 | 4.0:29.0 |
| 95% CI for Mean | (12.69, 15.00) | (12.39, 14.89) | (13.57, 17.10) | (12.59, 15.75) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16B
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 2 | | | | | | | | |
| Change from Study | | | | | | | | |
| Baseline | | | | | | | | |
| n | 64 | | 64 | | 48 | | 59 | |
| Mean (SD) | −2.17 | (3.35) | −1.89 | (3.36) | −0.94 | (3.44) | −1.32 | (3.09) |
| Median | −2.00 | | −2.00 | | −1.00 | | −1.00 | |
| Q1:Q3 | −4.0:0.0 | | −4.0:0.0 | | −3.0:1.0 | | −3.0:1.0 | |
| Min:Max | −11.0:5.0 | | −14.0:6.0 | | −11.0:6.0 | | −11.0:5.0 | |
| 95% CI for Mean | (−3.01, −1.34) | | (−2.73, −1.05) | | (−1.94, 0.06) | | (−2.13, −0.52) | |
| LS Mean (SE) | −2.11 | (0.40) | −1.95 | (0.39) | −0.83 | (0.45) | −1.34 | (0.41) |
| 95% CI | (−2.88, −1.33) | | (−2.72, −1.18) | | (−1.72, 0.05) | | (−2.15, −0.54) | |
| Week 4 | | | | | | | | |
| n | 67 | | 64 | | 46 | | 58 | |
| Mean (SD) | 12.85 | (5.12) | 12.98 | (5.39) | 15.54 | (6.42) | 14.29 | (6.70) |

| Median | 13.00 | 12.50 | 14.50 | 14.00 |
|---|---|---|---|---|
| Q1:Q3 | 10.0:16.0 | 9.0:17.0 | 11.0:20.0 | 9.0:18.0 |
| Min:Max | 1.0:25.0 | 2.0:28.0 | 2.0:35.0 | 3.0:29.0 |
| 95% CI for Mean | (11.60, 14.10) | (11.64, 14.33) | (13.64, 17.45) | (12.53, 16.06) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16C
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 4 | | | | | | | | |
| Change from Study | | | | | | | | |
| Baseline | | | | | | | | |
| n | 67 | | 64 | | 46 | | 58 | |
| Mean (SD) | −2.78 | (3.56) | −2.55 | (3.93) | −0.85 | (3.25) | −1.40 | (3.69) |
| Median | −3.00 | | −2.00 | | −1.00 | | −1.00 | |
| Q1:Q3 | −5.0:0.0 | | −4.0:0.0 | | −2.0:1.0 | | −3.0:0.0 | |
| Min:Max | −11.0:7.0 | | −16.0:8.0 | | −11.0:9.0 | | −13.0:8.0 | |
| 95% CI for Mean | (−3.65, −1.91) | | (−3.53, −1.57) | | (−1.81, 0.12) | | (−2.37, −0.43) | |
| LS Mean (SE) | −2.77 | (0.44) | −2.60 | (0.44) | −0.73 | (0.51) | −1.31 | (0.46) |
| 95% CI | (−3.63, −1.90) | | (−3.47, −1.73) | | (−1.74, 0.28) | | (−2.22, −0.40) | |
| Week 8 | | | | | | | | |
| n | 67 | | 64 | | 48 | | 55 | |
| Mean (SD) | 12.94 | (5.54) | 11.69 | (5.53) | 14.38 | (6.53) | 13.80 | (6.00) |

| Median | 13.00 | 11.50 | 14.00 | 13.00 |
|---|---|---|---|---|
| Q1:Q3 | 9.0:16.0 | 7.5:14.5 | 9.0:18.0 | 9.0:18.0 |
| Min:Max | 1.0:27.0 | 1.0:32.0 | 3.0:33.0 | 4.0:29.0 |
| 95% CI for Mean | (11.59, 14.29) | (10.31, 13.07) | (12.48, 16.27) | (12.18, 15.42) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16D
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 8 | | | | | | | | |
| Change from Study | | | | | | | | |
| Baseline | | | | | | | | |
| n | 67 | | 64 | | 48 | | 55 | |
| Mean (SD) | −2.69 | (4.27) | −3.84 | (4.57) | −1.79 | (3.79) | −2.02 | (3.93) |
| Median | −3.00 | | −3.00 | | −2.00 | | −1.00 | |
| Q1:Q3 | −6.0:0.0 | | −6.5:−1.0 | | −4.0:0.0 | | −4.0:0.0 | |
| Min:Max | −12.0:7.0 | | −19.0:5.0 | | −12.0:8.0 | | −13.0:4.0 | |
| 95% CI for Mean | (−3.73, −1.65) | | (−4.99, −2.70) | | (−2.89, −0.69) | | (−3.08, −0.96) | |
| LS Mean (SE) | −2.56 | (0.51) | −3.91 | (0.51) | −1.58 | (0.60) | −1.69 | (0.54) |
| 95% CI | (−3.56, −1.56) | | (−4.90, −2.91) | | (−2.75, −0.41) | | (−2.76, −0.63) | |
| Week 12 | | | | | | | | |
| n | 66 | | 64 | | 46 | | 54 | |
| Mean (SD) | 12.33 | (5.82) | 11.64 | (5.64) | 14.41 | (6.59) | 13.19 | (6.63) |

| Median | 12.00 | 11.00 | 13.50 | 13.00 |
|---|---|---|---|---|
| Q1:Q3 | 9.0:17.0 | 7.5:15.0 | 10.0:19.0 | 9.0:17.0 |
| Min:Max | 1.0:25.0 | 1.0:34.0 | 1.0:33.0 | 1.0:30.0 |
| 95% CI for Mean | (10.90, 13.77) | (10.23, 13.05) | (12.46, 16.37) | (11.38, 14.99) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16E
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 12 | | | | | | | | |
| Change from Study | | | | | | | | |
| Baseline | | | | | | | | |
| n | 66 | | 64 | | 46 | | 54 | |
| Mean (SD) | −3.20 | (4.35) | −3.89 | (4.13) | −1.96 | (4.05) | −2.50 | (4.02) |
| Median | −3.00 | | −4.00 | | −2.00 | | −2.00 | |
| Q1:Q3 | −6.0:0.0 | | −6.0:−1.0 | | −4.0:0.0 | | −4.0:0.0 | |
| Min:Max | −13.0:6.0 | | −18.0:4.0 | | −13.0:8.0 | | −14.0:3.0 | |
| 95% CI for Mean | (−4.27, −2.13) | | (−4.92, −2.86) | | (−3.16, −0.75) | | (−3.60, −1.40) | |
| LS Mean (SE) | −3.07 | (0.51) | −3.94 | (0.52) | −1.78 | (0.61) | −2.22 | (0.56) |
| 95% CI | (−4.07, −2.07) | | (−4.95, −2.93) | | (−2.97, −0.60) | | (−3.32, −1.12) | |
| Week 16 | | | | | | | | |
| n | 64 | | 62 | | 47 | | 52 | |
| Mean (SD) | 11.97 | (5.20) | 11.45 | (5.41) | 14.47 | (7.22) | 13.56 | (6.87) |

| Median | 11.50 | 11.00 | 13.00 | 13.00 |
|---|---|---|---|---|
| Q1:Q3 | 9.0:15.0 | 8.0:14.0 | 10.0:19.0 | 10.0:17.5 |
| Min:Max | 2.0:26.0 | 2.0:34.0 | 0.0:36.0 | 2.0:28.0 |
| 95% CI for Mean | (10.67, 13.27) | (10.08, 12.83) | (12.35, 16.59) | (11.65, 15.47) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16F
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 16 | | | | | | | | |
| Change from Study | | | | | | | | |
| Baseline | | | | | | | | |
| n | 64 | | 62 | | 47 | | 52 | |
| Mean (SD) | −3.61 | (3.79) | −4.00 | (4.37) | −1.64 | (4.18) | −2.17 | (4.27) |
| Median | −3.00 | | −4.00 | | −1.00 | | −1.00 | |
| Q1:Q3 | −5.0:−1.5 | | −7.0:−1.0 | | −4.0:1.0 | | −3.5:0.0 | |
| Min:Max | −14.0:5.0 | | −18.0:6.0 | | −14.0:12.0 | | −15.0:4.0 | |
| 95% CI for Mean | (−4.56, −2.66) | | (−5.11, −2.89) | | (−2.87, −0.41) | | (−3.36, −0.98) | |
| LS Mean (SE) | −3.27 | (0.54) | −4.11 | (0.53) | −1.44 | (0.62) | −1.73 | (0.60) |
| 95% CI | (−4.32, −2.22) | | (−5.15, −3.07) | | (−2.66, −0.22) | | (−2.91, −0.55) | |
| Week 20 | | | | | | | | |
| n | 64 | | 63 | | 48 | | 54 | |
| Mean (SD) | 11.98 | (5.23) | 11.76 | (6.27) | 14.19 | (6.75) | 13.35 | (6.51) |

| Median | 11.50 | 11.00 | 13.50 | 13.00 |
|---|---|---|---|---|
| Q1:Q3 | 8.0:16.0 | 7.0:16.0 | 9.0:18.0 | 9.0:17.0 |
| Min:Max | 2.0:23.0 | 2.0:35.0 | 1.0:36.0 | 2.0:30.0 |
| 95% CI for Mean | (10.68, 13.29) | (10.18, 13.34) | (12.23, 16.15) | (11.58, 15.13) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16G
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 20 | | | | | | | | |
| Change from Study | | | | | | | | |
| Baseline | | | | | | | | |
| n | 64 | | 63 | | 48 | | 54 | |
| Mean (SD) | −3.59 | (4.32) | −3.75 | (4.77) | −1.98 | (4.35) | −1.98 | (4.25) |
| Median | −3.50 | | −5.00 | | −2.00 | | −2.00 | |
| Q1:Q3 | −6.0:−1.0 | | −6.0:−1.0 | | −5.0:0.0 | | −5.0:1.0 | |
| Min:Max | −15.0:8.0 | | −19.0:7.0 | | −14.0:13.0 | | −14.0:6.0 | |
| 95% CI for Mean | (−4.67, −2.52) | | (−4.95, −2.55) | | (−3.24, −0.71) | | (−3.14, −0.82) | |
| LS Mean (SE) | −3.18 | (0.58) | −3.92 | (0.57) | −1.77 | (0.67) | −1.62 | (0.64) |
| 95% CI | (−4.31, −2.05) | | (−5.03, −2.81) | | (−3.07, −0.46) | | (−2.87, −0.37) | |
| Week 24 | | | | | | | | |
| n | 64 | | 64 | | 48 | | 52 | |
| Mean (SD) | 11.94 | (5.53) | 11.44 | (5.69) | 14.31 | (6.78) | 13.38 | (6.61) |

| Median | 11.00 | 11.00 | 14.00 | 12.50 |
|---|---|---|---|---|
| Q1:Q3 | 8.0:16.0 | 7.5:15.0 | 9.0:19.0 | 8.5:17.5 |
| Min:Max | 2.0:25.0 | 0.0:31.0 | 0.0:33.0 | 1.0:28.0 |
| 95% CI for Mean | (10.56, 13.32) | (10.02, 12.86) | (12.34, 16.28) | (11.54, 15.23) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

TABLE 1-16H
Key Secondary Endpoint: Summary and MMRM Statistical Analysis
of the Change of QMG Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| QMG Total Score | (N = 67) | | (N = 64) | | (N = 49) | | (N = 59) | |
| Week 24 | | | | | | | | |
| Change from Study | | | | | | | | |
| Baseline | | | | | | | | |
| n | 64 | | 64 | | 48 | | 52 | |
| Mean (SD) | −3.64 | (4.31) | −4.09 | (4.85) | −1.85 | (4.24) | −2.00 | (4.63) |
| Median | −4.00 | | −4.00 | | −2.00 | | −1.00 | |
| Q1:Q3 | −6.0:−1.0 | | −7.0:−1.0 | | −4.0:1.0 | | −5.0:1.0 | |
| Min:Max | −15.0:6.0 | | −18.0:7.0 | | −14.0:9.0 | | −15.0:5.0 | |
| 95% CI for Mean | (−4.72, −2.56) | | (−5.31, −2.88) | | (−3.08, −0.62) | | (−3.29, −0.71) | |
| LS Mean (SE) | −3.32 | (0.58) | −4.24 | (0.57) | −1.55 | (0.67) | −1.46 | (0.66) |

| 95% CI | (−4.46, −2.19) | | (−5.36, −3.11) | | (−2.86, −0.24) | | (−2.75, −0.17) |
|---|---|---|---|---|---|---|---|
| Difference: Active | | | | | | | |
| vs. Placebo | | | | | | | |
| LS Mean (SE) | −1.86 | (0.88) | −2.77 | (0.87) | −0.09 | (0.93) | |

| 95% CI | (−3.59, −0.13) | (−4.49, −1.06) | (−1.91, 1.74) |
|---|---|---|---|
| p-value | 0.0348 | 0.0015 | 0.9265 |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in QMG total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline QMG total score is included as a covariate in the model. The descriptive summary statistics are calculated prior to multiple imputation for ICEs.

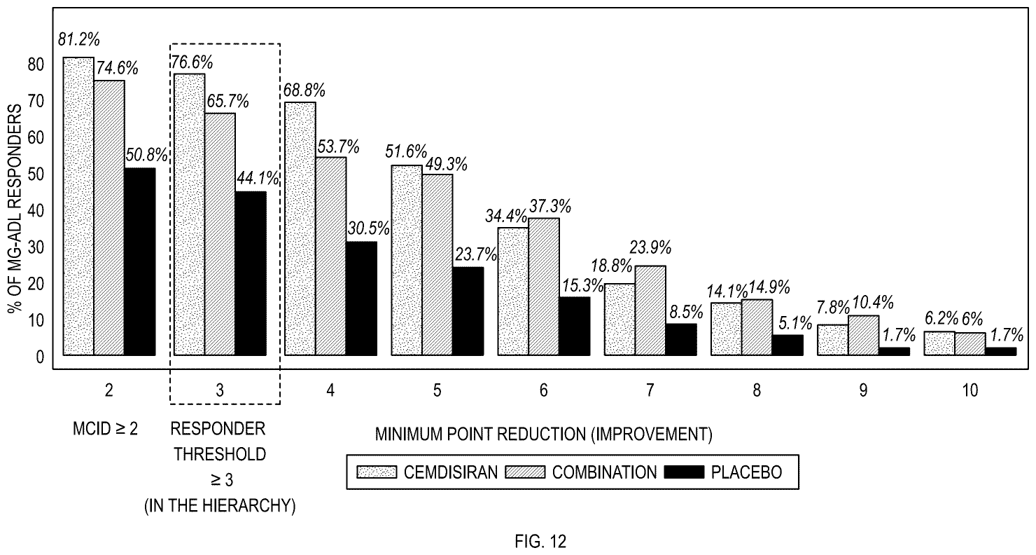

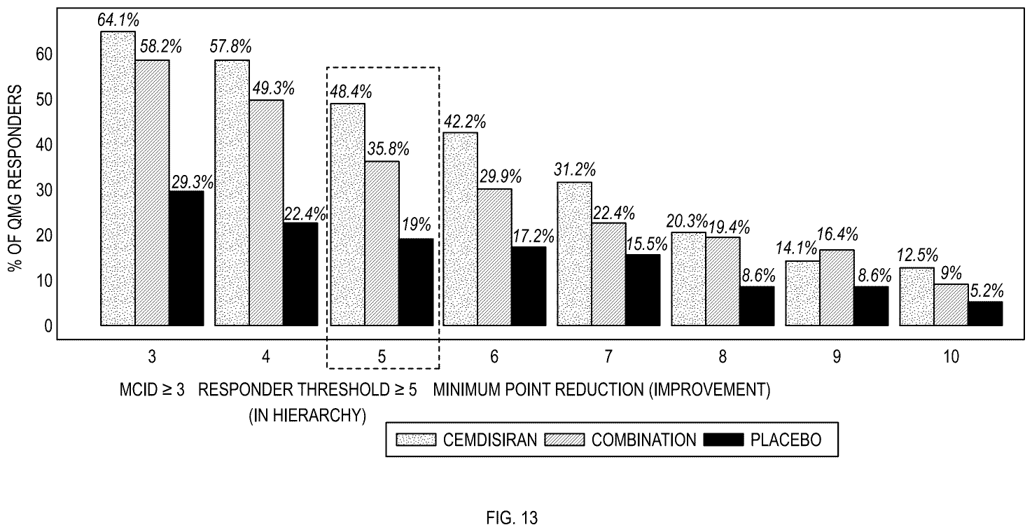

A higher proportion of participants achieved at least a 3-point reduction (improvement) in MG-ADL total score with Cemdisiran monotherapy (cemdi) or combination of Cemdisiran and Pozelimab (combo) than with placebo. See FIG. 12. A higher proportion of participants achieved at least a 5-point reduction (improvement) in QMG total score with cemdi or combo than with placebo. See FIG. 13.

The changes in MGC (Myasthenia Gravis Composite) and MG-QoL 15r (Myasthenia Gravis Quality of Life 15-item (revised)) over time are summarized in Table 1-17A-B and Table 1-18A-B.

Tables 1-17 C-J contain the Summary and MMRM statistical analysis of the change of MGC total score during the double-blind treatment period (DBTP) modified intent-to-treat analysis set (mITT). Tables 1-18 C-J contain a summary and MMRM statistical analysis of the change of MG-QOL15r total score during the double-blind treatment period (DBTP) modified intent-to-treat analysis set (mITT)

TABLE 1-17A
Least Square Mean (SE) of Change from Baseline in MGC

| | Mean | | | | | | | |
|---|---|---|---|---|---|---|---|---|
| | (SD) at | | | | | | | |
| Treatment | Baseline | Week 2 | Week 4 | Week 8 | Week 12 | Week 16 | Week 20 | Week 24 |
| Placebo | 17.51 | −2.43 | −2.84 | −3.64 | −4.61 | −3.86 | −3.44 | −3.45 |
| | (7.17) | (0.68) | (0.74) | (0.82) | (0.84) | (0.88) | (0.91) | (0.97) |
| Combination | 17.31 | −4.31 | −4.77 | −5.49 | −5.89 | −5.85 | −6.01 | −6.04 |
| | (6.64) | (0.65) | (0.69) | (0.77) | (0.76) | (0.81) | (0.85) | (0.88) |
| Cemdisiran | 17.21 | −4.49 | −5.22 | −6.55 | −7.55 | −7.57 | −7.69 | −7.78 |
| | (5.79) | (0.65) | (0.71) | (0.78) | (0.78) | (0.81) | (0.84) | (0.88) |
| Pozelimab | 18.44 | −1.64 | −2.13 | −3.34 | −3.47 | −3.30 | −3.57 | −3.19 |
| | (8.30) | (0.75) | (0.82) | (0.90) | (0.90) | (0.94) | (0.98) | (1.02) |

TABLE 1-17B
LS Mean Difference vs Placebo (SE) (MGC)

| Treatment | Week 24 |
|---|---|
| Placebo | |
| Combination | −2.60 (1.31); p-value = 0.0481 |
| Cemdisiran | −4.33 (1.31); p-value = 0.0009 |
| Pozelimab | 0.25 (1.40); p-value = 0.8557 |

P-value of 0.0481 is not significant based on alpha allocated of 0.0459 after interim analysis adjustment

TABLE 1-17C
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Study Baseline | | | | | | | | |
| n | 67 | | 63 | | 48 | | 59 | |
| Mean (SD) | 17.31 | (6.64) | 17.21 | (5.79) | 18.44 | (8.30) | 17.51 | (7.17) |
| Median | 17.00 | | 16.00 | | 18.00 | | 17.00 | |
| Q1:Q3 | 12.0:21.0 | | 13.0:22.0 | | 13.0:25.0 | | 11.0:22.0 | |
| Min:Max | 2.0:30.0 | | 6.0:33.0 | | 1.0:41.0 | | 0.0:33.0 | |
| 95% CI for Mean | (15.69, 18.93) | | (15.75, 18.67) | | (16.03, 20.85) | | (15.64, 19.38) | |
| Week 2 | | | | | | | | |
| n | 64 | | 64 | | 48 | | 59 | |
| Mean (SD) | 13.38 | (6.42) | 12.84 | (7.40) | 16.85 | (7.88) | 15.03 | (6.89) |

| Median | 13.00 | 11.00 | 17.00 | 16.00 |
|---|---|---|---|---|
| Q1:Q3 | 9.0:17.0 | 7.5:18.5 | 12.0:22.0 | 9.0:20.0 |
| Min:Max | 0.0:32.0 | 0.0:35.0 | 2.0:41.0 | 4.0:31.0 |
| 95% CI for Mean | (11.77, 14.98) | (10.99, 14.69) | (14.56, 19.14) | (13.24, 16.83) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17D
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 2 | | | | | | | | |
| Change from | | | | | | | | |
| Study Baseline | | | | | | | | |
| n | 64 | | 63 | | 47 | | 59 | |
| Mean (SD) | −4.41 | (6.02) | −4.30 | (5.64) | −1.96 | (5.84) | −2.47 | (4.44) |
| Median | −3.50 | | −4.00 | | −2.00 | | −2.00 | |
| Q1:Q3 | −8.0:0.0 | | −7.0:−1.0 | | −6.0:1.0 | | −5.0:0.0 | |
| Min:Max | −26.0:6.0 | | −21.0:11.0 | | −19.0:12.0 | | −18.0:8.0 | |
| 95% CI for Mean | (−5.91, −2.90) | | (−5.72, −2.88) | | (−3.67, −0.24) | | (−3.63, −1.32) | |
| LS Mean (SE) | −4.31 | (0.65) | −4.49 | (0.65) | −1.64 | (0.75) | −2.43 | (0.68) |
| 95% CI | (−5.57, −3.04) | | (−5.77, −3.21) | | (−3.12, −0.16) | | (−3.76, −1.11) | |
| Week 4 | | | | | | | | |
| n | 67 | | 64 | | 46 | | 57 | |
| Mean (SD) | 12.57 | (6.50) | 12.03 | (7.60) | 16.28 | (7.62) | 14.91 | (7.80) |

| Median | 12.00 | 10.50 | 16.00 | 14.00 |
|---|---|---|---|---|
| Q1:Q3 | 7.0:17.0 | 7.0:15.5 | 12.0:20.0 | 9.0:20.0 |
| Min:Max | 0.0:27.0 | 0.0:31.0 | 1.0:41.0 | 2.0:31.0 |
| 95% CI for Mean | (10.98, 14.15) | (10.13, 13.93) | (14.02, 18.54) | (12.84, 16.98) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17E
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 4 | | | | | | | | |
| Change from | | | | | | | | |
| Study Baseline | | | | | | | | |
| n | 67 | | 63 | | 45 | | 57 | |
| Mean (SD) | −4.75 | (6.22) | −5.02 | (5.72) | −2.56 | (6.76) | −3.07 | (5.37) |
| Median | −4.00 | | −4.00 | | −2.00 | | −2.00 | |
| Q1:Q3 | −8.0:0.0 | | −10.0:−1.0 | | −7.0:1.0 | | −5.0:0.0 | |
| Min:Max | −22.0:8.0 | | −23.0:5.0 | | −23.0:12.0 | | −24.0:6.0 | |
| 95% CI for Mean | (−6.26, −3.23) | | (−6.46, −3.58) | | (−4.59, −0.52) | | (−4.49, −1.65) | |
| LS Mean (SE) | −4.77 | (0.69) | −5.22 | (0.71) | −2.13 | (0.82) | −2.84 | (0.74) |
| 95% CI | (−6.12, −3.41) | | (−6.61, −3.83) | | (−3.74, −0.51) | | (−4.28, −1.39) | |
| Week 8 | | | | | | | | |
| n | 67 | | 64 | | 48 | | 55 | |
| Mean (SD) | 11.60 | (7.35) | 10.77 | (7.56) | 14.83 | (7.87) | 13.98 | (7.03) |

| Median | 10.00 | 9.00 | 14.00 | 15.00 |
|---|---|---|---|---|
| Q1:Q3 | 6.0:17.0 | 5.5:14.5 | 10.0:18.5 | 8.0:19.0 |
| Min:Max | 0.0:31.0 | 0.0:37.0 | 1.0:41.0 | 0.0:29.0 |
| 95% CI for Mean | (9.81, 13.39) | (8.88, 12.65) | (12.55, 17.12) | (12.08, 15.88) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17F
Secondary Endpoint: Summary and MMRM Statistical Analysis of
the Change of MGC Total Score During the Double-Blind Treatment
Period (DBTP) Modified Intent-to-Treat Analysis Set (mITT)

| MGC Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 8 | | | | | | | | |
| Change from | | | | | | | | |
| Study Baseline | | | | | | | | |
| n | 67 | | 63 | | 47 | | 55 | |
| Mean (SD) | −5.72 | (7.36) | −6.33 | (6.24) | −3.72 | (6.16) | −4.13 | (6.22) |
| Median | −6.00 | | −6.00 | | −2.00 | | −2.00 | |
| Q1:Q3 | −10.0:0.0 | | −9.0:−2.0 | | −8.0:0.0 | | −7.0:0.0 | |
| Min:Max | −24.0:9.0 | | −26.0:5.0 | | −22.0:12.0 | | −24.0:5.0 | |
| 95% CI for | (−7.51, −3.92) | | (−7.90, −4.76) | | (−5.53, −1.91) | | (−5.81, −2.45) | |
| Mean | | | | | | | | |
| LS Mean (SE) | −5.49 | (0.77) | −6.55 | (0.78) | −3.34 | (0.90) | −3.64 | (0.82) |
| 95% CI | (−6.99, −3.98) | | (−8.08, −5.03) | | (−5.10, −1.57) | | (−5.25, −2.03) | |
| Week 12 | | | | | | | | |
| n | 66 | | 64 | | 47 | | 54 | |
| Mean (SD) | 11.21 | (7.25) | 9.75 | (6.83) | 14.87 | (8.19) | 12.87 | (7.22) |

| Median | 11.00 | 8.50 | 14.00 | 12.50 |
|---|---|---|---|---|
| Q1:Q3 | 5.0:15.0 | 4.5:15.0 | 10.0:20.0 | 7.0:19.0 |
| Min:Max | 0.0:32.0 | 1.0:34.0 | 0.0:41.0 | 0.0:27.0 |
| 95% CI for | (9.43, 12.99) | (8.04, 11.46) | (12.47, 17.28) | (10.90, 14.84) |
| Mean | | | | |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17G
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 12 | | | | | | | | |
| Change from | | | | | | | | |
| Study Baseline | | | | | | | | |
| n | 66 | | 63 | | 46 | | 54 | |
| Mean (SD) | −5.97 | (7.38) | −7.32 | (6.18) | −3.96 | (6.10) | −5.19 | (6.51) |
| Median | −5.00 | | −6.00 | | −4.00 | | −3.50 | |
| Q1:Q3 | −11.0:0.0 | | −10.0:−4.0 | | −7.0:1.0 | | −9.0:−1.0 | |
| Min:Max | −24.0:6.0 | | −23.0:12.0 | | −19.0:9.0 | | −28.0:5.0 | |
| 95% CI for Mean | (−7.78, −4.16) | | (−8.87, −5.76) | | (−5.77, −2.15) | | (−6.96, −3.41) | |
| LS Mean (SE) | −5.89 | (0.76) | −7.55 | (0.78) | −3.47 | (0.90) | −4.61 | (0.84) |
| 95% CI | (−7.39, −4.39) | | (−9.08, −6.02) | | (−5.24, −1.69) | | (−6.26, −2.97) | |
| Week 16 | | | | | | | | |
| n | 64 | | 61 | | 47 | | 52 | |
| Mean (SD) | 10.92 | (6.27) | 9.84 | (7.32) | 14.91 | (9.13) | 13.38 | (8.24) |

| Median | 11.00 | 8.00 | 13.00 | 14.00 |
|---|---|---|---|---|
| Q1:Q3 | 6.0:14.5 | 4.0:15.0 | 9.0:20.0 | 7.0:17.0 |
| Min:Max | 0.0:29.0 | 0.0:37.0 | 0.0:41.0 | 0.0:37.0 |
| 95% CI for Mean | (9.35, 12.49) | (7.96, 11.71) | (12.23, 17.60) | (11.09, 15.68) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17H
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 16 | | | | | | | | |
| Change from | | | | | | | | |
| Study Baseline | | | | | | | | |
| n | 64 | | 60 | | 46 | | 52 | |
| Mean (SD) | −6.30 | (6.28) | −7.33 | (6.00) | −3.63 | (6.99) | −4.90 | (6.96) |
| Median | −6.00 | | −7.50 | | −2.50 | | −4.00 | |
| Q1:Q3 | −10.0:−2.0 | | −10.5:−3.0 | | −8.0:1.0 | | −9.0:−0.5 | |
| Min:Max | −24.0:6.0 | | −23.0:4.0 | | −20.0:15.0 | | −26.0:16.0 | |
| 95% CI for Mean | (−7.87, −4.73) | | (−8.88, −5.78) | | (−5.71, −1.55) | | (−6.84, −2.97) | |
| LS Mean (SE) | −5.85 | (0.81) | −7.57 | (0.81) | −3.30 | (0.94) | −3.86 | (0.88) |
| 95% CI | (−7.44, −4.26) | | (−9.17, −5.98) | | (−5.14, −1.46) | | (−5.59, −2.13) | |
| Week 20 | | | | | | | | |
| n | 64 | | 62 | | 48 | | 54 | |
| Mean (SD) | 10.84 | (7.34) | 9.79 | (7.93) | 14.50 | (9.00) | 13.02 | (7.03) |

| Median | 9.50 | 8.00 | 13.50 | 12.50 |
|---|---|---|---|---|
| Q1:Q3 | 5.5:17.0 | 4.0:15.0 | 9.0:19.0 | 7.0:18.0 |
| Min:Max | 0.0:32.0 | 0.0:37.0 | 0.0:41.0 | 0.0:29.0 |
| 95% CI for Mean | (9.01, 12.68) | (7.78, 11.81) | (11.89, 17.11) | (11.10, 14.94) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17I
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 20 | | | | | | | | |
| Change from | | | | | | | | |
| Study Baseline | | | | | | | | |
| n | 64 | | 62 | | 47 | | 54 | |
| Mean (SD) | −6.38 | (6.78) | −7.48 | (6.56) | −4.02 | (6.99) | −4.43 | (6.50) |
| Median | −6.50 | | −7.50 | | −4.00 | | −4.00 | |
| Q1:Q3 | −10.5:−1.0 | | −12.0:−4.0 | | −8.0:0.0 | | −8.0:−1.0 | |
| Min:Max | −24.0:6.0 | | −24.0:6.0 | | −21.0:13.0 | | −24.0:13.0 | |
| 95% CI for Mean | (−8.07, −4.68) | | (−9.15, −5.82) | | (−6.07, −1.97) | | (−6.20, −2.65) | |
| LS Mean (SE) | −6.01 | (0.85) | −7.69 | (0.84) | −3.57 | (0.98) | −3.44 | (0.91) |
| 95% CI | (−7.67, −4.35) | | (−9.33, −6.04) | | (−5.48, −1.65) | | (−5.21, −1.66) | |
| Week 24 | | | | | | | | |
| n | 64 | | 64 | | 48 | | 52 | |
| Mean (SD) | 10.83 | (7.51) | 9.67 | (7.47) | 14.63 | (9.36) | 13.00 | (7.63) |

| Median | 8.00 | 7.00 | 13.50 | 12.50 |
|---|---|---|---|---|
| Q1:Q3 | 6.0:15.0 | 4.5:15.5 | 7.0:20.0 | 6.0:20.0 |
| Min:Max | 0.0:34.0 | 0.0:32.0 | 0.0:41.0 | 0.0:28.0 |
| 95% CI for Mean | (8.95, 12.71) | (7.81, 11.54) | (11.91, 17.34) | (10.88, 15.12) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-17J
Secondary Endpoint: Summary and MMRM Statistical Analysis of
the Change of MGC Total Score During the Double-Blind Treatment
Period (DBTP) Modified Intent-to-Treat Analysis Set (mITT)

| | Combination | | Cemdisiran | | Pozelimab | | Placebo | |
|---|---|---|---|---|---|---|---|---|
| MGC Total Score | (N = 67) | | (N = 64) | | (N = 49) | | (N = 59) | |
| Week 24 | | | | | | | | |
| Change from | | | | | | | | |
| Study Baseline | | | | | | | | |
| n | 64 | | 63 | | 47 | | 52 | |
| Mean (SD) | −6.39 | (6.56) | −7.41 | (6.40) | −3.89 | (7.63) | −4.56 | (7.94) |
| Median | −6.00 | | −8.00 | | −3.00 | | −3.50 | |
| Q1:Q3 | −10.0:−1.5 | | −12.0:−3.0 | | −9.0:0.0 | | −9.0:1.0 | |
| Min:Max | −24.0:5.0 | | −22.0:10.0 | | −22.0:15.0 | | −27.0:11.0 | |
| 95% CI for Mean | (−8.03, −4.75) | | (−9.02, −5.80) | | (−6.13, −1.65) | | (−6.77, −2.35) | |
| LS Mean (SE) | −6.04 | (0.88) | −7.78 | (0.88) | −3.19 | (1.02) | −3.45 | (0.97) |

| 95% CI | (−7.76, −4.33) | | (−9.50, −6.05) | | (−5.18, −1.20) | | (−5.36, −1.54) |
|---|---|---|---|---|---|---|---|
| Difference: Active | | | | | | | |
| vs. Placebo | | | | | | | |
| LS Mean (SE) | −2.60 | (1.31) | −4.33 | (1.31) | 0.25 | (1.40) | |

| 95% CI | (−5.17, −0.02) | (−6.89, −1.77) | (−2.49, 3.00) |
|---|---|---|---|
| p-value | 0.0481 | 0.0009 | 0.8557 |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MGC total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MGC total score is included as a covariate in the model. The descriptive summary statistics are calculated prior to multiple imputation for ICEs.

TABLE 1-18A
Least Square Mean of Change from Baseline in MG-QoL15r

| | Mean | | | | | | | |
|---|---|---|---|---|---|---|---|---|
| | (SD) at | | | | | | | |
| Treatment | Baseline | Week 2 | Week 4 | Week 8 | Week 12 | Week 16 | Week 20 | Week 24 |
| Placebo | 16.37 | −1.38 | −1.14 | −2.01 | −2.33 | −2.59 | −1.97 | −2.26 |
| | (6.09) | (0.54) | (0.64) | (0.71) | (0.74) | (0.80) | (0.85) | (0.86) |
| Combination | 16.27 | −2.44 | −2.86 | −4.00 | −3.79 | −3.86 | −3.91 | −4.25 |
| | (5.61) | (0.50) | (0.59) | (0.65) | (0.67) | (0.73) | (0.76) | (0.76) |
| Cemdisiran | 15.88 | −1.43 | −2.67 | −4.08 | −4.31 | −4.52 | −4.65 | −4.68 |
| | (5.78) | (0.51) | (0.61) | (0.66) | (0.69) | (0.73) | (0.77) | (0.77) |
| Pozelimab | 16.37 | −1.65 | −1.26 | −0.92 | −1.76 | −1.49 | −0.70 | −1.58 |
| | (6.09) | (0.58) | (0.70) | (0.77) | (0.80) | (0.84) | (0.90) | (0.90) |

TABLE 1-18B
LS Mean Difference vs Placebo (SE) (MG-QoL15r)

| Treatment | Week 24 |
|---|---|
| Placebo | |
| Combination | −2.00 (1.15); p−value = 0.0825 |
| Cemdisiran | −2.43 (1.15); p−value = 0.0348 |
| Pozelimab | 0.67 (1.24); p−value = 0.5895 |

TABLE 1-18C
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| MGQOL15r Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Study Baseline | | | | | | | | |
| n | 64 | | 60 | | 46 | | 54 | |
| Mean (SD) | 16.27 | (5.61) | 15.88 | (5.78) | 16.02 | (6.70) | 16.37 | (6.09) |

| Median | 16.00 | 15.00 | 16.50 | 17.00 |
|---|---|---|---|---|
| Q1:Q3 | 12.0:21.0 | 12.0:20.0 | 11.0:22.0 | 12.0:21.0 |
| Min:Max | 3.0:29.0 | 2.0:30.0 | 1.0:28.0 | 1.0:28.0 |
| 95% CI for Mean | (14.87, 17.67) | (14.39, 17.38) | (14.03, 18.01) | (14.71, 18.03) |
| Week 2 | | | | |
| n | 64 | 63 | 48 | 59 |
| Mean (SD) | 14.08 (6.26) | 14.44 (5.95) | 14.17 (6.34) | 14.64 (6.30) |
| Median | 14.00 | 14.00 | 13.50 | 14.00 |
| Q1:Q3 | 10.0:18.5 | 11.0:18.0 | 12.0:19.0 | 12.0:19.0 |
| Min:Max | 1.0:27.0 | 1.0:26.0 | 0.0:26.0 | 1.0:26.0 |
| 95% CI for Mean | (12.52, 15.64) | (12.95, 15.94) | (12.33, 16.01) | (13.00, 16.29) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-QOL15r total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model. The descriptive summary statistics are calculated prior to multiple imputation for ICEs.

TABLE 1-18D
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| MGQOL15r Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 2 | | | | | | | | |
| Change from | | | | | | | | |
| Study Baseline | | | | | | | | |
| n | 61 | | 59 | | 45 | | 54 | |
| Mean (SD) | −2.61 | (4.26) | −1.39 | (3.77) | −1.64 | (4.81) | −1.46 | (3.54) |
| Median | −2.00 | | −1.00 | | −1.00 | | −1.50 | |
| Q1:Q3 | −6.0:1.0 | | −4.0:1.0 | | −3.0:1.0 | | −4.0:1.0 | |
| Min:Max | −20.0:4.0 | | −9.0:10.0 | | −17.0:16.0 | | −10.0:8.0 | |
| 95% CI for Mean | (−3.70, −1.52) | | (−2.37, −0.41) | | (−3.09, −0.20) | | (−2.43, −0.50) | |
| LS Mean (SE) | −2.44 | (0.50) | −1.43 | (0.51) | −1.65 | (0.58) | −1.38 | (0.54) |
| 95% CI | (−3.42, −1.45) | | (−2.43, −0.43) | | (−2.79, −0.50) | | (−2.43, −0.33) | |
| Week 4 | | | | | | | | |
| n | 67 | | 64 | | 46 | | 58 | |
| Mean (SD) | 13.49 | (6.72) | 13.06 | (6.42) | 14.59 | (6.81) | 14.64 | (7.22) |

| Median | 13.00 | 12.00 | 14.00 | 14.00 |
|---|---|---|---|---|
| Q1:Q3 | 9.0:18.0 | 9.0:16.5 | 11.0:19.0 | 10.0:20.0 |
| Min:Max | 0.0:29.0 | 1.0:29.0 | 0.0:28.0 | 0.0:27.0 |
| 95% CI for Mean | (11.85, 15.13) | (11.46, 14.67) | (12.57, 16.61) | (12.74, 16.54) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-QOL15r total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model. The descriptive summary statistics are calculated prior to multiple imputation for ICEs.

TABLE 1-18E
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| MGQOL15r Total | Combination | | Cemdisiran | | Pozelimab | | Placebo | |
|---|---|---|---|---|---|---|---|---|
| Score | (N = 67) | | (N = 64) | | (N = 49) | | (N = 59) | |
| Week 4 | | | | | | | | |
| Change from | | | | | | | | |
| Study Baseline | | | | | | | | |
| n | 64 | | 60 | | 43 | | 54 | |
| Mean (SD) | −2.88 | (5.37) | −2.62 | (4.94) | −1.56 | (4.22) | −1.22 | (3.96) |
| Median | −1.00 | | −2.00 | | −1.00 | | −1.00 | |
| Q1:Q3 | −7.5:1.0 | | −5.5:0.5 | | −3.0:1.0 | | −3.0:1.0 | |
| Min:Max | −17.0:10.0 | | −17.0:13.0 | | −14.0:9.0 | | −17.0:9.0 | |
| 95% CI for Mean | (−4.22, −1.53) | | (−3.89, −1.34) | | (−2.86, −0.26) | | (−2.30, −0.14) | |
| LS Mean (SE) | −2.86 | (0.59) | −2.67 | (0.61) | −1.26 | (0.70) | −1.14 | (0.64) |
| 95% CI | (−4.02, −1.70) | | (−3.86, −1.49) | | (−2.63, 0.11) | | (−2.39, 0.11) | |
| Week 8 | | | | | | | | |
| n | 67 | | 64 | | 48 | | 55 | |
| Mean (SD) | 12.22 | (7.01) | 11.67 | (6.64) | 15.08 | (6.73) | 14.16 | (6.78) |

| Median | 13.00 | 12.00 | 15.00 | 15.00 |
|---|---|---|---|---|
| Q1:Q3 | 7.0:17.0 | 7.0:15.0 | 11.0:20.0 | 9.0:19.0 |
| Min:Max | 0.0:29.0 | 0.0:29.0 | 0.0:29.0 | 0.0:27.0 |
| 95% CI for Mean | (10.51, 13.93) | (10.01, 13.33) | (13.13, 17.04) | (12.33, 16.00) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-QOL15r total score at all post-baseline treatment visits. The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model. The descriptive summary statistics are calculated prior to multiple imputation for ICEs.

TABLE 1-18F
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| | Combination | Cemdisiran | Pozelimab | Placebo |
|---|---|---|---|---|
| MGQOL15r Total | (N = 67) | (N = 64) | (N = 49) | (N = 59) |
| Score | | | | |
| Week 8 | | | | |
| Change from Study | 64 | 60 | 45 | 52 |
| Baseline | | | | |
| n | | | | |
| Mean (SD) | −4.19 (5.65) | −4.02 (5.90) | −0.91 (4.73) | −2.10 (4.58) |
| Median | −3.50 | −3.00 | 0.00 | −1.00 |
| Q1:Q3 | −8.5:0.0 | −9.0:0.0 | −4.0:2.0 | −4.0:0.0 |
| Min:Max | −18.0:6.0 | −19.0:13.0 | −13.0:11.0 | −16.0:8.0 |
| 95% CI for Mean | (−5.60, −2.78) | (−5.54, −2.49) | (−2.33, 0.51) | (−3.37, −0.82) |
| LS Mean (SE) | −4.00 (0.65) | −4.08 (0.66) | −0.92 (0.77) | −2.01 (0.71) |
| 95% CI | (−5.28, −2.72) | (−5.39, −2.78) | (−2.43, 0.59) | (−3.41, −0.62) |
| Week 12 | | | | |
| n | 66 | 64 | 48 | 54 |
| Mean (SD) | 12.38 (7.34) | 11.47 (6.57) | 14.19 (6.85) | 13.41 (7.00) |
| Median | 13.00 | 11.00 | 14.00 | 14.00 |
| Q1:Q3 | 8.0:18.0 | 7.5:15.5 | 10.0:18.5 | 9.0:19.0 |
| Min:Max | 0.0:27.0 | 0.0:27.0 | 0.0:29.0 | 0.0:27.0 |
| 95% CI for Mean | (10.58, 14.18) | (9.83, 13.11) | (12.20, 16.18) | (11.50, 15.32) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition.
LS Means (SE) and corresponding 95% CIs are based on a MMRM model where the response is observed changes in MG-QOL15r total score at all post-baseline treatment visits.
The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model.
The descriptive summary statistics are calculated prior to multiple imputation for ICEs.

TABLE 1-18G
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| | Combination | Cemdisiran | Pozelimab | Placebo |
|---|---|---|---|---|
| MGQOL15r Total | (N = 67) | (N = 64) | (N = 49) | (N = 59) |
| Score | | | | |
| Week 12 | | | | |
| Change from Study | 63 | 60 | 45 | 51 |
| Baseline | | | | |
| n | | | | |
| Mean (SD) | −3.92 (6.05) | −4.25 (5.77) | −1.78 (4.96) | −2.63 (4.65) |
| Median | −3.00 | −2.50 | −2.00 | −1.00 |
| Q1:Q3 | −8.0:0.0 | −8.5:0.0 | −4.0:1.0 | −4.0:1.0 |
| Min:Max | −20.0:12.0 | −17.0:5.0 | −15.0:13.0 | −19.0:5.0 |
| 95% CI for Mean | (−5.44, −2.40) | (−5.74, −2.76) | (−3.27, −0.29) | (−3.93, −1.32) |
| LS Mean (SE) | −3.79 (0.67) | −4.31 (0.69) | −1.76 (0.80) | −2.33 (0.74) |
| 95% CI | (−5.12, −2.47) | (−5.67, −2.96) | (−3.33, −0.20) | (−3.79, −0.87) |
| Week 16 | | | | |
| n | 64 | 63 | 47 | 52 |
| Mean (SD) | 12.00 (6.80) | 11.14 (6.47) | 14.28 (8.01) | 12.77 (7.66) |
| Median | 11.50 | 11.00 | 13.00 | 14.00 |
| Q1:Q3 | 8.0:15.0 | 6.0:15.0 | 8.0:20.0 | 7.5:17.5 |
| Min:Max | 0.0:27.0 | 0.0:28.0 | 0.0:30.0 | 0.0:28.0 |
| 95% CI for Mean | (10.30, 13.70) | (9.51, 12.77) | (11.92, 16.63) | (10.64, 14.90) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition.
LS Means (SE) and corresponding 95% CIs are based on a MMRM model where the response is observed changes in MG-QOL15r total score at all post-baseline treatment visits.
The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model.
The descriptive summary statistics are calculated prior to multiple imputation for ICEs.

TABLE 1-18H
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| | Combination | Cemdisiran | Pozelimab | Placebo |
|---|---|---|---|---|
| MGQOL15r Total | (N = 67) | (N = 64) | (N = 49) | (N = 59) |
| Score | | | | |
| Week 16 | | | | |
| Change from Study | 61 | 59 | 44 | 49 |
| Baseline | | | | |
| n | | | | |
| Mean (SD) | −4.10 (5.89) | −4.56 (6.33) | −1.64 (5.44) | −2.96 (4.70) |
| Median | −4.00 | −3.00 | −1.50 | −2.00 |
| Q1:Q3 | −8.0:0.0 | −9.0:0.0 | −4.0:1.0 | −5.0:0.0 |
| Min:Max | −15.0:13.0 | −22.0:6.0 | −17.0:12.0 | −20.0:5.0 |
| 95% CI for Mean | (−5.61, −2.59) | (−6.21, −2.91) | (−3.29, 0.02) | (−4.31, −1.61) |
| LS Mean (SE) | −3.86 (0.73) | −4.52 (0.73) | −1.49 (0.84) | −2.59 (0.80) |
| 95% CI | (−5.29, −2.44) | (−5.95, −3.08) | (−3.14, 0.17) | (−4.16, −1.02) |
| Week 20 | | | | |
| n | 64 | 64 | 48 | 54 |
| Mean (SD) | 12.05 (7.65) | 10.98 (6.62) | 15.04 (8.22) | 13.39 (7.21) |
| Median | 12.00 | 11.00 | 15.00 | 13.50 |
| Q1:Q3 | 6.0:16.5 | 6.0:14.0 | 8.5:20.0 | 9.0:19.0 |
| Min:Max | 0.0:30.0 | 0.0:28.0 | 0.0:30.0 | 0.0:27.0 |
| 95% CI for Mean | (10.14, 13.96) | (9.33, 12.64) | (12.66, 17.43) | (11.42, 15.36) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition. LS Means (SE) and corresponding 95% CIs are based on a MMRM model where the response is observed changes in MG-QOL15r total score at all post-baseline treatment visits.
The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model.
The descriptive summary statistics are calculated prior to multiple imputation for ICEs.

TABLE 1-18I
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| | Combination | Cemdisiran | Pozelimab | Placebo |
|---|---|---|---|---|
| MGQOL15r Total | (N = 67) | (N = 64) | (N = 49) | (N = 59) |
| Score | | | | |
| Week 20 | | | | |
| Change from Study | 61 | 60 | 45 | 50 |
| Baseline | | | | |
| n | | | | |
| Mean (SD) | −4.02 (6.79) | 4.58 (6.31) | −0.69 (5.86) | −2.40 (4.70) |
| Median | −4.00 | −3.00 | −1.00 | −1.00 |
| Q1:Q3 | −9.0:0.0 | −9.0:0.5 | −4.0:3.0 | −4.0:1.0 |
| Min:Max | −17.0:10.0 | −21.0:4.0 | −17.0:11.0 | −20.0:5.0 |
| 95% CI for Mean | (−5.75, −2.28) | (−6.21, −2.95) | (−2.45, 1.07) | (−3.74, −1.06) |
| LS Mean (SE) | −3.91 (0.76) | −4.65 (0.77) | −0.70 (0.90) | −1.97 (0.85) |
| 95% CI | (−5.41, −2.41) | (−6.16, −3.13) | (−2.46 ,1.07) | (−3.63, −0.31) |
| Week 24 | | | | |
| n | 64 | 64 | 48 | 53 |
| Mean (SD) | 11.70 (6.85) | 11.27 (6.84) | 14.33 (8.08) | 12.66 (7.21) |
| Median | 12.00 | 11.00 | 15.00 | 12.00 |
| Q1:Q3 | 6.5:17.0 | 6.0:15.0 | 7.0:20.0 | 10.0:17.0 |
| Min:Max | 0.0:25.0 | 0.0:29.0 | 0.0:30.0 | 0.0:27.0 |
| 95% CI for Mean | (9.99, 13.41) | (9.56, 12.97) | (11.99, 16.68) | (10.67, 14.65) |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition.
LS Means (SE) and corresponding 95% CIs are based on a MMRM model where the response is observed changes in MG-QOL15r total score at all post-baseline treatment visits.
The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model.
The descriptive summary statistics are calculated prior to multiple imputation for ICEs.

TABLE 1-18J
Secondary Endpoint: Summary and MMRM Statistical Analysis of the
Change of MG-QOL15r Total Score During the Double-Blind Treatment
Period (DBTP) Modified Intent-to-Treat Analysis Set (mITT)

| | Combination | Cemdisiran | Pozelimab | Placebo |
|---|---|---|---|---|
| MGQOL15r Total | (N = 67) | (N = 64) | (N = 49) | (N = 59) |
| Score | | | | |
| Week 24 | | | | |
| Change from Study | 61 | 60 | 45 | 49 |
| Baseline | | | | |
| n | | | | |
| Mean (SD) | −4.34 (6.24) | −4.47 (6.63) | −1.62 (5.82) | −2.94 (5.41) |
| Median | −4.00 | −3.00 | −1.00 | −2.00 |
| Q1:Q3 | −9.0:0.0 | −9.5:0.0 | −5.0:1.0 | −5.0:0.0 |
| Min:Max | −19.0:9.0 | −21.0:10.0 | −17.0:12.0 | −20.0:12.0 |
| 95% CI for Mean | (−5.94, −2.75) | (−6.18, −2.75) | (−3.37, 0.13) | (−4.49, −1.38) |
| LS Mean (SE) | −4.25 (0.76) | −4.68 (0.77) | −1.58 (0.90) | −2.26 (0.86) |
| 95% CI | (−5.74, −2.76) | (−6.19, −3.17) | (−3.35, 0.18) | (−3.94, −0.57) |
| Difference: Active | | | | |
| vs. Placebo | | | | |
| LS Mean (SE) | −2.00 (1.15) | −2.43 (1.15) | 0.67 (1.24) | |
| 95% CI | (−4.25, 0.26) | (−4.68, −0.17) | (−1.77, 3.11) | |
| p-value | 0.0825 | 0.0348 | 0.5895 | |

CI = confidence interval;
LS = least square;
Max = maximum;
Min = minimum;
MMRM = mixed model for repeated measures;
Q1 = first quartile;
Q3 = third quartile;
SD = standard deviation;
SE = standard error
The efficacy analysis is based on the first 245 randomized participants who met mITT definition.
LS Means (SE) and corresponding 95% CIs are based on a MMRM model where the response is observed changes in MG-QOL15r total score at all post-baseline treatment visits.
The stratification factors of MGFA class at screening and treatment with IST on day 1, study treatment, visit (weeks 2, 4, 8, 12, 16, 20, and 24), baseline-by-visit interaction, treatment-by-visit interaction are included as factors and baseline MG-QOL15r total score is included as a covariate in the model.
The descriptive summary statistics are calculated prior to multiple imputation for ICEs.

### Safety

Table 1-19A and Table 1-19B summarize adverse events of worsening of myasthenia (including crisis) and use of rescue therapy numerically lower in Cemdisiran monotherapy (Cemdisiran).

TABLE 1-19A
Descriptive Statistics of Number of Myasthenic Crises (Including
Impending Crisis) During the Double-Blind Treatment Period
(DBTP) Modified Intent-to-Treat Analysis Set (mITT)

| | Placebo | | Combination | | Cemdisiran | | Pozelimab | |
|---|---|---|---|---|---|---|---|---|
| | (N = 66) | | (N = 75) | | (N = 75) | | (N = 49) | |
| Number of participants with | 12 | (18.2%) | 6 | (8.0%) | 1 | (1.3%) | 4 | (8.2%) |
| at least one event, n (%) | | | | | | | | |
| Total number of events | 15 | | 8 | | 1 | | 4 | |
| Summary statistics | 12 | | 6 | | 1 | | 4 | |
| for total number of | | | | | | | | |
| events per | | | | | | | | |
| participant n | | | | | | | | |
| Mean (SD) | 1.3 | (0.5) | 1.3 | (0.5) | 1.0 | (—) | 1.0 | (0.0) |

| | | | | |
|---|---|---|---|---|
| Median | 1.0 | 1.0 | 1.0 | 1.0 |
| Q1:Q3 | 1:2 | 1:2 | 1:1 | 1:1 |
| Min:Max | 1:2 | 1:2 | 1:1 | 1:1 |

Max = maximum;
Min = minimum;
Q1 = first quartile;
Q3 = third quartile;
SD = standard deviation

TABLE 1-19B
Rescue Medications During the Double-Blind Treatment
Period (DBTP), Safety Analysis Set (SAF)

| | Placebo | | Combination | Cemdisiran | Pozelimab | Total | |
|---|---|---|---|---|---|---|---|
| | (N = 70) | | (N = 80) | (N = 78) | (N = 49) | (N = 277) | |
| Participants with at | 9 | (12.9%) | 5 (6.3%) | 1 (1.3%) | 1 (2.0%) | 16 | (5.8%) |
| least one rescue | | | | | | | |
| medication | | | | | | | |
| Rescue Therapy IVIG | 6 | (8.6%) | 4 (5.0%) | 1 (1.3%) | 1 (2.0%) | 12 | (4.3%) |
| PLEX or | 3 | (4.3%) | 1 (1.3%) | 0 | 0 | 4 | (1.4%) |
| comparable | | | | | | | |
| therapeutic apheresis | | | | | | | |
| (TA) | | | | | | | |

Myasthenic crisis—a serious, life-threatening, rapid worsening of MG and potential airway compromise from ventilatory or bulbar dysfunction;
PLEX—plasma exchange

The participant disposition based on mITT-primary is summarized in Table 1-20.

TABLE 1-20
Participants Disposition Based on mITT-Primary

| | Placebo | | Pozelimab | | Combination | | Cemdisiran | Total | |
|---|---|---|---|---|---|---|---|---|---|
| | (N = 59) | | (N = 49) | | (N = 67) | | (N = 64) | (N = 239) | |
| Participants who completed study | 53 | (90%) | 48 | (98%) | 64 | (95.5%) | 64 (100%) | 229 | (96%) |
| treatment during the DBTP, n(%) | | | | | | | | | |
| Participants who discontinued study | 6 | (10%) | 1 | (2%) | 3 | (4.5%) | 0 | 10 | (4%) |
| during DBTP, n(%) | | | | | | | | | |
| Reason for study discontinuation | | | | | | | | | |
| ADVERSE EVENT | 1 | (17%) | 1 | (100%) | | | | 2 | (20%) |
| PHYSICIAN DECISION | | | | | 1 | (33%) | | 1 | (10%) |
| WITHDRAWAL BY SUBJECT | 4 | (66%) | | | 2 | (67%) | | 6 | (60%) |
| LOST TO FOLLOW-UP | 1 | (17%) | | | | | | 1 | (10%) |

Treatment is associated with lower drop-out rate

The overall incidence of treatment emergent adverse events (TEAEs) was numerically lower in the Cemdisiran monotherapy arm (Cemdisiran). See Table 1-21. Adverse event of special interest (AESI) were:
Hypersensitivity reactions potentially related to study treatment
Injection site reactions Suspected Neisseria infection
Liver transaminase elevations as evidenced by 1 or more of the following criteria: ALT or AST >8×ULN, OR ALT or AST >5×ULN for >2 weeks, OR ALT or AST >3×ULN and total bilirubin >2×ULN (or INR >1.5)

TABLE 1-21
Summary of TEAEs during double blind treatment period (DBTP)

| | DBTP | | | | | | | |
|---|---|---|---|---|---|---|---|---|
| Participants with | Placebo | | Pozelimab | | Combination | | Cemdisiran | |
| at least one | (N = 70) | | (N = 49) | | (N = 80) | | (N = 78) | |
| TEAE | 54 | (77.1%) | 40 | (81.6%) | 65 | (81.3%) | 54 | (69.2%) |
| Severe TEAE | 7 | (10.0%) | 5 | (10.2%) | 8 | (10.0%) | 2 | (2.6%) |
| TE-SAE | 10 | (14.3%) | 5 | (10.2%) | 7 | (8.8%) | 2 | (2.6%) |
| AESI | 2 | (2.9%) | 1 | (2.0%) | 12 | (15.0%) | 8 | (10.3%) |
| Treatment-related TEAE | 16 | (22.9%) | 13 | (26.5%) | 32 | (40.0%) | 23 | (29.5%) |
| TEAE leading to study | 2 | (2.9%) | 1 | (2.0%) | 0 | | 0 | |
| treatment discontinuation | | | | | | | | |
| TEAE leading to death | 0 | | 0 | | 0 | | 0 | |
| TEAE leading to dose | 3 | (4.3%) | 1 | (2.0%) | 3 | (3.8%) | 1 | (1.3%) |
| interruption | | | | | | | | |

Note:
Of the two subjects in the placebo group who had a TEAE leading to study discontinuation, one subject completed the DBTP and then withdrew from the study.
SAE = serious adverse event;
TE = treatment-emergent;
TEAE = treatment-emergent adverse event
DBTP: Double Blind Treatment Period Events in combination were overall similar to placebo (DBTP)

There were no differences across treatment arms in treatment emergent adverse events (TEAEs). TEAEs were aligned with other C5 inhibitor's labels. See Table 1-22.

TABLE 1-22
Summary Of Treatment Emergent Adverse Events (TEAEs)

| | DBTP | | | | | | | |
|---|---|---|---|---|---|---|---|---|
| | Placebo | | Pozelimab | | Combination | | Cemdisiran | |
| Preferred Term | (N = 70) | | (N = 49) | | (N = 80) | | (N = 78) | |
| Number of participants with at | 54 | (77.1%) | 40 | (81.6%) | 65 | (81.3%) | 54 | (69.2%) |
| least one TEAE, n (%) | | | | | | | | |
| Upper respiratory tract | 8 | (11.4%) | 6 | (12.2%) | 6 | (7.5%) | 9 | (11.5%) |
| infection | | | | | | | | |
| Urinary tract infection | 2 | (2.9%) | 4 | (8.2%) | 5 | (6.3%) | 4 | (5.1%) |
| Nasopharyngitis | 3 | (4.3%) | 2 | (4.1%) | 2 | (2.5%) | 4 | (5.1%) |
| Headache | 7 | (10.0%) | 5 | (10.2%) | 9 | (11.3%) | 4 | (5.1%) |
| Rash | 1 | (1.4%) | 2 | (4.1%) | 2 | (2.5%) | 4 | (5.1%) |
| Injection site reaction | 1 | (1.4%) | 1 | (2.0%) | 6 | (7.5%) | 3 | (3.8%) |
| Diarrhoea | 5 | (7.1%) | 6 | (12.2%) | 11 | (13.8%) | 2 | (2.6%) |
| Arthralgia | 1 | (1.4%) | 1 | (2.0%) | 5 | (6.3%) | 1 | (1.3%) |
| Pain in extremity | 1 | (1.4%) | 0 | | 4 | (5.0%) | 1 | (1.3%) |
| Myasthenia gravis | 12 | (17.1%) | 4 | (8.2%) | 4 | (5.0%) | 1 | (1.3%) |
| Cough | 1 | (1.4%) | 1 | (2.0%) | 4 | (5.0%) | 1 | (1.3%) |

| | | | | | |
|---|---|---|---|---|---|
| Pruritus | 0 | 0 | 4 | (5.0%) | 0 |

TEAE: treatment-emergent adverse event;
DBTP: Double Blind Treatment Period;
PT: Preferred Term
Overview of adverse events - incidence rate of ≥5% by PT in any arm (DBTP)

There was a lower incidence of serious AEs in the Cemdisiran monotherapy arm (Cemdisiran) compared to other treatment arms. See Table 1-23.

TABLE 1-23
Overview of Serious TEAEs by PT - DBTP

| | Placebo | | Pozelimab | | Combination | | Cemdisiran | |
|---|---|---|---|---|---|---|---|---|
| Preferred Term | (N = 70) | | (N = 49) | | (N = 80) | | (N = 78) | |
| Number of participants with at least one | 10 | (14.3%) | 5 | (10.2%) | 7 | (8.8%) | 2 | (2.6%) |
| serious TEAE, n (%) | | | | | | | | |

| | | | | | |
|---|---|---|---|---|---|
| Oxygen saturation decreased | 0 | 0 | 0 | 1 | (1.3%) |
| Uterine leiomyoma* | 0 | 0 | 0 | 1 | (1.3%)* |
| Adenomyosis* | 0 | 0 | 0 | 1 | (1.3%)* |

| Pulmonary embolism | 0 | | 0 | 2 | (2.5%) | 0 |
|---|---|---|---|---|---|---|
| COVID-19 | 0 | | 0 | 1 | (1.3%) | 0 |
| Dengue fever | 0 | | 0 | 1 | (1.3%) | 0 |
| Atrial fibrillation | 0 | | 0 | 1 | (1.3%) | 0 |
| Diarrhoea | 0 | | 0 | 1 | (1.3%) | 0 |
| Muscular weakness | 0 | | 0 | 1 | (1.3%) | 0 |
| Myasthenia gravis crisis | 1 | (1.4%) | 0 | 1 | (1.3%) | 0 |

| | | | | | |
|---|---|---|---|---|---|
| Deep vein thrombosis | 0 | 0 | 1 | (1.3%) | 0 |

| COVID-19 pneumonia | 0 | | 1 | (2.0%) | 0 | 0 |
|---|---|---|---|---|---|---|
| Pneumonia mycoplasmal | 0 | | 1 | (2.0%) | 0 | 0 |
| Myasthenia gravis | 8 | (11.4%) | 1 | (2.0%) | 0 | 0 |

| | | | | | |
|---|---|---|---|---|---|
| Adrenal insufficiency | 0 | 1 | (2.0%) | 0 | 0 |
| Pyrexia | 0 | 1 | (2.0%) | 0 | 0 |
| Fall | 0 | 1 | (2.0%) | 0 | 0 |
| Multiple fractures | 0 | 1 | (2.0%) | 0 | 0 |
| Steroid diabetes | 0 | 1 | (2.0%) | 0 | 0 |

| | | | | | |
|---|---|---|---|---|---|
| Cellulitis | 1 | (1.4%) | 0 | 0 | 0 |
| Upper respiratory tract infection | 1 | (1.4%) | 0 | 0 | 0 |
| Urinary tract infection | 1 | (1.4%) | 0 | 0 | 0 |
| Dyspnoea | 1 | (1.4%) | 0 | 0 | 0 |
| Facial spasm | 1 | (1.4%) | 0 | 0 | 0 |

*The same patient experienced both Uterine Leiomyoma and Adenomyosis.
TEAE: treatment-emergent adverse event
DBTP: Double Blind Treatment Period
PT: Preferred Term;
Sorted by decreasing frequency of PT based on the Cemdisiran treatment group

There were no serious infections in the Cemdisiran monotherapy arm (Cemdisiran). See Table 1-24.

TABLE 1-24
Overview of serious infections by Preferred Term (PT) - DBTP

| | Placebo | Pozelimab | Combination | Cemdisiran |
|---|---|---|---|---|
| | (N = 70) | (N = 49) | (N = 80) | (N = 78) |
| Number of participants with at least one | 3 (4.3%) | 2 (4.1%) | 2 (2.5%) | 0 |
| TEAE, n (%) | | | | |
| COVID-19 | 0 | 0 | 1 (1.3%) | 0 |
| Dengue fever | 0 | 0 | 1 (1.3%) | 0 |
| COVID-19 pneumonia | 0 | 1 (2.0%) | 0 | 0 |
| Cellulitis | 1 (1.4%) | 0 | 0 | 0 |
| Pneumonia mycoplasmal | 0 | 1 (2.0%) | 0 | 0 |
| Upper respiratory tract infection | 1 (1.4%) | 0 | 0 | 0 |
| Urinary tract infection | 1 (1.4%) | 0 | 0 | 0 |

Sorted by decreasing frequency of PT based on the Cemdisiran treatment group

Injection site reactions were more frequent with Cemdisiran monotherapy (Cemdisiran) and combination of Cemdisiran and Pozelimab (Combination); all were mild/moderate and none led to discontinuation. See Table 1-25.

TABLE 1-25
Overview of Injection site reactions by PT - DBTP

| AESI Category | Placebo | Pozelimab | Combination | | Cemdisiran |
|---|---|---|---|---|---|
| Preferred Term | (N = 70) | (N = 49) | (N = 80) | | (N = 78) |
| Injection Site Reactions | 2 (2.9%) | 1 (2.0%) | 11 | (13.8%) | 6 (7.7%) |
| Injection site reaction | 1 (1.4%) | 1 (2.0%) | 6 | (7.5%) | 3 (3.8%) |
| Injection site erythema | 1 (1.4%) | 0 | 2 | (2.5%) | 0 |
| Injection site rash | 0 | 0 | 2 | (2.5%) | 0 |
| Erythema | 0 | 0 | 1 | (1.3%) | 0 |
| Injection site bruising | 0 | 0 | 1 | (1.3%) | 0 |
| Rash | 0 | 0 | 1 | (1.3%) | 2 (2.6%) |
| Skin reaction | 0 | 0 | 1 | (1.3%) | 0 |

| | | | | |
|---|---|---|---|---|
| Dermatitis | 0 | 0 | 0 | 1 (1.3%) |
| Dermatitis allergic | 0 | 0 | 0 | 1 (1.3%) |
| Injection site pruritus | 0 | 0 | 0 | 1 (1.3%) |

Sorted by decreasing frequency of SOC and PT based on the combination treatment group
AESI: Adverse events of special interest

### Clinical Pharmacology

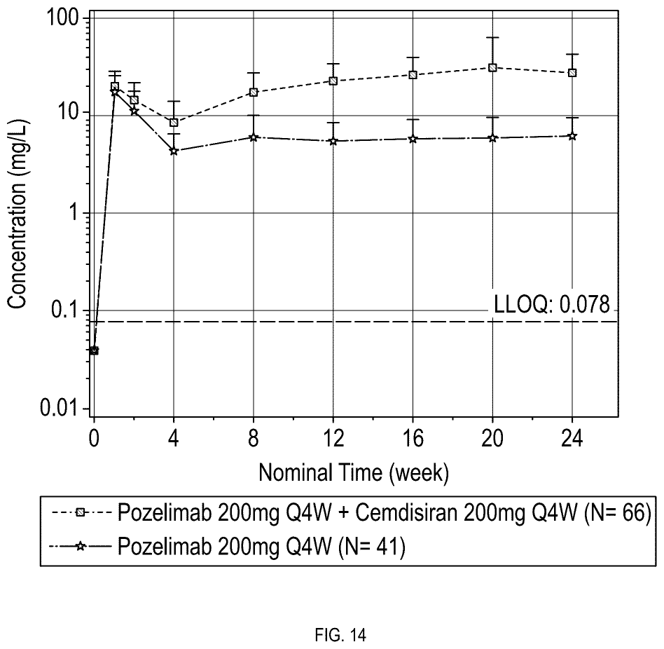

Exposure of Pozelimab was higher when administered in combination with Cemdisiran vs. Pozelimab monotherapy. Higher exposures in the presence of Cemdisiran after multiple dosing was likely due to reduced target-mediated clearance of Pozelimab, consequent to lower hepatic synthesis of C5. See FIG. 14.

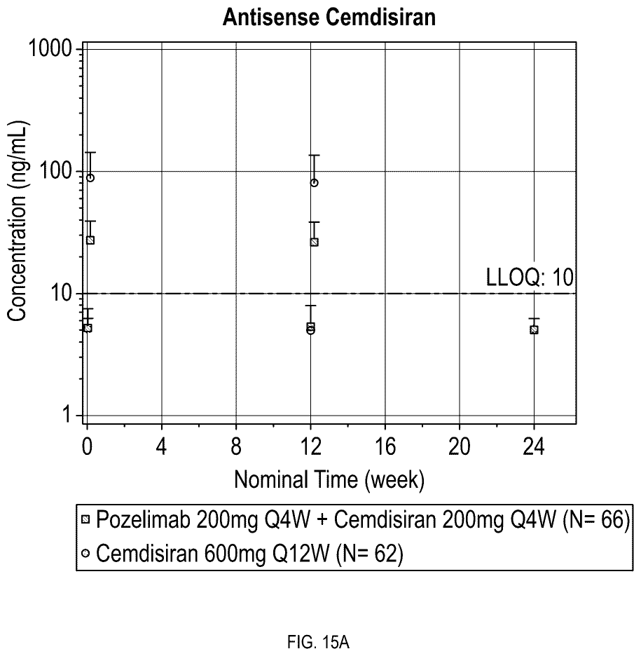

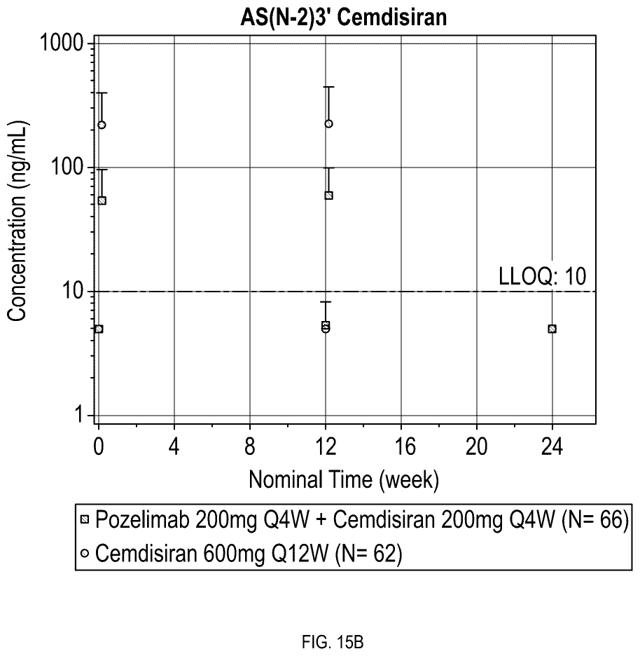

There was a dose proportional increase in exposure between the combination arm and the Cemdisrian monotherapy arm. See FIG. 15.

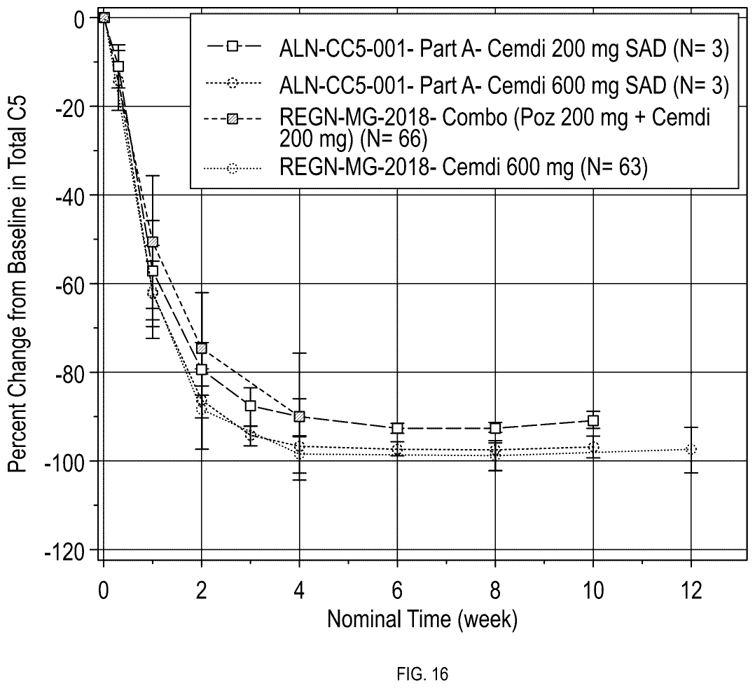

The mean (±SD) percent change from baseline in concentrations of total C5 in plasma by nominal time, treatment group and study (study ALN-CC5-001, Part A, single ascending dose (SAD) and R3918-MG-2018) are set forth in FIG. 16 and Table 1-26. There was consistent knockdown of total C5 by 600 mg Cemdisiran monotherapy between studies. The delay in attainment of maximum inhibition in the combination arm was because of slow clearance of the Pozelimab: C5 complex (comparing 200 mg Cemdisiran with combination of 200 mg Cemdisiran and 200 mg Pozelimab).

TABLE 1-26
Summary of Percent Change from Baseline in Total
C5 in plasma by Nominal Time, Treatment Group and
Study (Study ALN-CC5-001 and R3918-MG-2018, TTAS)

| ALN-CC5-001- | | | | | | | | |
|---|---|---|---|---|---|---|---|---|
| Part A- | | | | | | | | |
| Cemdi 200 | | | | | | | | |
| mg SD | | | | | REGN-MG-2018- | | | |
| (N = 3) | | | ALN-CC5-001- | | Combo | | REGN-MG-2018- | |
| Sampling | | | Part A- | | (Poz 200 mg + | | Cemdi 600 | |
| Time Post | | | Cemdi 600 | | Cemdi 200 mg) | | mg | |
| First Dose | | | mg SD (N = 3) | | (N = 66) | | (N = 63) | |
| (week) | n | Mean (SD) | n | Mean (SD) | n | Mean (SD) | n | Mean (SD) |
| 0.2857 | 3 | −10.9 (4.85) | 3 | −14.1 (6.72) | 0 | | 0 | |
| 1 | 3 | −56.9 (11.2) | 3 | −62.3 (7.35) | 66 | −50.6 (15.0) | 61 | −61.7 (10.5) |
| 2 | 3 | −79.3 (5.89) | 3 | −86.6 (3.52) | 64 | −74.5 (12.3) | 61 | −88.2 (9.08) |
| 3 | 3 | −87.7 (4.32) | 3 | −94.4 (2.22) | 0 | | 0 | |
| 4 | 3 | −90.2 (4.17) | 3 | −96.6 (2.21) | 65 | −90.0 (14.4) | 59 | −98.6 (4.07) |
| 6 | 3 | −92.6 (1.17) | 3 | −97.3 (1.53) | 0 | | 0 | |
| 8 | 3 | −92.4 (1.08) | 3 | −97.4 (1.51) | 0 | | 60 | −98.8 (3.40) |
| 10 | 3 | −90.7 (1.89) | 3 | −96.8 (2.44) | 0 | | 0 | |
| 12 | 0 | | 0 | | 0 | | 59 | −97.6 (5.07) |

n = Number of participants contributing to the statistical results;
TTAS = Total target analysis set (participants received at least one dose of Cemdisiran or Combination and with at least one post-dose C5 measurement);
SD = Standard deviation
Note:
Below LLOQ value is set to 0. Data after week 4 is excluded for REGN-MG-2018-Combo (Poz 200 mg + Cemdi 200 mg). Data after week 12 is excluded for REGN-MG-2018- Cemdi 600 mg.

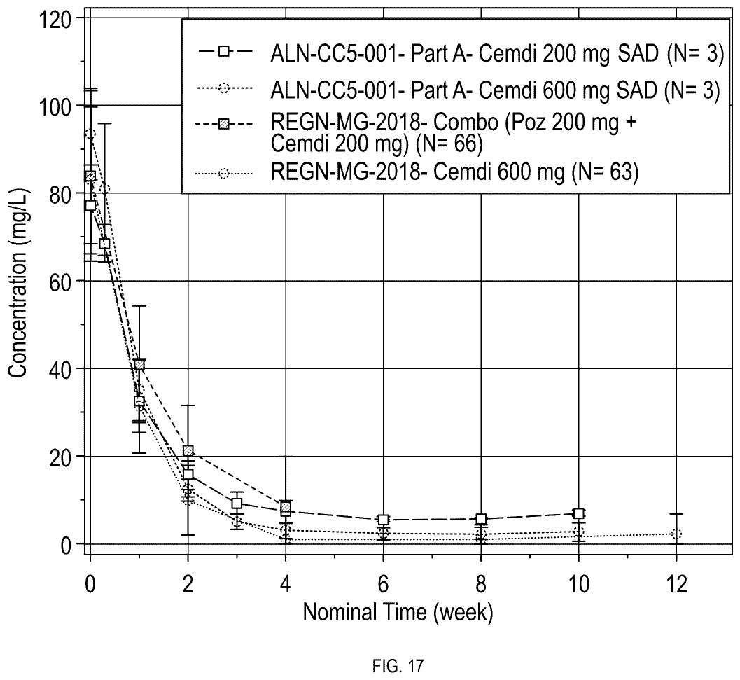

The mean (±SD) concentrations of total C5 in plasma by nominal time, treatment group and study (study ALN-CC5-001 and R3918-MG-2018) is set forth in FIG. 17. Consistent knockdown of total C5 by 600 mg Cemdisiran monotherapy between studies was observed. The delay in attainment of LOQ in the combination arm was because of slow clearance of the Pozelimab: C5 complex (comparing 200 mg Cemdisiran with combination of 200 mg Cemdisiran and 200 mg Pozelimab).

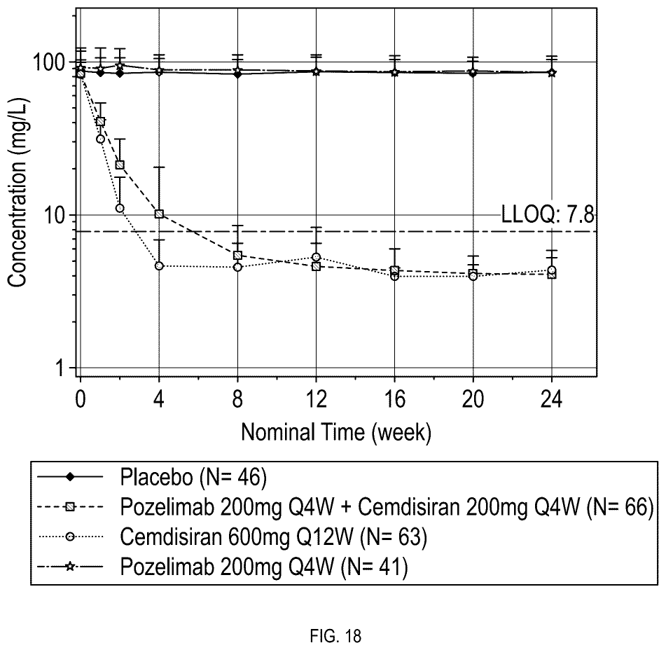

The decrease in total C5 concentration in plasma to below LOQ by week 4 in Cemdisiran arm vs. week 8 in the combination arm is summarized in FIG. 18.

A summary of Cemdisiran and Pozelimab anti-drug antibody is set forth in Table 1-27 and Table 1-28, respectively.

TABLE 1-27
Cemdisiran Anti-Drug-Antibody

| | Combination | | Cemdisiran | |
|---|---|---|---|---|
| ADA Status | n (%) | | n (%) | |
| Cemdisiran ADA | | | | |
| ADA Analysis Set | 62 | | 60 | |
| Negative | 61 | (98.4%) | 59 | (98.3%) |

| | | | |
|---|---|---|---|
| Pre-existing Immunoreactivity | 1 | (1.6%) | 0 |

| Treatment-Boosted Response | 0 | 0 | |
|---|---|---|---|
| Treatment-Emergent Response | 0 | 1 | (1.7%) |
| Persistent | 0 | 0 | |
| Transient | 0 | 1 | (1.7%) |
| Indeterminate | 0 | 0 | |
| TE & TB Maximum Titer Category | | | |
| Low (<1,000) | 0 | 1 | (1.7%) |

| | | |
|---|---|---|
| Moderate (1,000 to 10,000) | 0 | 0 |
| High (>10,000) | 0 | 0 |

n = Number of participants;
TE = Treatment-emergent;
TB = Treatment-boosted;
Q4W = Every 4 weeks;
Q12W = Every 12 weeks;
Combination = Pozelimab 200 mg Q4W + Cemdisiran 200 mg Q4W;
Cemdisiran = Cemdisiran 600 mg Q12W
Note:
Percentages are based on ADA analysis set for ADA status, subcategories and titer categories.
*One participant had positive ADA at week 24.

TABLE 1-28
Pozelimab Anti-Drug-Antibody

| | Combination | | Pozelimab | |
|---|---|---|---|---|
| ADA Status | n (%) | | n (%) | |
| Pozelimab ADA | | | | |
| ADA Analysis Set | 62 | | 41 | |
| Negative | 61 | (98.4%) | 40 | (97.6%) |

| | | | |
|---|---|---|---|
| Pre-existing Immunoreactivity | 0 | 1 | (2.4%) |

| Treatment-Boosted Response | 0 | | 0 |
|---|---|---|---|
| Treatment-Emergent Response | 1 | (1.6%) | 0 |
| Persistent | 0 | | 0 |
| Transient | 1 | (1.6%) | 0 |
| Indeterminate | 0 | | 0 |
| TE & TB Maximum Titer Category | | | |
| Low (<1,000) | 1 | (1.6%) | 0 |

| | | |
|---|---|---|
| Moderate (1,000 to 10,000) | 0 | 0 |
| High (>10,000) | 0 | 0 |

n = Number of participants;
TE = Treatment-emergent;
TB = Treatment-boosted;
Q4W = Every 4 weeks;
Combination = Pozelimab 200 mg Q4W + Cemdisiran 200 mg Q4W;
Pozelimab = Pozelimab 200 mg Q4W
Note:
Percentages are based on ADA analysis set for ADA status, subcategories and titer categories,
*One participant had positive ADA at week 12.

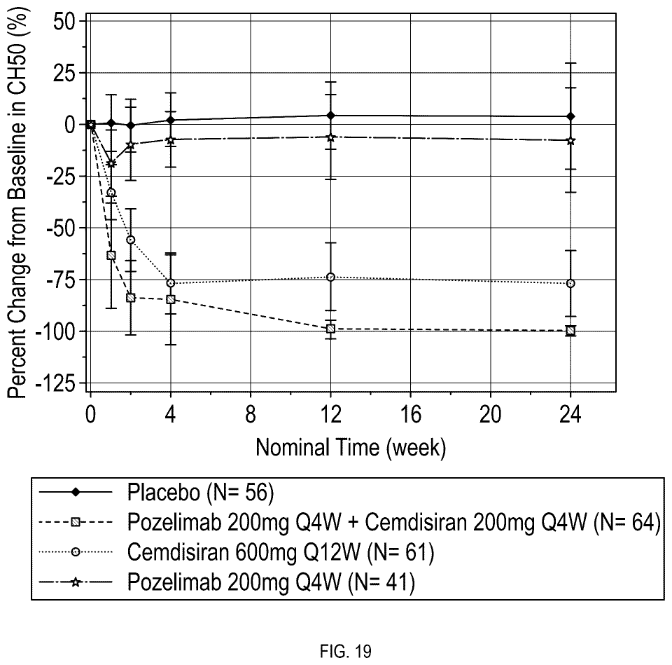

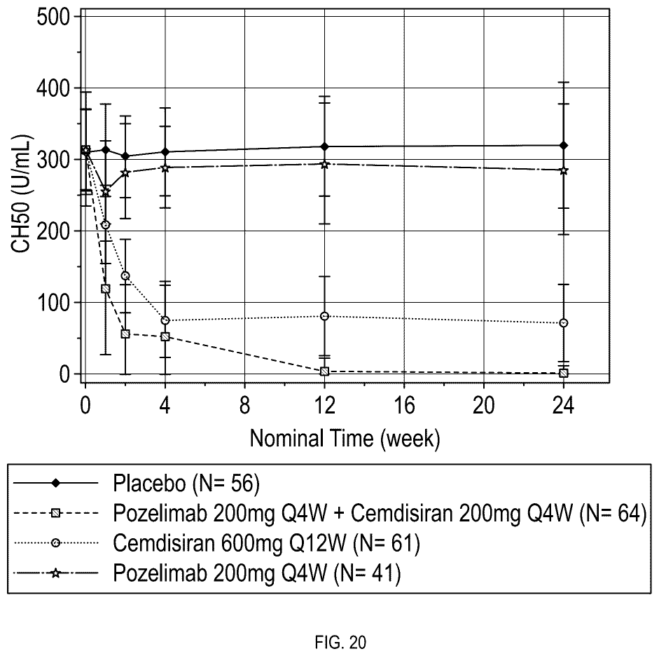

There was a greater CH50 inhibition in serum in the combination arm vs. the Cemdisiran monotherapy arm. The combination achieved consistent inhibition of the complement pathway; ˜100% change from baseline in CH50. See FIG. 19. The mean (±SD) CH50 in serum by nominal time in participants with gMG is summarized in FIG. 20.

### CONCLUSIONS

### Efficacy

Cemdisiran monotherapy met primary endpoint and key secondary endpoint:
   Reduction in MG-ADL total score at week 24 vs placebo (LS means; −4.52 vs −2.22, TRT Diff=−2.30 p=0.0005).
   Reduction in QMG total score at week 24 vs. placebo (LS means; −4.24 vs. −1.46, TRT Diff=−2.77 p=0.0015)
Pozelimab+Cemdisiran combination also met primary endpoint and key secondary endpoint.
   Reduction in MG-ADL total score at week 24 vs placebo (LS means −3.96 vs −2.22, TRT Diff=−1.74 p=0.0086).
   Reduction in QMG total score at week 24 vs. placebo (LS means; −3.32 vs. −1.46, TRT Diff=−1.86 p=0.0348)
Myasthenic crisis (including impending crisis), rescue therapy, and worsening of myasthenia were more favorable in Cemdisiran and combination than placebo.
Cemdisiran monotherapy was significant for all endpoints expected to be in a USPI, and was nominally significant for all other secondary endpoints.
Pozelimab monotherapy as dosed (200 mg Q4W) demonstrated no efficacy.
Contribution of components has not been established.

### Safety

Cemdisiran monotherapy, Pozelimab and Cemdisiran combination and Pozelimab monotherapy had a favorable safety profile with no new safety concerns identified.
The safety profile is similar to approved C5 inhibitors.
Injection site reactions were more common in Cemdisiran monotherapy and Pozelimab and Cemdisiran combination groups.
There were low incidence of treatment emergent ADA responses

### PK/PD

Cemdisiran reduced CH50 by ˜80% at WK24, with the Pozelimab+Cemdisiran combination achieving maximal reduction, while Pozelimab had minimal effect.
Exposures of Pozelimab and total C5 were within the range of PK/PD model-predicted exposures

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present disclosure is not to be limited in scope by the specific embodiments described herein. Indeed, various modifications of the disclosure in addition to those described herein will become apparent to those skilled in the art from the foregoing description and accompanying figures. Such modifications are intended to fall within the scope of the appended claims.

The following sequences have a length that is below the minimum length permitted under ST.26 format:

| SEQ ID NO | Sequence |
|---|---|
| 13 | aaggcgtct |
| 14 | Lys Ala Ser |
| 29 | gatgcatcc |
| 30 | Asp Ala Ser |
| 45 | aaggcgtct |
| 46 | Lys Ala Ser |
| 61 | gctgcatcc |
| 62 | Ala Ala Ser |
| 77 | gctgcatcc |
| 78 | Ala Ala Ser |
| 93 | gatgcatcg |
| 94 | Asp Ala Ser |
| 109 | gctgcatcc |
| 110 | Ala Ala Ser |
| 117 | gctgcatcc |
| 118 | Ala Ala Ser |
| 133 | gctgcatcc |
| 134 | Ala Ala Ser |
| 165 | actgcatcc |
| 166 | Thr Ala Ser |
| 181 | actgcatcc |
| 182 | Thr Ala Ser |
| 197 | gatgcatcc |
| 198 | Asp Ala Ser |
| 213 | aaggcgtct |
| 214 | Lys Ala Ser |
| 229 | gctgcatcc |
| 230 | Ala Ala Ser |
| 245 | gatgcatcc |
| 246 | Asp Ala Ser |
| 261 | gctgcatcc |
| 262 | Ala Ala Ser |
| 301 | gctgcatcc |
| 302 | Ala Ala Ser |
| 317 | gatgcatcc |
| 318 | Asp Ala Ser |
| 333 | ggtgcatcc |
| 334 | Gly Ala Ser |
| 349 | gatgcatcc |
| 350 | Asp Ala Ser |

We claim:

1. A method for
treating or preventing a C5-associated disease or disorder;
treating or preventing myasthenia gravis; or
treating generalized myasthenia gravis (gMG) in adult subjects who are anti-acetylcholine receptor (AChR) or anti-low-density lipoprotein receptor-related protein 4 (LRP4) antibody positive;
in a subject in need thereof comprising administering to the subject a combination of a therapeutically effective amount of an antibody or antigen-binding fragment thereof that binds specifically to C5 in association with a therapeutically effective amount of a C5 iRNA;
or
C5 iRNA monotherapy with does not include said antibody or fragment,
wherein the combination of the antibody or antigen-binding fragment thereof and the C5 iRNA or the C5 iRNA monotherapy is administered to the subject by:
(a) a subcutaneous injection or intravenous infusion of a co-formulation that comprises both (i) the anti-C5 antibody or antigen-binding fragment thereof and (ii) the C5 iRNA;
(b) subcutaneous injections or intravenous infusions of separate formulations that each comprise either (i) the anti-C5 antibody or antigen-binding fragment thereof or (ii) the C5 iRNA;
(c) a subcutaneous injection of an anti-C5 antibody or antigen-binding fragment thereof; or
(d) a subcutaneous injection or intravenous infusion of the C5 iRNA.

2. A method for treating or preventing a C5-associated disease or disorder in a subject in need thereof comprising administering, to the subject, one or more:
(a) approximately 200 mg (±20 mg) doses of the anti-C5 antibody or antigen-binding fragment thereof in association with approximately 200 mg (±20 mg) C5 iRNA or a molar equivalent amount thereof or 10-13 (±2) micromoles of C5 iRNA;
(b) approximately 200 mg (±20 mg) doses of C5 iRNA or a molar equivalent amount thereof or 10-13 (±2) micromoles of C5 iRNA;
(c) approximately 567 or 600 mg (±60 mg) doses of C5 iRNA or a molar equivalent amount thereof or about 32-35 or 33.8 (±4) micromoles of C5 iRNA;
(d) approximately 200 mg (±20 mg) doses of anti-C5 antibody or antigen-binding fragment thereof;
(e) approximately 200 mg (±20 mg) doses of the anti-C5 antibody or antigen-binding fragment thereof, about every 4 weeks (±7 days) in association with approximately 200 mg (±20 mg) C5 iRNA or a molar equivalent amount thereof or 10-13 (±2) micromoles C5 iRNA, about every 4 weeks (±7 days);
(f) approximately 200 mg (±20 mg) doses of anti-C5 antibody or antigen-binding fragment thereof about every 4 weeks (±7 days);
(g) approximately 200 mg (±20 mg) doses of C5 iRNA or a molar equivalent amount thereof or 10-13 (±2) micromoles C5 iRNA about every 4 weeks (±7 days);
(h) approximately 600 mg (±60 mg) doses of C5 iRNA or a molar equivalent amount thereof about every 12 weeks (±7 days) or 84 days (±7 days) or 3 months (±7 days);
(i) approximately 32-35 or 33.8 (±4) micromole doses of C5 iRNA about every 12 weeks (±7 days) or 84 days (±7 days) or 3 months (±7 days); or
(j) approximately 567 mg of the free form of a C5 iRNA which is in a salt form.

3. The method of claim 2 comprising administering one or more doses of approximately 32-35 or 33.8 (±4) micromoles Cemdisiran salt form.

4. The method of claim 2 comprising administering one or more doses of approximately 32-35 or 33.8 (±4) micromoles Cemdisiran salt form once every 12 weeks (±7 days).

5. The method of claim 2 comprising administering one or more doses of approximately 32-35 or 33.8 micromoles (±4) Cemdisiran salt form once every 84 days (±7 days).

6. The method of claim 2 comprising administering one or more doses of approximately 32-35 or 33.8 micromoles (±4) Cemdisiran salt form once every 3 months (±7 days).

7. The method of claim 2 comprising administering one or more doses of approximately 600 mg (±60 mg) Cemdisiran salt form.

8. The method of claim 2 comprising administering one or more doses of approximately 600 mg (±60 mg) Cemdisiran salt form once every 12 weeks (±7 days).

9. The method of claim 2 comprising administering one or more doses of approximately 600 mg (±60 mg) Cemdisiran salt form once every 84 days (±7 days).

10. The method of claim 2 comprising administering one or more doses of approximately 600 mg (±60 mg) Cemdisiran salt form once every 3 months (±7 days).

11. The method of claim 2 comprising administering one or more doses of approximately 567 mg (±57 mg) Cemdisiran free form which is in a salt form.

12. The method of claim 2 comprising administering one or more doses of approximately 567 mg (±57 mg) Cemdisiran free form which is in a salt form once every 12 weeks (±7 days).

13. The method of claim 2 comprising administering one or more doses of approximately 567 mg (±57 mg) Cemdisiran free form which is in a salt form once every 84 days (±7 days).

14. The method of claim 2 comprising administering one or more doses of approximately 567 mg (±57 mg) Cemdisiran free form which is in a salt form once every 3 months (±7 days).

15. The method of claim 2 comprising administering one or more doses of approximately 32-35 or 33.8 (±4) micromoles Cemdisiran Na⁺ salt form.

16. The method of claim 2 comprising administering one or more doses of approximately 32-35 or 33.8 (±4) micromoles Cemdisiran Na⁺ salt form once every 12 weeks (±7 days).

17. The method of claim 2 comprising administering one or more doses of approximately 32-35 or 33.8 micromoles (±4) Cemdisiran Na⁺ salt form once every 84 days (±7 days).

18. The method of claim 2 comprising administering one or more doses of approximately 32-35 or 33.8 micromoles (±4) Cemdisiran Na⁺ salt form once every 3 months (±7 days).

19. The method of claim 2 comprising administering one or more doses of approximately 600 mg (±60 mg) Cemdisiran Na⁺ salt form.

20. The method of claim 2 comprising administering one or more doses of approximately 600 mg (±60 mg) Cemdisiran Na⁺ salt form once every 12 weeks (±7 days).

21. The method of claim 2 comprising administering one or more doses of approximately 600 mg (±60 mg) Cemdisiran Na⁺ salt form once every 84 days (±7 days).

22. The method of claim 2 comprising administering one or more doses of approximately 600 mg (±60 mg) Cemdisiran Na⁺ salt form once every 3 months (±7 days).

23. The method of claim 2 comprising administering one or more doses of approximately 567 mg (±57 mg) Cemdisiran free form which is in a Na⁺ salt form.

24. The method of claim 2 comprising administering one or more doses of approximately 567 mg (±57 mg) Cemdisiran free form which is in a Na⁺ salt form once every 12 weeks (±7 days).

25. The method of claim 2 comprising administering one or more doses of approximately 567 mg (±57 mg) Cemdisiran free form which is in a Na⁺ salt form once every 84 days (±7 days).

26. The method of claim 2 comprising administering one or more doses of approximately 567 mg (±57 mg) Cemdisiran free form which is in a Na⁺ salt form once every 3 months (±7 days).

27. The method of claim 2 comprising administering one or more doses of an amount of Cemdisiran salt form that is the molar equivalent of 600 mg (±60 mg) Cemdisiran Na⁺ salt form.

28. The method of claim 2 comprising administering one or more doses of an amount of Cemdisiran salt form that is the molar equivalent of 600 mg (±60 mg) Cemdisiran Na⁺ salt form once every 12 weeks (±7 days).

29. The method of claim 2 comprising administering one or more doses of an amount of Cemdisiran salt form that is the molar equivalent of 600 mg (±60 mg) Cemdisiran Na⁺ salt form once every 84 days (±7 days).

30. The method of claim 2 comprising administering one or more doses of an amount of Cemdisiran salt form that is the molar equivalent of 600 mg (±60 mg) Cemdisiran Na⁺ salt form once every 3 months (±7 days).

31. The method of any one of the preceding claims wherein the Cemdisiran is administered by subcutaneous injection.

32. The method of any one of the preceding claims wherein the Cemdisiran is in a pharmaceutical formulation comprising water or saline.

33. The method of any one of the preceding claims wherein the Cemdisiran is administered from an on-body injection device, a pre-filled syringe or an autoinjector.

34. The method of any one of claims 2-33 wherein the disease or disorder is myasthenia gravis.

35. The method of any one of claims 2-34 wherein the disease or disorder is generalized myasthenia gravis.

36. The method of any one of the preceding claims, wherein one or more or 2 or more doses of the anti-C5 antibody or antigen-binding fragment thereof are administered to the subject subcutaneously; and/or one or more doses of the C5 iRNA are administered to the subject subcutaneously.

37. The method of any one of the preceding claims, wherein the anti-C5 antibody or antigen-binding fragment thereof and the C5 iRNA are in separate formulations wherein the C5 iRNA is administered before, after, or simultaneously with the anti-C5 antibody or antigen-binding fragment thereof.

38. The method of any one of the preceding claims, wherein the C5 iRNA and the anti-C5 antibody or antigen-binding fragment thereof are administered within 30 minutes or within 1 hour of each other.

39. The method of any one of the preceding claims, wherein the anti-C5 antibody or antigen-binding fragment thereof and the C5 iRNA are in a single co-formulation.

40. The method any one of the preceding claims, wherein the ratio of concentrations (mg/ml) of anti-C5 antibody or antigen-binding fragment thereof and the C5 iRNA in the co-formulation is 1:1.

41. The method of any one of the preceding claims, wherein the concentration of anti-C5 antibody or antigen-binding fragment thereof in the co-formulation is about 100 mg/ml (±10 mg/mg) and the concentration of the C5 iRNA in the co-formulation is about 100 mg/ml (±10 mg/mg).

42. The method of any one of the preceding claims, wherein the co-formulation is administered in a single approximately 2 ml (±0.2 mg) injection.

43. The method of any one of the preceding claims, wherein:
each dose of the anti-C5 antibody or antigen-binding fragment thereof is administered about every 4 weeks (±1, 2, 3, 4, 5, 6, or 7 days);
each dose of the C5 iRNA is administered about every 4 weeks (±3, 4, 5, 6, or 7 days);
the anti-C5 antibody or antigen-binding fragment thereof is administered simultaneously with the C5 iRNA;
the anti-C5 antibody or antigen-binding fragment thereof is administered before the C5 iRNA but during the same treatment session;
and/or
the C5 iRNA is administered before the anti-C5 antibody or antigen-binding fragment thereof but during the same treatment session.

44. The method of any one of the preceding claims, wherein the combination comprises:
(i) a co-formulation, comprising:
the C5 iRNA conjugated to a ligand that comprises one or more terminal N-Acetylgalactosamine (GalNAc) and/or N-acetylglucosamine (GlcNAc) residues,
the anti-C5 antibody or antigen-binding fragment thereof, and
a pharmaceutically acceptable carrier;
wherein the co-formulation has a pH of greater than or less than about 6; or
(ii) a first formulation comprising the anti-C5 antibody or antigen-binding fragment thereof and a pharmaceutically acceptable carrier; and a second formulation comprising the C5 iRNA conjugated to a ligand that comprises one or more terminal N-Acetylgalactosamine (GalNAc) and/or N-acetylglucosamine (GlcNAc) residues, and a pharmaceutically acceptable carrier.

45. The method of any one of the preceding claims, wherein after receiving two or more doses of the C5 iRNA separated by a 12-week interval, administering one or more doses of the C5 iRNA separated by an interval of greater than 12 weeks or less than 12 weeks.

46. The method of any one of the preceding claims, wherein the C5 iRNA and the antibody or antigen binding fragment are in a co-formulation that has a pH of about 6.5 or a pH within not less than 0.5 to 6.0 or of 6.5±0.2.

47. The method of any one of the preceding claims, wherein the C5 iRNA and the antibody or antigen binding fragment are in a co-formulation that comprises a buffer; a viscosity reducer; a stabilizer; and a non-ionic surfactant.

48. The method of any one of the preceding claims, wherein the C5 iRNA and the antibody or antigen binding fragment are in a co-formulation that comprises:
a buffer selected from a histidine-based buffer, a citrate-based buffer, a phosphate-based buffer an acetate-based buffer, and a combination thereof;
a viscosity reducer selected from a conjugate base or salt thereof of an acid, arginine, adipic acid, NaCl, lysine, proline, histidine, caffeine, phenylalanine, triethyl citrate, a dicarboxylic acid, an ester of citric acid, a xanthine, an inorganic salt, a D- or L-amino acid, (D- or L-) arginine, L-arginine HCl, (D- or L-) alanine, proline, (D- or L-) valine, glycine, (D- or L-) serine, (D- or L-) phenylalanine, (D- or L-) lysine, (D- or L-) glutamate and salts thereof, an inorganic salt, NaCl, pyridoxamine, L-ornithine, thiamine phosphoric acid ester chloride dihydrate, benzenesulfonic acid, pyridoxine, or a combination thereof;
a stabilizer which is a polyol; a sugar; trehalose, sorbitol, mannitol, taurine, propane sulfonic acid, L-proline, sucrose, glycerol, threitol, maltitol, polyethylene glycol (PEG), PEG3350, or a combination thereof; and/or
a non-ionic surfactant selected from a polyoxyethylene glycol alkyl ether; glucoside alkyl ether; polyoxyethylene glycol octylphenol ether; polyoxyethylene glycol alkylphenol ether; glycerol alkyl ester; polyoxyethylene glycol sorbitan alkyl ester; sorbitan alkyl ester; block copolymer of polypropylene glycol; block copolymer of polyethylene glycol; polysorbate; octaethylene glycol monododecyl ether; pentaethylene glycol monododecyl ether; polyoxypropylene glycol alkyl ether; decyl glucoside, lauryl glucoside, octyl glucoside; triton X-100; nonoxynol-9; glyceryl laurate; cocamide MEA, cocamide DEA, dodecyldimethylamine oxide; poloxamer; polyethoxylated tallow amine (POEA); polysorbate-20 (PS20); polysorbate-80 (PS80), and a combination thereof.

49. The method of any one of the preceding claims, wherein the C5 iRNA and the antibody or antigen binding fragment are in a co-formulation that comprises
about 50-100, 50-75, 10-35, 35-45, 20-50, 20, 25, 30, 35, 40, 45 or 50 mM buffer;
about 20-140, 20, 25, 30, 35, 40, 45, 50, 55, 60, 65, 70, 75, 80, 85, 90, 95, 100, 105, 110, 115, 120, 125, 130, 135 or 140 mM viscosity reducer;
about 0.8-3.6, 0.8, 0.9, 1.0, 1.25, 1.50, 2.0, 2.25, 2.50, 2.75, 3.00, 3.1, 3.2, 3.3, 3.4, 3.5 or 3.6% (w/v) stabilizer;
and/or
about 0.025, 0.05, 0.075, 0.1, 0.125, 0.15, or 0.175% (w/v) non-ionic surfactant.

50. The method of any one of the preceding claims, wherein the C5 iRNA and the antibody or antigen binding fragment are in a co-formulation that comprises about 96% or more anti-C5 antibody or antigen-binding fragment thereof purity as assessed by size exclusion chromatography after about 1 month of storage at 2-8° C.

51. The method of any one of the preceding claims, wherein the C5 iRNA and the antibody or antigen binding fragment are in a co-formulation that comprises about 94% or more C5 iRNA purity as assessed by anion exchange chromatography after about 1 month of storage at 2-8° C.

52. The method of any one of the preceding claims, wherein the C5 iRNA and the antibody or antigen binding fragment are in a co-formulation that comprises a 1:1 ratio of concentration of C5 iRNA to anti-C5 antibody or antigen-binding fragment thereof in milligrams per milliliter.

53. The method of any one of the preceding claims, wherein the C5 iRNA and the antibody or antigen binding fragment are in a co-formulation that comprises a viscosity reducer which is 75 mM arginine, 75 mM adipate, 75 mM NaCl, 75 mM lysine, 75 mM aspartate, 75 mM proline, 50 mM histidine, 50 mM caffeine, 50 mM phenylalanine, 75 mM triethyl citrate, and combinations thereof; wherein, if the buffer is histidine-based, the total histidine concentration of the co-formulation is 50 mM.

54. The method of any one of the preceding claims, wherein the anti-C5 antibody or antigen-binding fragment is Pozelimab which is expressed in a mammalian host cell and/or the Cemdisiran is chemically synthesized.

55. The method of claim 54, wherein the host cell is a Chinese hamster ovary cell.

56. The method of any one of the preceding claims, wherein the combination is administered as a co-formulation, which comprises:
no more than about 2.1 parts per million molar ratio of beta-hexosaminidase to anti-C5 antibody or antigen-binding fragment;
no more than about 0.170 micrograms/ml beta-hexosaminidase;
no more than about 0.04 micrograms/ml beta-hexosaminidase; and/or
about 0.04; 0.05; 0.06; 0.0605; 0.063; 0.07; 0.0765; 0.078; 0.08; 0.14; 0.141; 0.15; 0.1525; 0.166; or 0.17 micrograms/ml beta-hexosaminidase; or no more than any of such concentrations.

57. The method of any one of the preceding claims, wherein the anti-C5 antibody or antigen-binding fragment thereof comprises:
(i)
a heavy chain variable region (HCVR) that comprises the HCDR1, HCDR2 and HCDR3 of a HCVR that comprises the amino acid sequence set forth in SEQ ID NO: 2, and
a light chain variable region (LCVR) that comprises the LCDR1, LCDR2 and LCDR3 of a LCVR that comprises the amino acid sequence set forth in SEQ ID NO: 10;
(ii)
a HCVR that comprises the HCDR1, HCDR2 and HCDR3 of a HCVR that comprises the amino acid sequence set forth in SEQ ID NO: 18, and
a LCVR that comprises the LCDR1, LCDR2 and LCDR3 of a LCVR that comprises the amino acid sequence set forth in SEQ ID NO: 26;
(iii)
a HCVR that comprises the HCDR1, HCDR2 and HCDR3 of a HCVR that comprises the amino acid sequence set forth in SEQ ID NO: 34, and
a LCVR that comprises the LCDR1, LCDR2 and LCDR3 of a LCVR that comprises the amino acid sequence set forth in SEQ ID NO: 42;
(iv)
a HCVR that comprises the HCDR1, HCDR2 and HCDR3 of a HCVR that comprises the amino acid sequence set forth in SEQ ID NO: 50, and
a LCVR that comprises the LCDR1, LCDR2 and LCDR3 of a LCVR that comprises the amino acid sequence set forth in SEQ ID NO: 58;
(v)
a HCVR that comprises the HCDR1, HCDR2 and HCDR3 of a HCVR that comprises the amino acid sequence set forth in SEQ ID NO: 66, and
a LCVR that comprises the LCDR1, LCDR2 and LCDR3 of a LCVR that comprises the amino acid sequence set forth in SEQ ID NO: 74;
(vi)
a HCVR that comprises the HCDR1, HCDR2 and HCDR3 of a HCVR that comprises the amino acid sequence set forth in SEQ ID NO: 82, and
a LCVR that comprises the LCDR1, LCDR2 and LCDR3 of a LCVR that comprises the amino acid sequence set forth in SEQ ID NO: 90;
(vii)
a HCVR that comprises the HCDR1, HCDR2 and HCDR3 of a HCVR that comprises the amino acid sequence set forth in SEQ ID NO: 98, and
a LCVR that comprises the LCDR1, LCDR2 and LCDR3 of a LCVR that comprises the amino acid sequence set forth in SEQ ID NO: 106;
(viii)
a HCVR that comprises the HCDR1, HCDR2 and HCDR3 of a HCVR that comprises the amino acid sequence set forth in SEQ ID NO: 98, and
a LCVR that comprises the LCDR1, LCDR2 and LCDR3 of a LCVR that comprises the amino acid sequence set forth in SEQ ID NO: 114;
(ix)
a HCVR that comprises the HCDR1, HCDR2 and HCDR3 of a HCVR that comprises the amino acid sequence set forth in SEQ ID NO: 122, and
a LCVR that comprises the LCDR1, LCDR2 and LCDR3 of a LCVR that comprises the amino acid sequence set forth in SEQ ID NO: 106;
(x)
a HCVR that comprises the HCDR1, HCDR2 and HCDR3 of a HCVR that comprises the amino acid sequence set forth in SEQ ID NO: 98, and
a LCVR that comprises the LCDR1, LCDR2 and LCDR3 of a LCVR that comprises the amino acid sequence set forth in SEQ ID NO: 130;
(xi)
a HCVR that comprises the HCDR1, HCDR2 and HCDR3 of a HCVR that comprises the amino acid sequence set forth in SEQ ID NO: 138, and
a LCVR that comprises the LCDR1, LCDR2 and LCDR3 of a LCVR that comprises the amino acid sequence set forth in SEQ ID NO: 106;
(xii)
a HCVR that comprises the HCDR1, HCDR2 and HCDR3 of a HCVR that comprises the amino acid sequence set forth in SEQ ID NO: 146, and
a LCVR that comprises the LCDR1, LCDR2 and LCDR3 of a LCVR that comprises the amino acid sequence set forth in SEQ ID NO: 106;
(xiii)
a HCVR that comprises the HCDR1, HCDR2 and HCDR3 of a HCVR that comprises the amino acid sequence set forth in SEQ ID NO: 122, and
a LCVR that comprises the LCDR1, LCDR2 and LCDR3 of a LCVR that comprises the amino acid sequence set forth in SEQ ID NO: 130;
(xiv)
a HCVR that comprises the HCDR1, HCDR2 and HCDR3 of a HCVR that comprises the amino acid sequence set forth in SEQ ID NO: 146, and
a LCVR that comprises the LCDR1, LCDR2 and LCDR3 of a LCVR that comprises the amino acid sequence set forth in SEQ ID NO: 114;
(xv)
a HCVR that comprises the HCDR1, HCDR2 and HCDR3 of a HCVR that comprises the amino acid sequence set forth in SEQ ID NO: 146, and
a LCVR that comprises the LCDR1, LCDR2 and LCDR3 of a LCVR that comprises the amino acid sequence set forth in SEQ ID NO: 130;
(xvi)
a HCVR that comprises the HCDR1, HCDR2 and HCDR3 of a HCVR that comprises the amino acid sequence set forth in SEQ ID NO: 138, and
a LCVR that comprises the LCDR1, LCDR2 and LCDR3 of a LCVR that comprises the amino acid sequence set forth in SEQ ID NO: 130;
(xvii)
a HCVR that comprises the HCDR1, HCDR2 and HCDR3 of a HCVR that comprises the amino acid sequence set forth in SEQ ID NO: 154, and
a LCVR that comprises the LCDR1, LCDR2 and LCDR3 of a LCVR that comprises the amino acid sequence set forth in SEQ ID NO: 162;
(xviii)
a HCVR that comprises the HCDR1, HCDR2 and HCDR3 of a HCVR that comprises the amino acid sequence set forth in SEQ ID NO: 170, and
a LCVR that comprises the LCDR1, LCDR2 and LCDR3 of a LCVR that comprises the amino acid sequence set forth in SEQ ID NO: 178;
(xix)
a HCVR that comprises the HCDR1, HCDR2 and HCDR3 of a HCVR that comprises the amino acid sequence set forth in SEQ ID NO: 186, and
a LCVR that comprises the LCDR1, LCDR2 and LCDR3 of a LCVR that comprises the amino acid sequence set forth in SEQ ID NO: 194;
(xx)
a HCVR that comprises the HCDR1, HCDR2 and HCDR3 of a HCVR that comprises the amino acid sequence set forth in SEQ ID NO: 202, and
a LCVR that comprises the LCDR1, LCDR2 and LCDR3 of a LCVR that comprises the amino acid sequence set forth in SEQ ID NO: 210;
(xxi)
a HCVR that comprises the HCDR1, HCDR2 and HCDR3 of a HCVR that comprises the amino acid sequence set forth in SEQ ID NO: 218, and
a LCVR that comprises the LCDR1, LCDR2 and LCDR3 of a LCVR that comprises the amino acid sequence set forth in SEQ ID NO: 226;
(xxii)
a HCVR that comprises the HCDR1, HCDR2 and HCDR3 of a HCVR that comprises the amino acid sequence set forth in SEQ ID NO: 234, and
a LCVR that comprises the LCDR1, LCDR2 and LCDR3 of a LCVR that comprises the amino acid sequence set forth in SEQ ID NO: 242;
(xxiii)
a HCVR that comprises the HCDR1, HCDR2 and HCDR3 of a HCVR that comprises the amino acid sequence set forth in SEQ ID NO: 250, and
a LCVR that comprises the LCDR1, LCDR2 and LCDR3 of a LCVR that comprises the amino acid sequence set forth in SEQ ID NO: 258;
(xxiv)
a HCVR that comprises the HCDR1, HCDR2 and HCDR3 of a HCVR that comprises the amino acid sequence set forth in SEQ ID NO: 266, and
a LCVR that comprises the LCDR1, LCDR2 and LCDR3 of a LCVR that comprises the amino acid sequence set forth in SEQ ID NO: 258;
(xxv)
a HCVR that comprises the HCDR1, HCDR2 and HCDR3 of a HCVR that comprises the amino acid sequence set forth in SEQ ID NO: 274, and
a LCVR that comprises the LCDR1, LCDR2 and LCDR3 of a LCVR that comprises the amino acid sequence set forth in SEQ ID NO: 282;
(xxvi)
a HCVR that comprises the HCDR1, HCDR2 and HCDR3 of a HCVR that comprises the amino acid sequence set forth in SEQ ID NO: 290, and
a LCVR that comprises the LCDR1, LCDR2 and LCDR3 of a LCVR that comprises the amino acid sequence set forth in SEQ ID NO: 298;
(xxvii)
a HCVR that comprises the HCDR1, HCDR2 and HCDR3 of a HCVR that comprises the amino acid sequence set forth in SEQ ID NO: 306, and
a LCVR that comprises the LCDR1, LCDR2 and LCDR3 of a LCVR that comprises the amino acid sequence set forth in SEQ ID NO: 314;
(xxviii)
a HCVR that comprises the HCDR1, HCDR2 and HCDR3 of a HCVR that comprises the amino acid sequence set forth in SEQ ID NO: 322, and
a LCVR that comprises the LCDR1, LCDR2 and LCDR3 of a LCVR that comprises the amino acid sequence set forth in SEQ ID NO: 330;
or
(xxix)
a HCVR that comprises the HCDR1, HCDR2 and HCDR3 of a HCVR that comprises the amino acid sequence set forth in SEQ ID NO: 338, and
a LCVR that comprises the LCDR1, LCDR2 and LCDR3 of a LCVR that comprises the amino acid sequence set forth in SEQ ID NO: 346.

58. The method of any one of the preceding claims, wherein the anti-C5 antibody or antigen-binding fragment thereof comprises:
(i)
a heavy chain variable region (HCVR) comprising
an HCDR1 that comprises the amino acid sequence set forth in SEQ ID NO: 4,
an HCDR2 that comprises the amino acid sequence set forth in SEQ ID NO: 6,
an HCDR3 that comprises the amino acid sequence set forth in SEQ ID NO: 8, and
a light chain variable region (LCVR) comprising
an LCDR1 that comprises the amino acid sequence set forth in SEQ ID NO: 12,
an LCDR2 that comprises the amino acid sequence set forth in SEQ ID NO: 14,
an LCDR3 that comprises the amino acid sequence set forth in SEQ ID NO: 16;
(ii)
a HCVR comprising
an HCDR1 that comprises the amino acid sequence set forth in SEQ ID NO: 20,
an HCDR2 that comprises the amino acid sequence set forth in SEQ ID NO: 22,
an HCDR3 that comprises the amino acid sequence set forth in SEQ ID NO: 24, and
a LCVR comprising
an LCDR1 that comprises the amino acid sequence set forth in SEQ ID NO: 28,
an LCDR2 that comprises the amino acid sequence set forth in SEQ ID NO: 30,
an LCDR3 that comprises the amino acid sequence set forth in SEQ ID NO: 32;
(iii)
a HCVR comprising
an HCDR1 that comprises the amino acid sequence set forth in SEQ ID NO: 36,
an HCDR2 that comprises the amino acid sequence set forth in SEQ ID NO: 38,
an HCDR3 that comprises the amino acid sequence set forth in SEQ ID NO: 40, and
a LCVR comprising
an LCDR1 that comprises the amino acid sequence set forth in SEQ ID NO: 44,
an LCDR2 that comprises the amino acid sequence set forth in SEQ ID NO: 46,
an LCDR3 that comprises the amino acid sequence set forth in SEQ ID NO: 48;
(iv)
a HCVR comprising
an HCDR1 that comprises the amino acid sequence set forth in SEQ ID NO: 52,
an HCDR2 that comprises the amino acid sequence set forth in SEQ ID NO: 54,
an HCDR3 that comprises the amino acid sequence set forth in SEQ ID NO: 56, and
a LCVR comprising
an LCDR1 that comprises the amino acid sequence set forth in SEQ ID NO: 60,
an LCDR2 that comprises the amino acid sequence set forth in SEQ ID NO: 62,
an LCDR3 that comprises the amino acid sequence set forth in SEQ ID NO: 64;
(v)
a HCVR comprising
an HCDR1 that comprises the amino acid sequence set forth in SEQ ID NO: 68,
an HCDR2 that comprises the amino acid sequence set forth in SEQ ID NO: 70,
an HCDR3 that comprises the amino acid sequence set forth in SEQ ID NO: 72, and
a LCVR comprising
an LCDR1 that comprises the amino acid sequence set forth in SEQ ID NO: 76,
an LCDR2 that comprises the amino acid sequence set forth in SEQ ID NO: 78,
an LCDR3 that comprises the amino acid sequence set forth in SEQ ID NO: 80;
(vi)
a HCVR comprising
an HCDR1 that comprises the amino acid sequence set forth in SEQ ID NO: 84,
an HCDR2 that comprises the amino acid sequence set forth in SEQ ID NO: 86,
an HCDR3 that comprises the amino acid sequence set forth in SEQ ID NO: 88, and
a LCVR comprising
an LCDR1 that comprises the amino acid sequence set forth in SEQ ID NO: 92,
an LCDR2 that comprises the amino acid sequence set forth in SEQ ID NO: 94,
an LCDR3 that comprises the amino acid sequence set forth in SEQ ID NO: 96;
(vii)
a HCVR comprising
an HCDR1 that comprises the amino acid sequence set forth in SEQ ID NO: 100,
an HCDR2 that comprises the amino acid sequence set forth in SEQ ID NO: 102,
an HCDR3 that comprises the amino acid sequence set forth in SEQ ID NO: 104, and
a LCVR comprising
an LCDR1 that comprises the amino acid sequence set forth in SEQ ID NO: 108,
an LCDR2 that comprises the amino acid sequence set forth in SEQ ID NO: 110,
an LCDR3 that comprises the amino acid sequence set forth in SEQ ID NO: 112;
(viii)
a HCVR comprising
an HCDR1 that comprises the amino acid sequence set forth in SEQ ID NO: 100,
an HCDR2 that comprises the amino acid sequence set forth in SEQ ID NO: 102,
an HCDR3 that comprises the amino acid sequence set forth in SEQ ID NO: 104, and
a LCVR comprising
an LCDR1 that comprises the amino acid sequence set forth in SEQ ID NO: 116,
an LCDR2 that comprises the amino acid sequence set forth in SEQ ID NO: 118,
an LCDR3 that comprises the amino acid sequence set forth in SEQ ID NO: 120;
(ix)
a HCVR comprising
an HCDR1 that comprises the amino acid sequence set forth in SEQ ID NO: 124,
an HCDR2 that comprises the amino acid sequence set forth in SEQ ID NO: 126,
an HCDR3 that comprises the amino acid sequence set forth in SEQ ID NO: 128, and
a LCVR comprising
an LCDR1 that comprises the amino acid sequence set forth in SEQ ID NO: 108,
an LCDR2 that comprises the amino acid sequence set forth in SEQ ID NO: 110,
an LCDR3 that comprises the amino acid sequence set forth in SEQ ID NO: 112;
(x)
a HCVR comprising
an HCDR1 that comprises the amino acid sequence set forth in SEQ ID NO: 100,
an HCDR2 that comprises the amino acid sequence set forth in SEQ ID NO: 102,
an HCDR3 that comprises the amino acid sequence set forth in SEQ ID NO: 104, and
a LCVR comprising
an LCDR1 that comprises the amino acid sequence set forth in SEQ ID NO: 132,
an LCDR2 that comprises the amino acid sequence set forth in SEQ ID NO: 134,
an LCDR3 that comprises the amino acid sequence set forth in SEQ ID NO: 136;
(xi)
a HCVR comprising
an HCDR1 that comprises the amino acid sequence set forth in SEQ ID NO: 140,
an HCDR2 that comprises the amino acid sequence set forth in SEQ ID NO: 142,
an HCDR3 that comprises the amino acid sequence set forth in SEQ ID NO: 144, and
a LCVR comprising
an LCDR1 that comprises the amino acid sequence set forth in SEQ ID NO: 108,
an LCDR2 that comprises the amino acid sequence set forth in SEQ ID NO: 110,
an LCDR3 that comprises the amino acid sequence set forth in SEQ ID NO: 112;
(xii)
a HCVR comprising
an HCDR1 that comprises the amino acid sequence set forth in SEQ ID NO: 148,
an HCDR2 that comprises the amino acid sequence set forth in SEQ ID NO: 150,
an HCDR3 that comprises the amino acid sequence set forth in SEQ ID NO: 152, and
a LCVR comprising
an LCDR1 that comprises the amino acid sequence set forth in SEQ ID NO: 108,
an LCDR2 that comprises the amino acid sequence set forth in SEQ ID NO: 110,
an LCDR3 that comprises the amino acid sequence set forth in SEQ ID NO: 112;
(xiii)
a HCVR comprising
an HCDR1 that comprises the amino acid sequence set forth in SEQ ID NO: 124,
an HCDR2 that comprises the amino acid sequence set forth in SEQ ID NO: 126,
an HCDR3 that comprises the amino acid sequence set forth in SEQ ID NO: 128, and
a LCVR comprising
an LCDR1 that comprises the amino acid sequence set forth in SEQ ID NO: 132,
an LCDR2 that comprises the amino acid sequence set forth in SEQ ID NO: 134,
an LCDR3 that comprises the amino acid sequence set forth in SEQ ID NO: 136;
(xiv)
a HCVR comprising
an HCDR1 that comprises the amino acid sequence set forth in SEQ ID NO: 148,
an HCDR2 that comprises the amino acid sequence set forth in SEQ ID NO: 150,
an HCDR3 that comprises the amino acid sequence set forth in SEQ ID NO: 152, and
a LCVR comprising
an LCDR1 that comprises the amino acid sequence set forth in SEQ ID NO: 116,
an LCDR2 that comprises the amino acid sequence set forth in SEQ ID NO: 118,
an LCDR3 that comprises the amino acid sequence set forth in SEQ ID NO: 120;
(xv)
a HCVR comprising
an HCDR1 that comprises the amino acid sequence set forth in SEQ ID NO: 148,
an HCDR2 that comprises the amino acid sequence set forth in SEQ ID NO: 150,
an HCDR3 that comprises the amino acid sequence set forth in SEQ ID NO: 152, and
a LCVR comprising
an LCDR1 that comprises the amino acid sequence set forth in SEQ ID NO: 132,
an LCDR2 that comprises the amino acid sequence set forth in SEQ ID NO: 134,
an LCDR3 that comprises the amino acid sequence set forth in SEQ ID NO: 136;
(xvi)
a HCVR comprising
an HCDR1 that comprises the amino acid sequence set forth in SEQ ID NO: 140,
an HCDR2 that comprises the amino acid sequence set forth in SEQ ID NO: 142,
an HCDR3 that comprises the amino acid sequence set forth in SEQ ID NO: 144, and
a LCVR comprising
an LCDR1 that comprises the amino acid sequence set forth in SEQ ID NO: 132,
an LCDR2 that comprises the amino acid sequence set forth in SEQ ID NO: 134,
an LCDR3 that comprises the amino acid sequence set forth in SEQ ID NO: 136;
(xvii)
a HCVR comprising
an HCDR1 that comprises the amino acid sequence set forth in SEQ ID NO: 156,
an HCDR2 that comprises the amino acid sequence set forth in SEQ ID NO: 158,
an HCDR3 that comprises the amino acid sequence set forth in SEQ ID NO: 160, and
a LCVR comprising
an LCDR1 that comprises the amino acid sequence set forth in SEQ ID NO: 164,
an LCDR2 that comprises the amino acid sequence set forth in SEQ ID NO: 166,
an LCDR3 that comprises the amino acid sequence set forth in SEQ ID NO: 168;
(xviii)
a HCVR comprising
an HCDR1 that comprises the amino acid sequence set forth in SEQ ID NO: 172,
an HCDR2 that comprises the amino acid sequence set forth in SEQ ID NO: 174,
an HCDR3 that comprises the amino acid sequence set forth in SEQ ID NO: 176, and
a LCVR comprising
an LCDR1 that comprises the amino acid sequence set forth in SEQ ID NO: 180,
an LCDR2 that comprises the amino acid sequence set forth in SEQ ID NO: 182,
an LCDR3 that comprises the amino acid sequence set forth in SEQ ID NO: 184;
(xix)
a HCVR comprising
an HCDR1 that comprises the amino acid sequence set forth in SEQ ID NO: 188,
an HCDR2 that comprises the amino acid sequence set forth in SEQ ID NO: 190,
an HCDR3 that comprises the amino acid sequence set forth in SEQ ID NO: 192, and
a LCVR comprising
an LCDR1 that comprises the amino acid sequence set forth in SEQ ID NO: 196,
an LCDR2 that comprises the amino acid sequence set forth in SEQ ID NO: 198,
an LCDR3 that comprises the amino acid sequence set forth in SEQ ID NO: 200;
(xx)
a HCVR comprising
an HCDR1 that comprises the amino acid sequence set forth in SEQ ID NO: 204,
an HCDR2 that comprises the amino acid sequence set forth in SEQ ID NO: 206,
an HCDR3 that comprises the amino acid sequence set forth in SEQ ID NO: 208, and
a LCVR comprising
an LCDR1 that comprises the amino acid sequence set forth in SEQ ID NO: 212,
an LCDR2 that comprises the amino acid sequence set forth in SEQ ID NO: 214,
an LCDR3 that comprises the amino acid sequence set forth in SEQ ID NO: 216;
(xxi)
a HCVR comprising
an HCDR1 that comprises the amino acid sequence set forth in SEQ ID NO: 220,
an HCDR2 that comprises the amino acid sequence set forth in SEQ ID NO: 222,
an HCDR3 that comprises the amino acid sequence set forth in SEQ ID NO: 224, and
a LCVR comprising
an LCDR1 that comprises the amino acid sequence set forth in SEQ ID NO: 228,
an LCDR2 that comprises the amino acid sequence set forth in SEQ ID NO: 230,
an LCDR3 that comprises the amino acid sequence set forth in SEQ ID NO: 232;
(xxii)
a HCVR comprising
an HCDR1 that comprises the amino acid sequence set forth in SEQ ID NO: 236,
an HCDR2 that comprises the amino acid sequence set forth in SEQ ID NO: 238,
an HCDR3 that comprises the amino acid sequence set forth in SEQ ID NO: 240, and
a LCVR comprising
an LCDR1 that comprises the amino acid sequence set forth in SEQ ID NO: 244,
an LCDR2 that comprises the amino acid sequence set forth in SEQ ID NO: 246,
an LCDR3 that comprises the amino acid sequence set forth in SEQ ID NO: 248;
(xxiii)
a HCVR comprising
an HCDR1 that comprises the amino acid sequence set forth in SEQ ID NO: 252,
an HCDR2 that comprises the amino acid sequence set forth in SEQ ID NO: 254,
an HCDR3 that comprises the amino acid sequence set forth in SEQ ID NO: 256, and
a LCVR comprising
an LCDR1 that comprises the amino acid sequence set forth in SEQ ID NO: 260,
an LCDR2 that comprises the amino acid sequence set forth in SEQ ID NO: 262,
an LCDR3 that comprises the amino acid sequence set forth in SEQ ID NO: 264;
(xxiv)
a HCVR comprising
an HCDR1 that comprises the amino acid sequence set forth in SEQ ID NO: 268,
an HCDR2 that comprises the amino acid sequence set forth in SEQ ID NO: 270,
an HCDR3 that comprises the amino acid sequence set forth in SEQ ID NO: 272, and
a LCVR comprising
an LCDR1 that comprises the amino acid sequence set forth in SEQ ID NO: 260,
an LCDR2 that comprises the amino acid sequence set forth in SEQ ID NO: 262,
an LCDR3 that comprises the amino acid sequence set forth in SEQ ID NO: 264;
(xxv)
a HCVR comprising
an HCDR1 that comprises the amino acid sequence set forth in SEQ ID NO: 276,
an HCDR2 that comprises the amino acid sequence set forth in SEQ ID NO: 278,
an HCDR3 that comprises the amino acid sequence set forth in SEQ ID NO: 280, and
a LCVR comprising
an LCDR1 that comprises the amino acid sequence set forth in SEQ ID NO: 284,
an LCDR2 that comprises the amino acid sequence set forth in SEQ ID NO: 286,
an LCDR3 that comprises the amino acid sequence set forth in SEQ ID NO: 288;
(xxvi)
a HCVR comprising
an HCDR1 that comprises the amino acid sequence set forth in SEQ ID NO: 292,
an HCDR2 that comprises the amino acid sequence set forth in SEQ ID NO: 294,
an HCDR3 that comprises the amino acid sequence set forth in SEQ ID NO: 296, and
a LCVR comprising
an LCDR1 that comprises the amino acid sequence set forth in SEQ ID NO: 300,
an LCDR2 that comprises the amino acid sequence set forth in SEQ ID NO: 302,
an LCDR3 that comprises the amino acid sequence set forth in SEQ ID NO: 304;
(xxvii)
a HCVR comprising
an HCDR1 that comprises the amino acid sequence set forth in SEQ ID NO: 308,
an HCDR2 that comprises the amino acid sequence set forth in SEQ ID NO: 310,
an HCDR3 that comprises the amino acid sequence set forth in SEQ ID NO: 312, and
a LCVR comprising
an LCDR1 that comprises the amino acid sequence set forth in SEQ ID NO: 316,
an LCDR2 that comprises the amino acid sequence set forth in SEQ ID NO: 318,
an LCDR3 that comprises the amino acid sequence set forth in SEQ ID NO: 320;
(xxviii)
a HCVR comprising
an HCDR1 that comprises the amino acid sequence set forth in SEQ ID NO: 324,
an HCDR2 that comprises the amino acid sequence set forth in SEQ ID NO: 326,
an HCDR3 that comprises the amino acid sequence set forth in SEQ ID NO: 328, and
a LCVR comprising
an LCDR1 that comprises the amino acid sequence set forth in SEQ ID NO: 332,
an LCDR2 that comprises the amino acid sequence set forth in SEQ ID NO: 334,
an LCDR3 that comprises the amino acid sequence set forth in SEQ ID NO: 336; or
(xxix)
a HCVR comprising
an HCDR1 that comprises the amino acid sequence set forth in SEQ ID NO: 340,
an HCDR2 that comprises the amino acid sequence set forth in SEQ ID NO: 342,
an HCDR3 that comprises the amino acid sequence set forth in SEQ ID NO: 344, and
a LCVR comprising
an LCDR1 that comprises the amino acid sequence set forth in SEQ ID NO: 348,
an LCDR2 that comprises the amino acid sequence set forth in SEQ ID NO: 350,
an LCDR3 that comprises the amino acid sequence set forth in SEQ ID NO: 352.

59. The method of any one of the preceding claims, wherein the anti-C5 antibody or antigen-binding fragment thereof comprises:
(i)
a heavy chain variable region (HCVR) that comprises the amino acid sequence set forth in SEQ ID NO: 2, and
a light chain variable region (LCVR) that comprises the amino acid sequence set forth in SEQ ID NO: 10;
(ii)
a HCVR that comprises the amino acid sequence set forth in SEQ ID NO: 18, and
a LCVR that comprises the amino acid sequence set forth in SEQ ID NO: 26;
(iii)
a HCVR that comprises the amino acid sequence set forth in SEQ ID NO: 34, and
a LCVR that comprises the amino acid sequence set forth in SEQ ID NO: 42;
(iv)
a HCVR that comprises the amino acid sequence set forth in SEQ ID NO: 50, and
a LCVR that comprises the amino acid sequence set forth in SEQ ID NO: 58;
(v)
a HCVR that comprises the amino acid sequence set forth in SEQ ID NO: 66, and
a LCVR that comprises the amino acid sequence set forth in SEQ ID NO: 74;
(vi)
a HCVR that comprises the amino acid sequence set forth in SEQ ID NO: 82, and
a LCVR that comprises the amino acid sequence set forth in SEQ ID NO: 90;
(vii)
a HCVR that comprises the amino acid sequence set forth in SEQ ID NO: 98, and
a LCVR that comprises the amino acid sequence set forth in SEQ ID NO: 106;
(viii)
a HCVR that comprises the amino acid sequence set forth in SEQ ID NO: 98, and
a LCVR that comprises the amino acid sequence set forth in SEQ ID NO: 114;
(ix)
a HCVR that comprises the amino acid sequence set forth in SEQ ID NO: 122, and
a LCVR that comprises the amino acid sequence set forth in SEQ ID NO: 106;
(x)
a HCVR that comprises the amino acid sequence set forth in SEQ ID NO: 98, and
a LCVR that comprises the amino acid sequence set forth in SEQ ID NO: 130;
(xi)
a HCVR that comprises the amino acid sequence set forth in SEQ ID NO: 138, and
a LCVR that comprises the amino acid sequence set forth in SEQ ID NO: 106;
(xii)
a HCVR that comprises the amino acid sequence set forth in SEQ ID NO: 146, and
a LCVR that comprises the amino acid sequence set forth in SEQ ID NO: 106;
(xiii)
a HCVR that comprises the amino acid sequence set forth in SEQ ID NO: 122, and
a LCVR that comprises the amino acid sequence set forth in SEQ ID NO: 130;
(xiv)
a HCVR that comprises the amino acid sequence set forth in SEQ ID NO: 146, and
a LCVR that comprises the amino acid sequence set forth in SEQ ID NO: 114;
(xv)
a HCVR that comprises the amino acid sequence set forth in SEQ ID NO: 146, and
a LCVR that comprises the amino acid sequence set forth in SEQ ID NO: 130;
(xvi)
a HCVR that comprises the amino acid sequence set forth in SEQ ID NO: 138, and
a LCVR that comprises the amino acid sequence set forth in SEQ ID NO: 130;
(xvii)
a HCVR that comprises the amino acid sequence set forth in SEQ ID NO: 154, and
a LCVR that comprises the amino acid sequence set forth in SEQ ID NO: 162;
(xviii)
a HCVR that comprises the amino acid sequence set forth in SEQ ID NO: 170, and
a LCVR that comprises the amino acid sequence set forth in SEQ ID NO: 178;
(xix)
a HCVR that comprises the amino acid sequence set forth in SEQ ID NO: 186, and
a LCVR that comprises the amino acid sequence set forth in SEQ ID NO: 194;
(xx)
a HCVR that comprises the amino acid sequence set forth in SEQ ID NO: 202, and
a LCVR that comprises the amino acid sequence set forth in SEQ ID NO: 210;
(xxi)
a HCVR that comprises the amino acid sequence set forth in SEQ ID NO: 218, and
a LCVR that comprises the amino acid sequence set forth in SEQ ID NO: 226;
(xxii)
a HCVR that comprises the amino acid sequence set forth in SEQ ID NO: 234, and
a LCVR that comprises the amino acid sequence set forth in SEQ ID NO: 242;
(xxiii)
a HCVR that comprises the amino acid sequence set forth in SEQ ID NO: 250, and
a LCVR that comprises the amino acid sequence set forth in SEQ ID NO: 258;
(xxiv)
a HCVR that comprises the amino acid sequence set forth in SEQ ID NO: 266, and
a LCVR that comprises the amino acid sequence set forth in SEQ ID NO: 258;
(xxv)
a HCVR that comprises the amino acid sequence set forth in SEQ ID NO: 274, and
a LCVR that comprises the amino acid sequence set forth in SEQ ID NO: 282;
(xxvi)
a HCVR that comprises the amino acid sequence set forth in SEQ ID NO: 290, and
a LCVR that comprises the amino acid sequence set forth in SEQ ID NO: 298;
(xxvii)
a HCVR that comprises the amino acid sequence set forth in SEQ ID NO: 306, and
a LCVR that comprises the amino acid sequence set forth in SEQ ID NO: 314;
(xxviii)
a HCVR that comprises the amino acid sequence set forth in SEQ ID NO: 322, and
a LCVR that comprises the amino acid sequence set forth in SEQ ID NO: 330; or
(xxix)
a HCVR that comprises the amino acid sequence set forth in SEQ ID NO: 338, and
a LCVR that comprises the amino acid sequence set forth in SEQ ID NO: 346.

60. The method of any one of the preceding claims, wherein the anti-C5 antibody or antigen-binding fragment comprises a heavy chain including the amino acid sequence:
(SEQ ID NO: 358)
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;
optionally, lacking the C-terminal lysine;
and a light chain including the amino acid sequence:
(SEQ ID NO: 359)
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

61. The method of any one of the preceding claims, wherein the anti-C5 antibody or antigen-binding fragment thereof is Pozelimab.

62. The method of any one of the preceding claims, wherein the C5 iRNA comprises an RNA strand that is complementary to an mRNA transcribed from the C5 gene sense strand DNA sequence AAGCAAGATATTTTTATAATA (SEQ ID NO: 407; nucleotides 782-802 of SEQ ID NO: 360).

63. The method of any one of the preceding claims, wherein the C5 iRNA is a double-stranded ribonucleic acid (dsRNA) agent comprising a sense strand and an antisense strand;
wherein the antisense strand comprises a region of complementarity including at least 17 contiguous nucleotides differing by no more than 3 nucleotides from the nucleotide sequence of 5′UAUUAUAAAAAUAUCUUGCUUUU3′ (SEQ ID NO: 364); and wherein the dsRNA agent comprises at least one modified nucleotide;
or
wherein the sense strand comprises the nucleotide sequence of 5′-AAGCAAGAUAUUUUUAUAAUA-3′,
and the antisense strand comprises the nucleotide sequence of of 5′-UAUUAUAAAAAUAUCUUGCUUUU-3′.

64. The method of any one of the preceding claims, wherein the C5 iRNA is a double-stranded ribonucleic acid (dsRNA) agent including a sense strand and an antisense strand;
wherein the antisense strand comprises 5′-asasGfcAfaGfaUfAfUfuUfuuAfuAfaua-3′, and the antisense strand comprises 5′-usAfsUfuAfuaAfaAfauaUfcUfuGfcuususudTdT-3′;
wherein
a, g, c and u are 2′-O-methyl (2′-OMe) A, G, C, and U, respectively;
Af, Gf, Cf and Uf are 2′-fluoro A, G, C and U, respectively;
dT is a deoxy-thymine nucleotide;
s is a phosphorothioate linkage; and
wherein the sense strand is conjugated at the 3′-terminus to the ligand

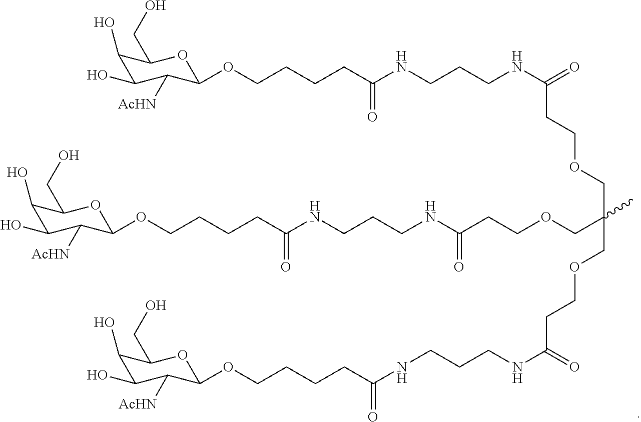 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

65. The method of any one of the preceding claims wherein the C5 iRNA is a duplex comprising [(2S,4R)-1-{1-[(2-acetamido-2-deoxy-β-Dgalactopyranosyl)oxy]-16,16-bis({3-[(3-{5-[(2-acetamido-2-deoxy-β-D-galactopyranosyl)oxy]pentanamido}propyl)amino]-3-oxopropoxy}methyl)-5,11,18-trioxo-14-oxa-6,10,17-triazanonacosan-29-oyl}-4-hydroxypyrrolidin-2-yl]methyl hydrogen all-P-ambo-2′-O-methyl-P-thioadenylyl-(3′→5′)-2′-Omethyl-P-thioadenylyl-(3′→5′)-2′-deoxy-2′-fluoroguanylyl-(3′→5′)-2′-O-methylcytidylyl-(3′→5′)-2′-deoxy-2′-fluoroadenylyl-(3′→5′)-2′-O-methyladenylyl-(3′→5′)-2′-deoxy-2′-fluoroguanylyl-(3′→5′)-2′-O-methyladenylyl-(3′→5′)-2′-deoxy-2′-fluorouridylyl-(3′→5′)-2′-deoxy-2′-fluoroadenylyl-(3′→5′)-2′-deoxy-2′-fluorouridylyl-(3′→5′)-2′-O-methyluridylyl-(3′→5′)-2′-deoxy-2′-fluorouridylyl-(3′→5′)-2′-O-methyluridylyl-(3′→5′)-2′-Omethyluridylyl-(3′→5′)-2′-deoxy-2′-fluoroadenylyl-(3′→5′)-2′-Omethyluridylyl-(3′→5′)-2′-deoxy-2′-fluoroadenylyl-(3′→5′)-2′-Omethyladenylyl-(3′→5′)-2′-O-methyluridylyl-(3′→5′)-2′-Omethyl-3′-adenylate and all-P-ambo-thymidylyl-(5′→3′)-thymidylyl-(5′→3′)-2′-O-methyl-P-thiouridylyl-(5′→3′)-2′-Omethyl-P-thiouridylyl-(5′→3′)-2′-O-methyluridylyl-(5′→3′)-2′-Omethyluridylyl-(5′→3′)-2′-O-methylcytidylyl-(5′→3′)-2′-deoxy-2′-fluoroguanylyl-(5′→3′)-2′-O-methyluridylyl-(5′→3′)-2′-deoxy-2′-fluorouridylyl-(5′→3′)-2′-O-methylcytidylyl-(5′→3′)-2′-deoxy-2′-fluorouridylyl-(5′→3′)-2′-O-methyladenylyl-(5′→3′)-2′-Omethyluridylyl-(5′→3′)-2′-O-methyladenylyl-(5′→3′)-2′-deoxy-2′-fluoroadenylyl-(5′→3′)-2′-O-methyladenylyl-(5′→3′)-2′-deoxy-2′-fluoroadenylyl-(5′→3′)-2′-O-methyladenylyl-(5′→3′)-2′-Omethyluridylyl-(5′→3′)-2′-deoxy-2′-fluoroadenylyl-(5′→3′)-2′-Omethyluridylyl-(5′→3′)-2′-deoxy-2′-fluoro-P-thiouridylyl-(5′→3′)-2′-deoxy-2′-fluoro-P-thioadenylyl-(5′→3′)-2′-O-methyluridine.

66. The method of any one of the preceding claims, wherein the C5 iRNA is Cemdisiran.

67. The method of claim 66, wherein the Cemdisiran is the Na⁺ salt form, another salt form or free form.

68. The method of any one of the preceding claims, wherein the anti-C5 antibody or antigen-binding fragment thereof is Pozelimab and the C5 iRNA is Cemdisiran.

69. The method of any one of the preceding claims, wherein the C5-associated disease or disorder is selected from adult respiratory distress syndrome (ARDS); age-related macular degeneration (AMD); allergy; Alport's syndrome; Alzheimer's disease; an autoimmune disease; an immune complex disorder; an inflammatory disorder; angiopathic thrombosis and protein-losing enteropathy); asthma; atherosclerosis; bronchoconstriction; bullous pemphigoid; C3 glomerulopathy; capillary leak syndrome; CHAPLE disease (CD55 deficiency with hyperactivation of complement); a chemical injury due to irritant gasses and/or chemicals; chronic obstructive pulmonary disease (COPD); complement activation due to a burn; complement activation due to frostbite; complement activation due to obesity; complement activation due to sepsis; Crohn's disease; diabetes; diabetic macular edema (DME); diabetic nephropathy; diabetic retinopathy; a disorder of inappropriate or undesirable complement activation; dry AMD; dyspnea; emphysema; epilepsy; fibrogenic dust disease; geographic atrophy (GA); glomerulopathy; Goodpasture's Syndrome; Guillain-Barre Syndrome; a hemodialysis complication; hemolytic anemia; hemoptysis; hereditary angioedema; hyperacute allograft rejection; hypersensitivity pneumonitis; immune complex-associated inflammation; an infectious disease; inflammation of an autoimmune disease; inherited CD59 deficiency; an injury due to inert dusts and/or minerals; interleukin-2 induced toxicity during IL-2 therapy; a lung disease or disorder; lupus nephritis; membranoproliferative glomerulonephritis; membranoproliferative nephritis; mesenteric artery reperfusion after aortic reconstruction; multiple sclerosis; myasthenia gravis; myocardial infarction; a neurological disorder; neuromyelitis optica; ocular angiogenesis; an ocular disease; an organic dust disease; a parasitic disease; Parkinson's disease; paroxysmal nocturnal hemoglobinuria (PNH); pneumonia; a post-ischemic reperfusion condition; a proteinuric kidney disease; progressive kidney failure; psoriasis; pulmonary embolisms and infarcts; pulmonary fibrosis; pulmonary vasculitis; a renal disorder; renal ischemia; renal ischemia-reperfusion injury; rheumatoid arthritis; schizophrenia; SLE nephritis; a smoke injury; stroke; systemic inflammatory response in post-pump syndrome due to cardiopulmonary bypass or renal bypass; systemic lupus erythematosus (SLE); a thermal injury; a traumatic brain injury; uveitis; vasculitis; wet AMD; xenograft rejection; and combinations thereof.

70. The method of claim 69, wherein the C5-associated disease or disorder is myasthenia gravis.

71. The method of claim 70, wherein the myasthenia gravis is generalized myasthenia gravis, ocular myasthenia gravis, Class I myasthenia gravis, Class II myasthenia gravis, Class IIa myasthenia gravis, Class IIb myasthenia gravis, Class III myasthenia gravis, Class IIIa myasthenia gravis, Class IIIb myasthenia gravis, Class IV myasthenia gravis, Class IVa myasthenia gravis, Class IVb myasthenia gravis, Class V myasthenia gravis, Early-onset myasthenia gravis, Late-onset myasthenia gravis, Thymoma-associated myasthenia gravis, myasthenia gravis with anti-MuSK antibodies, Ocular myasthenia gravis characterized by symptoms from periocular muscles, and/or myasthenia gravis with no detectable AChR and MuSK antibodies.

72. The method of any one of the preceding claims, wherein the subject has been diagnosed with myasthenia gravis.

73. A method for treating or preventing a C5-associated disease or disorder in a subject in need thereof comprising administering, to the subject, a dose of C5 iRNA wherein
one week after said administering, the subject serum CH50 decreases by about 33%,
two weeks after said administering, the subject serum CH50 decreases by about 56%,
four week after said administering, the subject serum CH50 decreases by about 77%;
relative to before the administration.

74. A method for treating or preventing a C5-associated disease or disorder in a subject in need thereof comprising administering, to the subject, a dose of C5 iRNA wherein the subject serum CH50 decreases by at least about 33, 56, 74, 75 or 77% relative to before treatment initiation.

75. A method for treating or preventing a C5-associated disease or disorder in a subject in need thereof comprising administering, to the subject, a dose of C5 iRNA wherein the subject serum CH50 decreases by no more than about 70, 71, 72, 73, 74, 75, 76, 77, 78, 79 or 80% relative to before treatment initiation.

76. The method of any one of claims 74-75 wherein the CH50 decreases by 1, 2, 4, 8, 16, 20 or 24 weeks after treatment initiation.

77. The method of any one of the preceding claims, wherein the subject has been diagnosed with myasthenia gravis by a method comprising an electrophysiologic test, a repetitive nerve stimulation (RNS) test, a single-fiber electromyography (SFEMG) test, an Edrophonium (Tensilon) test, an Ice-pack test, Chest computed tomography (CT), and/or Magnetic resonance imaging (MRI).

78. The method of any one of the preceding claims, further comprising diagnosing the subject with myasthenia gravis.

79. The method of any one of the preceding claims, further comprising diagnosing the subject with myasthenia gravis by a method comprising an electrophysiologic test, a repetitive nerve stimulation (RNS) test, a single-fiber electromyography (SFEMG) test, an Edrophonium (Tensilon) test, an Ice-pack test, Chest computed tomography (CT) and/or Magnetic resonance imaging (MRI).

80. The method of any one of the preceding claims, wherein the subject has myasthenia gravis and one or more criteria selected from: (a) a history of abnormal neuromuscular transmission by repetitive nerve stimulation; (b) a history of abnormal neuromuscular transmission by single-fiber electromyography; (c) a history of a positive anticholinesterase test; and (d) improvement in symptoms with anticholinesterase treatment.

81. The method of any one of the preceding claims, wherein the subject has myasthenia gravis and is positive for anti-acetylcholinesterase (anti-AChR) antibodies or anti-Lipoprotein receptor-related protein 4 (anti-LRP4) antibodies.

82. The method of any one of the preceding claims, further comprising administering to the subject one or more further therapeutic agents.

83. The method of claim 82, wherein the one or more further therapeutic agents is selected from a chemotherapeutic agent, an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and an anti-thrombotic agent.

84. The method of any one of claims 82-83, wherein the further therapeutic agent is not
an anti-CD20 antibody,
an anti-FcRN antibody,
and/or
an androgen deprivation therapy.

85. The method of any one of the preceding claims, further comprising administering to the subject an initial loading dose of C5 iRNA and/or antibody or fragment thereof that binds specifically to C5.

86. The method of any one of the preceding claims, wherein the loading dose is administered intravenously.

87. The method of any one of the preceding claims, which is continued up until or terminated when:
The subject becomes pregnant;
The subject contracts a serious meningococcal infection;
The subject suffers a liver impairment as confirmed by a repeat test on 1 or more of the following criteria and no other reason can be found to explain the following lab abnormalities:
ALT (Alanine aminotransferase) or AST (Aspartate aminotransferase) level >8×ULN (upper limit of normal), or
ALT or AST level >5×ULN for >2 weeks, or
ALT or AST level >3×ULN and total bilirubin >2×ULN (or INR (international normalized ratio)>1.5);
and/or
The subject makes a suicide attempt.

88. The method of any one of the preceding claims, where the subject has received one or more of:
an anticholinesterase agent, an immunosuppressive drug, a C5 complement inhibitor, a neonatal Fc receptor blocker, prednisone, tacrolimus, rituximab, eculizumab, ravulizumab, efgartigimod, rozanolixizumab, pyridostigmine, plasmapheresis (PLEX) and/or intravenous IgG.

89. The method of any one of the preceding claims, wherein, during said treatment, the subject is monitored for:
Meningococcal infection;
Serious infection;
Sepsis;
Embryofetal toxicity;
Immunogenicity;
Anti-drug antibody formation;
Any clinical Consequences of large Drug-Target-Drug immune complexes in subjects who switch from Eculizumab or Ravulizumab to Pozelimab;
Liver transaminase elevations; and/or
Injection site reaction;
and, optionally, terminating treatment is one or more are observed.

90. The method of any one of the preceding claims, wherein the subject achieves one or more of:
Improvement in Myasthenia Gravis-Activities of Daily Living (MG-ADL) total score from baseline to week 24;
Improvement in Quantitative Myasthenia Gravis (QMG) score from baseline to week 24;
≥3-point improvement from baseline to week 24 on the MG-ADL;
≥5-point improvement from baseline to week 24 on the QMG;
At least a 2-point MG-ADL improvement on 2 or more consecutive assessments on the MG-ADL spanning 4 or more weeks during the double-blind treatment period (DBTP);
Minimal symptom expression (MSE), defined by a score of 0 to 1 on the MG-ADL at week 24;
Improvement in the Myasthenia Gravis Composite (MGC) total score from baseline to week 24;
Improvement in Myasthenia Gravis Quality of Life (MG-QOL15r) total score from baseline to week 24;
Improvement point threshold of ≥2, 4, 5, 6, 7, 8, 9, or 10 on MG-ADL at week 24;
Improvement point threshold of ≥3, 4, 6, 7, 8, 9, or 10 on QMG at week 24;
Reduced incidence and severity of treatment-related adverse events (TEAEs), serious adverse events (SAEs), and adverse events of special interest (AESIs) in comparison to placebo through week 24;
Improvement from baseline in MG-ADL total score by visit;
Improvement from baseline in QMG total score by visit;
Reduced time to achievement of a 3-point improvement from baseline on the MG-ADL in comparison to placebo;
Reduced time to achievement of a 5-point improvement from baseline on the QMG in comparison to placebo;
Improvement from baseline in the physical performance measures of hand grip strength, supine leg lift, supine head lift, onset of dysarthria, and all timed items of the QMG;
Improvement from baseline in individual domains (ocular, bulbar, respiratory, and gross motor/limb impairment) of the MG-ADL;
Improvement from baseline in sentinel domains (ocular, facial, bulbar, gross motor, axial, and respiratory) of the QMG;
Improvement from baseline in continuous variable measures from the MGC;
Improvement from baseline in timed step count, cadence, and other exploratory measures using a wearable device;
Increased total MG-ADL score and secondary efficacy endpoints in comparison to placebo through week 48 and through week 120;
Improvement in patient-reported fatigue as measured by the Neuro-QoL-Fatigue from baseline to week 24;
Improvement from baseline in patient-reported health status as measured by the EuroQol-5 Dimensions-5 Levels (EQ-5D-5L);
Reduced number of myasthenic crises (including impending crises), days of hospitalization due to MG, and/or procedures related to MG from time periods prior to randomization;
Improvement from baseline and percent change from baseline in alternative pathway hemolytic activity assay (AH50) and total C5 over time;
Reduced use of rescue therapy from baseline to week 24;
Increase in corticosteroid dosage from baseline to week 24;
Decrease in corticosteroid dosage from baseline to week 24;
Reduced incidence and severity of adverse events through the end of study in comparison to placebo;
Improvement from baseline in Modified MGFA Post-Intervention Status (MG-PIS) at week 24, week 48, and week 120;
A percent change in serum CH50 by week 1, week 2, week 4, week 12 or week 24, after treatment initiation with Pozelimab monotherapy (200 mg, q4w) of about −18.49, −9.19, −7.06, −6 or −7.31, U/ml, respectively;
A percent change in serum CH50 by week 1, week 2, week 4, week 12 or week 24 after treatment initiation with Pozelimab (PZ) and Cemdisiran (CMD) combination (200 mg PZ+200 CMD, q4w) of about −63.31, −83.53, −84.62, −98.91 or −99.57, U/ml, respectively;
A percent change in serum CH50 by week 1, week 2, week 4, week 12 or week 24 after treatment initiation with Cemdisiran monotherapy (600 mg, q12w) of about −32.62, −55.75, −76.77, −73.53 or −76.61, U/ml, respectively;
A change in MG-ADL total score by week 2, 4, 8, 12, 16, 20 or 24 after treatment initiation with Pozelimab monotherapy (200 mg, q4w) of about −1.31, −1.98, −2.59, −2.66, −2.80, −2.59 or −2.77, respectively;
A change in MG-ADL total score by week 2, 4, 8, 12, 16, 20 or 24 after treatment initiation with Pozelimab (PZ)+Cemdisiran (CMD) combination (200 mg PZ+200 mg CMD, q4w) of about −2.56, −3.08, −3.38, −3.76, −3.75, −3.91 or −3.96, respectively;
A change in MG-ADL total score by week 2, 4, 8, 12, 16, 20 or 24 after treatment initiation with Cemdisiran monotherapy (600 mg, q12w) of about −2.50, −3.22, −3.96, −4.31, −4.14, −4.59 or −4.52, respectively;
An absolute MD-ADL score, by week, 2, 4, 8, 12, 16, 20 or 24 after treatment initiation with Pozelimab (200 mg, q4w), of about 8.50, 7.87, 7.08, 7.13, 6.94, 7.15 or 6.92, respectively;
An absolute MD-ADL score, by week, 2, 4, 8, 12, 16, 20 or 24 after treatment initiation with Pozelimab (PZ)+Cemdisiran (CMD) (200 mg PZ+200 mg CMD, q4w), of about 6.91, 6.25, 5.84, 5.45, 5.33, 5.22 or 5.08, respectively;
An absolute MD-ADL score, by week, 2, 4, 8, 12, 16, 20 or 24 after treatment initiation with Cemdisiran (600 mg, q12w), of about 6.52, 5.81, 5.08, 4.70, 4.81, 4.44 or 4.53, respectively;
A change in QMG total score by week 2, 4, 8, 12, 16, 20 or 24 after treatment initiation with Pozelimab monotherapy (200 mg, q4w) of about −0.83, −0.73, −1.58, −1.78, −1.44, −1.77 or −1.55, respectively;
A change in QMG total score by week 2, 4, 8, 12, 16, 20 or 24 after treatment initiation with Pozelimab (PZ) and Cemdisiran (CMD) combination (200 mg PZ+200 CMD, q4w) of about −2.11, −2.77, −2.56, −3.07, −3.27, −3.18 or −3.32, respectively;
A change in QMG total score by week 2, 4, 8, 12, 16, 20 or 24 after treatment initiation with Cemdisiran monotherapy (600 mg, q12w) of about −1.95, −2.60, −3.91, −3.94, −4.11, −3.92 or −4.24, respectively;
An absolute MD-ADL score, by week, 2, 4, 8, 12, 16, 20 or 24 after treatment initiation with Pozelimab monotherapy (200 mg, q4w), of about 15.33, 15.54, 14.38, 14.41, 14.47, 14.19 or 14.31, respectively;
An absolute MD-ADL score, by week, 2, 4, 8, 12, 16, 20 or 24 after treatment initiation with Pozelimab (PZ)+Cemdisiran (CMD) (200 mg PZ+200 mg CMD, q4w) (200 mg, q4w), of about 13.84, 12.85, 12.94, 12.33, 11.97, 11.98 or 11.94, respectively;
An absolute MD-ADL score, by week, 2, 4, 8, 12, 16, 20 or 24 after treatment initiation with Cemdisiran monotherapy (600 mg, q12w), of about 13.64, 12.98, 11.69, 11.64, 11.45, 11.76 or 11.44, respectively;
A change in MGC score by week 2, 4, 8, 12, 16, 20 or 24 after treatment initiation with Pozelimab (PZ)+Cemdisiran (CMD) combination (200 mg PZ+200 mg CMD, q4w) of about −4.31, −4.77, −5.49, −5.89, −5.85, −6.01 or −6.04, respectively;
A change in MGC score by week 2, 4, 8, 12, 16, 20 or 24 after treatment initiation with Cemdisiran monotherapy (600 mg, q12w) of about −4.49, −5.22, −6.55, −7.55, −7.57, −7.69 or −7.78, respectively;
A change in MGC score by week 2, 4, 8, 12, 16, 20 or 24 after treatment initiation with Pozelimab (PZ) monotherapy (200 mg, q4w) of about −1.64, −2.13, −3.34, −3.47, −3.30, −3.57 or −3.19, respectively;
A change in MG-QoL15r score by week 2, 4, 8, 12, 16, 20 or 24 after treatment initiation with Pozelimab (PZ)+Cemdisiran (CMD) combination (200 mg PZ+200 mg CMD, q4w) of about −2.44, −2.86, −4.00, −3.79, −3.86, −3.91 or −4.25, respectively;
A change in MG-QoL15r score by week 2, 4, 8, 12, 16, 20 or 24 after treatment initiation with Cemdisiran monotherapy (600 mg, q12w) of about −1.43, −2.67, −4.08, −4.31, −4.52, −4.65 or −4.68, respectively; and/or
A change in MG-QoL15r score by week 2, 4, 8, 12, 16, 20 or 24 after treatment initiation with Pozelimab (PZ) monotherapy (200 mg, q4w) of about −1.65, −1.26, −0.92, −1.76, −1.49, −0.70 or −1.58, respectively.

91. A method for treating generalized myasthenia gravis (gMG) in an adult patient who is anti-acetylcholine receptor (AChR) or anti-low-density lipoprotein receptor-related protein 4 (LRP4) antibody positive comprising administering 600 mg Cemdisiran sodium form by subcutaneous injection every 12 weeks or 3 months wherein the Cemdisiran Na⁺ form is represented by the chemical structure: 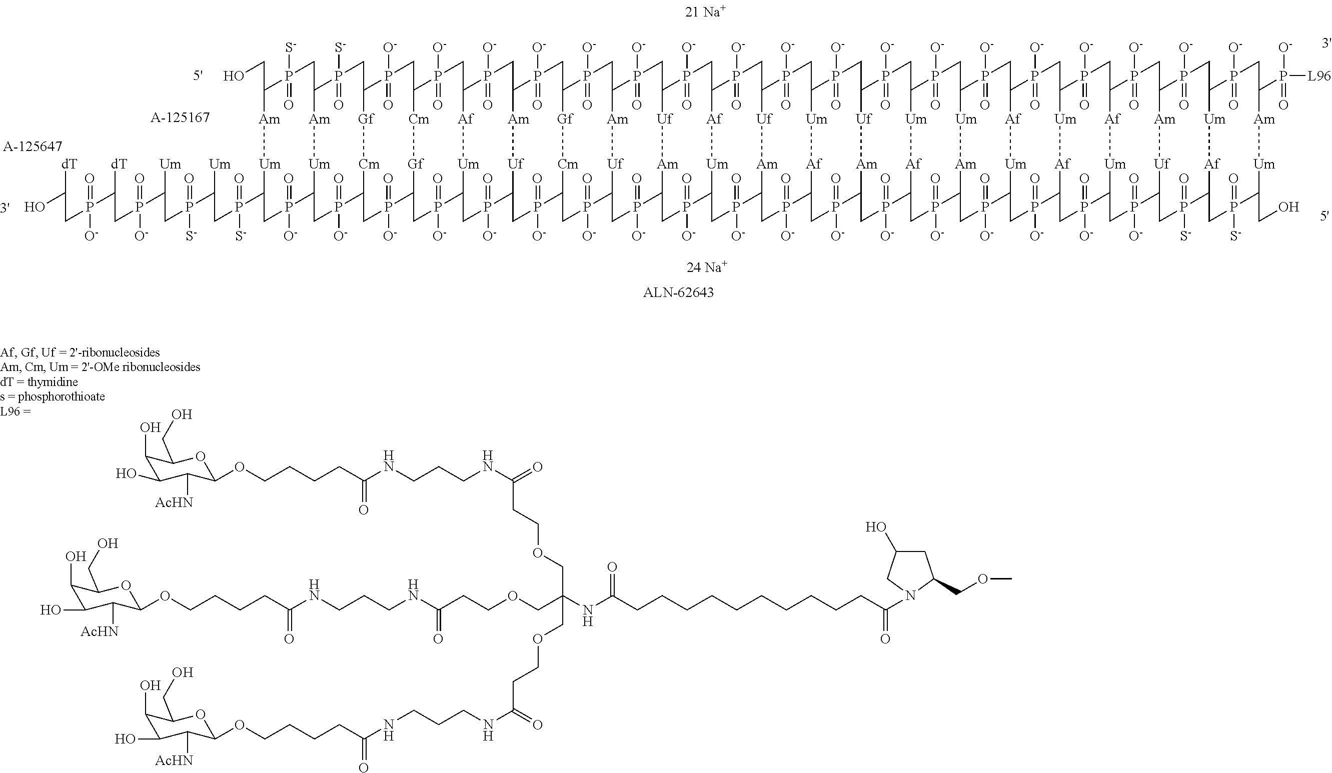
wherein the Cemdisiran Na⁺ form is in an aqueous pharmaceutical formulation in one or more vials.

100. The method of claim 99 wherein the aqueous pharmaceutical formulation comprises the Cemdisiran Na⁺ form, Water for Injection, USP and sodium hydroxide and/or phosphoric acid, pH ˜7.0.

101. The method of claim 100 wherein the aqueous pharmaceutical formulation comprising the Cemdisiran Na+ form is in a sterile, preservative-free, clear, colorless-to-yellow solution supplied in glass vials, free from visible particulates.

102. The method of any one of claims 99-101 wherein each vial contains 200 mg Cemdisiran Na⁺ form in 1 mL with Water for Injection, USP and sodium hydroxide and/or phosphoric acid, pH ˜7.0.

103. The method of any one of claims 99-102 wherein the Cemdisiran Na⁺ form is administered in 2 subcutaneous injections with a volume of 1.5 ml each or 3 subcutaneous injections with 1 ml each.

104. The method of any one of claims 99-103 wherein the full 600 mg Cemdisiran Na⁺ form dose is in three separate vials.

105. The method of any one of claims 99-104, wherein the vials are refrigerated, comprising remove the vials from refrigeration and allowing the vials to sit for at least 15 minutes at 20° C. to 25° C. (68° F. to 77° F.) before use.

106. The method of any one of claims 99-105 comprising inspecting the aqueous composition, visually, for particulate matter and discoloration prior to administration.

107. The method of any one of claims 99-106 comprising discarding the aqueous pharmaceutical formulation if it is cloudy, discolored or contains particulate matter.

108. The method of any one of claims 99-107 comprising gently swirling the vials comprising the aqueous pharmaceutical formulation in an upright position but not shaking the vials.

109. The method of any one of claims 99-108 comprising subcutaneously administering the Cemdisiran Na⁺ form with a 25 gauge to 27 gauge, ½ inch or ⅝-inch stainless steel needle with Luer-Lok.

110. The method of any one of claims 99-109 comprising withdrawing 3 ml of the aqueous pharmaceutical formulation, comprising the Cemdisiran Na+ form, with a 21 gauge withdrawal needle with Luer-Lok, equally into two syringes each containing 1.5 mL or 2 syringes with 1 ml each or a single syringe with a full dose.

111. The method of claim 110 comprising changing the withdrawal needle on each syringe to an injection needle fulfilling the following criteria: 25 gauge to 27 gauge and ½ or ⅝-inch stainless steel needle with Luer-Lok.

112. The method of any one of claims 99-111 comprising administering 2 subcutaneous injections consecutively, each at a different injection site, into the thigh, back of the upper arm, or abdomen, on opposite sides or at least 2 cm apart, but not injecting into moles, scars, or areas where the skin is tender, bruised, red, hard, or not intact.

113. The method of any one of claims 99-112 comprising observing the subject for 30 minutes following completion of the first 600 mg Cemdisiran Na⁺ form dose.

114. The method of any one of claims 99-113 comprising administering the subcutaneous injection within 4 hours of preparation.

115. The method of any one of claims 99-114, for treating generalized myasthenia gravis (gMG) in an adult patient who is anti-acetylcholine receptor (AChR) or anti-low-density lipoprotein receptor-related protein 4 (LRP4) antibody positive comprising:
Inspecting three vials, each containing an aqueous pharmaceutical formulation comprising 200 mg Cemdisiran Na⁺ form in 1 mL with Water for Injection, USP and sodium hydroxide and/or phosphoric acid, pH to ˜7.0, visually for particulate matter and discoloration (other than colorless-to-yellow solution) prior to administration and discarding any vial if the formulation is cloudy, discolored or contains particulate matter;
Gently swirling the vials in an upright position;
Withdrawing the required injection volume of formulation from the vials into an appropriately sized syringe using a 21 gauge withdrawal needle, with Luer-Lok;
Changing the syringe needle to an injection needle fulfilling the following criteria: 25 gauge to 27 gauge and ½ or ⅝-inch stainless steel needle with Luer-Lok; and
Within 4 hours, administering, 600 mg Cemdisiran Na⁺ form in one or more subcutaneous injections, each at a different injection site, into the thigh, back of the upper arm, or abdomen, on opposite sides or at least 2 cm apart, but not inject into moles, scars, or areas where the skin is tender, bruised, red, hard, or not intact; every 3 months;
wherein the Cemdisiran Na⁺ form is represented by the chemical structure: [chemical structure]
